(19) United States
(12) Patent Application Publication
(10) Pub. No.: US 2021/0238187 A1
(43) Pub. Date: Aug. 5, 2021

(54) AZIRIDINE CONTAINING EPOTHILONE ANALOGS, METHODS OF SYNTHESIS, METHODS OF TREATMENT, AND DRUG CONJUGATES

(71) Applicant: WILLIAM MARSH RICE UNIVERSITY, Houston, TX (US)

(72) Inventors: Kyriacos C NICOLAOU, Houston, TX (US); Derek RHOADES, Bellwefontaine, OH (US); Yangping WANG, Shandong Province (CN)

(73) Assignee: WILLIAM MARSH RICE UNIVERSITY, Houston, TX (US)

(21) Appl. No.: 16/604,837

(22) PCT Filed: Apr. 11, 2018
(86) PCT No.: PCT/US2018/027133

§ 371 Date: Oct. 11, 2019

Related U.S. Application Data
(60) Provisional application No. 62/484,262, filed on Apr. 11, 2017.

(51) Int. Cl. C07D 491/044; A61K 47/68

(52) U.S. Cl. CPC C07D 491/044; A61K 47/6803

(57) ABSTRACT

In one aspect, the present disclosure provides epothilone analogs of the formula: (I) wherein the variables are as defined herein. In another aspect, the present disclosure also provides methods of preparing the compounds disclosed herein. In another aspect, the present disclosure also provides pharmaceutical compositions and methods of use of the compounds disclosed herein. Additionally, drug conjugates with cell targeting moieties of the compounds are also provided.

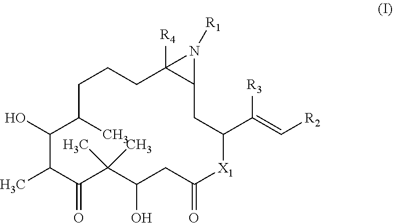

This application claims the benefit of priority to U.S. Provisional Patent Application No. 62/484,262, filed on Apr. 11, 2017, the entire contents of which are hereby incorporated by reference.

This invention was made with government support under Grant No: AI055475 awarded by the National Institute of Health (NIH). The government has certain rights in the invention.

The development of this disclosure was funded in part by the Welch Foundation under Grant No. C-1819.

### BACKGROUND

### 1. Field

This disclosure relates to the fields of medicine, pharmacology, chemistry, and oncology. In particular, new compounds, compositions, methods of treatment, and methods of synthesis relating to aziridine containing analogs of epothilone are disclosed.

### 2. Related Art

Natural products, epothilones A, B, C, and D (1-4), have been the subject of numerous synthetic studies including both the development of total synthesis routes and semisynthesis expeditions from the natural product and key intermediates (Nicolaou & Snyder, 2003, Altmann et al., 2007, Altmann et al., 2009, Pfeiffer et al., 2009, Altmann et al., 2011, Pfeiffer et al., 2012, Altmann et al., 2014, and Schiess et al., 2015). All of this synthetic efforts have led to several drug candidates including one which recently approval as a clinical agent for the treatment of metastatic or locally advanced breast cancer (5, ixabepilone, marketed as Ixempra®). This agent is produced via semisynthesis route from the naturally occurring epothilone B (2) (Vandat, 2008 and Borzilleri et al., 2000). Other notable epothilone B analogues are the methylthio epothilone B (ABJ879, 6), the aminomethyl epothilone B (BMS-310705, 7), the C12-C13 aziridinyl epothilone A analogue (8) (WO 9954319 A1, Regueiro-Ren et al., 2001, WO 02098868 A1, US 20070276018 A1, WO 2007140297 A1, WO 2007140298 A1, and WO 2008147941 A1), and its N-alkylated derivative (BMS-748285, 9) (US 20070275904 A1, Kim et al., 2011, Gokhale et al., 2013), with both of the aziridine analogs entering clinical trials but neither exhibited an appropriate therapeutic window to obtain approval (Sessa et al., 2007). Furthermore, BMS-748285 (9) was conjugated to a folate as part of a targeted chemotherapeutic, but was abandoned after an early clinical trial (Peethambaram et al., 2015). Both ixabepilone (5) and 12,13-aziridinyl epothilone A (8) were prepared via semisynthesis from the natural product, epothilone B (Borzilleri et al., 2000) (2) and epothilone A (WO 9954319 A1, Regueiro-Ren et al., 2001, WO 02098868 A1, US 20070276018 A1, WO 2007140297 A1, WO 2007140298 A1, and WO 2008147941 A1) (1), respectively.

The ability to formulate highly cytotoxic compound into antibody-drug conjugates (ADCs) and other targeted therapeutic approaches offer a way to improve the therapeutic index of those compounds whose high potencies preclude them from being viable drugs due to toxicity issues (Chari et al., 2014 and Srinivasarao et al., 2015). Thus, there remains a need for highly potent chemotherapeutic agents which can acts as the chemical payload for these targeted therapeutic approaches.

### SUMMARY

The present disclosure provides compounds of the formula:

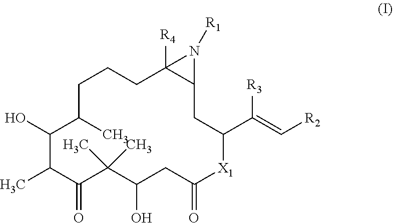

wherein:
X₁ is —O— or —NRₐ—; wherein
   Rₐ is hydrogen or alkyl_{(C≤8)}, cycloalkyl_{(C≤8)}, or a substituted version of either of these groups;

R₁ is hydrogen or alkyl_{(C≤8)}, cycloalkyl_{(C≤8)}, alkenyl_{(C≤8)}, alkynyl_{(C≤8)}, alkanediyl_{(C≤6)}, cycloalkyl_{(C≤8)}, or a substituted version of any of these groups;
R₂ is heteroaryl_{(C≤12)}, heteroarenediyl_{(C≤8)}-R_{d}, or a substituted version of either of these groups; wherein:
   R_{d} is alkyl_{(C≤12)}, aryl_{(C≤12)}, aralkyl_{(C≤12)}, heteroaryl_{(C≤12)}, heteroaralkyl_{(C≤12)}, or a substituted version of either of these groups;
R₃ is hydrogen or alkyl_{(C≤12)}, cycloalkyl_{(C≤12)}, or a substituted version of any of either group;
R₄ is alkyl_{(C≤12)}, cycloalkyl_{(C≤12)}, or a substituted version of any of either group;
   or a pharmaceutically acceptable salt thereof.

In some embodiments, the compound is further defined as:

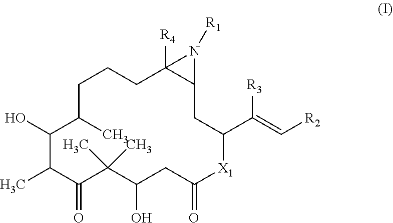

wherein:
X₁ is —O— or —NRₐ—; wherein
   Rₐ is hydrogen or alkyl_{(C≤8)}, cycloalkyl_{(C≤8)}, or a substituted version of either of these groups;
R₁ is hydrogen or alkyl_{(C≤8)}, cycloalkyl_{(C≤8)}, alkenyl_{(C≤8)}, alkynyl_{(C≤8)}, -alkanediyl_{(C≤6)}-cycloalkyl_{(C≤8)}, or a substituted version of any of these groups;
R₂ is heteroaryl_{(C≤12)}, -heteroarenediyl_{(C≤8)}-R_{d}, or a substituted version of either of these groups; wherein:
   R_{d} is alkyl_{(C≤12)}, aryl_{(C≤12)}, aralkyl_{(C≤12)}, heteroaryl_{(C≤12)}, heteroaralkyl_{(C≤12)}, or a substituted version of either of these groups;
R₃ and R₄ are each independently hydrogen or alkyl_{(C≤12)}, cycloalkyl_{(C≤12)}, or a substituted version of any of either group;
   provided that when R₄ is hydrogen and R₂ is 2-methylthiazol-4-yl, then R₁ is not hydrogen or hydroxyethyl;
   or a pharmaceutically acceptable salt thereof.

In some embodiments, the compound is further defined as:

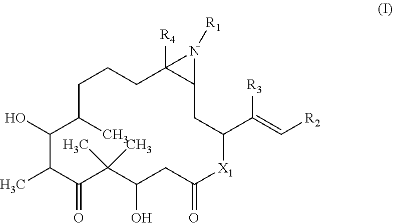

wherein:
X₁, R₃, and R₄ are as defined above;
R₁ is hydrogen or alkyl_{(C≤8)}, cycloalkyl_{(C≤8)}, alkenyl_{(C≤8)}, alkynyl_{(C≤8)}, -alkanediyl_{(C≤6)}-cycloalkyl_{(C≤8)}, or a substituted version of any of these groups; and
R₂ is

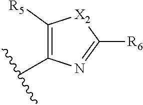

wherein:
   X₂ is —O— or —NR_{b}—; wherein
      R_{b} is hydrogen or alkyl_{(C≤8)}, cycloalkyl_{(C≤8)}, or a substituted version of either of these groups;
      R₅ is hydrogen, amino, halo, hydroxy, alkyl_{(C1-3)}, substituted alkyl_{(C1-3)}, alkoxy_{(C1-3)}, or substituted alkoxy_{(C1-3)}; and
      R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)}; or

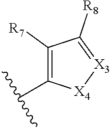

wherein:
   X₃ and X₄ are each independently —O— or —NR_{c}—; wherein
      R_{c} is absent, hydrogen or alkyl_{(C≤8)}, cycloalkyl_{(C≤8)}, aryl_{(C≤8)}, aralkyl_{(C≤8)}, heteroaryl_{(C≤8)}, heteroaralkyl_{(C≤8)}, or a substituted version of any of these groups; provided that when R_{c} is absent, then atom to which it is bound is a part of a double bond; and provided that when atom to which R_{c} is bound, then R_{c} is absent; and
      R₇ and R₈ are each independently hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, substituted alkoxy_{(C1-3)}, alkylthio_{(C1-3)}, or substituted alkylthio_{(C1-3)}; or

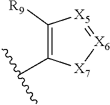

wherein:
   X₅, X₆, and X₇ are each independently —O—, —S—, or —NR_{d}—; wherein
      R_{d} is absent, hydrogen or alkyl_{(C≤8)}, cycloalkyl_{(C≤8)}, or a substituted version of either of these groups; provided that when R_{d} is absent, then atom to which it is bound is a part of a double bond; and provided that when atom to which R_{d} is bound, then R_{d} is absent; and
   R₉ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; or
      a compound wherein:
X₁, R₃, and R₄ are as defined above;
R₁ is hydrogen or alkyl_{(C≤8)}, cycloalkyl_{(C≤8)}, alkenyl_{(C≤8)}, alkynyl_{(C≤8)}, -alkanediyl_{(C≤6)}-cycloalkyl_{(C≤8)}, or a substituted version of any of these groups; and
R₂ is:

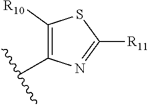

wherein:
   R₁₀ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)};
   R₁₁ is alkyl_{(C≤8)}, heteroaryl_{(C≤8)}, or substituted heteroaryl_{(C≤8)}; and
   a compound wherein:
X₁, R₃, and R₄ are as defined above;
R₁ is cycloalkyl_{(C≤8)}, alkenyl_{(C≤8)}, alkynyl_{(C≤8)}, or a substituted version of any of these groups; and
R₂ is:

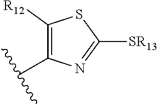

wherein:
   R₁₂ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
   R₁₃ is alkyl_{(C≤6)} or substituted alkyl_{(C≤6)};
      provided that when R₂ is

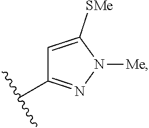 [SMILES: *c1cc(SC)n(C)n1]

then R¹ is not 2-hydroxyethyl;
or a pharmaceutically acceptable salt thereof.

In some embodiments, the compound is further defined as:

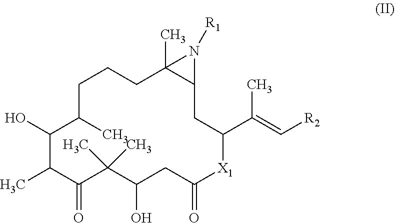

wherein:
R₁, R₂, and X₁ are as defined above;
   or a pharmaceutically acceptable salt thereof.

In some embodiments, the compound is further defined as:

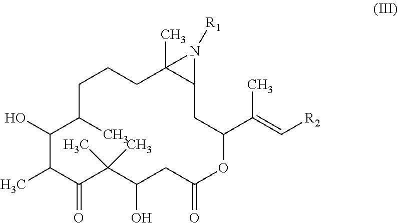

wherein:
R₁ and R₂ are as defined above;
   or a pharmaceutically acceptable salt thereof.

In some embodiments, the compound is further defined as:

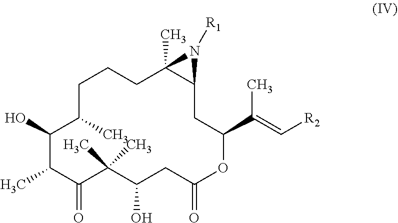

wherein:
R₁ and R₂ are as defined above;
   or a pharmaceutically acceptable salt thereof.

In some embodiments, the compound is further defined as:

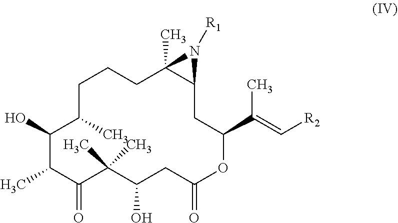

wherein:
R₁ is hydrogen or alkyl_{(C≤8)}, cycloalkyl_{(C≤8)}, alkenyl_{(C≤8)}, alkynyl_{(C≤8)} alkanediyl_{(C≤6)}-cycloalkyl_{(C≤8)}, or a substituted version of any of these groups; and
R₂ is

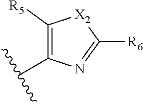

wherein:
X₂ is —O— or —NR_{b}—; wherein
   R_{b} is hydrogen or alkyl_{(C≤8)}, cycloalkyl_{(C≤8)}, or a substituted version of either of these groups;
   R₅ is hydrogen, amino, halo, hydroxy, alkyl_{(C1-3)}, substituted alkyl_{(C1-3)}, alkoxy_{(C1-3)}, or substituted alkoxy_{(C1-3)}; and
   R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)}; or
      or a pharmaceutically acceptable salt thereof.

In other embodiments, the compound is further defined as:

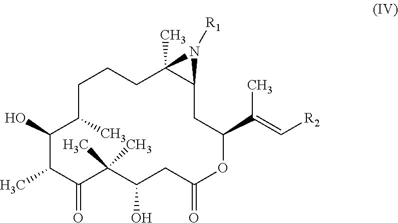

wherein:
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is

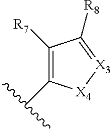

wherein:
   X₃ and X₄ are each independently —O— or —NR_{c}—; wherein
      R_{c} is absent, hydrogen or alkyl_{(C≤8)}, cycloalkyl_{(C≤8)}, aryl_{(C≤8)}, aralkyl_{(C≤8)}, heteroaryl_{(C≤8)}, heteroaralkyl_{(C≤8)}, or a substituted version of any of these groups; provided that when R is absent, then atom to which it is bound is a part of a double bond; and provided that when atom to which R_{c} is bound, then R_{c} is absent; and
   R₇ and R₈ are each independently hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; or
      provided that when R₂ is

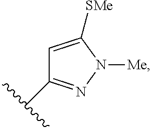 [SMILES: *c1cc(SC)n(C)n1]

then R₁ is not 2-hydroxyethyl;
or a pharmaceutically acceptable salt thereof.

In other embodiments, the compound is further defined as:

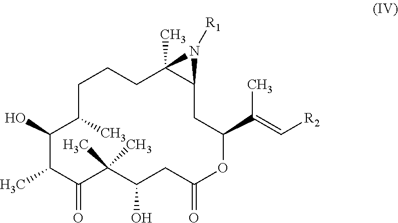

wherein:
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is

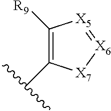

wherein:
   X₅, X₆, and X₇ are each independently —O—, —S—, or —NR_{d}—; wherein
      R_{d} is absent, hydrogen or alkyl_{(C≤8)}, cycloalkyl_{(C≤8)}, or a substituted version of either of these groups; provided that when R_{d} is absent, then atom to which it is bound is a part of a double bond; and provided that when atom to which R_{d} is bound, then R_{d} is absent; and
   R₉ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)};
      or a pharmaceutically acceptable salt thereof.

In other embodiments, the compound is further defined as:

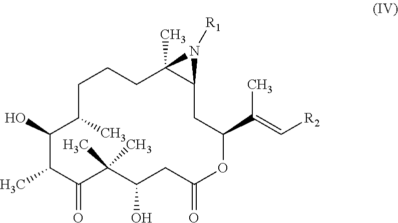

wherein:
is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is:

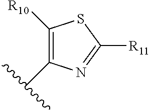

wherein:
   R₁₀ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
   R₁₁ is alkyl_{(C≤8)}, heteroaryl_{(C≤8)}, or substituted heteroaryl_{(C≤8)};
      or a pharmaceutically acceptable salt thereof.

In other embodiments, the compound is further defined as:

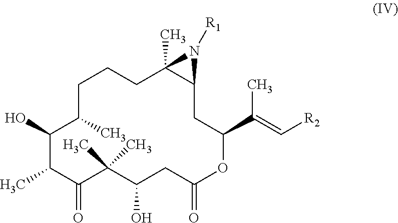

wherein:
R₁ is cycloalkyl_{(C≤8)}, alkenyl_{(C≤8)}, alkynyl_{(C≤8)}, or a substituted version of any of these groups; and R₂ is:

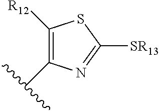

wherein:
   R₁₂ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
   R₁₃ is alkyl_{(C≤6)} or substituted alkyl_{(C≤6)};
      or a pharmaceutically acceptable salt thereof.

In some embodiments, R₃ is alkyl_{(C≤6)} such as methyl. In some embodiments, R₄ is alkyl_{(C≤6)} such as methyl. In some embodiments, X₁ is —O—.

In some embodiments, R₁ is hydrogen. In other embodiments, R₁ is alkyl_{(C≤6)} such as —CH₂CH(CH₂)₂. In other embodiments, R₁ is substituted alkyl_{(C≤6)} such as 2-hydroxyethyl, 2-azidoethyl, 2-mercaptoethyl, 2-aminoethyl, or 2-acetoxyethyl. In other embodiments, R₁ is alkanediyl_{(C≤6)}-cycloalkyl_{(C≤8)}. In other embodiments, R₁ is alkyne_{(C≤6)} such as —CH₂CCH.

In some embodiments, R₂ is:

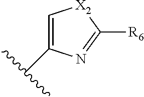

wherein:
X₂ is —O— or —NR_{b}—; wherein
   R_{b} is hydrogen or alkyl_{(C≤8)}, cycloalkyl_{(C≤8)}, or a substituted version of either of these groups; and
R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)}.

In other embodiments, R₂ is:

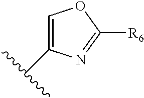

wherein:
X₂ is —O— or —NR_{b}—; wherein
   R_{b} is hydrogen or alkyl_{(C≤8)}, cycloalkyl_{(C≤8)}, or a substituted version of either of these groups; and
R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)}.

In some embodiments, R₆ is alkyl_{(C≤6)} such as methyl. In other embodiments, R₆ is substituted alkyl_{(C≤6)}. In other embodiments, R₆ is alkylthio_{(C≤6)} such as SCH₃. In other embodiments, R₆ is substituted alkylthio_{(C≤6)}.

In other embodiments, R₂ is:

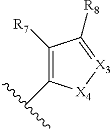

wherein:
X₃ and X₄ are each independently —O— or —NR_{c}—; wherein
   R_{c} is absent, hydrogen or alkyl_{(C≤8)}, cycloalkyl_{(C≤8)}, aryl_{(C≤8)}, aralkyl_{(C≤8)}, heteroaryl_{(C≤8)}, heteroaralkyl_{(C≤8)}, or a substituted version of any of these groups; provided that when R_{c} is absent, then atom to which it is bound is a part of a double bond; and provided that when atom to which R_{c} is bound, then R_{c} is absent; and
R₇ and R₈ are each independently hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, substituted alkoxy_{(C1-3)}, alkylthio_{(C1-3)}, or substituted alkylthio_{(C1-3)}.

In some embodiments, R₈ is alkyl_{(C1-3)} such as methyl. In other embodiments, R₈ is substituted alkyl_{(C1-3)}. In other embodiments, R₈ is aryl_{(C≤8)}. In other embodiments, R₈ is substituted aryl_{(C≤8)} such as 4-fluorophenyl. In other embodiments, R₈ is alkylthio_{(C1-3)} such as —SCH₃. In other embodiments, R₈ is substituted alkylthio_{(C1-3)}.

In some embodiments, R_{c} is aryl_{(C≤8)}. In other embodiments, R_{c} is substituted aryl_{(C≤8)} such as 4-aminophenyl, 3-fluoro-4-aminophenyl, or 3-trifluoromethyl-4-aminophenyl. In other embodiments, R_{c} is heteroaralkyl_{(C≤8)}. In other embodiments, R_{c} is substituted heteroaralkyl_{(C≤8)} such as pyrazolylmethyl.

In other embodiments, R₂ is:

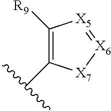

wherein:
X₅, X₆, and X₇ are each independently —O—, —S—, or —NR_{d}—; wherein
   R_{d} is absent, hydrogen or alkyl_{(C≤8)}, cycloalkyl_{(C≤8)}, or a substituted version of either of these groups; provided that when R_{d} is absent, then atom to which it is bound is a part of a double bond; and provided that when atom to which R_{d} is bound, then R_{d} is absent; and
R₉ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}.

In some embodiments, R_{d} is absent or hydrogen. In some embodiments, R₉ is alkyl_{(C1-3)} or substituted alkyl_{(C1-3)}.

In other embodiments, R₂ is:

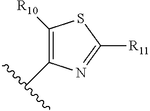

wherein:
R₁₀ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₁ is alkyl_{(C≤8)}, heteroaryl_{(C≤8)}, or substituted heteroaryl_{(C≤8)}.

In some embodiments, R₁₀ is hydrogen.

In some embodiments, R₁₁ is alkyl_{(C≤6)} such as methyl. In other embodiments, R₁₁ is heteroaryl_{(C≤8)}. In other embodiments, R₁₁ is substituted heteroaryl_{(C≤8)} such as 4-methylthiazol-2-yl.

In other embodiments, R₂ is:

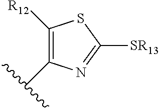

wherein:
R₁₂ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₃ is alkyl_{(C≤6)} or substituted alkyl_{(C≤6)}.

In some embodiments, R₁₂ is hydrogen. In some embodiments, R₁₃ is alkyl_{(C≤6)} such as methyl.

In some embodiments, the compound is further defined as:

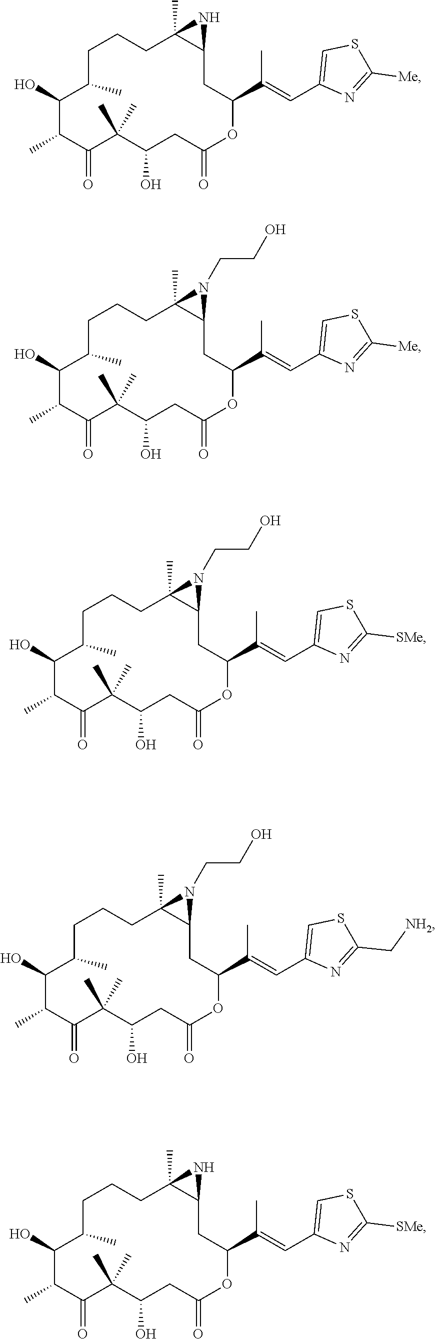

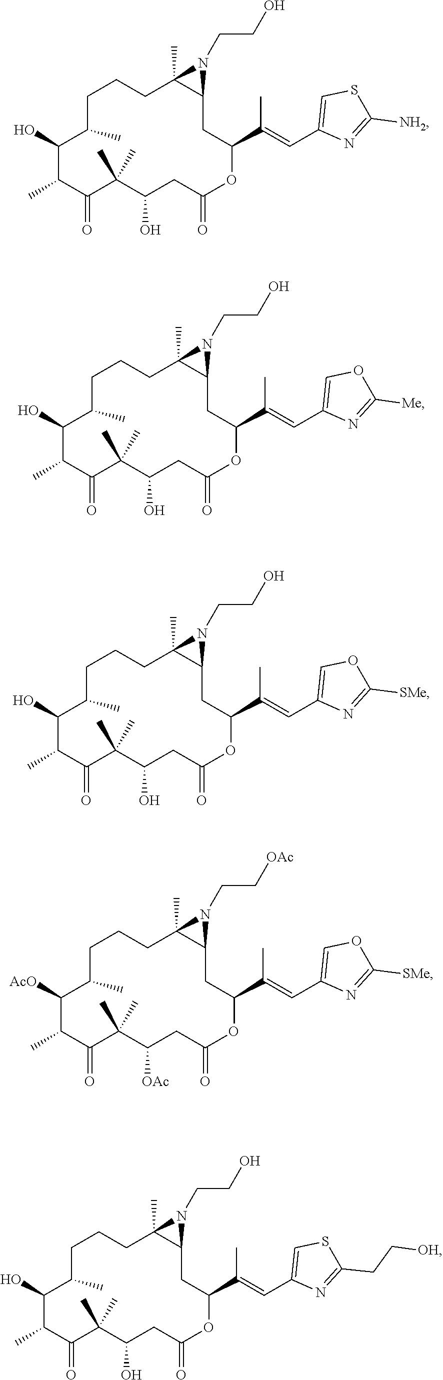

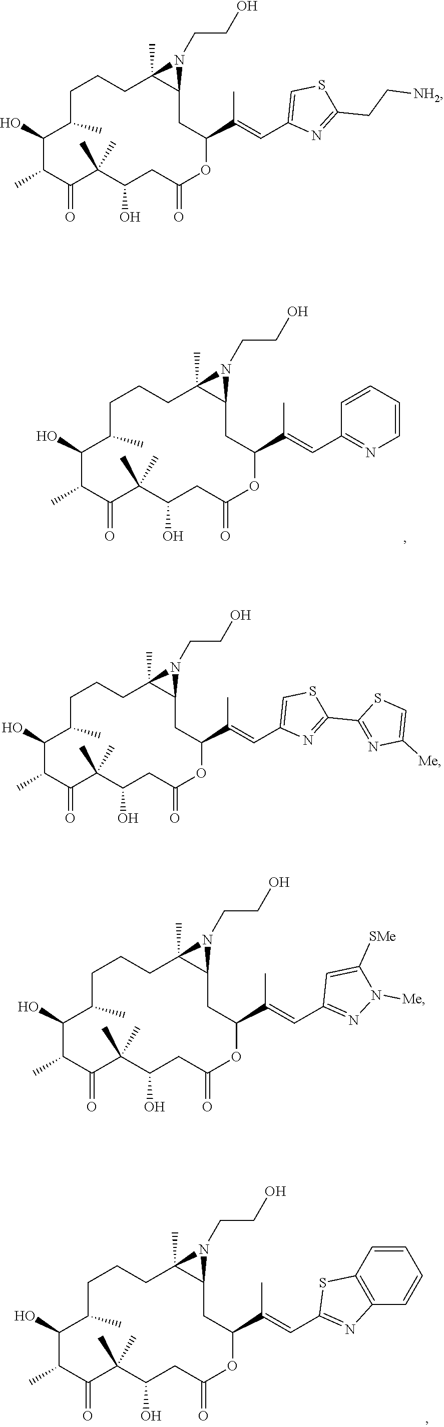

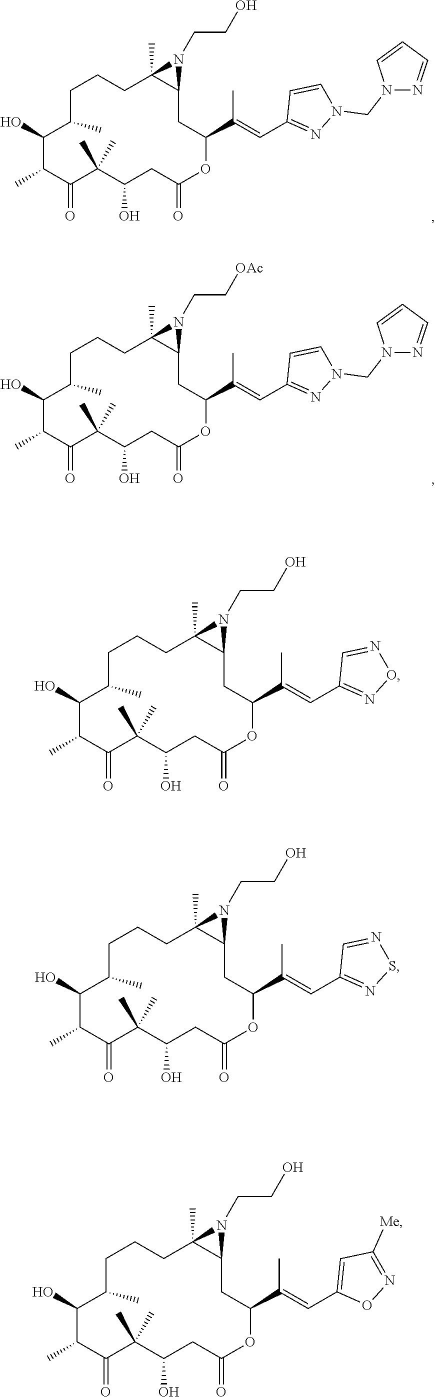

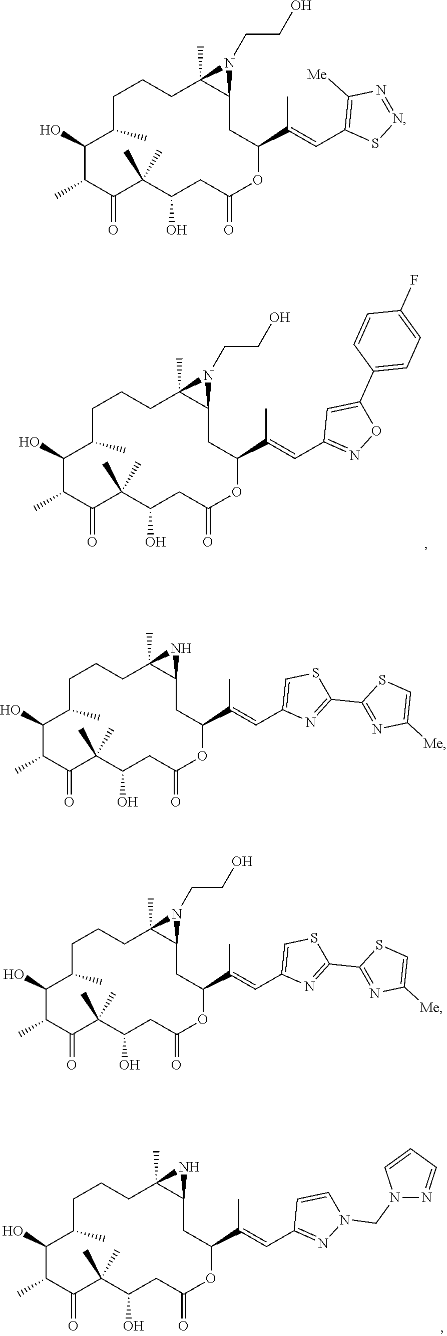

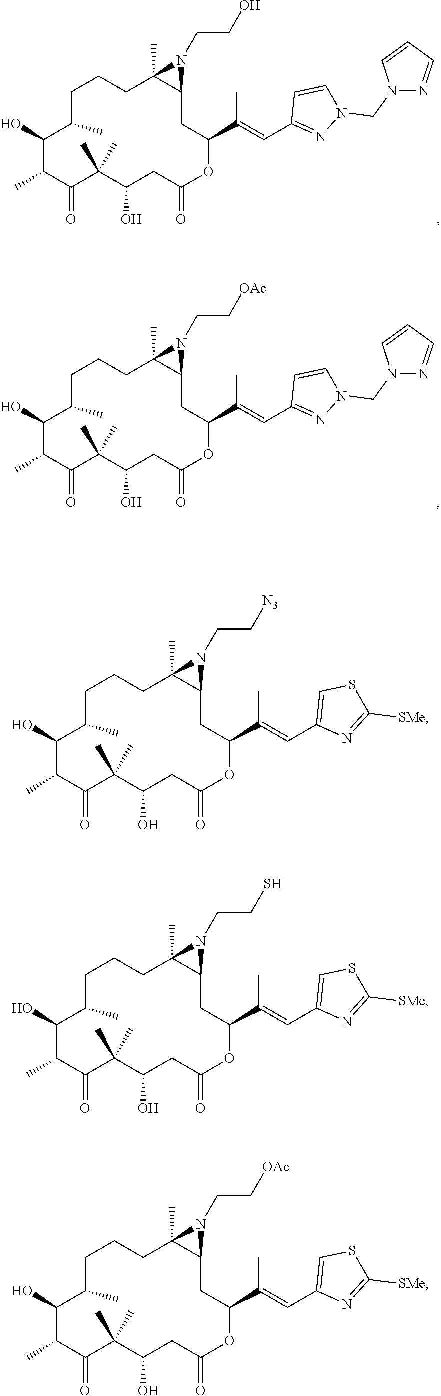

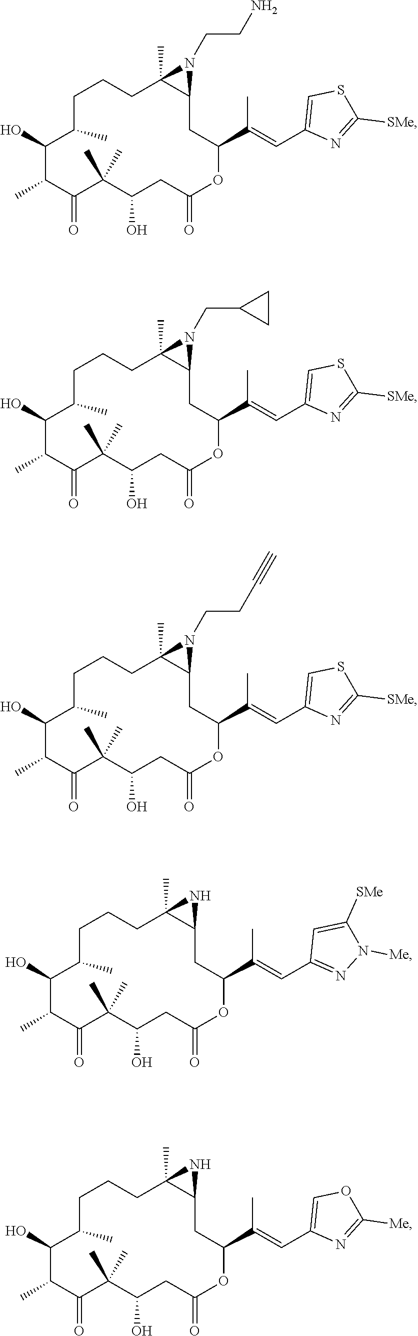

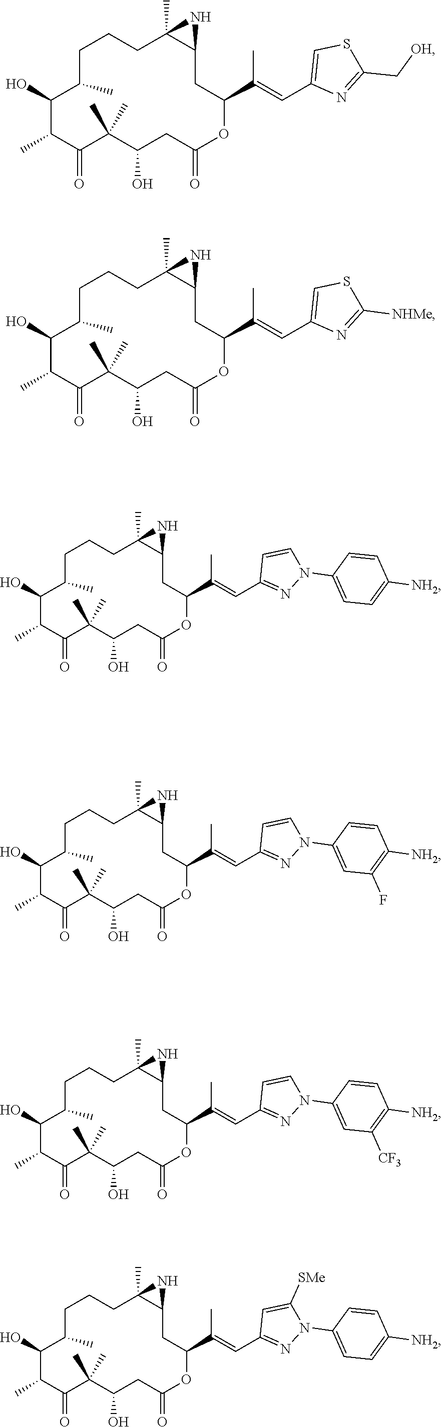

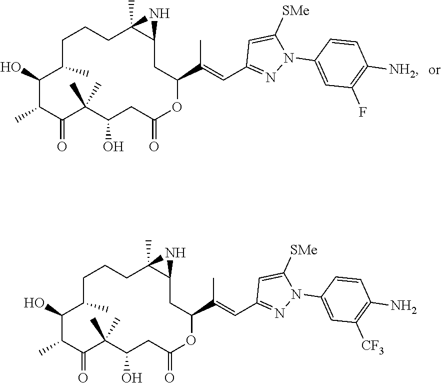

or a pharmaceutically acceptable salt thereof.

In some embodiments, the compound is further defined as:

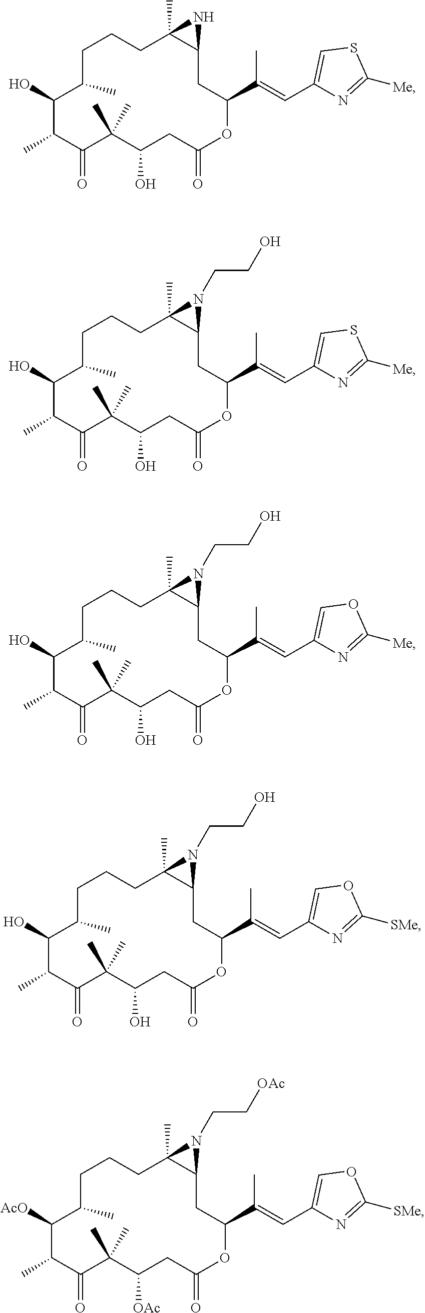

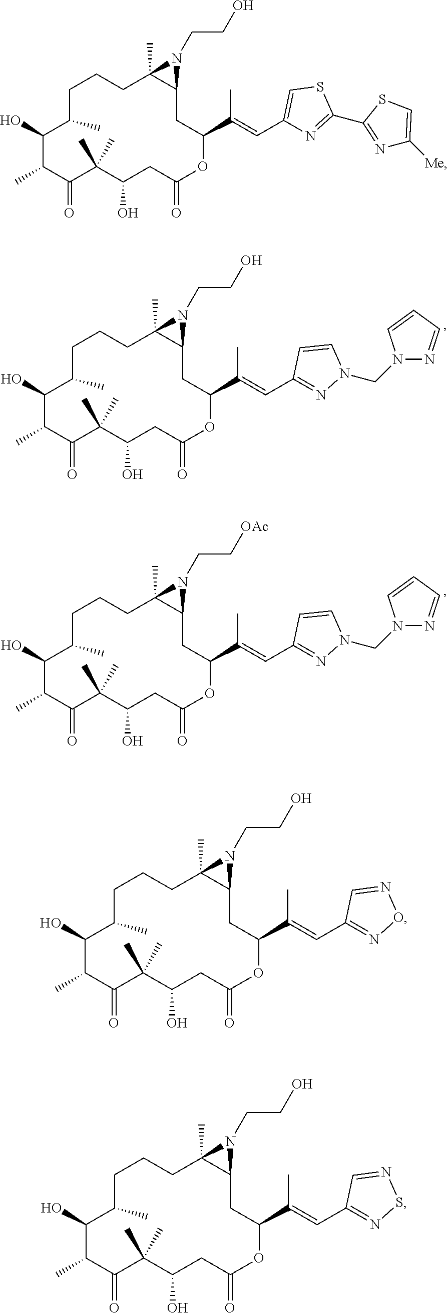

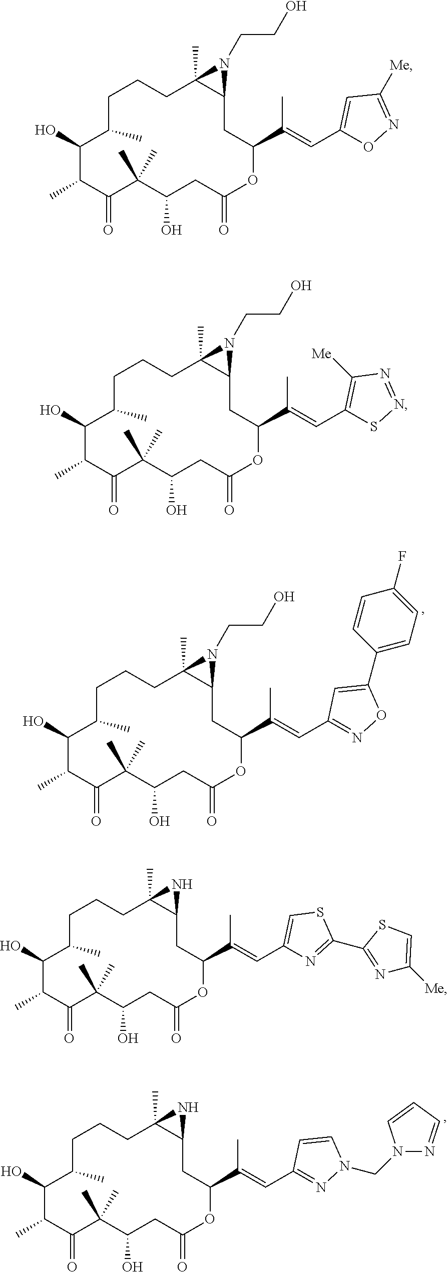

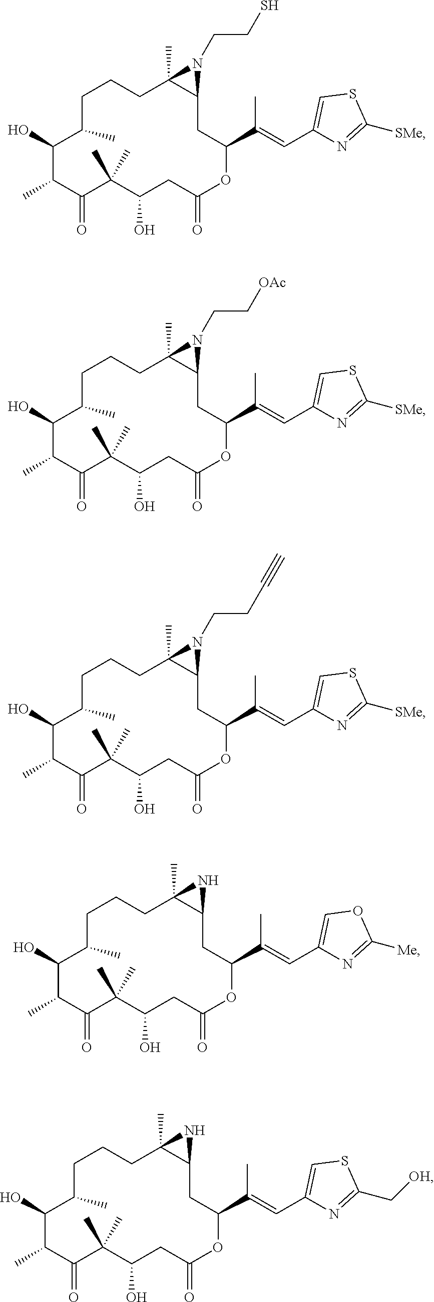

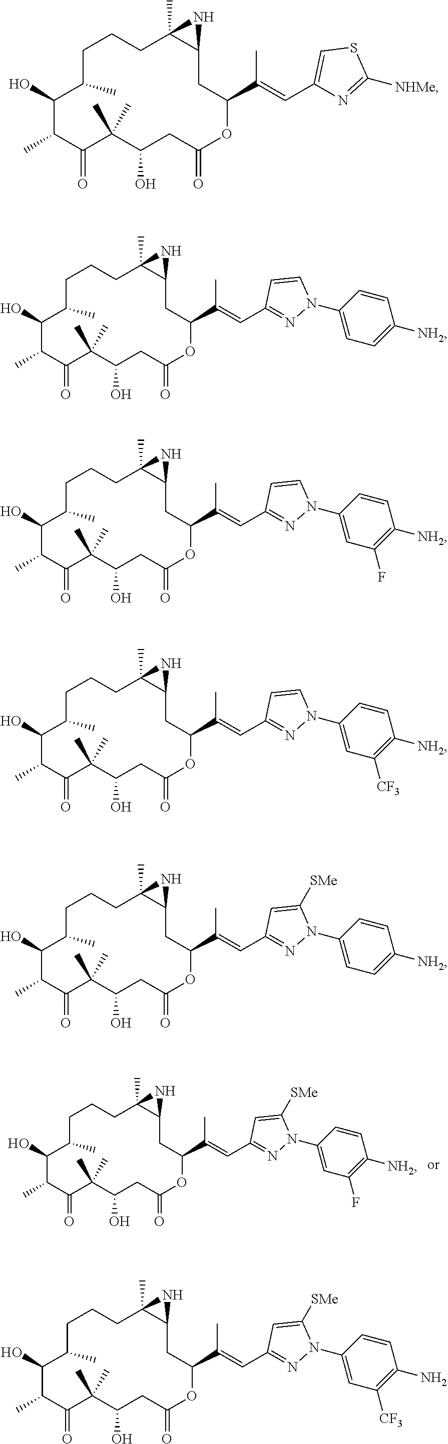

or a pharmaceutically acceptable salt thereof.

In some embodiments, the compounds is further defined as:

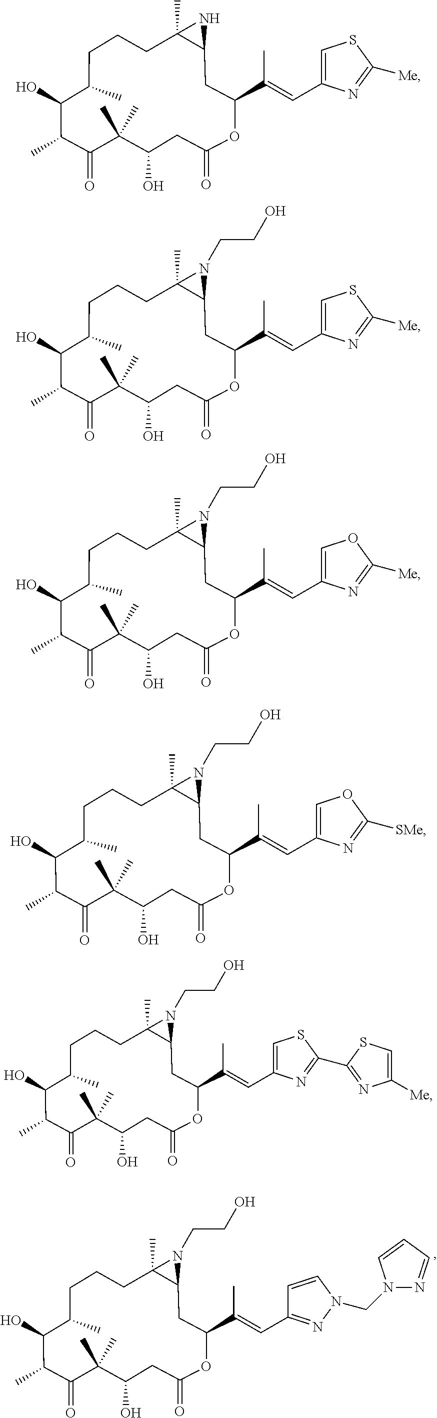

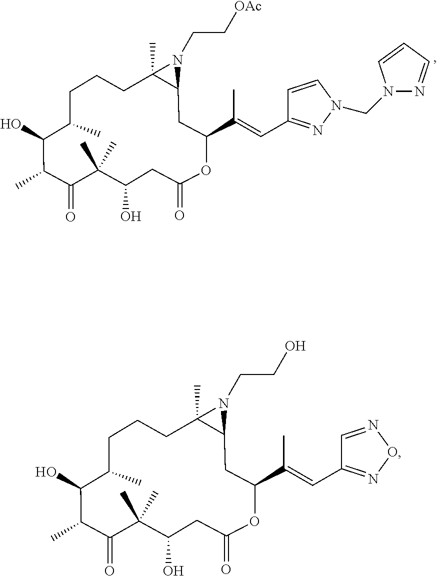

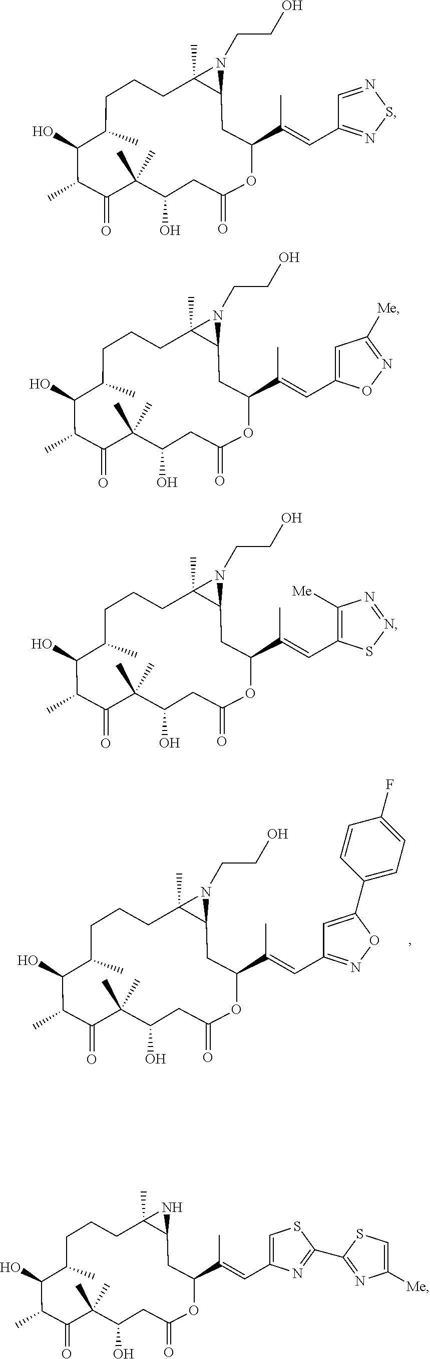

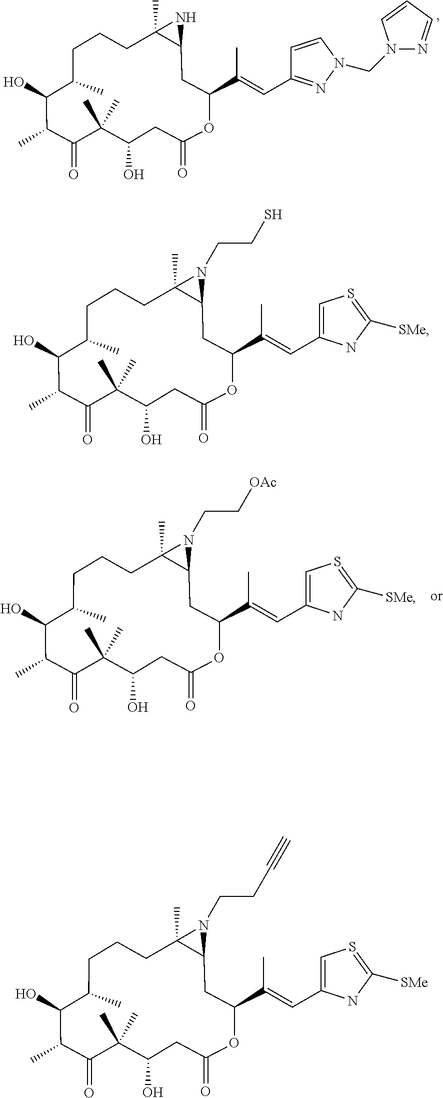

or a pharmaceutically acceptable salt thereof.

In another aspect, the present disclosure provides compounds of the formula:

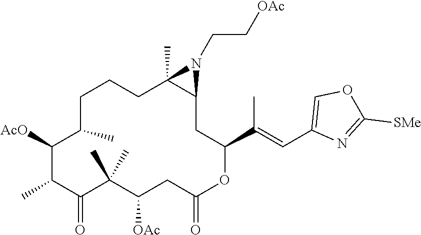

or a pharmaceutically acceptable salt thereof.

In still another aspect, the present disclosure provides pharmaceutical compositions comprising:
(a) a compound of the present disclosure; and
(b) a pharmaceutically acceptable carrier.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical composition is formulated as a unit dose.

In yet another aspect, the present disclosure provides methods of treating a disease or disorder in a patient comprising administering to the patient in need thereof a therapeutically effective amount of a compound or composition of the present disclosure.

In some embodiments, the disease or disorder is cancer such as carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some embodiments, the method further comprises a second cancer therapy such as surgery, a second chemotherapeutic agent, a radiotherapy, or an immunotherapy. In some embodiments, the patient is a mammal such as a human. In some embodiments, the method comprises administering the compound once. In other embodiments, the method comprises administering the compound two or more times.

In still another aspect, the present disclosure provides an antibody drug conjugate comprising:
(a) an antibody; and
(b) a compound of the present disclosure.

In some embodiments, the antibody and the compound are connected through a linker such as an enzymatically degradable linker. In some embodiments, the antibody comprises two or more compounds conjugated to the antibody.
In still another aspect, the present disclosure provides methods of preparing a compound of the formula:

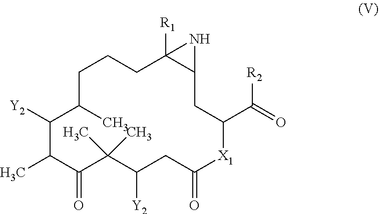

wherein:
X₁ is —O— or —NRₐ—; wherein
   Rₐ is hydrogen, a monovalent amine protecting group, or alkyl_{(C≤8)}, cycloalkyl_{(C≤8)}, alkanediyl_{(C≤6)}-cycloalkyl_{(C≤8)}, aralkyl_{(C≤8)}, or a substituted version of either of these groups;
Y₁ and Y₂ are each independently amino, hydroxy, or alkoxy_{(C≤8)}, aralkoxy_{(C≤8)}, acyloxy_{(C≤8)}, alkylamino_{(C≤8)}, dialkylamino_{(C≤8)}, amido_{(C≤8)}, or a substituted version of any of these groups, or —OR_{c}, wherein:
   R_{c} is a hydroxy protecting group;
R₁ and R₂ are each independently hydrogen or alkyl_{(C≤12)}, cycloalkyl_{(C≤12)}, alkenyl_{(C≤12)}, alkynyl_{(C≤12)}, aryl_{(C≤12)}, or a substituted version of any of these groups; and
   comprising reacting a compound of the formula:

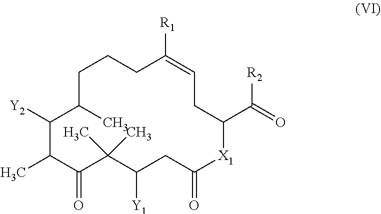

wherein:
X₁, Y₁, Y₂, R₁, and R₂ are as defined above;
   with O-(2,4-dinitrophenyl)hydroxylamine in the presence of a Rh catalyst.

In some embodiments, the Rh catalyst is a Rh(II) catalyst such as Rh₂(esp)₂. In some embodiments, the Rh catalyst is present at a mole percentage from about 0.25% to about 5%. In other embodiments, the mole percentage is about 2%.

In some embodiments, the method comprises adding a ratio of the compound of formula VI to the O-(2,4-dinitrophenyl)hydroxylamine from about 1:1 to about 1:5. In other embodiments, the ratio is about 1:1.5.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description.

FIGS. 1A-1H show the killing assay for compounds 1-4, Paclitaxel, and MMAE (FIG. 1A), MMAE and 8-12 (FIG. 1B), MMAE and 13-17 (FIG. 1C), MMAE and 18-22 (FIG. 1D), MMAE and 23-27 (FIG. 1E), MMAE and 28-32 (FIG. 1F), MMAE and 33-37 (FIG. 1G), and MMAE, 38-40, 73, 79, and 81 (FIG. 1H) for MES SA cells.

FIGS. 2A-2H show the killing assay for compounds 1-4, Paclitaxel, and MMAE (FIG. 2A), MMAE and 8-12 (FIG. 2B), MMAE and 13-17 (FIG. 2C), MMAE and 18-22 (FIG. 2D), MMAE and 23-27 (FIG. 2E), MMAE and 28-32 (FIG. 2F), MMAE and 33-37 (FIG. 2G), and MMAE, 38-40, 73, 79, and 81 (FIG. 2H) for MES SA DX cells.

FIGS. 3A-3H show the killing assay for compounds 1-4, Paclitaxel, and MMAE (FIG. 3A), MMAE and 8-12 (FIG. 3B), MMAE and 13-17 (FIG. 3C), MMAE and 18-22 (FIG. 3D), MMAE and 23-27 (FIG. 3E), MMAE and 28-32 (FIG. 3F), MMAE and 33-37 (FIG. 3G), and MMAE, 38-40, 73, 79, and 81 (FIG. 3H) for HEK 293T cells.

FIGS. 4A & 4B show the X-ray crystal structures of compounds 10 (FIG. 4A) and 39 (FIG. 4B).

### DESCRIPTION OF ILLUSTRATIVE EMBODIMENTS

The present disclosure provides derivatives of epothilone which contain an aziridine ring at the 12,13 position. These compounds may be used in the treatment of cancer, including used as the chemical payload in an antibody drug conjugate. In some aspects, the compounds show improved activity or other pharmacological characteristics relative to known epothilones.

Also provided herein are methods of preparing the aziridine containing epothilone analogs from the double bond containing starting material. Using Ess-Kürti-Falck aziridination conditions, the methyl ketone derivatives could be converted into an aziridine containing epothilone analog. In order to obtain the analog, after introduction of the aziridine group, the methyl ketone was reacted with a phosphonate ylide under Horner-Wadsworth-Emmons conditions to yield the final product.

### I. COMPOUNDS AND FORMULATIONS THEREOF

A. Compounds

The compounds provided by the present disclosure are shown, for example, above in the summary section and in the examples and claims below. They may be made using the methods outlined in the Examples section. The aziridine containing epothilone analogs described herein can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The aziridine containing epothilone analogs described herein may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent the aziridine containing epothilone analogs described herein will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

The aziridine containing epothilone analogs described herein may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the aziridine containing epothilone analogs described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The epothilone analogs described herein may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the aziridine containing epothilone analogs described herein may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the epothilone analogs described herein are within the scope of the disclosure. It will also be appreciated by those skilled in organic chemistry that many organic compounds can exist in more than one crystalline form. For example, crystalline form may vary from solvate to solvate. Thus, all crystalline forms of the epothilone analogs described herein are within the scope of the present disclosure.

B. Formulations

In some embodiments of the present disclosure, the aziridine containing epothilone analogs are included a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the epothilone analogs described herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### II. CANCER AND OTHER HYPERPROLIFERATIVE DISEASES

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the aziridine containing epothilone analogs described herein may be used to lead to decreased cell counts and as such can potentially be used to treat a variety of types of cancer lines. In some aspects, it is anticipated that the aziridine containing epothilone analogs described herein may be used to treat virtually any malignancy.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### III. CELL TARGETING MOIETIES

In some aspects, the present disclosure provides compounds conjugated directly or through linkers to a cell targeting moiety. In some embodiments, the conjugation of the compound to a cell targeting moiety increases the efficacy of the compound in treating a disease or disorder. Cell targeting moieties according to the embodiments may be, for example, an antibody, a growth factor, a hormone, a peptide, an aptamer, a small molecule such as a hormone, an imaging agent, or cofactor, or a cytokine. For instance, a cell targeting moiety according the embodiments may bind to a liver cancer cell such as a Hep3B cell. It has been demonstrated that the gp240 antigen is expressed in a variety of melanomas but not in normal tissues. Thus, in some embodiments, the compounds of the present disclosure may be used in conjugates with an antibody for a specific antigen that is expressed by a cancer cell but not in normal tissues.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda, et al., 2004). Another example is the cell targeting agents described in U.S. Patent Publication No. 2004/005647 and in Winthrop, et al. (2003) that bind to MUC-1, an antigen that is expressed on a variety cancer types. Thus, it will be understood that in certain embodiments, cell targeting constructs according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Additionally, the cell targeting moiety that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the conjugates of the present disclosure and a cell (Campbell, et al., 1991; Weitman, et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R+ cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles, 2008; Zhou, et al., 2011 and Burkly, et al., 2007, incorporated herein by reference).

A skilled artisan recognizes that there are a variety of known cytokines, including hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)).

Furthermore, the Fc portion of the heavy chain of an antibody may be used to target Fc receptor-expressing cells such as the use of the Fc portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, cell targeting moieties are antibodies or avimers. Antibodies and avimers can be generated against virtually any cell surface marker thus, providing a method for targeted to delivery of GrB to virtually any cell population of interest. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### IV. THERAPIES

A. Pharmaceutical Formulations and Routes of Administration

Where clinical applications are contemplated, it will be necessary to prepare pharmaceutical compositions in a form appropriate for the intended application. In some embodiments, such formulation with the aziridine containing epothilone analogs of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the aziridine containing epothilone analogs described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA's Division of Biological Standards and Quality Control of the Office of Compliance and Biologics Quality.

B. Methods of Treatment

In particular, the compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the aziridine containing epothilone analogs used to induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the epothilone analogs may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

C. Combination Therapies

It is envisioned that the epothilone analogs described herein may be used in combination therapies with one or more cancer therapies or a compound which mitigates one or more of the side effects experienced by the patient. It is common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the aziridine containing epothilone analogs described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of cancer therapies that may be used combination with the compounds of the present disclosure.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ, IL-1, GM-CSF and TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurance of the tumor. Additionally, the compounds of the present disclosure can also be used in a neoadjuvant setting.

5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA's Division of Biological Standards and Quality Control of the Office of Compliance and Biologics Quality.

It also should be pointed out that any of the foregoing therapies may prove useful by themselves in treating cancer.

### V. SYNTHETIC METHODS

In some aspects, the aziridine containing epothilone analogs of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the epothilone analogs described herein.

B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; “hydrazine” means —NHNH₂; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “hydroxysulfonyl” means —SO₃H, “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

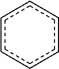

includes

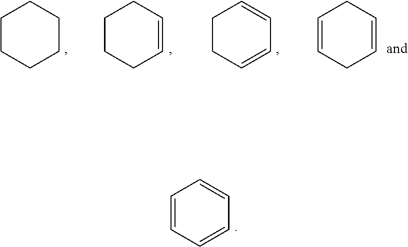

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

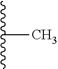

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

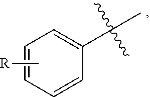

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

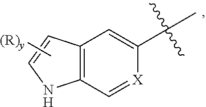

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals CH), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≤n)” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. For example, “alkoxy_{(C≤10)}” designates those alkoxy groups having from 1 to 10 carbon atoms. (Cn-n′) defines both the minimum (n) and maximum number (n′) of carbon atoms in the group. Similarly, “alkyl_{(C2-10)}” designates those alkyl groups having from 2 to 10 carbon atoms. Unless specified otherwise, any chemical group or compound class listed in a claim set without a carbon atom limit has a carbon atom limit of less than or equal to twelve.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group.

In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “aromatic” when used to modify a compound or a chemical group refers to a planar unsaturated ring of atoms with 4n+2 electrons in a fully conjugated cyclic 7E system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, Tr or isopropyl), —CH₂CH₂CH₂CH₃ (n-Bu), —CH(CH₃)CH₂CH₃ (sec-butyl), —CH₂CH(CH₃)₂ (isobutyl), C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂—, are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound HR, wherein R is alkyl as this term is defined above. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forms part of one or more non-aromatic ring structures, a cyclo or cyclic structure, no carbon-carbon double or triple bonds, and no atoms other than carbon and hydrogen. Non-limiting examples of cycloalkyl groups include: —CH(CH₂)₂ (cyclopropyl), cyclobutyl, cyclopentyl, or cyclohexyl. The term “cycloalkanediyl” when used without the “substituted” modifier refers to a divalent saturated aliphatic group with one or two carbon atom as the point(s) of attachment, said carbon atom(s) forms part of one or more non-aromatic ring structures, a cyclo or cyclic structure, no carbon-carbon double or triple bonds, and no atoms other than carbon and hydrogen.

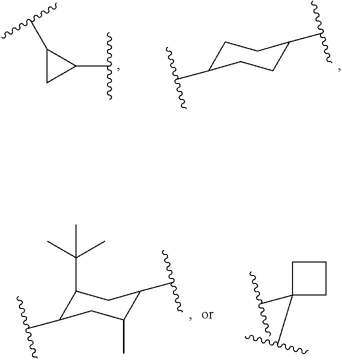 [SMILES: *C1CC1* ; *C1CCC(*)CC1 ; *C1CC(C(C)(C)C)C(*)CC1C ; *C1(*)CCC1]

are non-limiting examples of cycloalkanediyl groups. A “cycloalkane” refers to the compound HR, wherein R is cycloalkyl as this term is defined above. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted cycloalkyl groups: —C(OH)(CH₂)₂,

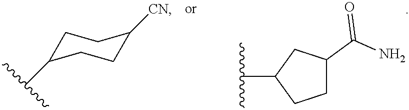 [SMILES: *C1CCC(C#N)CC1 ; *C1CCC(C(N)=O)C1]

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, and —CH═CHCH₂—, are non-limiting examples of alkenediyl groups. It is noted that while the alkenediyl group is aliphatic, once connected at both ends, this group is not precluded from forming part of an aromatic structure. The terms “alkene” and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —CH═CHF, —CH═CHC₁ and —CH═CHBr, are non-limiting examples of substituted alkenyl groups.

The term “alkynyl” when used without the “substituted” modifier refers to a monovalent unsaturated aliphatic group with a carbon atom as the point of attachment, a linear or branched, acyclic structure, at least one carbon-carbon triple bond, and no atoms other than carbon and hydrogen. As used herein, the term alkynyl does not preclude the presence of one or more non-aromatic carbon-carbon double bonds. The groups, —CCH, —CCCH₃, and —CH₂CCCH₃, are non-limiting examples of alkynyl groups. An “alkyne” refers to the compound HR, wherein R is alkynyl. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

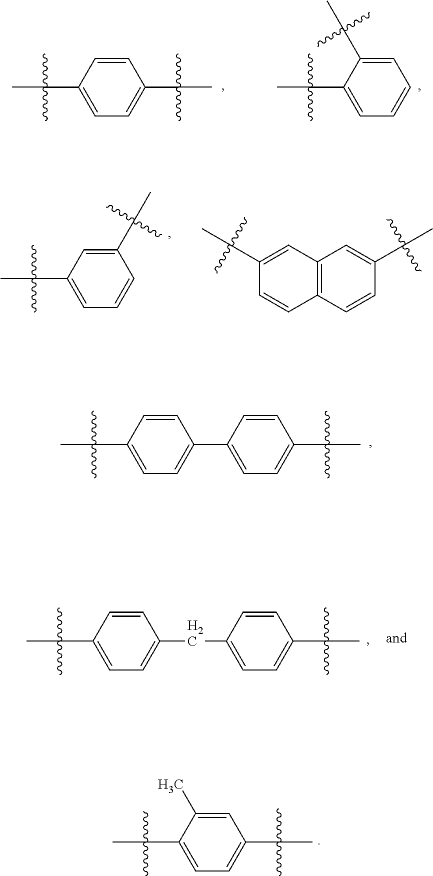

An “arene” refers to the compound HR, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group alkanediylaryl, in which the terms alkanediyl and aryl are each used in a manner consistent with the definitions provided above. Non-limiting examples of aralkyls are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl, isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. As the term is used herein, the term heteroaryl includes pyrimidine base and base analogs. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, aralkyl, and/or heteroaralkyl groups (carbon number limitation permitting) attached to the aromatic ring or aromatic ring system. Non-limiting examples of heteroarenediyl groups include:

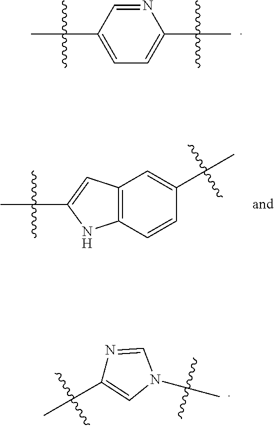 [SMILES: *c1ccc(*)nc1 ; *c1ccc2[nH]c(*)cc2c1 ; *c1cn(*)cn1]

A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —₈(O)₂₀H, or —S(O)₂NH₂.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of heteroaralkyls are: N-pyrazolylmethyl or quinolylmethyl. When the term heteroaralkyl is used with the “substituted” modifier one or more hydrogen atom from the alkanediyl and/or the heteroaryl group has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-nitropyrimidinyl)-methyl, and 4-chloro-2-quinolyl-eth-1-yl.

The term “acyl” when used without the “substituted” modifier refers to the group —C(O)R, in which R is a hydrogen, alkyl, cycloalkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. An “anhydride” is a group of the formula ROR′, wherein R and R′ are acyl groups as defined above. When any of these terms are used with the “substituted” modifier one or more hydrogen atom (including a hydrogen atom directly attached the carbonyl or thiocarbonyl group, if any) has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can each independently be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “cycloalkylamino”, “alkenylamino”, “cycloalkenylamino”, “alkynylamino”, “arylamino”, “aralkylamino”, “heteroarylamino”, “heterocycloalkylamino” and “alkylsulfonylamino” when used without the “substituted” modifier, refers to groups, defined as NHR, in which R is alkoxy, cycloalkyl, alkenyl, cycloalkenyl, alkynyl, aryl, aralkyl, heteroaryl, heterocycloalkyl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups NHC(O)OCH₃ and NHC(O)NHCH₃ are non-limiting examples of substituted amido groups.

The term “alkoxy” when used without the “substituted” modifier refers to the group OR, in which R is an alkyl, as that term is defined above. Non-limiting examples include: OCH₃ (methoxy), OCH₂CH₃ (ethoxy), OCH₂CH₂CH₃, OCH(CH₃)₂ (isopropoxy), and OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group SR, in which R is an alkyl and acyl, respectively. The term “alkylthiodiyl” refers to the divalent group —S-alkanediyl-, —S-alkanediyl-S—, or -alkanediyl-S-alkanediyl-. The term “alcohol” corresponds to an alkane, as defined above, wherein at least one of the hydrogen atoms has been replaced with a hydroxy group. The term “ether” corresponds to an alkane or cycloalkane, as defined above, wherein at least one of the hydrogen atoms has been replaced with an alkoxy or cycloalkoxy group. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylsilyl” when used without the “substituted” modifier refers to the groups —SiR₃, respectively, in which each R is an alkyl, as that term is defined above. The terms “alkenylsilyl”, “alkynylsilyl”, “arylsilyl”, “aralkylsilyl”, “heteroarylsilyl”, and “heterocycloalkylsilyl” are defined in an analogous manner. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The terms “phosphine” and “phosphane” are used synonymously herein. When used without the “substituted” modifier these terms refer to a compound of the formula PR₃, wherein each R is independently hydrogen, alkyl, cycloalkyl, alkenyl, aryl, or aralkyl, as those terms are defined above. Non-limiting examples include PMe₃, PPh₃, and PCy₃ (tricyclohexylphosphine). The terms “trialkylphosphine” and “trialkylphosphane” are also synonymous. Such groups are a subset of phosphine, wherein each R is an alkyl group. The term “diphosphine” when used without the “substituted” modifier refers to a compound of the formula R₂—P-L-P—R₂, wherein each R is independently hydrogen, alkyl, cycloalkyl, alkenyl, aryl, or aralkyl, and wherein L is alkanediyl, cycloalkanediyl, alkenediyl, or arenediyl. When any of these terms is used with the “substituted” modifier one or more hydrogen atom attached to a carbon atom has been independently replaced by —OH, —F, —Cl, —Br, —I, —NH₂, —NO₂, —N₃, —CO₂H, —CO₂CH₃, —CN, —SH, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

As indicated above in some aspects the cell-targeting moiety is an antibody. As used herein, the term “antibody” is intended to include immunoglobulins and fragments thereof which are specifically reactive to the designated protein or peptide, or fragments thereof. Suitable antibodies include, but are not limited to, human antibodies, primatized antibodies, de-immunized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, antibody-like molecules (e.g., anticalins), and antibody fragments. As used herein, the term “antibodies” also includes intact monoclonal antibodies, polyclonal antibodies, single domain antibodies (e.g., shark single domain antibodies (e.g., IgNAR or fragments thereof)), multispecific antibodies (e.g., bi-specific antibodies) formed from at least two intact antibodies, and antibody fragments so long as they exhibit the desired biological activity. Antibody polypeptides for use herein may be of any type (e.g., IgG, IgM, IgA, IgD and IgE). Generally, IgG and/or IgM are preferred because they are the most common antibodies in the physiological situation and because they are most easily made in a laboratory setting. As used herein the term antibody also encompasses an antibody fragment such as a portion of an intact antibody, such as, for example, the antigen-binding or variable region of an antibody. Examples of antibody fragments include Fab, Fab′, F(ab′)2, Fc and Fv fragments; triabodies; tetrabodies; linear antibodies; single-chain antibody molecules; and multi specific antibodies formed from antibody fragments. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. An oxygen linked antibody is an antibody which has a chemical function group such that the linkage between the antibody and the linker or compound is joined via an oxygen atom. Similarly, a nitrogen linked antibody is an antibody which has a chemical function group such that the linkage between the antibody and the linker or compound is joined via an nitrogen atom.

A “metal” in the context of this application is a transition metal or a metal of groups I or II. It may also be an element of Group 13 such as, but not limited to, boron and aluminum.

A “linker” in the context of this application is divalent chemical group which may be used to join one or more molecules to the compound of the instant disclosure. Linkers may also be an amino acid chain wherein the carboxy and amino terminus serve as the points of attachment for the linker. In some embodiments, the linker contains a reactive functional group, such as a carboxyl, an amide, a amine, a hydroxy, a mercapto, an aldehyde, or a ketone on each end that be used to join one or more molecules to the compounds of the instant disclosure. In some non-limiting examples, —CH₂CH₂CH₂CH₂—, —C(O)CH₂CH₂CH₂—, —OCH₂CH₂NH, —NHCH₂CH₂NH—, and —(OCH₂CH₂)ₙ—, wherein n is between 1-1000, are linkers.

An “amine protecting group” is well understood in the art. An amine protecting group is a group which prevents the reactivity of the amine group during a reaction which modifies some other portion of the molecule and can be easily removed to generate the desired amine Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH or PG_{DA}N wherein PG_{MA} is a monovalent amine protecting group, which may also be described as a “monvalently protected amino group” and PG_{DA} is a divalent amine protecting group as described above, which may also be described as a “divalently protected amino group”.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected hydroxy group is a group of the formula PG_{H}O— wherein PG_{H} is a hydroxyl protecting group as described above.

A “thiol protecting group” is well understood in the art. A thiol protecting group is a group which prevents the reactivity of the mercapto group during a reaction which modifies some other portion of the molecule and can be easily removed to generate the desired mercapto group. Thiol protecting groups can be found at least in Greene and Wuts, 1999, which is incorporated herein by reference. Some non-limiting examples of thiol protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected thiol group is a group of the formula PG_{T}S— wherein PG_{T} is a thiol protecting group as described above.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2′, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its (R) form, (S) form, or as a mixture of the (R) and (S) forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1 Synthesis of Aziridine Epothilone Analogs

A. Preparation of Aziridine Epothilone B Analogs

Inspired by the required presence of a basic nitrogen atom in a certain position in highly active epothilones and motivated by the necessity for suitable functional groups as points of attachment for conjugation studies (Nicolaou & Snyder, 2003, Altmann et al., 2007, Altmann et al., 2009, Pfeiffer et al., 2009, Altmann et al., 2011, Pfeiffer et al., 2012, Altmann et al., 2014, Schiess et al., 2015 and WO 9825929 A1, WO 9967252 A2, WO 9967253 A2, WO 03018002 A2, WO 2004014919 A1, WO 2007062288 A2, Nicolaou et al., 2015, US 2016057093), focus was directed towards epothilone analogues in which the epoxide moiety of epothilone B is replaced with an aziridine moiety. Given that most synthetic routes to epothilones proceed through the corresponding C12-C13 olefin, and mindful of the power of the recently disclosed Ess-Kürti-Falck aziridination (Jat et al., 2014), experimentation to test whether epothilones C and D (3 and 4, Scheme 1) could serve as substrates for this reaction was initiated. It should be noted that the synthesis of C12-C13 aziridinyl epothilone B has not been reported, although the corresponding aziridinyl epothilone A (8) was previously prepared from epothilones A and C (1 and 3, Scheme 2, panel A), albeit through a lengthy process (WO 9954319 A1, Regueiro-Ren et al., 2001, WO 02098868 A1, US 20070276018, WO 2007140297 A1, WO 2007140298 A1, WO 2008147941 A1). In contrast, and as shown in Scheme 1, preparation of both the aziridinyl epothilone A analogue 8 (70% yield) and aziridinyl epothilone B analogue 10 (66% yield) was accomplished through the Ess-Kürti-Falck method [O-(2,4-dinitrophenyl) hydroxylamine (DPH), Rh₂(esp)₂ cat (Jat et al., 2014)] directly and in one step from epothilones C and D (3 and 4, Scheme 3), respectively. Furthermore, the aziridination reaction was found to be completely regio- and stereoselective, leading to the desired epothilone configuration as proven by comparison of the NMR data of aziridine 8 with those reported by Bristol-Myers Squibb (BMS) for the same compound (WO 9954319 A1, Regueiro-Ren et al., 2001, WO 02098868 A1, US 20070276018, WO 2007140297 A1, WO 2007140298 A1, WO 2008147941 A1). Assignment of the β-configuration for aziridine 10 and the other aziridine compounds obtained in this study was accomplished by analogy. These results contrasts with the epoxidation reaction [DMDO (dimethyldioxirane) or TFDO (methyhtrifluoro-methylidioxirane)], which generates epothilone B (2) from its corresponding olefinic precursor, epothilone D (4), a process that displayed moderate diasteroselectivity (ca. 5:1 dr)(Nicolaou et al., 1997). Subsequent alkylation of these aziridines with 2-bromoethanol (DPH, K₂CO₃) afforded N-hydroxyethyl aziridinyl epothilones A (9, 97% yield) and B (11, 95% yield), respectively, as shown in Scheme 3. The primary hydroxyl group on these structures may serve as a convenient functionality to attach linkers for potential ligation to antibodies and other delivery systems.

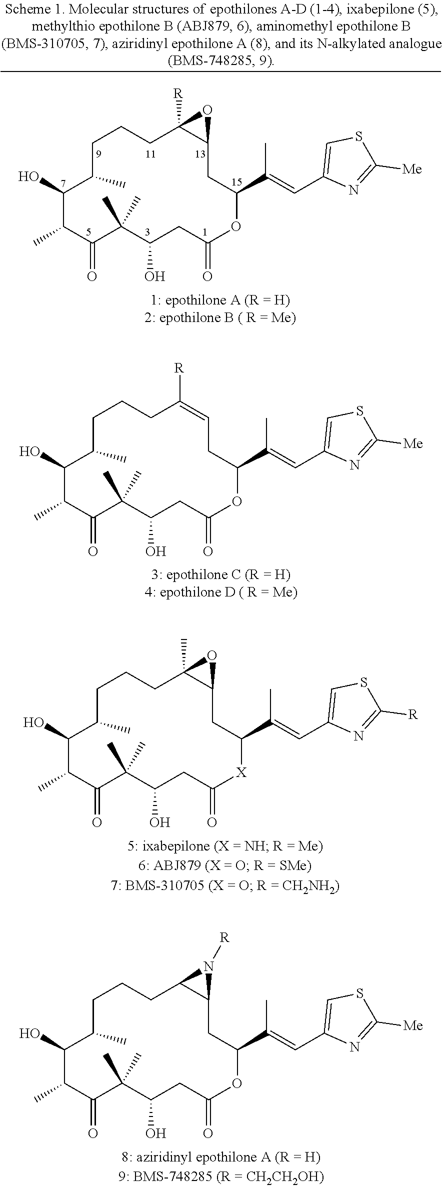

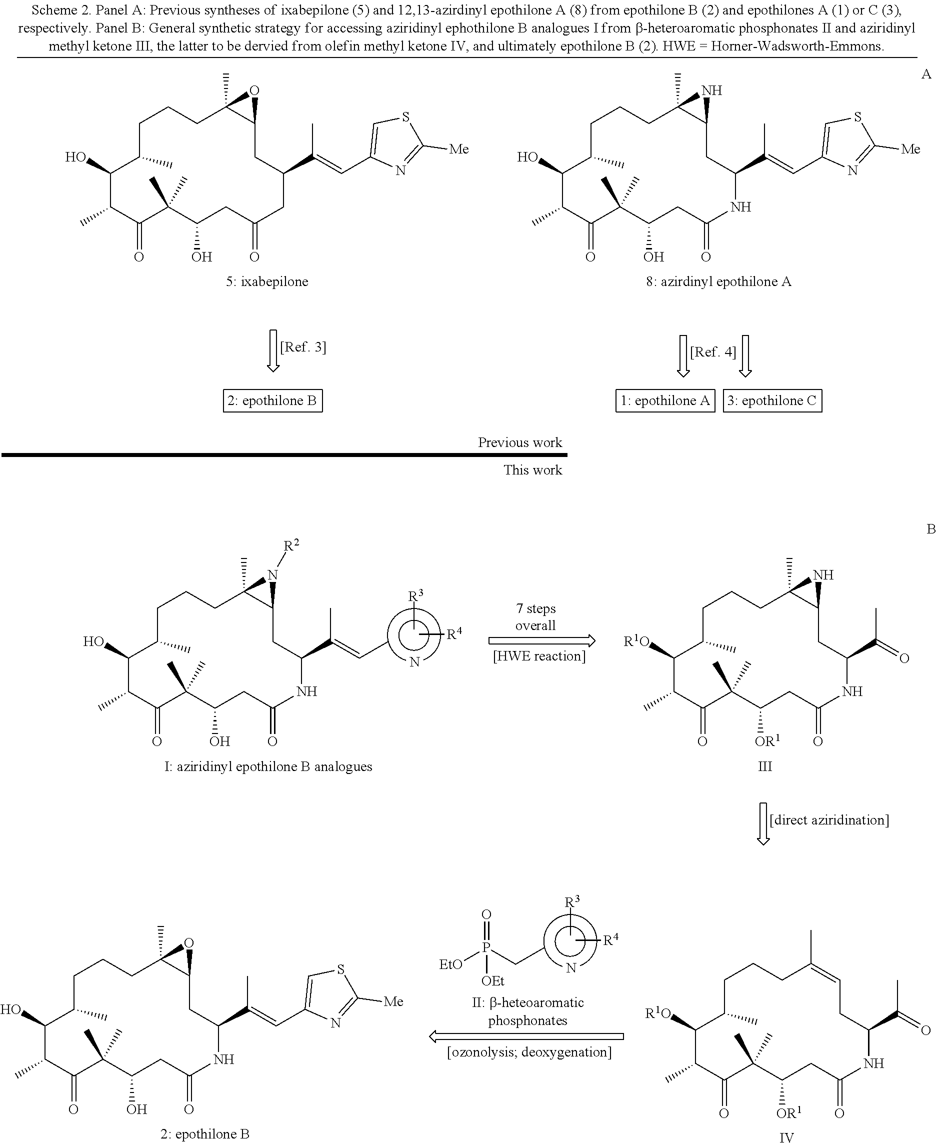

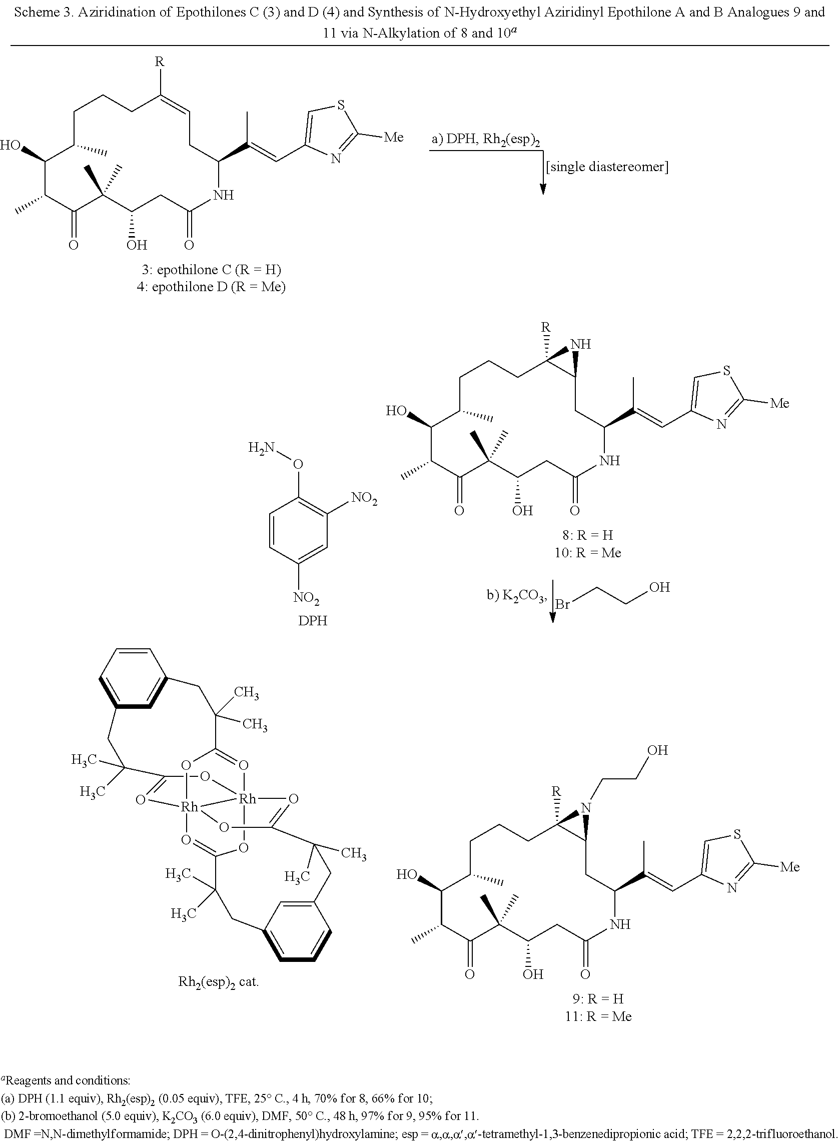

B. Attempted Stille Coupling Approach for the Synthesis of Aziridinyl Epothilone B Side Chain Analogues.

Having established the feasibility of the aziridination reaction with these rather complex substrates and demonstrated its excellent regio- and stereoselectivity as well as its tolerance of the thiazole moiety, experimentation was initiated to probe its applicability to other substrates such as those featuring a vinyl iodide moiety (e.g., 71; for its ability to serve as a precursor to a wide range of analogues) and various heterocyclic side chains (e.g., N-methyl-5-methylthiopyrazole; for its ability to impart high potency)(Nicolaou et al., 2006) as depicted in Scheme 2. Thus, triol iodide precursor 69, readily available using previously published methods (Nicolaou et al., 1999), was converted to iodide 71 (NaBH₃CN, 80% yield) via bis-iodide 70, the latter obtained from 69 through its tosylate counterpart (Ts₂O, Et₃N, DMAP; then TBAI, 88% yield). Iodide 71, however, left much to be desired as a substrate for aziridination under the Ess-Kürti-Falck conditions, and aziridine 72 could not be obtained in meaningful quantities. To circumvent this problem, substrate 73 was prepared through Stille coupling of vinyl iodide precursor 71 with pyrazolyl stannane 74 (Nicolaou et al., 2006) [Pd₂(dba)₃ cat., CuI, AsPh₃, 67% yield] as shown in Scheme 2. However, exposure of this substrate to the aziridination reaction failed to produce the desired aziridine (40). Without wishing to be bound by any theory, it is believed that the failure of this reaction was due to oxidative degradation of the electron-rich methylthiopyrazole moiety (Zenzola et al., 2016).

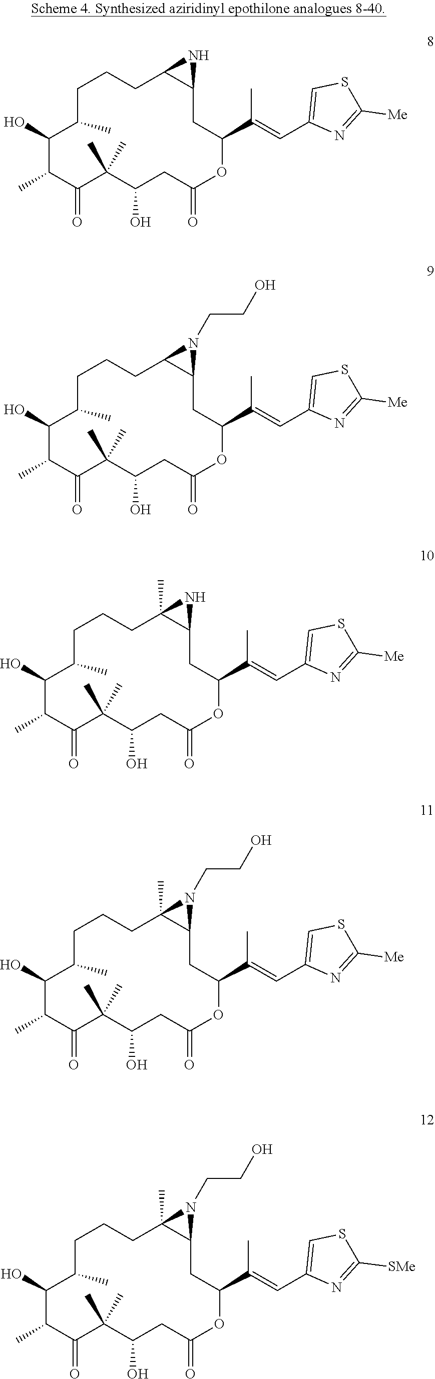

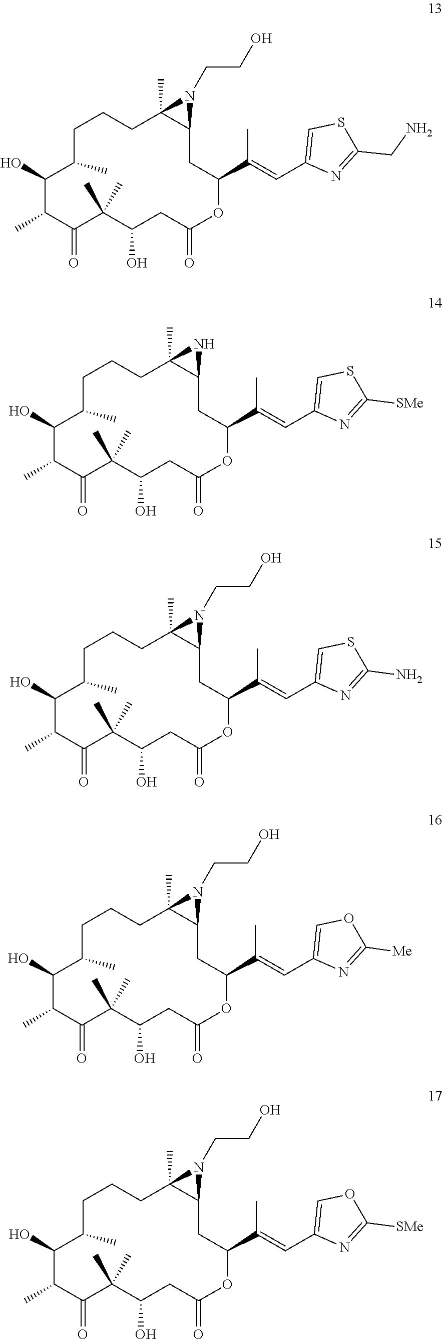

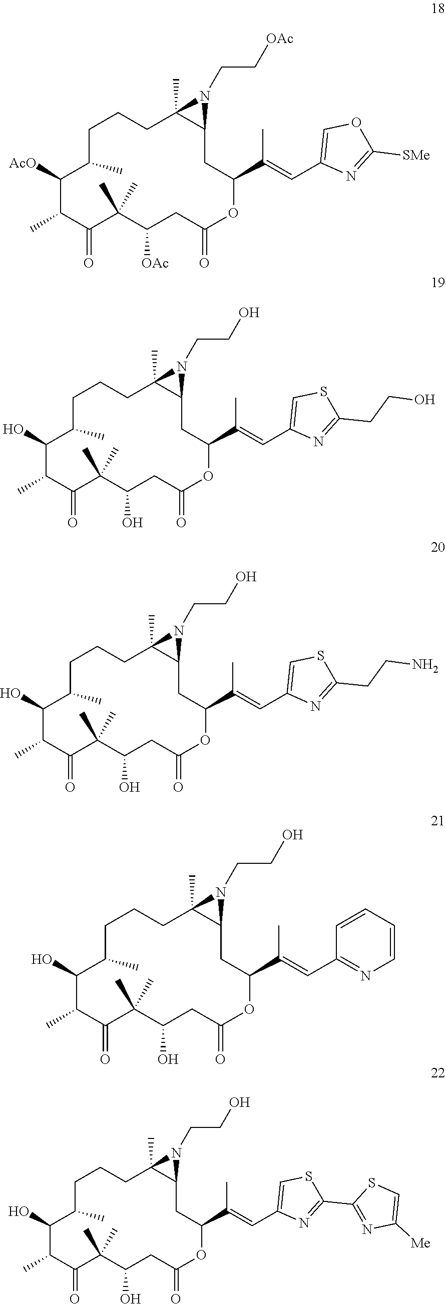

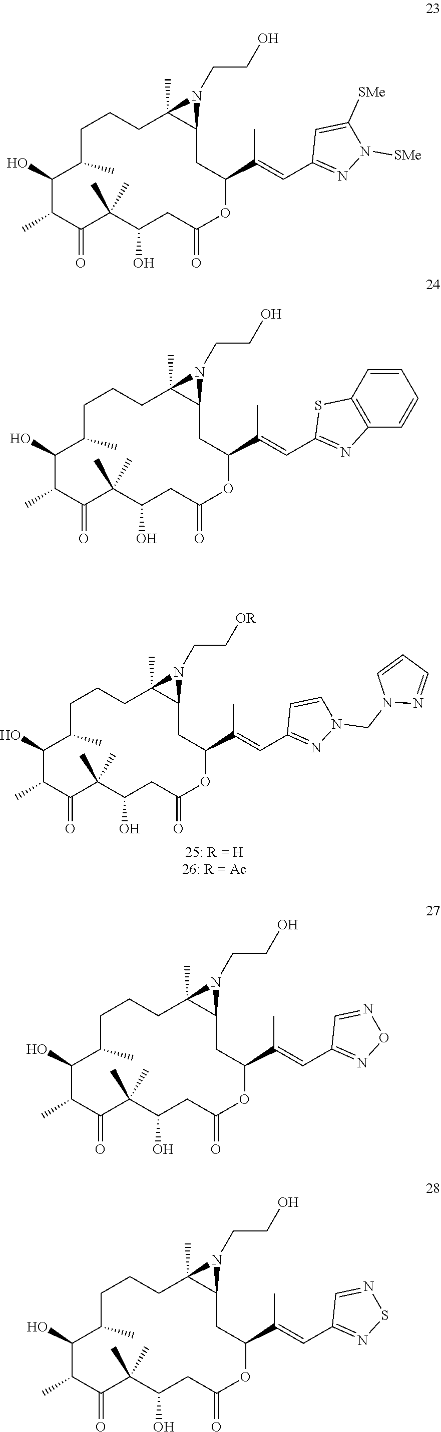

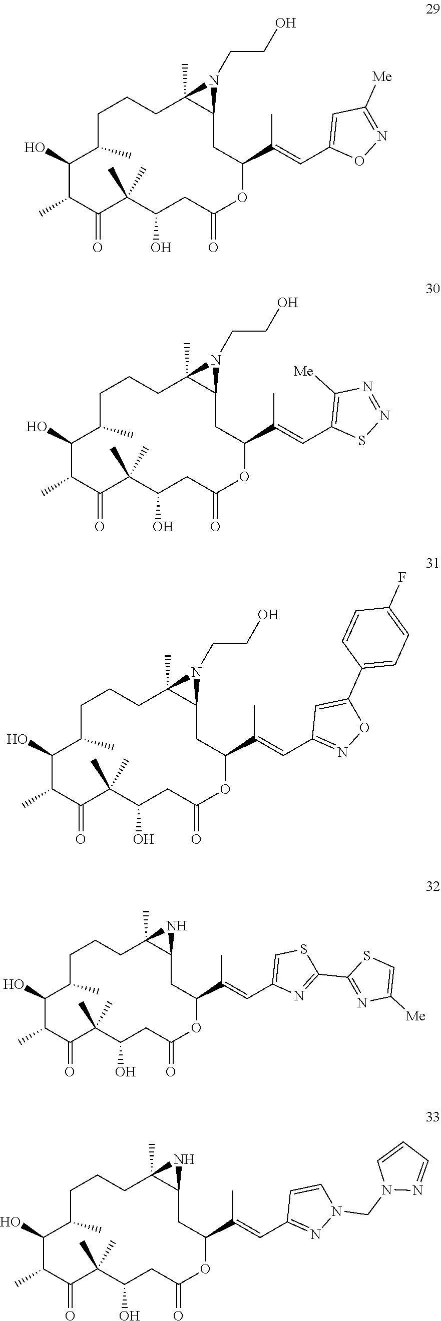

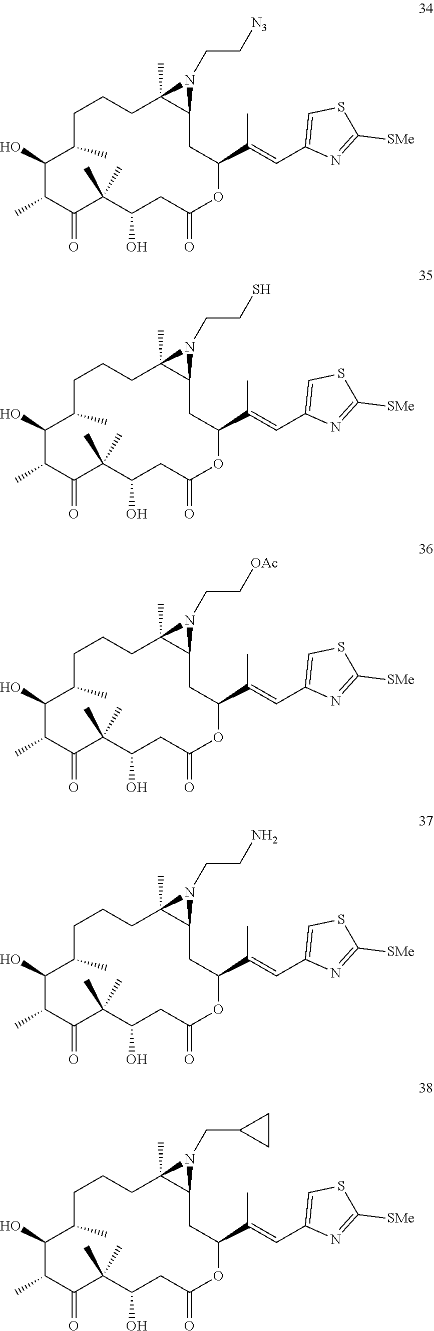

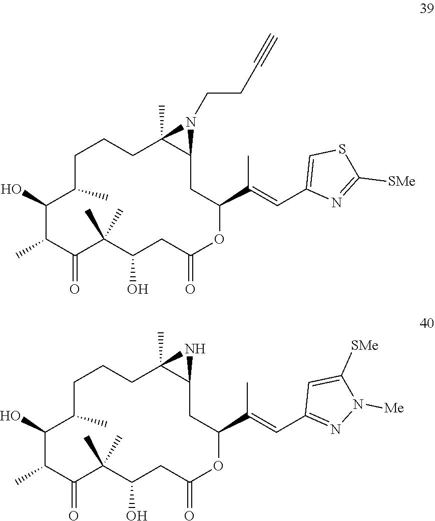

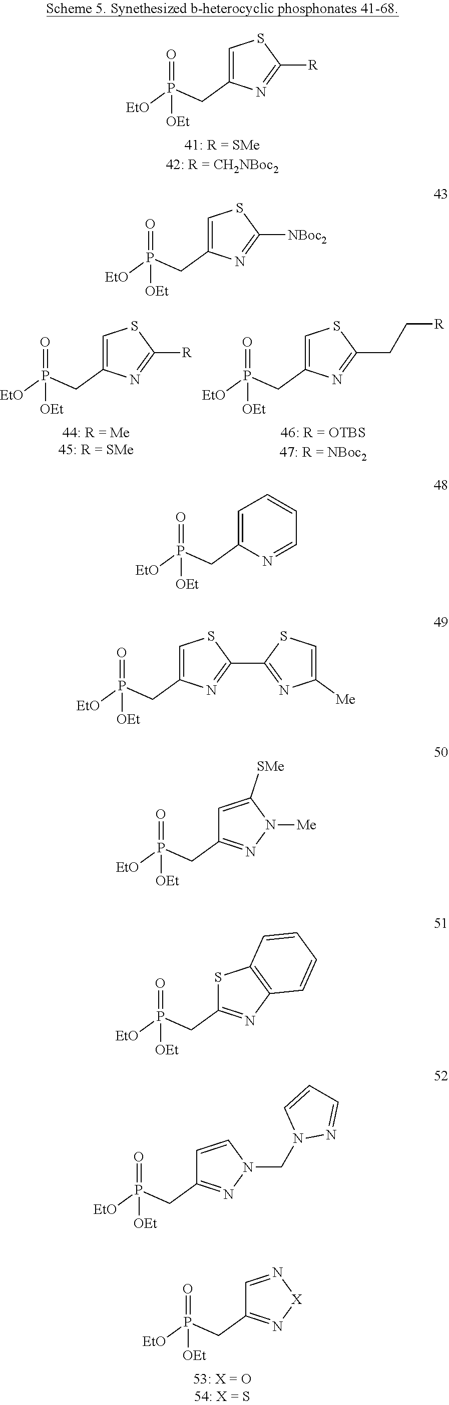

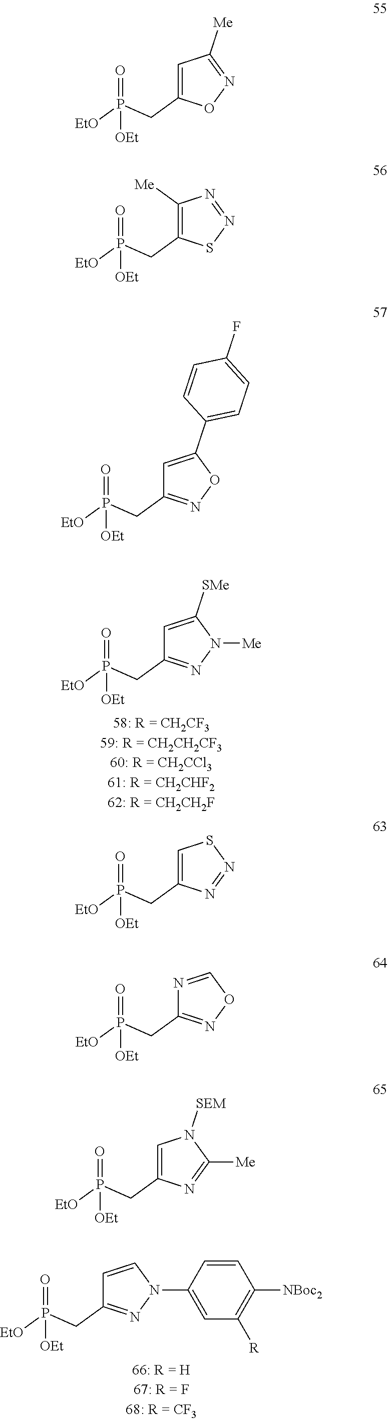

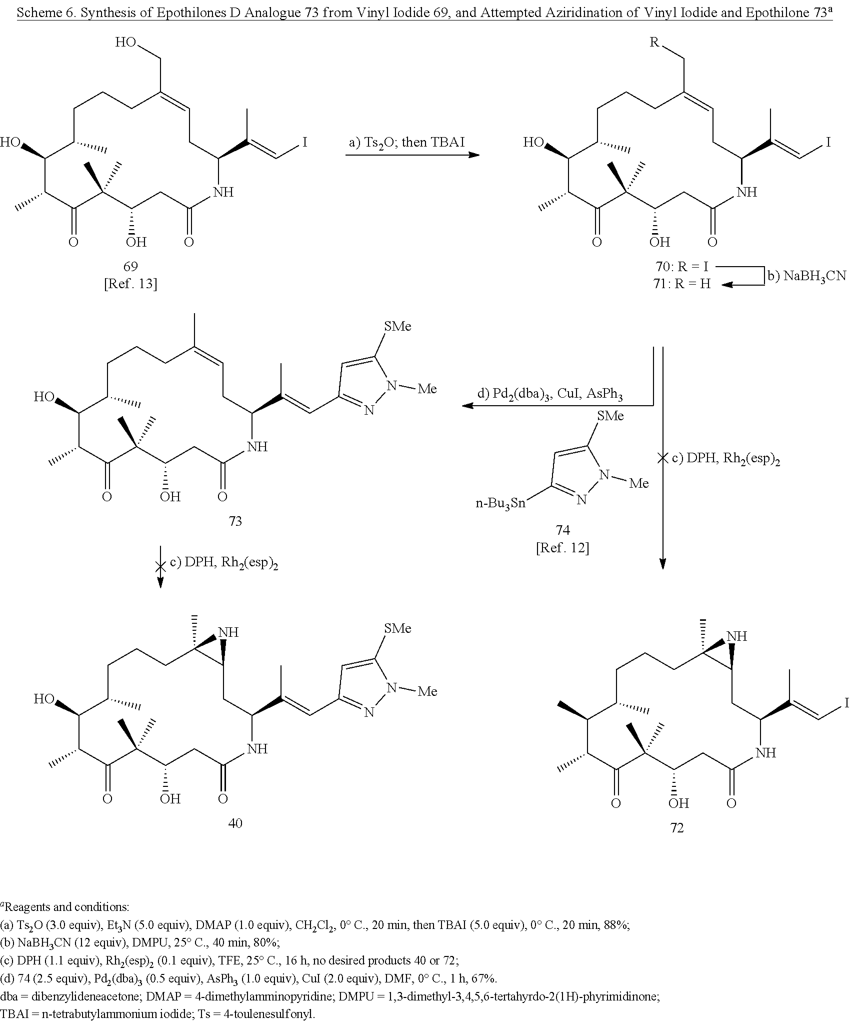

C. Development of a New Synthetic Route to Substituted Aziridinyl Epothilone B Analogues.

Attention shifted to the rupture of the side chain of readily available (via fermentation) epothilone B (2), and deoxygenation of its epoxide moiety to provide a viable precursor onto which both the aziridine moiety and various side chains could be installed. Previous studies by Wide (Sefkow et al., 1998, Höfle et al., 1999) and Bristol-Myers Squibb (BMS) scientists (Johnson et al., 2000) indicated that the former objective could be accomplished through ozonolysis, whereas the latter could be realized through an in situ generated reducing metal (e.g., W or Mg/Ti).

Thus, and as shown in Scheme 3, epothilone B (2) was converted by ozonolysis (O₃; then Me2S) to methyl ketone 75 (94% yield), which was then exposed to TESOTf and 2,6-lutidine to afford bis-TES ether 76 in 84% yield. Compounds 75 and 76 were tested in the subsequent deoxygenation of the epoxide moiety with WCl₆/n-BuLi, revealing their viability as substrates for the preparation of the required olefinic methyl ketones 77 [85% yield, (Z):(E) ca. 5:1] and 78 [86% yield, (Z) only], respectively. The latter proved to be the preferred substrate due to its exclusive geometrical selectivity for the desired (Z) isomer. The aziridination of both substrates 77 or 78 also proved successful under the standard conditions (Jat et al., 2014), with 77 affording aziridine 79 in 87% yield, and 78 furnishing aziridine 80 in 90% yield, both with complete stereoselectivity. Compound 80 was converted to epothilone B analogue 10 (Scheme 3), whose NMR spectroscopic data matched those of a sample derived from epothilone B (Johnson et al., 2000) (or epothilone D) as described in Scheme 1, thereby supporting the stereochemical assignment of the aziridination of methyl ketones 77 and 78. The subsequent alkylation of aziridines 79 (free hydroxyl groups) and 80 (TES-protected hydroxyl groups) with 2-bromoethanol (with or without TBS-protection of the hydroxyl group) and K₂CO₃ [79+2-bromoethanol→81 (29% yield); 80+2-bromoethanol-TBS ether→82 (90% yield)], however, distinguished the TES-protected compound (i.e., 80) and 2-bromoethanol TBS ether as the preferred substrates in terms of overall yield and selectivity. Precursor 82 was then coupled with side chain phosphonate 41 under the influence of n-BuLi to furnish, through the ensuing stereoselective Horner-Wadsworth-Emmons (HWE) olefination ((a) Homer et al., 1958 (b) Horner et al., 1959 (c) Wadsworth et al., 1961 (d) Wadsworth et al., 1965 and (a) Maryanoff et al., 1989 (b) Nicolaou et al., 1997 (c) Gu et al., 2012 (d) Blakemore et al., 2014 (e) Bisceglia & Orelli, 2015, the expected protected aziridinyl epothilone B analogue 83 in 60% yield. From the latter, desilylated product aziridinyl methylthio epothilone B analogue 12 was liberated by treatment with HF·py (79% yield). In a similar manner, precursor 82 was coupled with phosphonate 42, this time under the influence of NaHMDS, to afford protected aziridinyl epothilone B analogue 84 (68% yield). Global deprotection (HF·py; then TFA) provided aziridinyl aminomethyl epothilone B analogue 13 in 48% overall yield, as summarized in Scheme 7.

This synthetic route clearly offers distinct advantages over previously described routes (e.g., 8 ((WO 9954319 A1, Regueiro-Ren et al., 2001, WO 02098868 A1, US 20070276018, WO 2007140297 A1, WO 2007140298 A1, WO 2008147941 A1) and 9 (US 20070275904 A1, Kim et al., 2011, Gokhale et al., 2013), Scheme 1) due to its versatility and the practical availability of the common intermediate (i.e., methyl ketone 82, Scheme 7), from which it can diverge to a wide range of imaginable side chain aziridinyl epothilone B analogues. Furthermore, this synthetic work represents the first application of the Ess-Kürti-Falck aziridination on substrates of high complexity, an observation that bodes well for its future application for the synthesis of complex natural products and designed molecules.

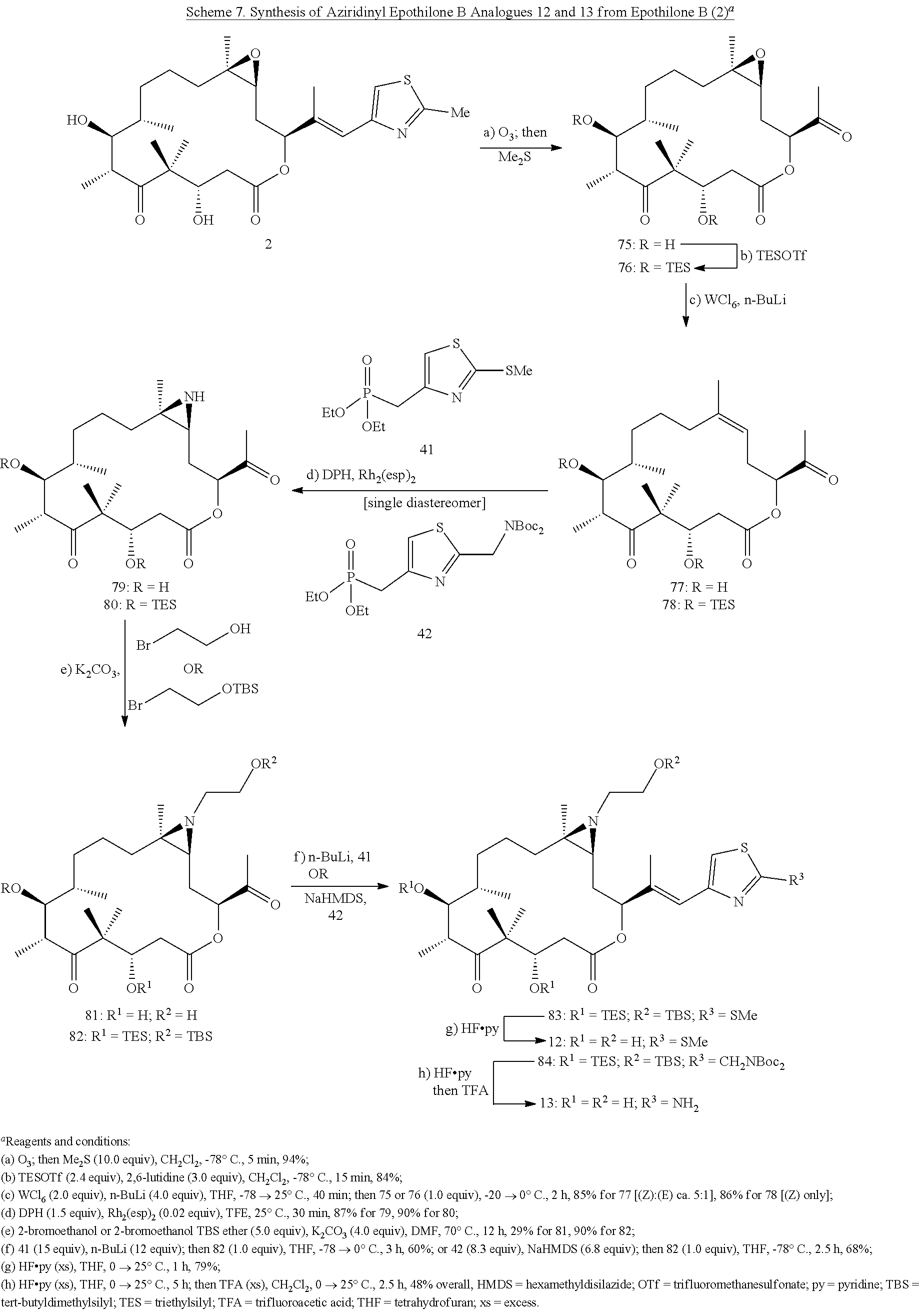

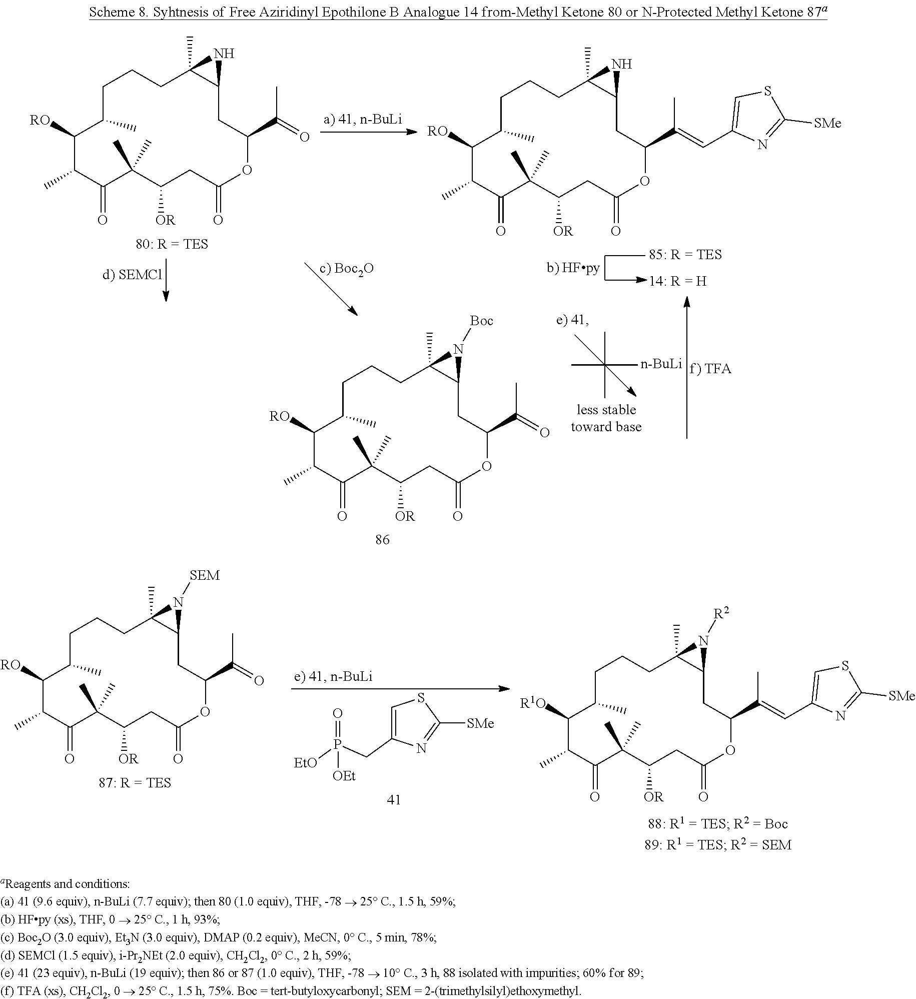

D. Synthetic Route Extension to Free Aziridinyl Epothilone B Analogues.

Experimentation was initiated to investigate the direct attachment of epothilone side chains using the unprotected (80) aziridinyl or protected (86 and 87) aziridinyl methyl ketones as substrates, as summarized in Scheme 4. It was observed that the free aziridine bis-TES protected methyl ketone 80 could serve as a substrate for the HWE reaction with methylthiothiazolylphosphonate 41 (n-BuLi, 59% yield) to afford, with complete geometrical control [(E):(Z)>98:2], the corresponding protected aziridinyl epothilone B 85, whose desilylation (HF·py, 93% yield) led smoothly to the targeted aziridinyl methylthio epothilone B analogue 14. Narrow substrate scope prompted a screen of various protecting groups. Thus, further experimentation with Boc-protected aziridinyl methyl ketone 86 (prepared from 80 and Boc₂O in the presence of Et₃N and DMAP, 78% yield, Scheme 8) as a substrate for the HWE olefination reaction did not give the expected product (i.e., 88), demonstrating its unsuitability to serve fruitfully, presumably due to activation of the aziridine moiety imparted by the carbamate group. An attempted HWE reaction with a 4-methoxybenzyl (PMB)-protected aziridine moiety also proved unsuccessful. On the other hand, the use of the SEM group as a protective device on the nitrogen atom (substrate 87, prepared from 80 as indicated in Scheme 4) led to success in the HWE olefination reaction with phosphonate 41, furnishing the corresponding aziridinyl epothilone B derivative 89 in 60% yield as a single geometrical isomer [(E):(Z)>98:2]. Global desilylation of the latter (TFA) gave the targeted aziridinyl methylthio epothilone B analogue 14 in 75% yield. These findings broadened the scope of this synthetic strategy toward other designed free aziridinyl epothilone B analogues (see Schemes 11 and 17 below).

E. Molecular Design of Aziridinyl Epothilone B Analogues.

Having developed efficient and stereoselective synthetic strategies and protocols for the construction of aziridinyl epothilones with appropriate side chains, as demonstrated with the synthesis of 12,13-aziridinyl epothilone B analogues 8-14 (Schemes 3, 7 and 8), these strategies were applied to the synthesis of a variety of additional aziridinyl epothilone B analogues. During the course of these efforts a novel modification to the HWE olefination reaction was developed. To this end, 26 additional analogues (15-40, Scheme 7) were developed as well as a series of corresponding β-heteroaromatic phosphonates (see Scheme 5). In designing this 12,13-aziridinyl family of epothilones, previous and encouraging results were noted from Nicolaou (Nicolaou et al., 1998, Nicolaou et al., 2001, Nicolaou et al., 2001, Nicolaou et al., 2002), Altmann (Cachoux et al., 2006 Kuzniewski et al., 2008, Altmann et al., 2010, Schiess et al., 2011, Gaugaz et al., 2014), and BMS (Sefkow et al., 1998, Hale et al., 1999), which indicated that the epoxide moiety could be replaced with other isosteres, such as the cyclopropane or aziridine structural motifs. The previous aziridination efforts, however, ended with the epothilone A explorations due to synthetic methodology limitations at the time (WO 9954319 A1, Regueiro-Ren et al., 2001, WO 02098868 A 1, US 20070276018, WO 2007140297 A 1, WO 2007140298 A 1, WO 2008147941 A 1). Since epothilone B exhibits higher potencies than epothilone A in general (ca. 10-fold) (Nicolaou & Snyder, 2003, Altmann et al., 2007, Altmann et al., 2009, Pfeiffer et al., 2009, Altmann et al., 2011, Pfeiffer et al., 2012, Altmann et al., 2014, Schiess et al., 2015), it was desirable to direct efforts toward analogues of the former structure, a goal that has been accomplished and broadly expanded upon with the installation of the aziridine moiety and a variety of heterocyclic side chains into the epothilone B precursor molecule. With regards to the side chains, previous studies from the Nicolaou laboratories that suggested, for example, the methylthiopyrazole (Nicolaou et al., 2006) and the methylthiothiazole (Nicolaou et al., 2002) moieties as potency-enhancing structural motifs. The requirement of a basic heteroatom (e.g., N) capable of acting as a hydrogen bond acceptor as it is found in the natural epothilones was maintained in all designs. A number of new ideas for binding optimization were incorporated as described below. Finally, and in order to fulfill the requirement of sites for linker attachment or direct conjugation to appropriate delivery systems, various additional functional groups were installed, such as primary hydroxyl and amino moieties, among others, into the designed molecules. Further rationale for certain epothilone designs is discussed with their syntheses below.

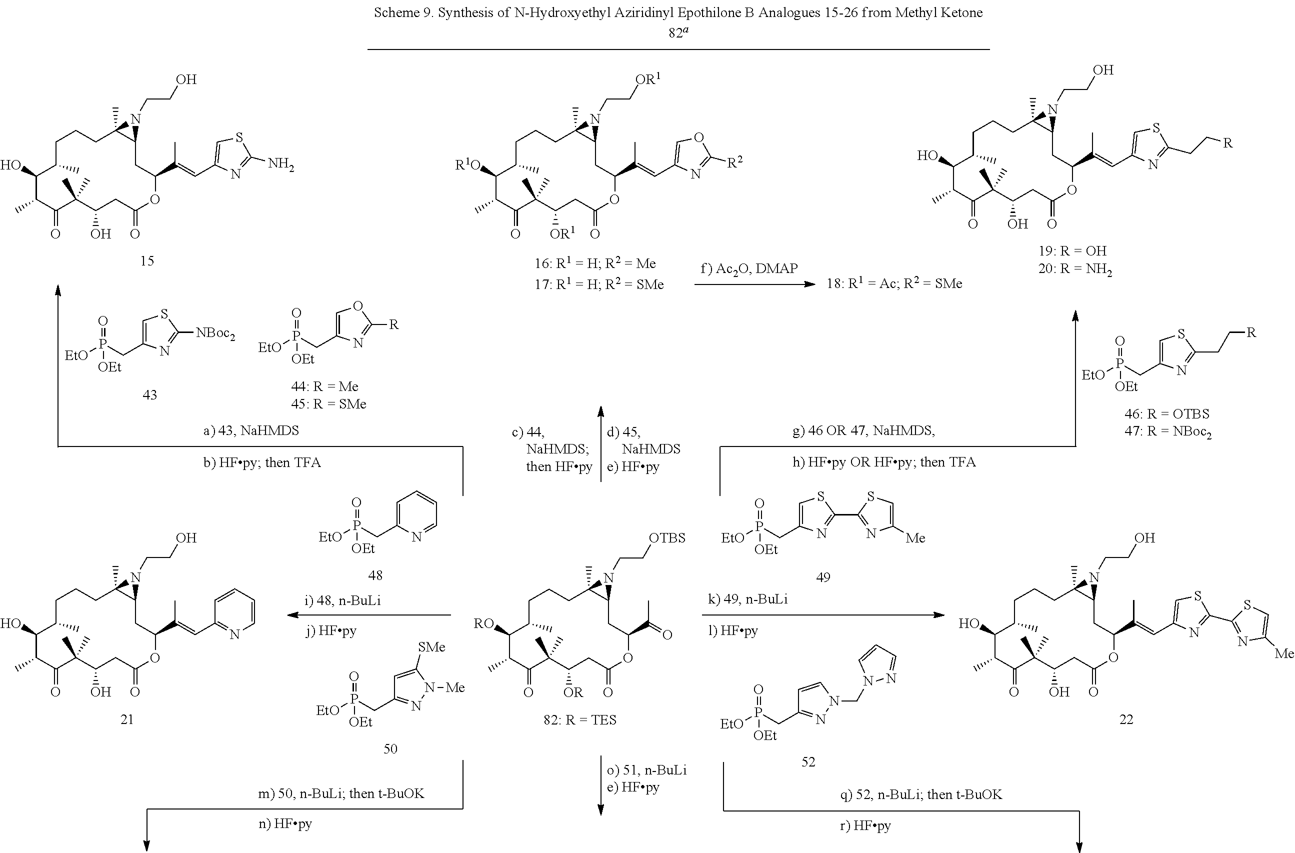

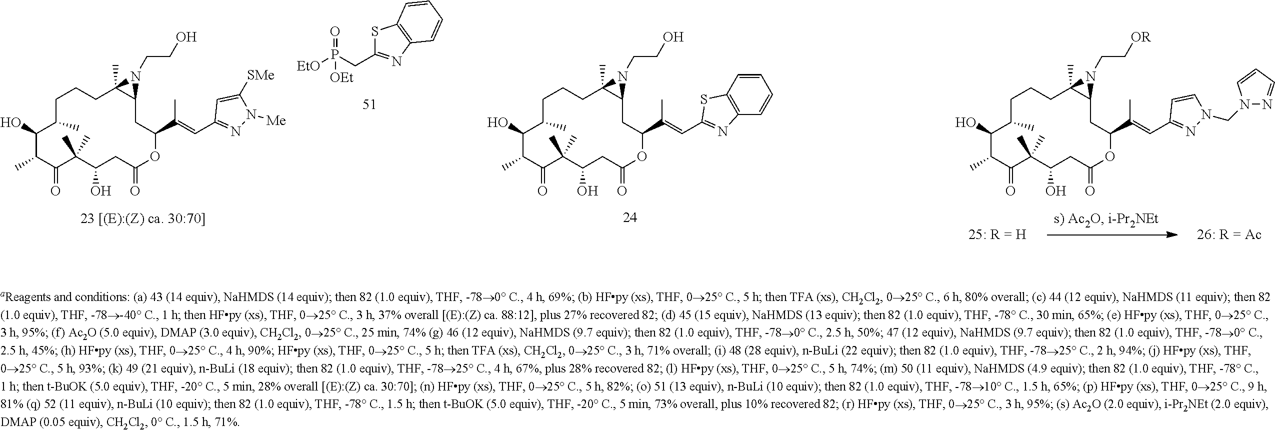

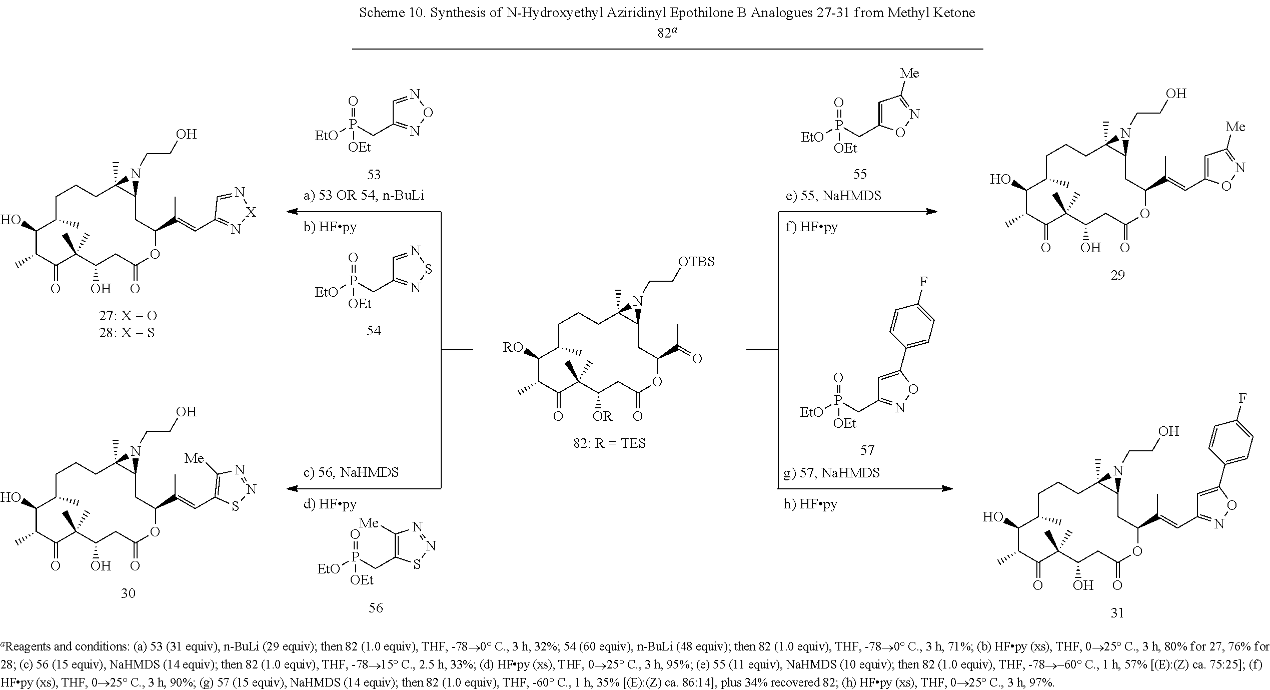

F. Synthesis of Designed N-Hydroxyethyl Aziridinyl Epothilone B Analogues 15-31.

The synthesis of designed aziridinyl epothilone B analogues 15-31 (Scheme 4) relied on the optimal intermediates and conditions described above for the synthesis of the aforementioned aziridinyl epothilone B analogues (i.e., 12-14, Schemes 7 and 8). Scheme 9 summarizes the construction of N-hydroxyethyl aziridinyl epothillone B analogues 15-26 from protected methyl ketone 82 and the corresponding phosphonates 43-52 through the two step sequence of olefination (NaHMDS or n-BuLi, see Scheme 9) followed by global deprotection (HF·py or HF·py; then TFA) in overall yields ranging from 32-87% as indicated in Scheme 9. Analogues possessing the pyridine (21), benzothiazole (24), and N-methyl-5-methylthiopyrazole (23) were synthesized (see Scheme 9). In addition, the aminothiazole (15), hydroxyethylthiazole (19), and aminoethylthiazole (20) containing analogues represent new modifications to the natural product side chain, while the analogues containing the underexplored methyloxazole (16) (The attempted HWE reaction with a 4-methoxybenzyl (PMB)-protected aziridine moiety also proved unsuccessful.) (Nicolaou et al., 1997) and unknown methylthiooxazole (17) structural motifs were also prepared. Furthermore, the design and synthesis of bis-thiazolyl analogue 22 and 1,1-dipyrazolylmethyl analogue 25 were based on the hypothesis that a well-positioned second basic nitrogen atom in the vicinity of the thiazole nitrogen atom (hypothesized for hydrogen bonding of epothilones to (3-tubulin) (Carlomagno et al., 2003, Nettles et al., 2004, Reese et al., 2007, Prota et al., 2013) might result in an increase of potencies for these compounds. Lastly, the peracetylated (18) and monoacetylated (26) analogues were prepared by standard acetylation conditions from analogues 17 and 25, respectively, in order to explore the likelihood of enhanced potency as a result of increased cellular membrane permeability (Rautio et al., 2008, Huttunen et al., 2011).

Scheme 10 summarizes the preparation of N-hydroxyethyl aziridinyl epothilone B analogues 27-31 possessing unnatural heterocyclic moieties (i.e., oxadiazole 27, thiadiazoles 28 and 30, and isoxazoles 29 and 31) from the protected methyl ketone 82 and phosphonates 53-57, respectively, in an effort to further probe structure-activity relationships (SARs) within the side chain region. In a similar fashion to the synthesized analogues described in Scheme 9, this protocol relied on the standard olefination (NaHMDS or n-BuLi)/global deprotection (HF·py) strategy, providing good overall yields, as indicated in Scheme 10. It is noteworthy that while the majority of the HWE reactions afforded the desired (E) isomer exclusively [(E):(Z)>98:2], oxazole [analogue 16, Scheme 9, (E):(Z) ca. 88:12] and isoxazole [analogues 29 and 31, Scheme 10, (E):(Z) ca. 75:25 and 86:14, respectively] phosphonates (i.e., 44, 55, and 57, respectively) also rendered a small amount of the corresponding epothilone (Z) isomer, but were still highly diastereoselective. Through these observations it became apparent that the electron density of the heterocyclic moiety plays a role in determining the stereochemical outcome of this reaction (discussed further below).

G. Synthesis of Designed Free Aziridinyl Epothilone B Analogues 32 and 33.

The aforementioned protecting group strategy for the construction of free aziridinyl epothilone B analogues (i.e., Scheme 8) was applied accordingly to the synthesis of analogues 32 and 33 from SEM-protected methyl ketone precursor 87, as shown in Scheme 11. Thus, olefination of 87 with phosphonates 49 and 52 (n-BuLi) followed by deprotection (TFA) delivered free aziridinyl epothilone B analogues 32 (29% overall yield) and 33 (28% overall yield), respectively, as indicated in Scheme 11. The lower yields were compensated by the recovery of considerable amounts of starting material (i.e., 87) for these challenging substrates (see caption, Scheme 11).

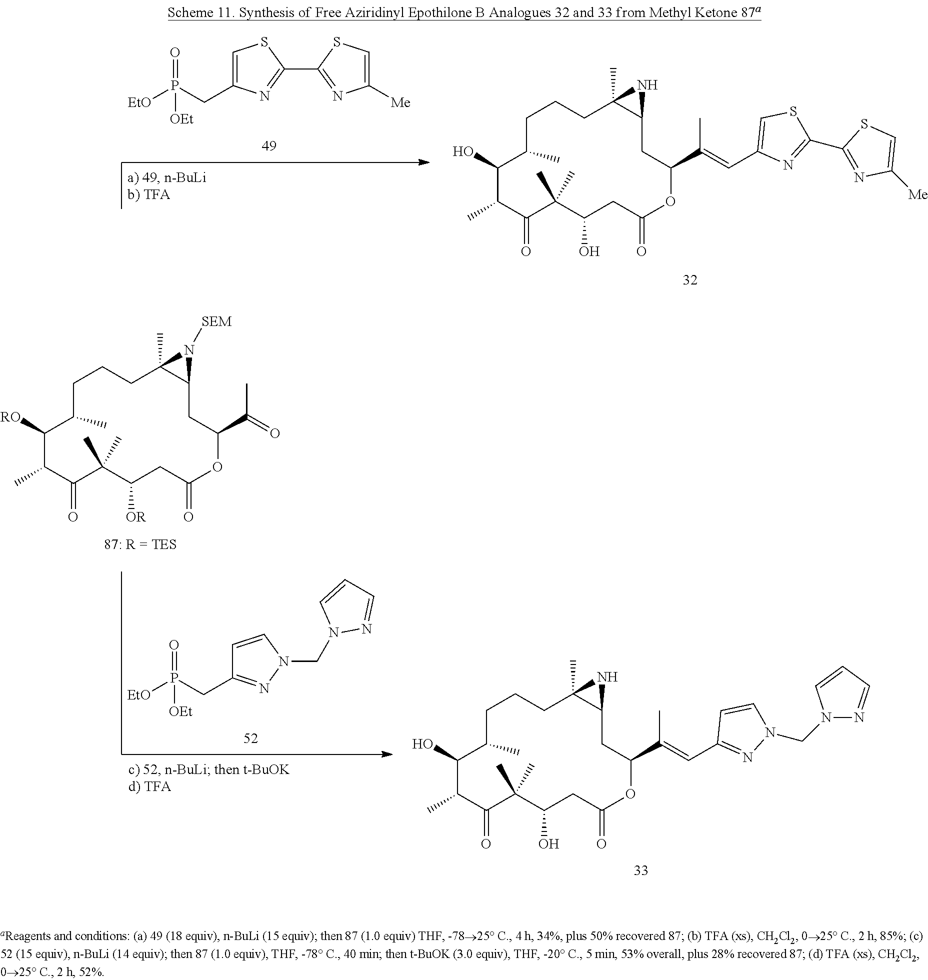

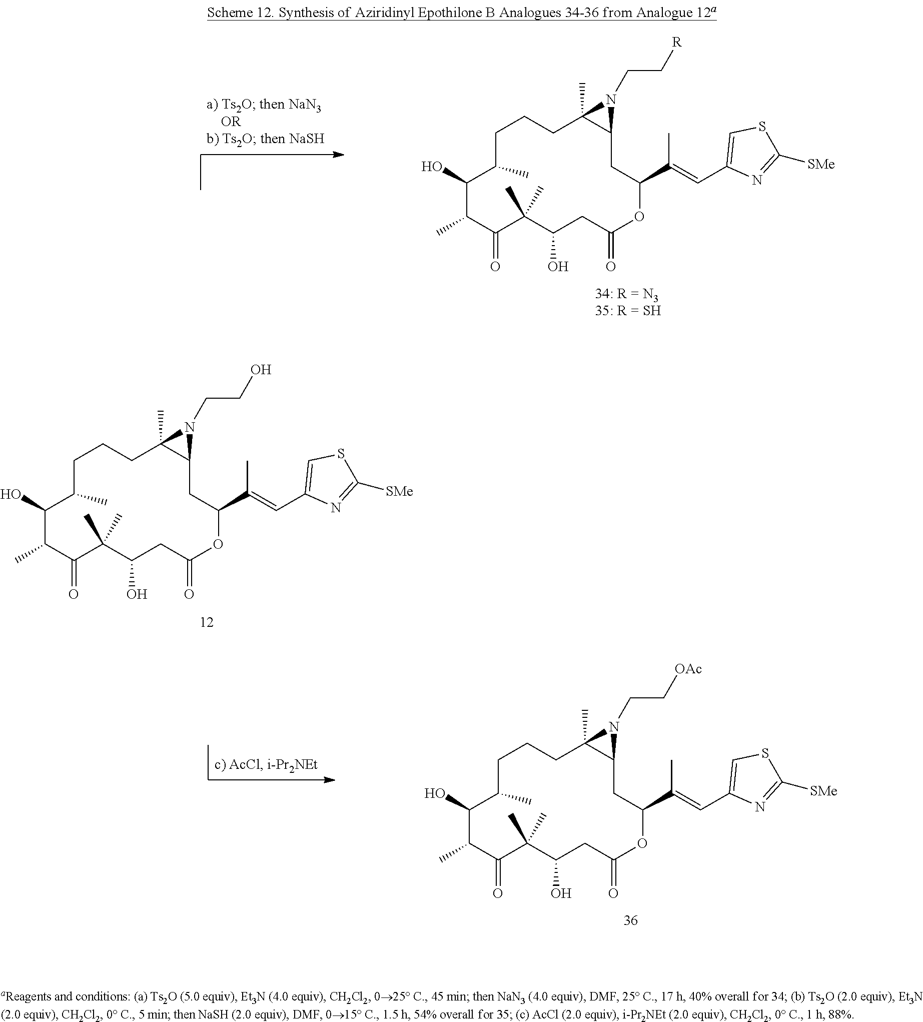

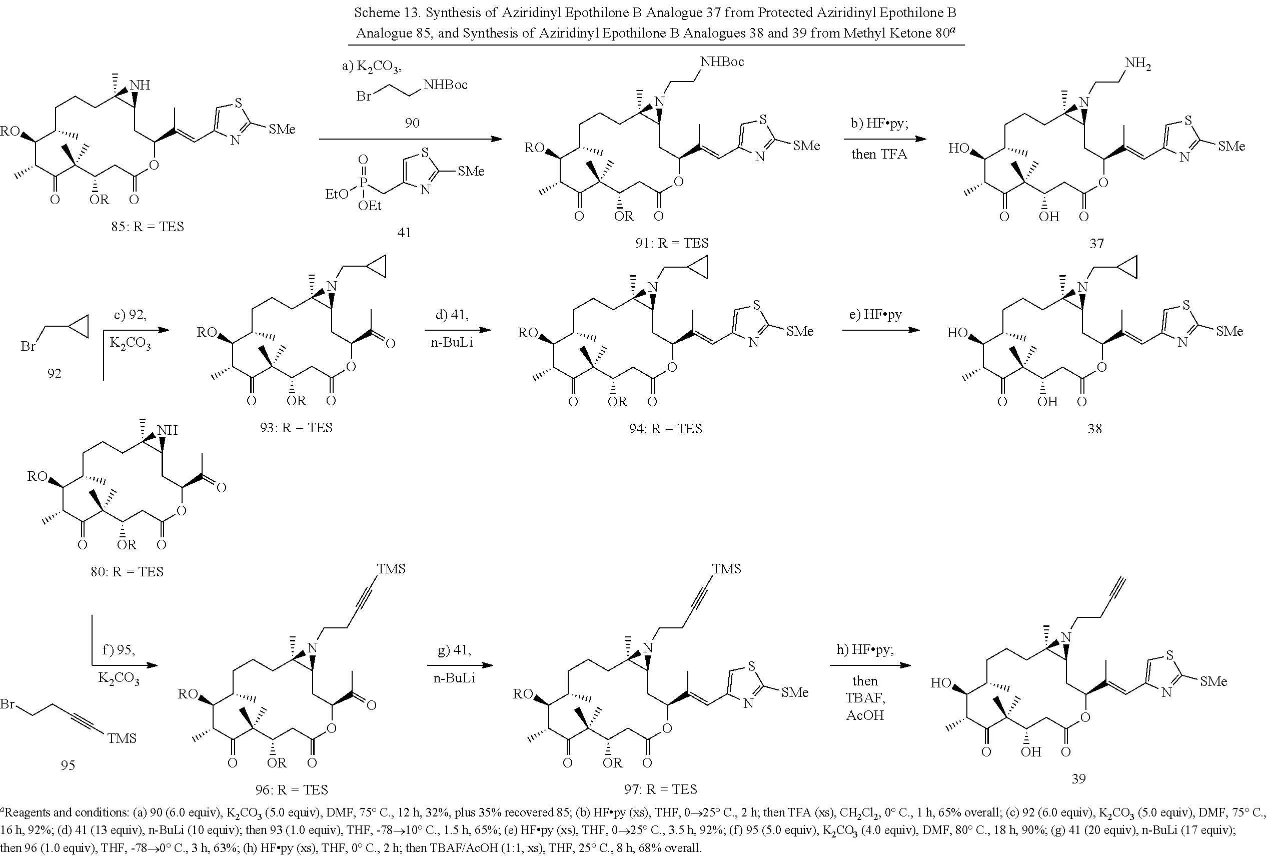

H. Synthesis of Designed N-Substituted Aziridinyl Epothilone B Analogues 34-39.

To further explore the SARs of this new library of epothilones, and in order to install a variety of potential conjugation sites, a series of analogues with different functional groups on the aziridine ring was synthesized, while maintaining a highly potent moiety in the side chain region (i.e., methylthiothiazole). The results of these endeavors are depicted in Schemes 12 and 13. Thus, and as shown in Scheme 12, N-azidoethyl (34), N-thioethyl (35), and N-acetoxyethyl (36) aziridinyl epothilone B analogues were prepared from N-hydroxyethyl aziridinyl epothilone 12 (prepared as described above, Scheme 7) through selective functionalization of the primary hydroxyl group [selective tosylation with Ts₂O followed by tosylate displacement with NaN₃ (34, 40% overall yield) or NaSH (35, 54% overall yield); and selective acetylation of 12 with AcCl, i-Pr₂NEt (36, 88% yield), see Scheme 12].

The syntheses of N-aminoethyl, N-cyclopropylmethyl, and N-homopropargylic aziridinyl epothilone B analogues 37-39 are summarized in Scheme 13. Thus, N-alkylation of bis-TES protected aziridinyl epothilone B analogue 85 (for its synthesis, see Scheme 8) with bromide 90 led to precursor 91 (K₂CO₃, 32% yield), whose global deprotection (HE·py; then TFA, 65% overall yield) afforded the targeted analogue 37 equipped with a primary amino group appropriate for conjugation to suitable delivery systems. The synthesis of cyclopropyl analogue 38 started with aziridinyl methyl ketone 80 and bromide 92, whose coupling under basic conditions (K₂CO₃, 92% yield) furnished intermediate methyl ketone 93. Olefination of the latter with phosphonate 41 (n-BuLi, 65% yield) then led exclusively to precursor 94 [(E):(Z)>98:2], whose desilylation with HF·py afforded the targeted analogue 38 in 92% yield. A similar pathway from the same precursor (i.e., aziridinyl methyl ketone 80), employing homopropargylic bromide 95 as the alkylating agent (K₂CO₃, 90% yield) provided methyl ketone intermediate 96. The latter, upon olefination with the same phosphonate (i.e., 41) under similar conditions, furnished TMS-protected acetylenic precursor 97 (63% yield) from which the desired analogue 39 was generated by global deprotection (HF·py; then TBAF/AcOH, 68% overall yield) as shown in Scheme 13.

I. Observation and Isolation of Stable β-Hydroxyphosphonate Adducts.

A feature of the HWE reaction observed in the course of these investigations was the formation of stable β-hydroxyphosphonate adducts with certain substrates (Schemes 14 and 15) (Petrova et al., 1990, Amer et al., 1991, Angelova et al., 1992, Petrova et al., 1992, Vassilev et al., 1993, Vassilev et al., 1994, Mizuno et al., 1998, Modro et al., 1998, Takaki et al., 2000, Tsuge et al., 1988 and Harusawa et al., 2002). Thus, and as shown in Scheme 14, treatment of methyl ketone substrates 82 and 87 with the carbanion generated from phosphonate 52 and n-BuLi provided β-hydroxyphosphonate adducts 98 and 99, respectively. These adducts were observed by thin layer chromatography and high resolution mass spectrometry (HRMS), but were not purified. Rather, the crude material was subsequently treated with t-BuOK to furnish protected analogues 100 (73% overall from 82, plus 10% recovered 82) and 101 (53% overall from 87, plus 28% recovered 87), respectively, with exclusive (E) geometry [(E):(Z)>98:2](The corresponding 2-fluoroethoxyphosphonate of compound 52 did not perform adequately in the HWE reaction, and only starting materials were recovered. This suggests that the success of 2-fluoroethoxyphosphonates in the HWE reaction between ketones and β-heteroaromatic phosphonates is highly dependent on the electronic nature of the heterocyclic moiety). This result is reminiscent of the work of Seyden-Penne and Bottin-Strzalko, who reported that the diethyl ester of phenylmethylphosphonic acid with benzaldehyde afforded only trans-stilbene (Deschamps et al., 1972 and Bottin-Strzalko & Seyden-Penne 1984). However, further experimentation showed that the base-induced elimination of β-hydroxyphosphonates derived from β-heteroaromatic phosphonates and methyl ketones does not always provide high (E) selectivity, as is discussed below.

Thus, and as shown in Scheme 15, treatment of phosphonate 50 with NaHMDS followed by transfer of the resulting carbanion into a solution of methyl ketone 82 delivered adduct 102 as an mixture of diastereoisomers (ca. 3:7), which proved chromatographically stable, allowing its isolation in pure form (but still as an inseparable diastereomeric mixture) in 37% yield. Subsequently, treatment of this mixture (102) with t-BuOK produced protected aziridinyl epothilone B analogue 103 (75% yield) as an inseparable mixture of geometrical isomers [(E)-103:(Z)-103 ca. 3:7, Scheme 15]. The conserved diastereomeric ratio (ca. 3:7) of 102 and 103 is in accordance with the classical mechanistic studies of β-ketophosphonates (Horner et al., 1958, Horner et al., 1959, Wadsworth et al., 1961, Wadsworth et al., 1965, Denmark & Dorow 1990, Zarges et al., 1991, Narasaka et al., 1993, Brandt et al., 1998, and Ando 1999) (i.e., the 3:7 dr for 102 corresponds to the 3:7 synlanti adduct distribution, see Scheme 16). Studies by Petrova and co-workers have defined this conserved ratio phenomenon as ‘chemical proof’ of the relative configurations of the two β-hydroxyphosphonate adducts (Petrova et al., 1990). This observation suggests that equilibration to the syn β-hydroxyphosphonate does not occur with this particular substrate (see Scheme 16), a result that mirrors previous studies from Tsuge and co-workers (Tsuge et al., 1988), who reported the reaction of β-furanylphosphonates with methyl ketones to provide olefins with geometrical ratios matching the diastereomeric ratio of the corresponding β-hydroxyphosphonate adducts. Interestingly, the major diastereoisomer of adduct 102 is presumably the anti isomer, a result that stands in contrast to previous reports from Petrova, who observed that the reaction of benzylphosphonates with aldehydes or ketones displayed marked syn selectivity (Petrova et al., 1990 and Petrova et al., 1992).

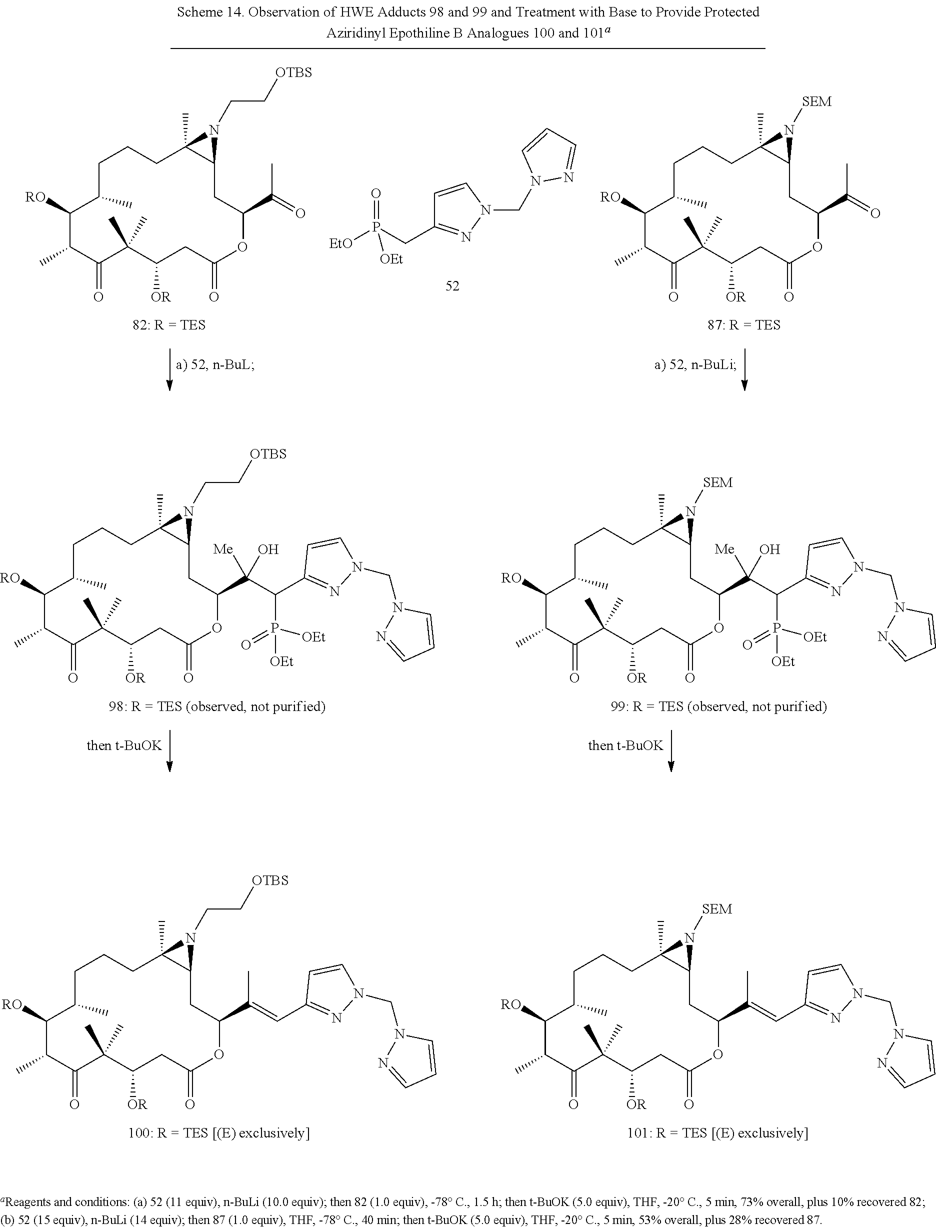

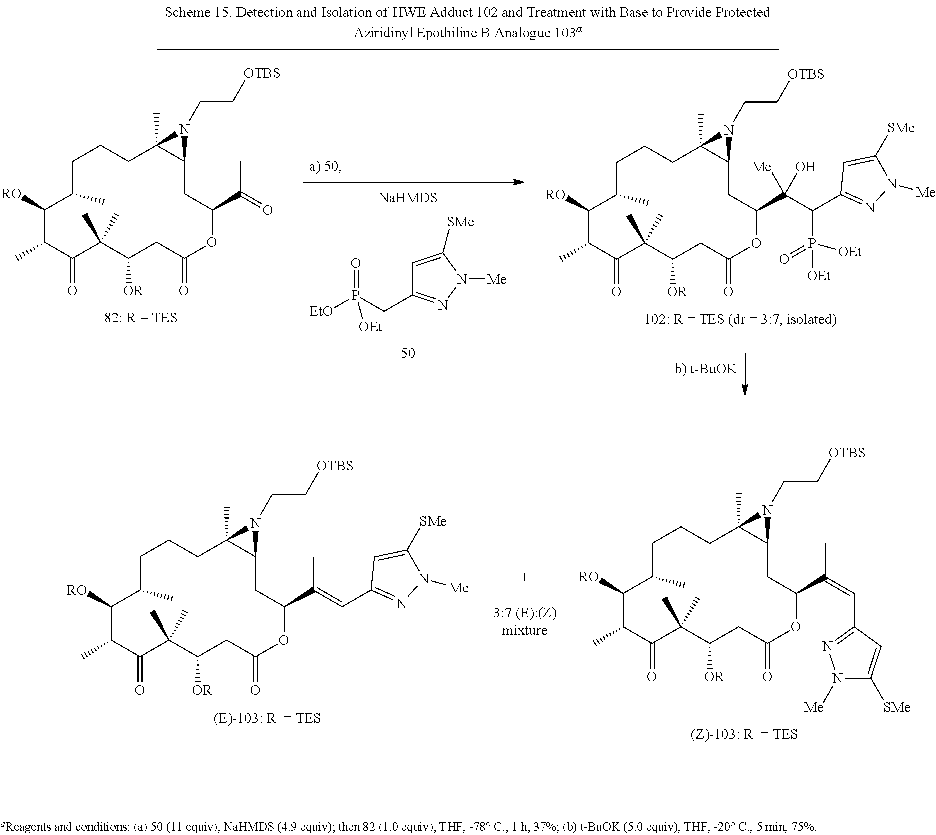

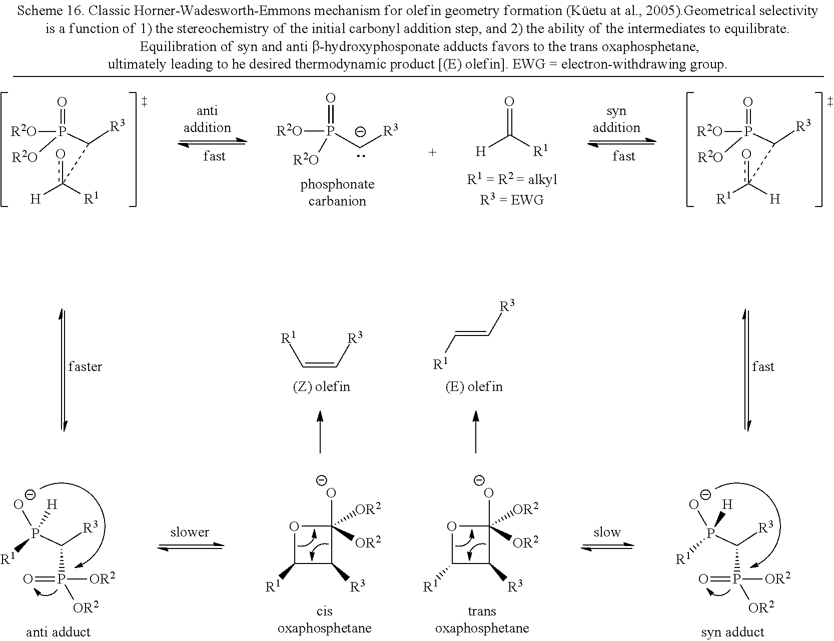

J. Ethoxy Group Substituent Effects on the HWE Reaction of 13-Heteroaromatic Phosphonates with Methyl Ketones 82 and 87.

As described above, the majority of the standard ethoxyphosphonates (i.e., 41-49, 51-57) performed well in their reactions with the corresponding methyl ketone epothilone precursors, both in terms of yield and geometrical selectivity. The lack of selectivity and room for yield improvement of the standard ethoxyphosphonate 50 employed for the synthesis of the coveted N-methyl-5-methylthiopyrazole analogue 23 (see Scheme 15) prompted investigation in search of a more stereoselective HWE reaction for this β-heteroaromatic phosphonate. It was suspected that the latter predicament was due to an unfavorable imbalance between the electron-donating nature of the electron-rich methylthiopyrazole moiety and the electron-withdrawing ability of the phosphonate group. To verify this hypothesis, phosphonate 50 was subjected to the action of a series of bases in different solvents, and its relative deprotonation was assessed by D₂O quenching of the resulting anion and subsequent ¹H NMR spectroscopic analysis. These results are listed in Table 1. The use of NaHMDS at 0 to 25° C., which has been suitable for the deprotonation of several phosphonates discussed above, was ineffective (25% D incorporation after 0.5 h), even at ambient temperature (Table 1, entry 1). More surprisingly, treatment of phosphonate 50 with t-BuLi or n-BuLi in a variety of solvents, in the absence or presence of HMPA (see Table 1, entries 2-4) at −78° C. led only to partial deprotonation, as evidenced from low deuterium incorporation (25-45%) in our experiments. In contrast, exposure of phosphonate 50 to Schlosser's base (n-BuLi/t-BuOK) (Schlosser 1988) in THF at −78° C. or KH in DMF at 25° C. led to 80% and 85% deuterium incorporation, respectively, indicating a high degree of deprotonation (Table 1, entries 5 and 6, respectively). However, these conditions proved too harsh for the methyl ketone substrate (i.e., 82), precluding the formation of the expected product due to substrate decomposition.

TABLE 1
Deuterium Labeling Studies of Phosphonate 50

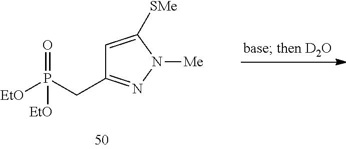 [SMILES: CCOP(=O)(Cc1cc(SC)n(C)n1)OCC]

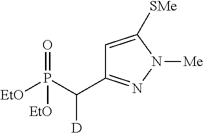 [SMILES: [2H]C(c1cc(SC)n(C)n1)P(=O)(OCC)OCC]

| entry | base | solvent | ϑ (° C.) | t (h) | % D^{c} |
|---|---|---|---|---|---|
| 1 | NaHMDS^{a} | THF | 0 → 25 | 0.5 | 25 |
| 2 | t-BuLi^{a} | Et₂O | −78 | 0.3 | 45 |
| 3 | n-BuLi^{a} | THF/HMPA | −78 | 2.5 | 30 |
| 4 | t-BuLi^{a} | hexanes/HMPA | −78 | 2 | 35 |
| 5 | n-BuLi/t-BuOK^{b} | THF | −78 | 0.3 | 80 |
| 6 | KH^{b} | THF | 25 | 2 | 85 |

^{a}Reactions were performed using 0.8 equiv of base.
^{b}Reactions were performed with excess base.
^{c}Approximate D incorporation, as determined by ¹H NMR spectroscopic analysis of crude reaction mixtures.

TABLE 2
Ethoxy Group Substituent Effects For the Synthesis of Protected Aziridinyl Epothilone
B Analogue 103

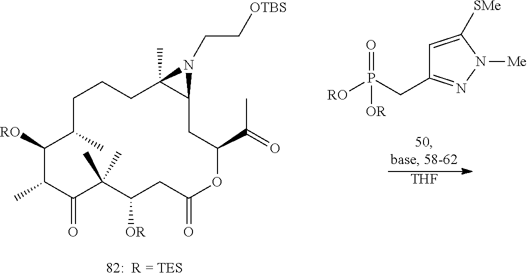

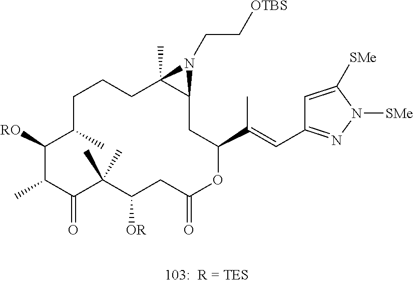

| | | % D^{a} | | | | [(S):(Z)] |
|---|---|---|---|---|---|---|
| entry | R | [t (h)] | base^{b} | ϑ (° C.) | t (h) | (yield) |
| 1 | CH₂CH₃ (50) | 25 (0.3) | n-BuLi | −78 | 0.2 | —^{c} |
| 2 | CH₂CH₃ (50) | 45 (0.5) | NaHMDS | −78 | 1 | —^{d} |
| 3 | CH₂CF₃ (58) | 70 (0.5) | n-BuLi | −78 → 25 | 2 | 1:1 (70%) |
| 4 | CH₂CH₂CF₃ (59) | 40 (0.3) | n-BuLi | −78 → 25 | 2 | —^{c} |
| 5 | CH₂CCl₃ (60) | 20 (0.4) | n-BuLi | −78 → 25 | 3 | —^{c} |
| 6 | CH₂CCl₃ (60) | 30 (1.5) | NaHMDS | −78 → 25 | 2.5 | —^{c} |
| 7 | CH₂CHF₂ (61) | 80 (0.3) | NaHMDS | −78 → 25 | 2 | 1:1 (52) |
| 8 | CH₂CH₂F (62) | 50 (0.5) | NaHMDS | −78 | 0.5 | —^{c} |

^{a}Approximate D incorporation as determined by ¹H NMR spectroscopic analysis of crude sample after quenching the phosphonate/base mixture after the designated time with D₂O. See Supporting Information for details.
^{b}Reactions were performed using 0.8 equiv base.
^{c}Unidentified mixture.
^{d}Formation of β-hydroxyphosphonate adducts (102, see Scheme 11).
^{e}Decomposition of phosphonate observed.

These failures (see Table 1 and Table 2, entries 1 and 2) prompted an exploration of an alternative avenue to activate the phosphonate (i.e., 50) by decreasing its pKₐ in order to enhance its reactivity. Reasoning that electron-withdrawing residues on the alkyl groups of the phosphonate moiety would accomplish this goal, we investigated a number of fluorinated and chlorinated derivatives, as shown in Table 2. After unsuccessful attempts with standard phosphonate 50, 2,2,2-trifluoroethoxyphosphonate 58 (adopted from the Still-Gennari method (Still-Gennari 1983)) was employed, which reacted with methyl ketone 82 in the presence of n-BuLi to afford the desired product (i.e., 103) in 70% yield, but as a 1:1 geometrical ratio of inseparable olefinic isomers at the newly formed trisubstituted olefinic bond (Table 2, entry 3). Despite its impact on organic synthesis, the traditional Still-Gennari stereoselective olefination reaction with β-heteroaromatic phosphonates has not been reported to our knowledge. Furthermore, a systematic exploration of the effect of various halogen substituents on diethoxyphosphonates in any context outside the original Still-Gennari protocol has not been undertaken to the best of our knowledge. As such, the observations with fluorinated phosphonate 58 and methyl ketone 82 (Table 2, entry 3) prompted investigation of the reactivities of additional phosphonates containing fluorine and chlorine substituents in search of a stereoselective olefination of methyl ketone substrates 82 and 87.

3,3,3-trifluoropropyloxy- and 2,2,2-trichloroethoxyphosphonates 59 and 60 were prepared but they did not perform well in their intended coupling with substrate 82, as shown in Table 2 (entries 4-6). The 2,2-difluorophosphonate 61 in combination with NaHMDS reacted smoothly with methyl ketone 82 at −78° C. to give the desired olefinic product (i.e., 103) in 52% yield, albeit as a mixture of geometrical isomers [(E):(Z) ca. 1:1, Table 2, entry 7], echoing the result with the 2,2,2-trifluoroethoxyphosphonate 58 (Table 2, entry 3). Lastly, 2-fluoroethoxyphosphonate 62 was employed (Table 2, entry 8). While its attempted reaction with NaHMDS and substrate 82 led to its decomposition (Table 2, entry 8), this phosphonate provided, upon further experimentation, a solution to the geometrical selectivity desired for aziridinyl epothilone B 103, as discussed below.

Scheme 17 (panel A) shows experiments that led to the isolation and identification of the intermediate β-hydroxyphosphonates (i.e., 104, inseparable mixture of diastereoisomers, dr ca. 3:7) formed from methyl ketone 87 and phosphonate 62 [synthesized in 78% overall yield from phosphonate 50 by stepwise treatment with TMSCl, (COCl)₂, 2-fluoropropanol, as summarized in Scheme 17]. Thus, reaction of 62 with n-BuLi at −78° C. for 10 min, followed by addition of the resulting carbanion into a solution of methyl ketone 87 and then quenching the reaction mixture after 1 h at −78° C. afforded adduct 104 in 28% yield (plus 42% recovered 87) as an inseparable mixture of diastereoisomers (dr ca. 3:7), which proved chromatographically stable. Exposure of this adduct (104) to t-BuOK delivered the desired protected epothilone analogue 105 in 63% yield and with exclusive (E) olefinic geometry [(E):(Z)>98:2]. Further refinement of these conditions led to a practical, one step generation of the olefinic product (105) from the reaction of methyl ketone 87 and phosphonate 62 as summarized in Scheme 17 (panel B). Thus, treatment of methyl ketone 87 with the carbanion generated from phosphonate 62 and n-BuLi at −78 to 0° C. provided, after global deprotection (TFA), aziridinyl epothilone B analogue 40 (52% overall yield) in a highly stereoselective fashion [(E):(Z) ca. 92:8]. Similarly, and as shown in Scheme 17 (panel B), methyl ketone 82 reacted (at −78 to −40° C.) with the anion of phosphonate 62, generated in the presence of n-BuLi (−78° C.) to afford precursor (E)-103 (62% yield), whose global desilylation with HF·py furnished the targeted aziridinyl epothilone B analogue (E)-23 in 82% yield. In these cases, and in contrast to the result in Scheme 17 (panel A; HWE reaction of 87 plus 62 quenched at −78° C.), the HWE coupling between methyl ketones 87 or 82 and phosphonate 62 was allowed to warm to 0° C. or −40° C. (Scheme 12, panel B), inducing the corresponding in situ formed β-hydroxyphosphonates (e.g., 104) to undergo elimination to provide directly protected aziridinyl epothilone B analogues 105 and (E)-103, respectively.

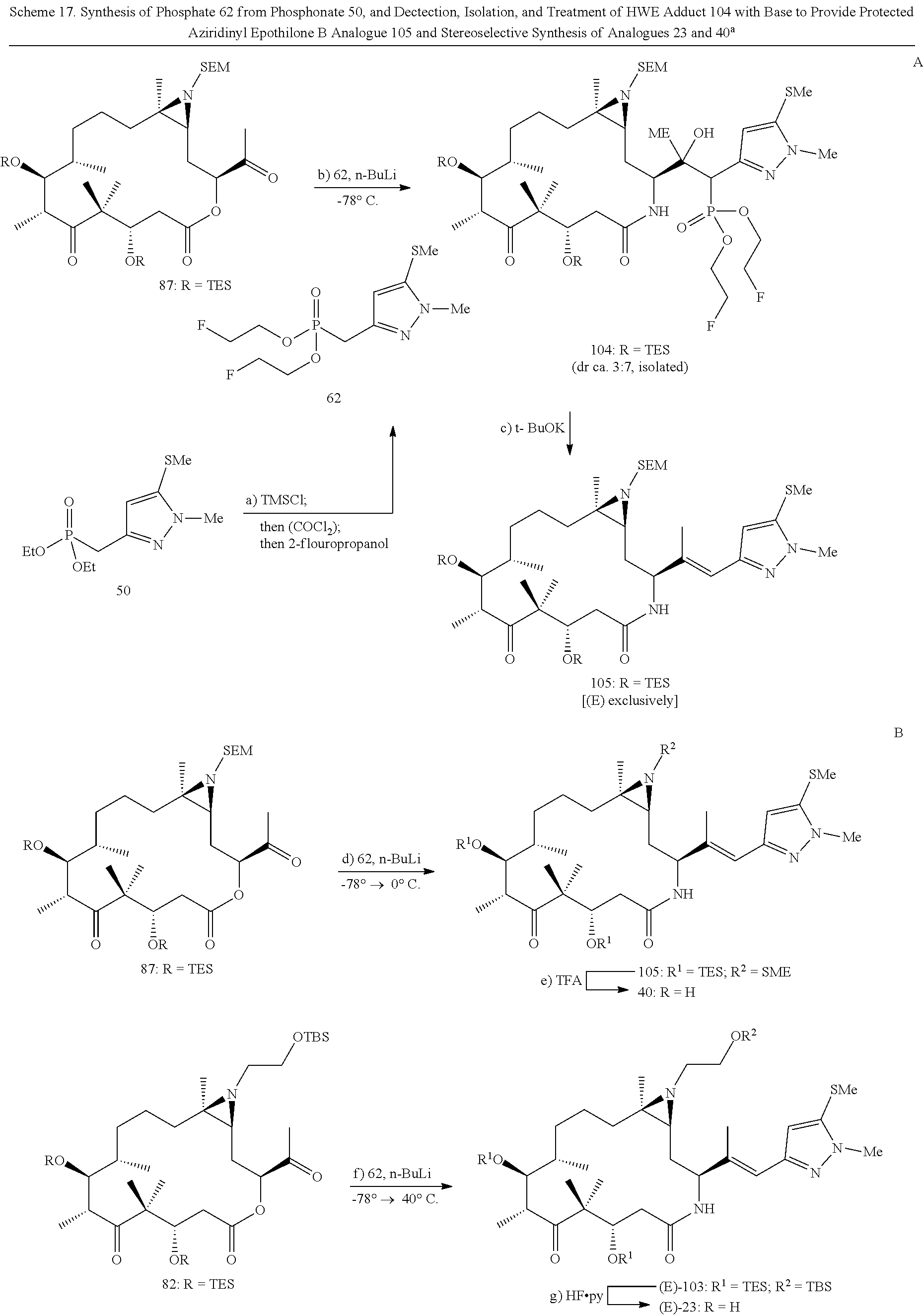

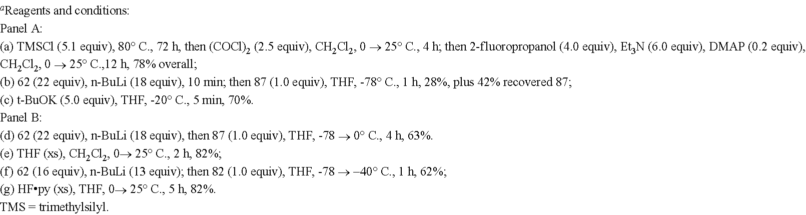

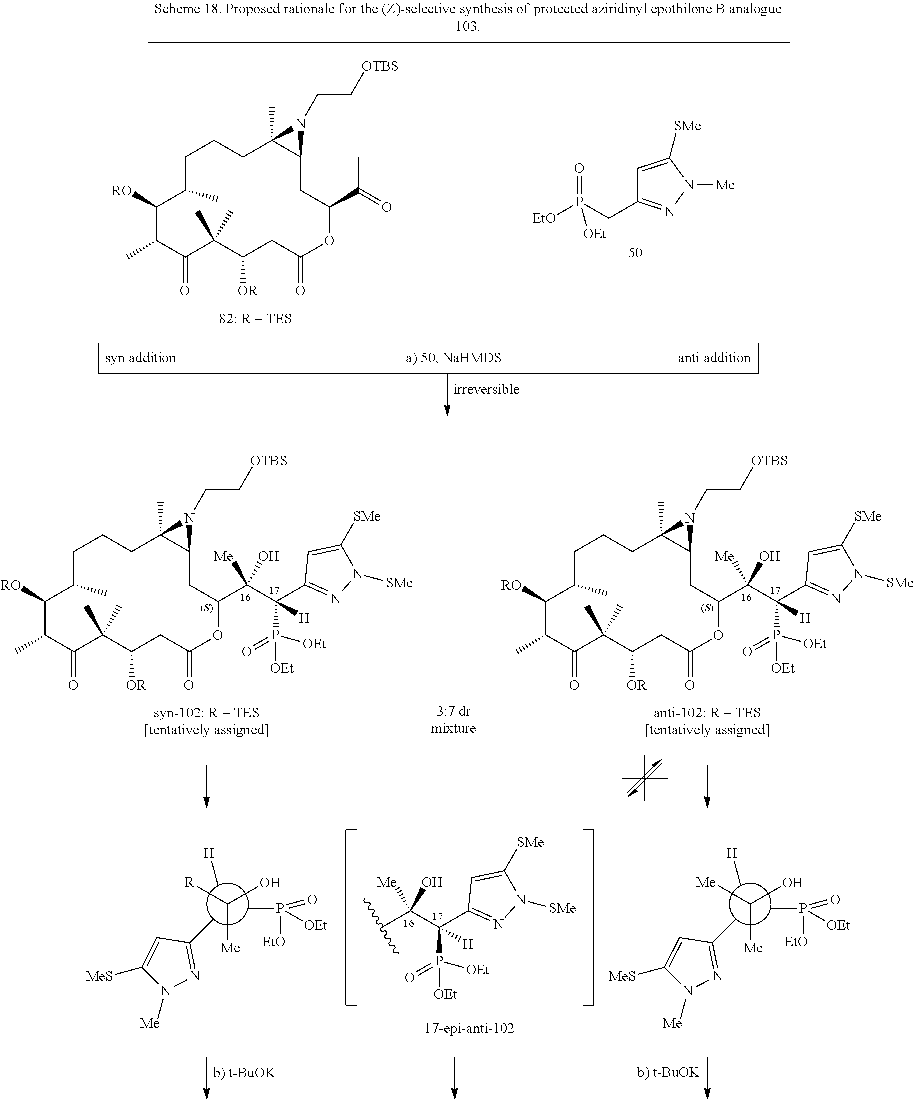

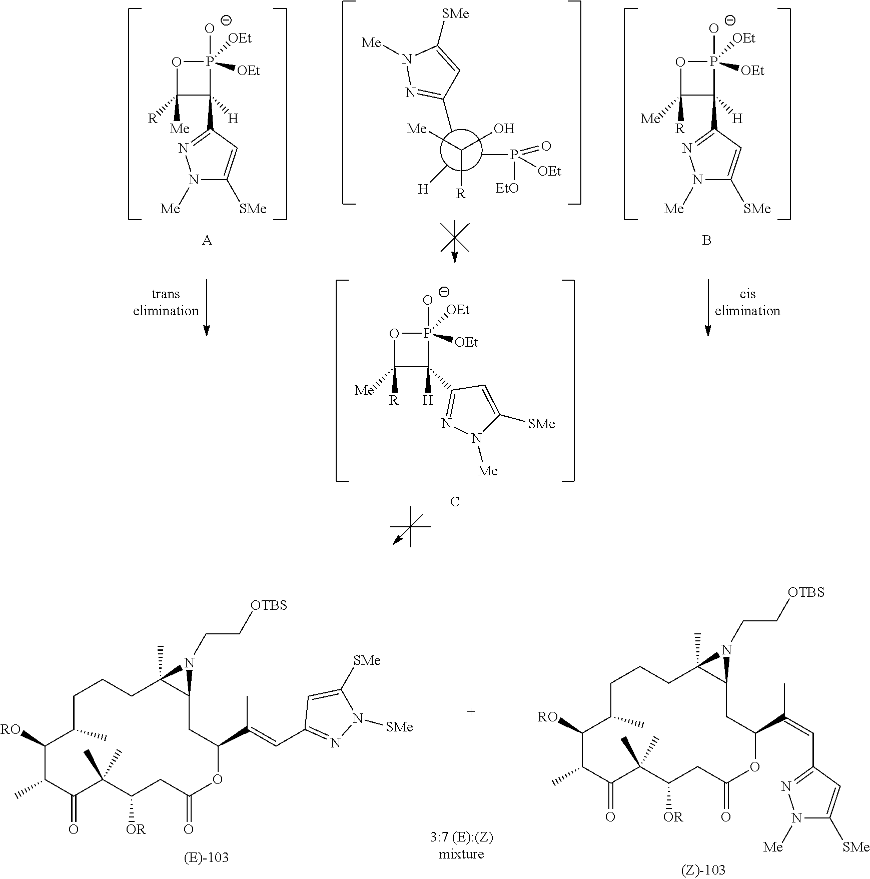

Collectively, the results shown in Scheme 17 demonstrate the thermodynamic control of this reaction, with higher temperatures favoring equilibration of the reaction intermediates and/or other thermodynamic factors (e.g., pKa effects), such as epimerization at the phosphorous-bearing carbon center, that promote (E) olefinic bond formation with high stereoselectivity [(E):(Z)>98:2]. Based on these results, mechanistic rationales for the different stereochemical outcomes of the reactions of phosphonates 50 [(E):(Z) ca. 3:7] and 62 [(E):(Z)>98:2] with methyl ketone substrates 82 and 87 (Schemes 18 and 19, respectively) are proposed. Thus, and as shown in Scheme 18, substrate 82 reacts with the carbanion generated from ethoxyphosphonate 50 (NaHMDS) to afford a mixture of syn and anti I3-hydroxyphosphonates (syn-102 and anti-102, dr ca. 3:7). As can be seen from their Newman projections, these dia stereomers are poised to form, under the influence of t-BuOK, the corresponding oxaphosphetane intermediates A and B, respectively, which collapse to the (E)- and (Z)-olefinic products through a trans and cis elimination, respectively (see Scheme 18), and with preservation of the initial diastereoselectivity [(E):(Z) ca. 3:7]. It is the stability of diastereoisomers syn-102 and anti-102 toward C17 epimerization under the reaction conditions and/or the resistance of these adducts to revert back to starting materials (i.e., 82 and 50) that accounts for their stereospecific conversion to the corresponding (E) and (Z) olefins. Had one or both of these events occurred [e.g., Scheme 18, anti-102 equilibrating with syn-102 (via reversal back to 82 and 50, respectively) or with 17-epi-anti-102 (via epimerization) and their conversion to olefin (E)-103], then the conversion of these β-hydroxyphosphonate adducts would have been disturbed.

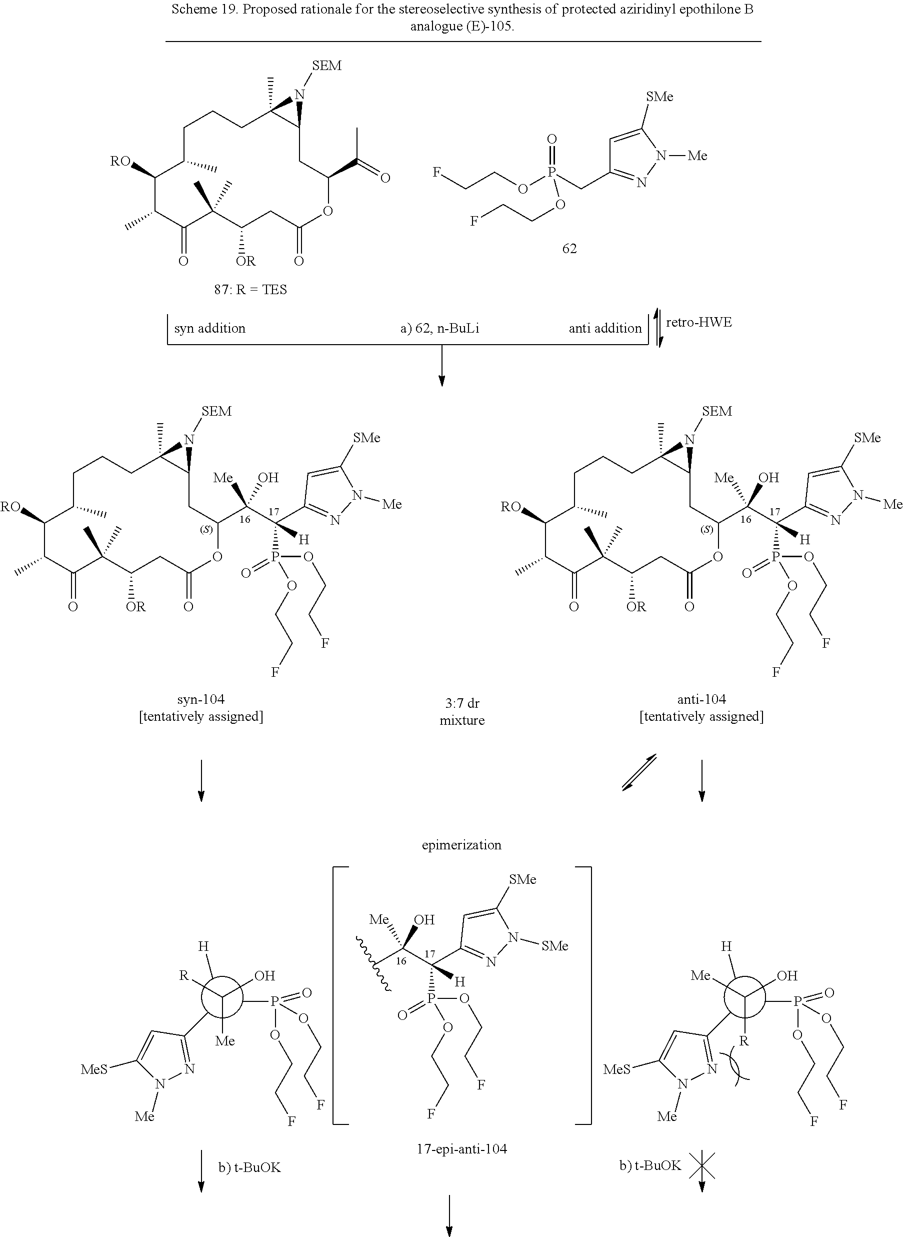

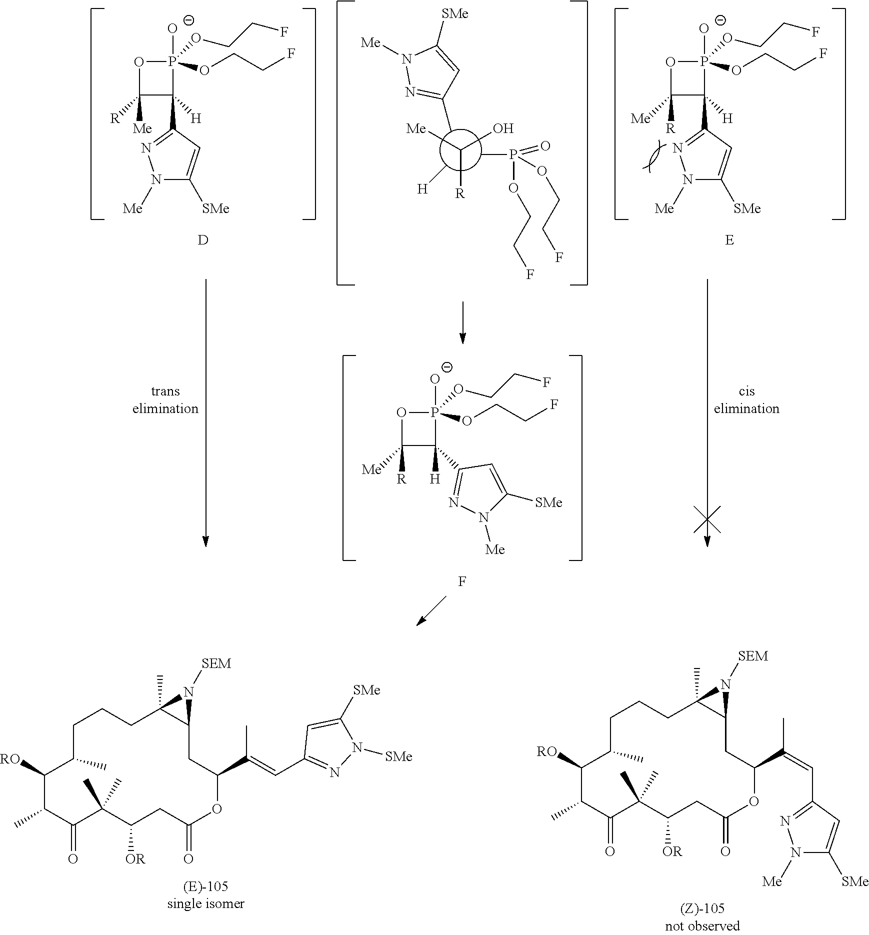

Similar to the ethoxyphosphonate 50, and as shown in Scheme 19, the bis(2-fluoroethoxy)phosphonate 62 also reacts with methyl ketone 87, in this instance under the influence of n-BuLi, to initially afford β-hydroxyphosphonates syn-104 and anti-104 in a similar ratio (dr ca. 3:7, isolated, structures tentatively assigned). To explain the exclusive conversion of this mixture of adducts, after further treatment with t-BuOK, to (E)-olefinic product (E)-105 it is envisioned that epimerization of the anti-diastereoisomer (anti-104) to 17-epi-anti-104 (see Newman projection) occurs, which finds its way to (E)-olefin (E)-105 via oxaphosphetane F, the latter undergoing trans elimination. It is noted that oxaphosphetane intermediates D and F (Scheme 19) represent enantiomeric transition states, thus providing the same desired (E) olefinic product upon trans elimination.

It is proposed that the facile equilibration of anti-104 and 17-epi-anti-104 is a consequence of the electron-withdrawing effect of the fluorine residues on phosphonate 62, which renders the 17H more acidic than its counterpart in the ethoxyphosphonate 50, which apparently does not undergo epimerization under similar conditions (see Scheme 18). However, a possibility that cannot be excluded is that these electron-withdrawing groups cause reversibility to occur (i.e., retro-HWE) in the system, thereby promoting the thermodynamically favorable formation of the (E) olefin (see Scheme 16).

This exploration resulted in a unique application of the HWE reaction of β-heteroaromatic 2-fluoroethoxyphosphonate 62 in the synthesis of olefinic heterocyclic compounds [e.g., (E)-103 and (E)-105]. Further applications of these types of phosphonates, including other β-hetereocyclic and β-ketophosphonates, are expected to find applications in synthetic organic and medicinal chemistry. While an exceptionally broad substrate scope was observed in terms of heterocyclic moieties throughout the investigations, it should be noted that phosphonates 63-68 proved recalcitrant, thus far, toward delivering the desired olefination products. Specifically, reaction of methyl ketone 82 with thiadiazolyl phosphonate 63 and oxadiazolyl phosphonate 64 produced an unidentifiable mixture of side products, while imidazolyl phosphonate 65 and N-arylpyrazolyl phosphonates 66-68 resulted only in recovered starting materials.

### Example 2 General Methods and Materials

All reactions were carried out under an argon atmosphere with dry solvents under anhydrous conditions, unless otherwise noted. Dry acetonitrile (MeCN), diethyl ether (Et₂O), dimethylformamide (DMF), methylene chloride (CH₂Cl₂), tetrahydrofuran (THF), triethylamine (Et₃N), and toluene were obtained by passing commercially available pre-dried, oxygen-free formulations through activated alumina columns. Yields refer to chromatographically and spectroscopically (′H NMR) homogeneous materials, unless otherwise stated. Reagents were purchased at the highest commercial quality and used without further purification, unless otherwise stated. Reactions were monitored by thin-layer chromatography (TLC) carried out on S-2 0.25 mm E. Merck silica gel plates (60F-254) using UV light as visualizing agent and an acidic aqueous solution of p-anisaldehyde, an aqueous solution of cerium sulfate, or a basic aqueous solution of potassium permanganate and heat as developing agents. E. Merck silica gel (60, particle size 0.040-0.063 mm) was used for flash column chromatography. NMR spectra were recorded on a Bruker DRX-600 instrument and calibrated using residual undeuterated solvent (CD₂Cl₂: δ_{H}=5.32 ppm, δ_{C}=53.84 ppm; CDCl₃: δ_{H}=7.26 ppm, δ_{C}=77.16 ppm; C₆D₆: δ_{H}=7.16 ppm, δ_{C}=128.06 ppm) as an internal reference. The following abbreviations were used to designate multiplicities: s=singlet, d=doublet, t=triplet, q=quartet, m=multiplet, qd=quartet of doublets, dd=doublet of doublets, ddd=doublet of doublet of doublets, dddd=doublet of doublet of doublet of doublets, dt=doublet of triplets, dq=doublet of quartets, ddq=doublet of doublet of quartets, br=broad. Infrared (IR) spectra were recorded on a PerkinElmer 100 FT-IR spectrometer. High-resolution mass spectra (HRMS) were recorded on an Agilent ESI-TOF (time of flight) mass spectrometer using MALDI (matrix-assisted laser desorption ionization) or ESI (electrospray ionization). Optical rotations were recorded on a POLARTRONIC M100 polarimeter at 589 nm, and are reported in units of 10⁻¹ (deg cm² g⁻¹).

### Example 3 Compound Characterization

### (1S,3S,7S,10R,11S,12S,16R)-7,11-Dihydroxy-8,8,10,12-tetramethyl-3-[(1E)-1-(2-methyl-1,3-thiazol-4-yl)prop-1-en-2-yl]-4-oxa-17-azabicyclo[14.1.0]heptadecane-5,9-dione (8)

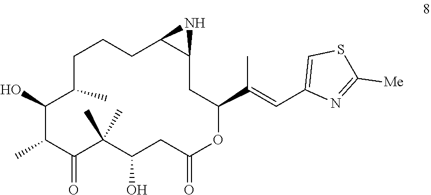

To a stirred solution of epothilone C (3; 50 mg, 0.11 mmol, 1.0 equiv) in 2,2,2-trifluoroethanol (1.1 mL) at 25° C. was added O-(2,4-dinitrophenyl)hydroxylamine (23 mg, 0.12 mmol, 1.1 equiv), followed by bis[rhodium(α,α,α′,α′,-tetramethyl-1,3-benzenedipropionic acid)] (4.0 mg, 5.3 μmot, 0.05 equiv). After 4 h, the reaction mixture was diluted with ethyl acetate (5 mL), and washed with a saturated aqueous solution of sodium bicarbonate (10 mL). The two phases were separated, and the aqueous layer was extracted with ethyl acetate (3×3 mL). The combined organic layers were dried with anhydrous sodium sulfate and concentrated under reduced pressure. The obtained residue was purified by flash column chromatography (silica gel, 4→11% methanol in dichloromethane) to afford pure epothilone 8 as a white amorphous solid (38 mg, 77 μmot, 70% yield). 8: R_{f}=0.22 (silica gel, 7% methanol in dichloromethane); [α]_{D}²²=−45.7 (c=1.4, CHCl₃); FT-IR (film) vₘₐₓ 3437, 3312, 2929, 2874, 1731, 1688, 1507, 1466, 1453, 1368, 1344, 1296, 1254, 1176, 1144, 1085, 1040, 1009, 978, 934, 911, 883, 855, 833, 731, 674, 647, 608 cm⁻¹; ¹H NMR (600 MHz, CDCl₃) δ=6.97 (s, 1H), 6.66 (s, 1H), 5.59 (m, 1H), 4.18 (dd, J=10.7, 3.9 Hz, 1H), 3.82 (dd, J=6.7, 3.6 Hz, 1H), 3.30 (qd, J=6.9, 6.9 Hz, 1H), 2.71 (s, 3H), 2.53 (dd, J=12.8, 10.8 Hz, 1H), 2.44 (dd, J=12.8, 3.9 Hz, 1H), 2.06 (s, 3H), 2.06-2.04 (m, 1H), 1.94-1.90 (m, 2H), 1.85-1.73 (m, 2H), 1.61-1.54 (m, 2H), 1.52-1.46 (m, 2H), 1.40 (s, 3H), 1.33-1.22 (m, 3H), 1.13 (d, J=6.9 Hz, 3H), 1.04 (s, 3H), 0.94 (d, J=7.0 Hz, 3H) ppm; ¹³C NMR (151 MHz, CDCl₃) δ=220.1, 171.0, 164.9, 152.2, 136.5, 119.0, 115.7, 76.3, 75.8, 75.3, 52.3, 44.7, 37.9, 34.5, 34.0, 29.9, 29.7, 27.8, 25.8, 24.6, 22.7, 19.1, 18.9, 17.3, 16.2, 15.0 ppm; HRMS (ESI) calcd for C₂₆H₄₁N₂O₅S⁺ [M+H]⁺ 493.2731, found 493.2723.

### (1S,3S,7S,10R,11S,12S,16R)-7,11-Dihydroxy-17-(2-hydroxyethyl)-8,8,10,12-tetramethyl-3-[(1E)-1-(2-methyl-1,3-thiazol-4-yl)prop-1-en-2-yl]-4-oxa-17-azabicyclo[14.1.0]heptadecane-5,9-dione (9)

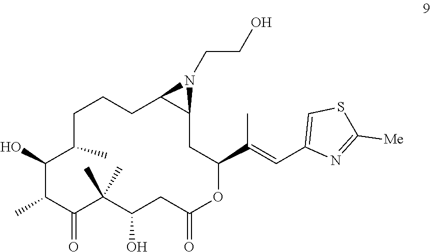

To a stirred solution of epothilone 8 (12 mg, 24 μmol, 1.0 equiv) in dimethylformamide (0.2 mL) at 25° C. was added 2-bromoethanol 42 (8.5 μL 0.12 mmol, 5.0 equiv), followed by potassium carbonate (7.2 mg, 52 μmol, 6.0 equiv). The reaction mixture was heated to 50° C. for 48 h, and then allowed to cool to 25° C. The reaction mixture was diluted with ethyl acetate (2.5 mL) and washed with water (2.5 mL). The two phases were separated, and the aqueous layer was extracted with ethyl acetate (3×1 mL). The combined organic layers were backwashed with brine (2 mL), dried with anhydrous magnesium sulfate, and concentrated under reduced pressure. The obtained residue was purified by flash column chromatography (silica gel, 12% methanol in dichloromethane) to afford pure epothilone 9 as a white amorphous solid (12 mg, 23 μmol, 97% yield). 9: R_{f}=0.18 (silica gel, 7% methanol in dichloromethane); [α]_{D}²²=−54.7 (c=1.0, CHCl₃); FT-IR (film) vₘₐₓ 3380, 2934, 2876, 1731, 1688, 1507, 1465, 1371, 1292, 1256, 1189, 1151, 1055, 1007, 980, 912, 731, 645 cm⁻¹; ¹H NMR (600 MHz, CDCl₃) δ=6.97 (s, 1H), 6.60 (s, 1H), 5.54 (m, 1H), 4.00 (dd, J=10.3, 1.4 Hz, 1H), 3.81 (dd, J=7.4, 2.8 Hz, 1H), 3.78-3.70 (m, 2H), 3.28 (qd, J=7.1, 7.1 Hz, 1H), 2.81-2.79 (m, 1H), 2.71 (s, 3H), 2.52 (dd, J=13.4, 10.4 Hz, 1H), 2.43 (dd, J=13.3, 1.8 Hz, 1H), 2.21-2.18 (m, 1H), 2.11 (s, 3H), 2.09-2.00 (m, 2H), 1.75-1.67 (m, 2H), 1.57-1.50 (m, 5H), 1.36-1.34 (m, 2H), 1.29-1.22 (m, 2H), 1.15 (d, J=6.9 Hz, 3H), 1.06 (s, 3H), 0.97 (d, J=7.0 Hz, 3H) ppm; ¹³C NMR (151 MHz, CDCl₃) δ=220.1, 171.2, 165.0, 152.2, 135.9, 119.0, 116.2, 76.4, 75.7, 63.4, 61.6, 52.1, 45.4, 44.9, 39.3, 38.7, 34.1, 30.3, 29.7, 27.5, 25.4, 24.7, 22.2, 19.2, 18.8, 17.6, 16.0, 15.1 ppm; HRMS (ESI) calcd for C₂₅H₄₄N₂O₆SNa⁺ [M+Na]⁺ 559.2812, found 559.2816.

### (1S,3S,7S,10R,11S,12S,16R)-7,11-Dihydroxy-8,8,10,12,16-pentamethyl-3-[(1E)-1-(2-methyl-1,3-thiazol-4-yl)prop-1-en-2-yl]-4-oxa-17-azabicyclo[14.1.0]heptadecane-5,9-dione (10)

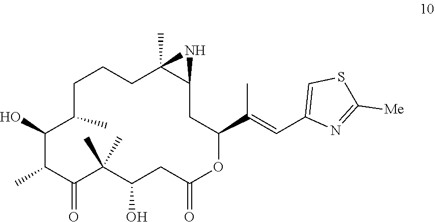

To a stirred solution of epothilone D (4; 50 mg, 0.10 mmol, 1.0 equiv) in 2,2,2-trifluoroethanol (1.1 mL) at 25° C. was added O-(2,4-dinitrophenyl)hydroxylamine (23 mg, 0.12 mmol, 1.1 equiv), followed by bis[rhodium(α,α,α′,α′,-tetramethyl-1,3-benzenedipropionic acid)] (4 mg, 5.3 μmol, 0.05 equiv). After 4 h, the reaction mixture was diluted with ethyl acetate (5 mL) and washed with a saturated aqueous solution of sodium bicarbonate (10 mL). The two phases were separated, and the aqueous layer was extracted with ethyl acetate (3×3 mL). The combined organic layers were dried with anhydrous sodium sulfate and concentrated under reduced pressure. The obtained residue was purified by flash column chromatography (silica gel, 4→11% methanol in dichloromethane) to afford pure epothilone 10 as a white amorphous solid (33 mg, 66 nmol, 66% yield). 10: R_{f}=0.24 (silica gel, 7% methanol in dichloromethane); [α]_{D}²²=−35.5 (c=0.60, CHCl₃); FT-IR (film) vₘₐₓ 3294, 2958, 2930, 2876, 1730, 1687, 1598, 1557, 1503, 1452, 1383, 1292, 1256, 1179, 1148, 1042, 1009, 980, 915, 882, 834, 731, 669, 648 cm⁻¹; ¹H NMR (600 MHz, CDCl₃) δ=6.98 (s, 1H), 6.63 (s, 1H), 5.54 (dd, J=3.9, 3.9 Hz, 1H), 4.15 (ddd, J=10.5, 3.5, 3.5 Hz, 1H), 3.80 (dd, J=5.3, 4.2 Hz, 1H), 3.35 (dq, J=6.5, 6.5 Hz, 1H), 2.71 (s, 3H), 2.52 (dd, J=12.8, 10.6 Hz, 1H), 2.42 (dd, J=12.9, 3.5 Hz, 1H), 2.07 (s, 3H), 2.06 (s, 1H), 1.96-1.76 (m, 4H), 1.52-1.42 (m, 5H), 1.39 (s, 3H), 1.29-1.25 (m, 3H), 1.24 (s, 3H), 1.13 (d, J=6.9 Hz, 3H), 1.04 (s, 3H), 0.97 (d, J=6.9 Hz, 3H) ppm; ¹³C NMR (151 MHz, CDCl₃) δ=220.8, 171.1, 165.1, 152.3, 136.7, 119.2, 116.0, 76.4, 75.8, 74.9, 52.6, 44.5, 39.1, 38.5, 35.4, 31.4, 30.4, 29.9, 29.4, 25.5, 22.3, 22.2, 19.5, 19.3, 17.6, 16.3, 14.7 ppm; HRMS (ESI) calcd for C₂₇H₁₃N₂O₅S⁺ [M+H]⁺ 507.2887, found 507.2903.

### (1S,3S,7S,10R,11S,12S,16R)-7,11-Dihydroxy-17-(2-hydroxyethyl)-8,8,10,12,16-pentamethyl-3-[(1E)-1-(2-methyl-1,3-thiazol-4-yl)prop-1-en-2-yl]-4-oxa-17-azabicyclo[14.1.0]heptadecane-5,9-dione (11)

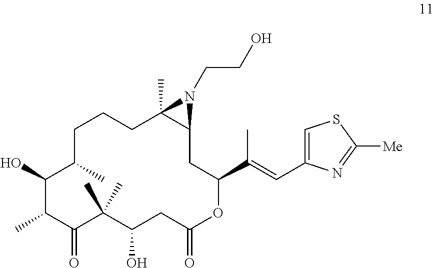

To a stirred solution of epothilone 10 (15 mg, 30 nmol, 1.0 equiv) in dimethylformamide (0.8 mL) at 25° C. was added 2-bromoethanol (11 μL 0.15 mmol, 5.0 equiv), followed by potassium carbonate (22 mg, 0.16 mmol, 6.0 equiv). The reaction mixture was heated to 50° C. for 48 h, and then allowed to cool to 25° C. The reaction mixture was diluted with ethyl acetate (2.5 mL) and washed with water (2.5 mL). The two phases were separated, and the aqueous layer was extracted with ethyl acetate (3×1 mL). The combined organic layers were backwashed with brine (2 mL), dried with anhydrous magnesium sulfate, and concentrated under reduced pressure. The obtained residue was purified by flash column chromatography (silica gel, 12% methanol in dichloromethane) to afford pure epothilone 11 as a white amorphous solid (15 mg, 29 nmol, 95% yield). 11: R_{f}=0.19 (silica gel, 7% methanol in dichloromethane); [α]_{D}²²=−42.3 (c=1.0, CHCl₃); FT-IR (film) vₘₐₓ 3369, 2929, 1730, 1685, 1465, 1374, 1263, 1152, 1053, 1009, 980, 882 em⁻¹; ¹H NMR (600 MHz, CDCl₃) δ=6.97 (s, 1H), 6.60 (s, 1H), 5.54 (dd, J=3.9, 3.9 Hz, 1H), 4.00 (dd, J=10.5, 3.5 Hz, 1H), 3.81 (dd, J=5.3, 4.2 Hz, 1H), 3.78-3.70 (m, 2H), 3.28 (dq, J=6.5, 6.5 Hz, 1H), 2.81-2.79 (m, 1H), 2.71 (s, 3H), 2.52 (dd, J=12.8, 10.6 Hz, 1H), 2.43 (dd, J=12.9, 3.5 Hz, 1H), 2.21-2.18 (m, 1H), 2.11 (s, 3H), 2.09-2.00 (m, 2H), 1.75-1.67 (m, 2H), 1.57-1.50 (m, 5H), 1.36 (s, 3H), 1.34-1.25 (m, 3H), 1.24 (s, 3H), 1.15 (d, J=6.9 Hz, 3H), 1.06 (s, 3H), 0.97 (d, J=6.9 Hz, 3H) ppm; ¹³C NMR (151 MHz, CDCl₃) δ=220.2, 171.4, 165.1, 152.4, 136.0, 119.1, 116.4, 76.6, 75.9, 63.5, 61.8, 52.2, 45.6, 45.1, 39.4, 38.9, 34.3, 30.4, 29.9, 27.7, 25.5, 24.8, 22.4, 19.4, 18.9, 17.8, 16.1, 15.2 ppm; HRMS (ESI) calcd for C₂₉H₄₆N₂O₆SNa⁺ [M+Na]⁺ 573.2974, found 573.2982.

### (4S,7R,8S,9S,13E,16S)-4,8-Dihydroxy-13-(iodomethyl)-16-[(1E)-1-iodoprop-1-en-2-yl]-5,5,7,9-tetramethyloxacyclohexadec-13-ene-2,6-dione (70)

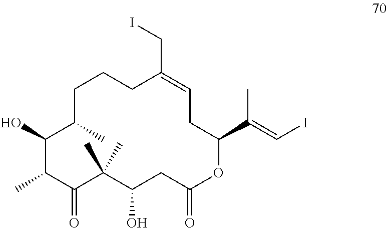

To a stirred solution of allylic alcohol 69 (9.6 mg, 18 μmol, 1.0 equiv) in dichloromethane (0.5 mL) at 0° C. was added triethylamine (13 μL, 90 μmol, 5.0 equiv), followed by p-toluenesulfonic anhydride (18 mg, 54 μmol, 3.0 equiv) and 4-dimethylaminopyridine (2.2 mg, 18 μmol, 1.0 equiv). After 20 min, the reaction mixture was diluted with dry acetone (3 mL), and concentrated under reduced pressure until ca. 1.5 mL of solvent remained. Then n-tetrabutylammonium iodide (33 mg, 90 μmol, 5.0 equiv) was added to the reaction mixture with stirring at 0° C. After 20 min, the reaction mixture was quenched with water (10 mL), and allowed to warm to 25° C. The mixture was extracted with ethyl acetate (3×10 mL), and the combined organic layers were dried with anhydrous sodium sulfate and concentrated under reduced pressure. The obtained residue was purified by flash column chromatography (silica gel, 10→30% ethyl acetate in hexanes) to afford pure bis-iodide 70 (10 mg, 15 μmol, 88% yield) as a colorless oil. 70: R_{f}=0.24 (silica gel, 25% ethyl acetate in hexanes); [α]_{D}²²=−28.2 (c=0.39, CH₂Cl₂); FT-IR (neat) vₘₐₓ 3488, 2924, 2854, 1735, 1686, 1464, 1378, 1259, 1147, 1046, 1007, 977, 884, 792, 737, 688, 668 cm⁻¹; ¹H NMR (600 MHz, C₆D₆) δ=6.44 (s, 1H), 5.17 (dd, J=9.6, 5.4 Hz, 1H), 5.12 (dd, J=9.6, 2.4 Hz, 1H), 3.85 (ddd, J=10.8, 7.8, 3.0 Hz, 1H), 3.63 (d, J=9.0 Hz, 1H), 3.57 (q, J=3.6 Hz, 1H), 3.54 (d, J=9.0 Hz, 1H), 2.76 (qd, J=6.0, 3.0 Hz, 1H), 2.68 (br s, 1H), 2.22 (ddd, J=13.2, 6.6, 6.6 Hz, 1H), 2.18-2.11 (m, 2H), 2.07 (ddd, J=13.2, 6.6, 6.6 Hz, 1H), 1.95 (dd, J=14.4, 3.0 Hz, 1H), 1.69 (s, 3H), 1.60 (d, J=7.2 Hz, 1H), 1.52 (dd, J=14.4, 4.8 Hz, 1H), 1.44-1.28 (m, 2H), 1.02 (d, J=6.6 Hz, 3H), 1.01-0.84 (m, 2H), 0.91 (d, J=6.6 Hz, 3H), 0.80 (s, 3H), 0.78 (s, 3H) ppm; ¹³C NMR (151 MHz, C₆D₆) δ=219.1, 169.2, 145.6, 140.2, 125.0, 80.4, 76.7, 74.3, 72.8, 53.1, 42.1, 39.5, 38.5, 32.4, 31.6, 28.6, 25.5, 22.3, 21.0, 18.5, 15.9, 13.9, 12.2 ppm; HRMS (ESI) calcd for C₂₃H₃₆I₂O₅Na⁺ [M+Na]⁺ 669.0544, found 669.0570.

### (4S,7R,8S,9S,13Z,16S)-4,8-Dihydroxy-16-[(1E)-1-iodoprop-1-en-2-yl]-5,5,7,9,13-pentamethyl-oxacyclohexadec-13-ene-2,6-dione (71)

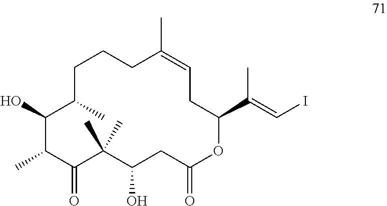

To a stirred solution of bis-iodide 70 (10.0 mg, 15.5 μmol, 1.0 equiv) in 1,3-dimethyl-3,4,5,6-tetrahydro-2(1H)-pyrimidinone (0.5 mL) at 25° C. was added sodium cyanoborohydride (12.4 mg, 0.186 mmol, 12 equiv). After 40 min, the reaction mixture was quenched with water (5 mL), diluted with ethyl acetate (50 mL), and washed with brine (3×10 mL). The organic layer was dried with anhydrous sodium sulfate and concentrated under reduced pressure. The obtained residue was purified by flash column chromatography (silica gel, 10→30% ethyl acetate in hexanes) to afford pure vinyl iodide 71 (6.5 mg, 12 μmol, 80% yield) as a colorless oil. 71: R_{f}=0.36 (silica gel, 30% ethyl acetate in hexanes); [α]_{D}²²=−27.6 (c=0.38, CH₂Cl₂); FT-IR (neat) vₘₐₓ 3478, 2924, 2853, 1732, 1686, 1463, 1377, 1261, 1146, 1007, 976, 741, 614 cm⁻¹; ¹H NMR (600 MHz, C₆D₆) δ=6.46 (s, 1H), 5.26 (dd, J=9.6, 2.4 Hz, 1H), 4.99 (dd, J=9.0, 5.4 Hz, 1H), 3.91 (ddd, J=10.8, 7.8, 3.0 Hz, 1H), 3.63 (q, J=3.0 Hz, 1H), 2.82 (qd, J=6.6, 3.0 Hz, 1H), 3.71 (br s, 1H), 2.40 (ddd, J=15.6, 10.2, 10.2 Hz, 1H), 2.20 (dd, J=15.6, 10.8 Hz, 1H), 2.18-2.12 (m, 1H), 1.99 (dd, J=15.6, 3.0 Hz, 1H), 1.82 (dd, J=15.6, 4.2 Hz, 1H), 1.76 (s, 3H), 1.74-1.69 (m, 1H), 1.65 (d, J=7.8 Hz, 1H), 1.56 (s, 3H), 1.44-1.28 (m, 2H), 1.14-0.80 (m, 2H), 1.05 (d, J=6.6 Hz, 3H), 0.98 (d, J=6.6 Hz, 3H), 0.81 (s, 3H). 0.80 (s, 3H) ppm; ¹³C NMR (151 MHz, C₆D₆) δ=219.1, 169.4, 146.2, 138.7, 120.6, 80.1, 77.6, 74.5, 72.8, 53.1, 42.2, 39.6, 38.8, 32.1, 31.8, 31.7, 25.9, 23.0, 22.3, 21.0, 18.5, 16.0, 14.0 ppm; HRMS (ESI) calcd for C₂₃H₃₇IO₅Na⁺ [M+Na]⁺ 543.1578, found 543.1562.

### (4S,7R,8S,9S,13Z,16S)-4,8-Dihydroxy-5,5,7,9,13-pentamethyl-16-{(1E)-1-[1-methyl-5-(methylsulfanyl)-1H-pyrazol-3-yl]prop-1-en-2-yl}oxacyclohexadec-13-ene-2,6-dione (73)

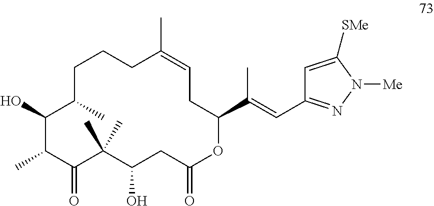

To a stirred suspension of tris(dibenzylideneacetone)dipalladium (37.8 mg, 41.3 μmol, 0.5 equiv), copper iodide (31.4 mg, 165 μmol, 2.0 equiv), and triphenylarsine (25.3 mg, 82.6 μmol, 1.0 equiv) in dimethylformamide (0.5 mL) at 0° C. was added a solution of vinyl iodide 71 (43.0 mg, 82.6 μmol, 1.0 equiv) and pyrazolyl stannane 74 (88.0 mg, 211 μmol, 2.5 equiv) in dimethylformamide (0.2 mL) via cannula, and the original flask was rinsed thoroughly with dimethylformamide (3×0.2 mL). After 1 h, the reaction mixture was diluted with ethyl acetate (5 mL), filtered through a short pad of Celite®, and rinsed thoroughly with ethyl acetate (15 mL). The filtrate was then washed with water (5 mL) and brine (5 mL), and the two phases were separated. The aqueous layer was extracted with ethyl acetate (3×5 mL), and the combined organic layers were dried with anhydrous sodium sulfate and concentrated under reduced pressure. The obtained residue was purified by flash column chromatography (silica gel, 5→50% ethyl acetate in hexanes) to afford pure epothilone 73 (29.0 mg, 55.7 μmol, 67% yield) as a white foam. 73: R_{f}=0.38 (silica gel, 50% ethyl acetate in hexanes); [c(]2=75.7 (c=0.70, CH₂Cl₂); FT-IR (film) vₘₐₓ 3415, 2973, 2932, 1731, 1692, 1491, 1467, 1455, 1382, 1329, 1284, 1251, 1174, 1150, 1119, 1048, 1011, 977, 959, 879, 859, 843, 812, 794, 722, 695 cm⁻¹; ¹H NMR (600 MHz, C₆D₆) δ=6.82 (s, 1H), 6.27 (s, 1H), 5.44 (dd, J=10.1, 1.2 Hz, 1H), 5.17 (dd, J=9.8, 5.2 Hz, 1H), 4.24 (dd, J=11.0, 2.8 Hz, 1H), 3.74 (dd, J=3.5, 3.5 Hz, 1H), 3.39 (s, 3H), 3.09 (br s, 1H), 2.98 (qd, J=6.8, 2.8 Hz, 1H), 2.63 (dt, J=15.1, 9.8 Hz, 1H), 2.41 (dd, J=14.9, 11.1 Hz, 1H), 2.29-2.24 (m, 1H), 2.10 (dd, J=14.8, 2.8 Hz, 1H), 2.07 (d, J=1.3 Hz, 3H), 1.86-1.78 (m, 2H), 1.76 (s, 3H), 1.62 (br s, 3H), 1.61-1.56 (m, 1H), 1.25-1.17 (m, 3H), 1.12 (d, J=6.8 Hz, 3H), 1.04 (d, J=6.9 Hz, 3H), 1.03 (s, 3H), 1.01 (s, 3H) ppm; ¹³C NMR (151 MHz, C₆D₆) δ=219.7, 170.1, 148.9, 138.3, 137.8, 136.4, 121.6, 118.4, 109.8, 79.2, 74.6, 72.7, 53.6, 53.4, 42.2, 40.0, 38.7, 36.1, 32.8, 32.0, 31.7, 25.9, 23.1, 22.8, 18.5, 16.1, 15.9, 13.9 ppm; HRMS (ESI) calcd for C₂₈H₄₅N₂O₅S⁺ [M+H]⁺ 521.3044 found 521.3034.

### (1S,3S,7S,10R,11S,12S,16R)-3-Acetyl-7,11-dihydroxy-8,8,10,12,16-pentamethyl-4,17-dioxabicyclo[14.1.0]heptadecane-5,9-dione (75)

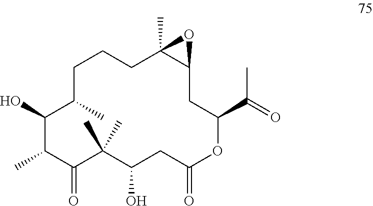

To a stirred solution of epothilone B (2, 122 mg, 24.0 μmol, 1.0 equiv) in dichloromethane (5 mL) at −78° C. was bubbled through freshly generated ozone. After the color of the reaction mixture changed to light blue (ca. 5 min), it was quenched with dimethyl sulfide (0.180 mL, 2.45 mmol, 10.0 equiv), and allowed to warm to 25° C. After 1 h, the solvent was removed under reduced pressure, and the obtained residue was purified by flash column chromatography (silica gel, 40→70% ethyl acetate in hexanes) to afford pure methyl ketone 75 (93.0 mg, 22.5 μmol, 94% yield) as an amorphous solid. 75: R_{f}=0.26 (silica gel, 40% hexanes in ethyl acetate); [α]_{D}²²=+12.7 (c=0.60, CH₂Cl₂); FT-IR (film) vₘₐₓ 3473, 2960, 2937, 2879, 1746, 1723, 1689, 1465, 1423, 1368, 1284, 1250, 1180, 1145, 1076, 1010, 980, 957, 916, 733, 672 cm⁻¹; ¹H NMR (600 MHz, CDCl₃) δ=5.31 (dd, J=10.2, 1.8 Hz, 1H), 4.31 (ddd, J=10.8, 4.8, 3.0 Hz, 1H), 4.10 (d, J=4.8 Hz, 1H), 3.70 (ddd, J=3.6, 3.6, 3.6 Hz, 1H), 3.25 (qd, J=6.6, 5.4 Hz, 1H), 2.82 (dd, J=9.0, 3.0 Hz, 1H), 2.57 (br s, 1H), 2.54 (dd, J=14.4, 10.8 Hz, 1H), 2.34 (ddd, J=15.0, 3.0, 1.8 Hz, 1H), 2.28 (s, 3H), 2.27 (dd, J=15.0, 3.0 Hz, 1H), 1.79-1.72 (m, 2H), 1.69-1.63 (m, 1H), 1.49-1.43 (m, 1H), 1.44-1.37 (m, 1H), 1.42 (s, 3H), 1.36-1.25 (m, 2H), 1.29 (s, 3H), 1.20 (d, J=6.6 Hz, 3H), 1.09 (s, 3H), 0.99 (d, J=7.2 Hz, 3H) ppm; ¹³C NMR (151 MHz, CDCl₃) δ=220.6, 205.0, 170.7, 76.8, 74.5, 71.7, 62.5, 62.2, 53.4, 42.7, 40.0, 37.4, 32.9, 31.3, 29.0, 26.4, 23.3, 22.6, 22.5, 18.0, 17.3, 14.4 ppm; HRMS (ESI) calcd for C₂₂H₃₆O₇Na⁺ [M+Na]⁺ 435.2353, found 435.2351.

### (4S,7R,8S,9S,13Z,16S)-16-Acetyl-4,8-dihydroxy-5,5,7,9,13-pentamethyloxacyclohexadec-13-ene-2,6-dione (77)

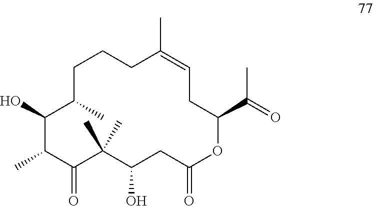

To a stirred suspension of tungsten hexachloride (0.163 g, 0.413 mmol, 2.0 equiv) in tetrahydrofuran (2 mL) at −78° C. was added n-butyllithium (1.6 M in hexanes, 0.52 mL, 0.83 mmol, 4.0 equiv) dropwise. After 10 min, the reaction mixture was allowed to slowly warm to 25° C., and stirred for an additional 30 min. Then the reaction mixture was cooled to 20° C., a solution of methyl ketone 75 (85.4 mg, 0.207 mmol, 1.0 equiv) in tetrahydrofuran (1 mL) was added dropwise, and the reaction mixture was allowed to slowly warm to 0° C. After 2 h, the reaction mixture was quenched with saturated aqueous ammonium chloride solution (10 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by flash column chromatography (silica gel, 10→50% ethyl acetate in hexanes) to afford pure olefinic methyl ketone 77 (57.0 mg, 0.144 mmol, 70% yield) as a colorless oil and its (E) isomer (12.3 mg, 31.0 μmol, 15% yield). 77: R_{f}=0.28 (silica gel, 40% ethyl acetate in hexanes); [α]_{D}²²=+14.2 (c=0.12, CH₂Cl₂); FT-IR (neat) vₘₐₓ 3466, 2933, 1746, 1718, 1690, 1465, 1404, 1365, 1276, 1258, 1183, 1146, 1062, 1007, 978, 939, 880, 750 cm⁻¹; ¹H NMR (600 MHz, CDCl₃) δ=5.21 (dd, J=10.2, 2.4 Hz, 1H), 5.08 (dd, J=9.6, 5.4 Hz, 1H), 4.38 (ddd, J=11.4, 4.2, 3.0 Hz, 1H), 4.17 (d, J=4.2 Hz, 1H), 3.62 (dd, J=5.4, 2.4 Hz, 1H), 3.19 (ddd, J=13.2, 6.6, 1.8 Hz, 1H), 2.89 (s, 1H), 2.57 (ddd, J=15.0, 9.6, 9.6 Hz, 1H), 2.49 (d, J=15.0 Hz, 1H), 2.47 (dd, J=15.0, 10.8 Hz, 1H), 2.26-2.22 (m, 1H), 2.25 (s, 3H), 2.16 (dd, J=15.0, 2.4 Hz, 1H), 1.90 (ddd, J=12.6, 6.0, 6.0 Hz, 1H), 1.75-1.70 (m, 1H), 1.69 (s, 3H), 1.68-1.64 (m, 1H), 1.43 (s, 3H), 1.28-1.22 (m, 2H), 1.21-1.17 (m, 1H), 1.20 (d, J=6.6 Hz, 3H), 1.06 (s, 3H), 0.98 (d, J=7.2 Hz, 3H) ppm; ¹³C NMR (151 MHz, CDCl₃) δ=221.1, 206.2, 170.4, 140.1, 119.3, 78.8, 74.6, 71.2, 53.7, 41.2, 40.3, 39.2, 32.0, 31.8, 28.8, 26.4, 25.7, 23.2, 23.1, 16.2, 15.6, 14.0 ppm; HRMS (ESI) calcd for C₂₂H₃₆O₆Na⁺ [M+Na]⁺ 419.2404, found 419.2408.

### (1S,3S,7S,10R,11S,12S,16R)-3-Acetyl-7,11-dihydroxy-8,8,10,12,16-pentamethyl-4-oxa-17-azabicyclo[14.1.0]heptadecane-5,9-dione (79)

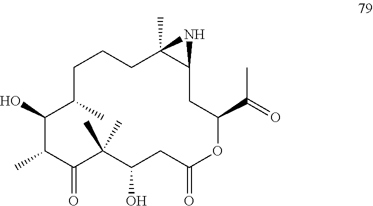

To a stirred solution of olefinic methyl ketone 77 (47.0 mg, 0.119 mmol, 1.0 equiv) in 2,2,2-trifluoroethanol (0.5 mL) at 25° C. was added O-(2,4-dinitrophenyl)hydroxylamine (35.5 mg, 0.180 mmol, 1.5 equiv), followed by bis[rhodium(α,α,α′,α′,-tetramethyl-1,3-benzenedipropionic acid)] (1.8 mg, 2.4 μmol, 0.02 equiv). After 30 min, the reaction mixture was diluted with dichloromethane (30 mL), and washed with a saturated aqueous solution of sodium bicarbonate (3×10 mL) and brine (10 mL). The two phases were separated, and the organic layer was dried with anhydrous sodium sulfate and concentrated under reduced pressure. The obtained residue was purified by flash column chromatography (silica gel, 5→30% methanol in ethyl acetate) to afford pure aziridine 79 (42.5 mg, 0.103 mmol, 87% yield) as a white amorphous solid. 79: R_{f}=0.22 (silica gel, 20% methanol in ethyl acetate); [α]_{D}²²=2.1 (c=0.33, CH₂Cl₂); FT-IR (film) vₘₐₓ 3460, 3298, 2957, 2927, 1741, 1722, 1687, 1464, 1421, 1367, 1283, 1258, 1173, 1147, 1075, 1056, 1008, 980, 939, 858, 735 cm⁻¹; ¹H NMR (600 MHz, C₆D₆) δ=5.10 (dd, J=6.6, 3.0 Hz, 1H), 4.36 (dd, J=10.8, 3.6 Hz, 1H), 3.80 (dd, J=6.0, 3.6 Hz, 1H), 3.22 (dq, J=6.6, 6.6 Hz, 1H), 2.48 (dd, J=13.8, 10.8 Hz, 1H), 2.24 (dd, J=13.8, 3.6 Hz, 1H), 1.81-1.76 (m, 1H), 1.65 (s, 3H), 1.58-1.54 (m, 1H), 1.53-1.48 (m, 1H), 1.42-1.26 (m, 7H), 1.25 (s, 3H), 1.10 (d, J=7.2 Hz, 3H), 1.03 (s, 3H), 0.99 (d, J=7.2 Hz. 3H), 0.78 (s. 3H) ppm; ¹³C NMR (151 MHz, C₆D₆) δ=219.2, 204.8, 170.6, 77.7, 76.2, 73.2, 52.8, 44.3, 39.9, 39.0, 38.0, 36.1, 32.4, 29.7, 28.8, 25.9, 25.9, 23.0, 20.6, 20.3, 17.7, 15.3 ppm; HRMS (ESI) calcd for C₂₂H₃₇NO₆Na⁺ [M+Na]⁺ 434.2513, found 434.2512.

### (1S,3S,7S,10R,11S,12S,16R)-3-Acetyl-7,11-dihydroxy-17-(2-hydroxyethyl)-8,8,10,12,16-penta-methyl-4-oxa-17-azabicyclo[14.1.0]heptadecane-5,9-dione (81)

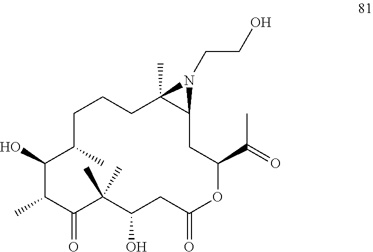

To a stirred solution of aziridine 79 (68.8 mg, 167 μmol, 1.0 equiv) in dimethylformamide (0.5 mL) at 25° C. was added 2-bromoethanol (104 mg, 0.835 mmol, 5.0 equiv), followed by potassium carbonate (92.5 mg, 0.669 mmol, 4.0 equiv), and the reaction mixture was heated to 70° C. After 12 h, the reaction mixture was allowed to cool to 25° C., and quenched with water (3.5 mL). The mixture was extracted with ethyl acetate (3×2 mL) and the combined organic layers were dried with anhydrous sodium sulfate and concentrated under reduced pressure. The obtained residue was purified by flash column chromatography (silica gel, 5→10% methanol in dichloromethane) to afford pure N-hydroxyethyl aziridine 81 (22.0 mg, 48.3 μmol, 29% yield) as a white amorphous solid. 81: R_{f}=0.38 (silica gel, 10% methanol in dichloromethane); [α]_{D}²²=−14.3 (c=0.14, CH₂Cl₂); FT-IR (film) vₘₐₓ 3406, 2934, 2878, 1742, 1720, 1688, 1466, 1421, 1368, 1255, 1179, 1148, 1068, 1008, 981, 956, 751, 711 cm⁻¹; ¹H NMR (600 MHz, CD₂Cl₂) δ=5.25 (dd, J=9.6, 1.8 Hz, 1H), 4.25 (dd, J=10.8, 3.0 Hz, 1H), 3.70 (dd, J=6.0, 3.6 Hz, 1H), 3.65 (dd, J=6.0, 3.6 Hz, 1H), 3.63 (dd, J=6.0, 3.6, Hz, 1H), 3.25 (dq, J=6.6, 6.6 Hz, 1H), 2.72 (ddd, J=12.6, 6.6, 4.2 Hz, 1H), 2.50 (ddd, J=12.6, 6.0, 4.2 Hz, 1H), 2.48 (dd, J=14.4, 10.8 Hz, 1H), 2.32 (dd, J=14.4, 2.4 Hz, 1H), 2.45 (s, 3H), 2.21 (ddd, J=15.6, 4.2, 2.4 Hz, 1H), 1.60-1.58 (m, 4H), 1.48-1.43 (m, 2H), 1.40 (s, 3H), 1.35-1.32 (m, 3H), 1.16 (d, J=7.2 Hz, 3H), 1.15 (s, 3H), 1.03 (s, 3H), 0.96 (d, J=7.2 Hz, 3H) ppm; ¹³C NMR (151 MHz, CD₂Cl₂) δ=220.3, 205.8, 171.1, 78.4, 75.4, 72.1, 62.4, 54.7, 53.2, 49.6, 44.0, 43.7, 39.9, 36.7, 35.8, 30.5, 29.8, 26.5, 22.6, 21.9, 18.6, 17.5, 16.1, 14.6 ppm; HRMS (ESI) calcd for C₂₄H₄₂NO₇⁺ [M+H]⁺ 456.2956, found 456.2967.

### (1S,3S,7S,10R,11S,12S,16R)-3-Acetyl-8,8,10,12,16-pentamethyl-7,11-bis[(triethylsilyl)oxy]-4,17-dioxabicyclo[14.1.0]heptadecane-5,9-dione (76)

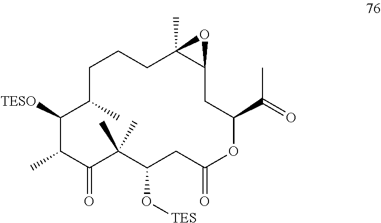

To a stirred solution of methyl ketone 75 (0.150 g, 0.364 mmol, 1.0 equiv) in dichloromethane (5 mL) at −78° C. was added 2,6-lutidine (0.13 mL, 1.1 mmol, 3.0 equiv), followed by triethylsilyl trifluoromethanesulfonate (0.20 mL, 0.87 mmol, 2.4 equiv). After 15 min, the reaction mixture was quenched with water (10 mL), and allowed to warm to 25° C. The two phases were separated, the aqueous layer was extracted with ethyl acetate (3×8 mL), and the combined organic layers were dried with anhydrous sodium sulfate and concentrated under reduced pressure. The obtained residue was purified by flash column chromatography (silica gel, 5→15% ethyl acetate in hexanes) to afford pure bis-TES ether 76 (0.196 g, 0.306 mmol, 84% yield) as a white foam. 76: R_{f}=0.37 (silica gel, 20% ethyl acetate in hexanes); [ci]2=14.0 (c=1.00, CH₂Cl₂); FT-IR (film) vₘₐₓ 2955, 2913, 2877, 1749, 1734, 1696, 1459, 1414, 1381, 1308, 1240, 1196, 1157, 1106, 1080, 1064, 1040, 1010, 985, 916, 859, 836, 783, 737, 676 cm⁻¹; ¹H NMR (600 MHz, CDCl₃) δ=5.01 (dd, J=10.2, 1.8 Hz, 1H), 4.04 (dd, J=10.2, 2.4 Hz, 1H), 3.91 (d, J=9.0 Hz, 1H), 3.04 (dq, J=9.6, 6.6 Hz, 1H), 2.94 (dd, J=16.2, 2.4 Hz, 1H), 2.86 (dd, J=10.2, 4.2 Hz, 1H), 2.77 (dd, J=16.2, 4.2 Hz, 1H), 2.37 (dd, J=16.2, 2.4 Hz, 1H), 2.24 (s, 3H), 1.76-1.68 (m, 2H), 1.63-1.58 (m, 1H), 1.55-1.45 (m, 2H), 1.42-1.38 (m, 1H), 1.30 (s, 3H), 1.27-1.23 (m, 1H), 1.25 (s, 3H), 1.17 (s, 3H), 1.10 (d, J=6.6 Hz, 3H), 1.07-1.04 (m, 1H), 1.00 (t, J=7.8 Hz, 9H), 0.99 (d, J=7.2 Hz, 3H), 0.98-0.95 (m, 1H), 0.93 (t, J=7.8 Hz, 9H), 0.67 (q, J=7.8 Hz, 6H), 0.61 (q, J=7.8 Hz, 6H) ppm; ¹³C NMR (151 MHz, CDCl₃) δ=215.2, 203.4, 171.8, 80.3, 76.5, 76.3, 62.5, 62.2, 53.5, 48.6, 39.4. 36.8, 32.1, 31.1, 30.3, 26.0, 24.9, 24.7, 23.7, 22.6, 19.7, 17.8, 7.3, 7.1, 5.7, 5.4 ppm; HRMS (ESI) calcd for C₃₄H₆₄O₇Si₂Na⁺ [M+Na]⁺ 663.4083, found 663.4057.

### (4S,7R,8S,9S,13Z,16S)-16-Acetyl-5,5,7,9,13-pentamethyl-4,8-bis[(triethylsilyl)oxy]oxacyclohexadec-13-ene-2,6-dione (78)

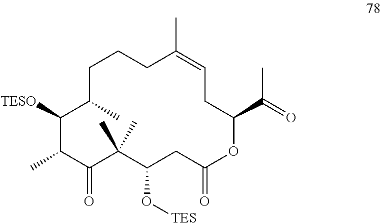

To a stirred suspension of tungsten hexachloride (0.496 g, 1.25 mmol, 2.0 equiv) in tetrahydrofuran (7 mL) at −78° C. was added n-butyllithium (1.6 M in hexanes, 1.56 mL, 2.50 mmol, 4.0 equiv) dropwise. After 10 min, the reaction mixture was allowed to slowly warm to 25° C., and stirred for an additional 30 min. The reaction mixture was cooled to 20° C., a solution of bis-TES ether 76 (0.401 g, 0.626 mmol, 1.0 equiv) in tetrahydrofuran (4 mL) was added dropwise, and the reaction mixture was allowed to slowly warm to 0° C. After 2 h, the reaction mixture was quenched with saturated aqueous ammonium chloride solution (10 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by flash column chromatography (silica gel, 2.5→30% ethyl acetate in hexanes) to afford pure olefinic methyl ketone 78 (0.335 g, 0.536 mmol, 86% yield) as a colorless oil. 78: R_{f}=0.21 (silica gel, 10% diethyl ether in hexanes); [α]_{D}²²=18.2 (c=1.00, CH₂Cl₂); FT-IR (neat) vₘₐₓ 2953, 2912, 2877, 1747, 1731, 1696, 1459, 1414, 1381, 1365, 1307, 1275, 1263, 1240, 1198, 1159, 1110, 1062, 1042, 1018, 984, 859, 835, 783, 744, 674 cm⁻¹; ¹H NMR (600 MHz, CDCl₃) δ=5.16 (dd, J=7.8, 7.8 Hz, 1H), 4.84 (dd, J=10.2, 1.8 Hz, 1H), 4.04 (dd, J=10.2, 1.8 Hz, 1H), 3.91 (d, J=9.0 Hz, 1H), 3.01 (dq, J=9.6, 6.6 Hz, 1H), 2.91 (dd, J=16.2, 1.8 Hz, 1H), 2.76 (dd, J=16.2, 10.8 Hz, 1H), 2.53 (ddd, J=15.0, 10.2, 10.2 Hz, 1H), 2.41 (dd, J=14.4, 10.8 Hz, 1H), 2.24 (dd, J=14.4, 7.2 Hz, 1H), 2.19 (s, 3H), 1.76-1.66 (m, 2H), 1.69 (s, 3H), 1.57-1.49 (m, 2H), 1.22 (s, 3H), 1.14 (s, 3H), 1.10-1.00 (m, 2H), 1.09 (d, J=6.6 Hz, 3H), 0.98 (t, J=7.8 Hz, 9H), 0.97 (d, J=6.6 Hz, 3H), 0.88 (t, J=7.8 Hz, 9H), 0.65 (q, J=7.8 Hz, 6H), 0.55 (q, J=7.8 Hz, 6H) ppm; ¹³C NMR (150 MHz, CDCl₃) δ=215.2, 204.4, 171.9, 142.4, 117.7, 80.1, 79.8, 76.6, 53.6, 48.2, 39.3, 37.6, 32.3, 31.4, 28.6, 27.5, 26.3, 25.1, 23.7, 23.2, 19.2, 17.7, 7.4, 7.0, 5.8, 5.4 ppm; HRMS (ESI) calcd for C₃₄H₆₄O₆Si₂Na⁺ [M+Na]⁺ 647.4134, found 647.4134.

### (1S,3S,7S,10R,11S,12S,16R)-3-Acetyl-8,8,10,12,16-pentamethyl-7,11-bis[(triethylsilyl)oxy]-4-oxa-17-azabicyclo[14.1.0]heptadecane-5,9-dione (80)

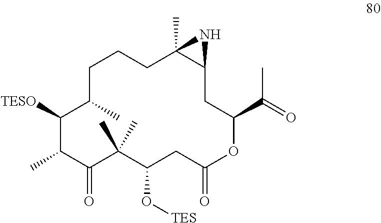

To a stirred solution of olefinic methyl ketone 78 (0.320 g, 0.512 mmol, 1.0 equiv) in 2,2,2-trifluoroethanol (3 mL) at 25° C. was added O-(2,4-dinitrophenyl)hydroxylamine (153 mg, 0.768 mmol, 1.5 equiv), followed by bis[rhodium(α,α,α′,α′,-tetramethyl-1,3-benzenedipropionic acid)] (7.8 mg, 10.2 μmol, 0.02 equiv). After 30 min, the reaction mixture was diluted with dichloromethane (60 mL), and washed with a saturated aqueous solution of sodium bicarbonate (3×15 mL) and brine (20 mL). The two phases were separated, and the organic layer was dried with anhydrous sodium sulfate and concentrated under reduced pressure. The obtained residue was purified by flash column chromatography (silica gel, 2.5→5% methanol in dichloromethane) to afford pure aziridine 80 (0.296 g, 0.462 mmol, 90% yield) as a pale yellow foam. 80: R_{f}=0.29 (silica gel, 5% methanol in ethyl acetate); [α]_{D}²²=−14.5 (c=0.64, CH₂Cl₂); FT-IR (film) vₘₐₓ 2953, 2918, 2877, 1747, 1732, 1696, 1460, 1414, 1382, 1307, 1240, 1199, 1157, 1107, 1067, 1043, 1018, 985, 858, 835, 783, 736, 675 cm⁻¹; ¹H NMR (600 MHz, C₆D₆) δ=4.90 (dd, J=9.0, 1.8 Hz, 1H), 4.18 (d, J=9.6 Hz, 1H), 4.07 (dd, J=9.0, 3.0 Hz, 1H), 2.88 (dq, J=10.2, 6.6 Hz, 1H), 2.76-2.68 (m, 2H), 1.94 (d, J=16.2 Hz, 1H), 1.83-1.78 (m, 1H), 1.76-1.65 (m, 2H), 1.72 (s, 3H), 1.60-1.53 (m, 1H), 1.51-1.45 (m, 1H), 1.41-1.35 (m, 3H), 1.26-1.19 (m, 1H), 1.19 (d, J=6.6 Hz, 3H), 1.15 (s, 3H), 1.09-1.04 (m, 24H), 0.79-0.71 (m, 12H), 0.67 (s, 3H) ppm; ¹³C NMR (151 MHz, C₆D₆) δ=213.9, 202.3, 171.7, 80.8, 78.2, 76.7, 53.1, 48.3, 42.4, 39.4, 39.3, 36.9, 33.7, 31.43, 31.37, 25.7, 25.4, 25.2, 25.0, 22.8, 20.0, 17.7, 7.5, 7.3, 6.0, 5.8 ppm; HRMS (ESI) calcd for C₃₄H₆₆NO₆Si₂⁺ [M+H]⁺ 640.4423, found 640.4442.

### (1S,3S,7S,10R,11S,12S,16R)-3-Acetyl-17-(2-{[tert-butyl(dimethyl)silyl]oxy}ethyl)-8,8,10,12,16-pentamethyl-7,11-bis[(triethylsilyl)oxy]-4-oxa-17-azabicyclo[14.1.0]heptadecane-5,9-dione (82)

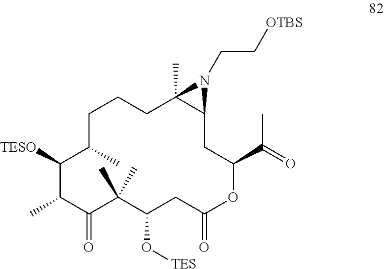

To a stirred solution of aziridine 80 (105 mg, 0.164 mmol, 1.0 equiv) in dimethylformamide (0.8 mL) at 25° C. was added (2-bromoethoxy)-tert-butyldimethylsilane (196 mg, 0.820 mmol, 5.0 equiv), followed by potassium carbonate (90.7 mg, 0.656 mmol, 4.0 equiv), and the reaction mixture was heated to 70° C. After 12 h, the reaction mixture was allowed to cool to 25° C., and quenched with water (3.5 mL). The mixture was extracted with ethyl acetate (3×2 mL), and the combined organic layers were dried with anhydrous sodium sulfate and concentrated under reduced pressure. The obtained residue was purified by flash column chromatography (silica gel, 10→40% ethyl acetate in hexanes) to afford pure N-alkylated aziridine 82 (0.118 g, 0.148 mmol, 90% yield) as a pale yellow foam. 82: R_{f}=0.31 (silica gel, 30% ethyl acetate in hexanes); [α]_{D}²²=−6.9 (c=0.26, CH₂Cl₂); FT-IR (film) vₘₐₓ 2953, 2931, 2877, 1748, 1734, 1697, 1462, 1414, 1382, 1361, 1307, 1250, 1196, 1158, 1109, 1079, 1042, 1008, 985, 835, 780, 737, 667 cm⁻¹; ¹H NMR (600 MHz, C₆D₆) δ=4.94 (dd, J=9.0, 1.8 Hz, 1H), 4.19 (d, J=9.6 Hz, 1H), 4.05 (dd, J=7.8, 4.8 Hz, 1H), 3.84 (ddd, J=9.6, 6.6, 6.6 Hz, 1H), 3.77 (ddd, J=10.2, 5.4, 5.4 Hz, 1H), 2.85 (dq, J=9.6, 6.6 Hz, 1H), 2.75-2.71 (m, 2H), 2.42 (ddd, J=12.6, 6.6, 6.6 Hz, 1H), 2.19 (d, J=16.2 Hz, 1H), 1.86-1.76 (m, 2H), 1.83 (s, 3H), 1.72-1.59 (m, 3H), 1.48-1.36 (m, 2H), 1.25-1.10 (m, 3H), 1.21 (d, J=7.2 Hz, 3H), 1.16 (s, 3H), 1.11-1.06 (m, 18H), 1.04 (m, J=6.6 Hz, 3H), 1.00 (s, 9H), 0.83-0.77 (m, 6H), 0.72 (q, J=7.8 Hz, 6H), 0.68 (s, 3H), 0.10 (s, 6H) ppm; ¹³C NMR (151 MHz, C₆D₆) δ=213.9, 202.5, 171.9, 80.8, 78.1, 76.8, 64.3, 54.6, 53.1, 50.7, 48.3, 42.8, 39.4, 36.9, 35.9, 31.7, 31.6, 26.2, 25.5, 25.1, 25.0, 23.0, 20.0, 18.5, 17.8, 15.5, 7.4, 7.3, 6.0, 5.8, −5.12, −5.13 ppm; HRMS (ESI) calcd for C₄₂H₈₄NO₇Si₃⁺ [M+H]⁺ 798.5550, found 798.5541.

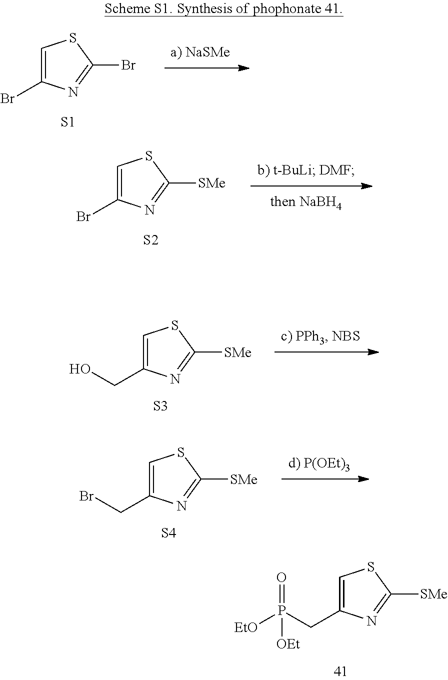

### 4-Bromo-2-(methylsulfanyl)-1,3-thiazole (S2)

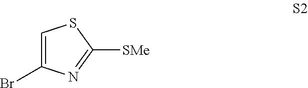

To a stirred solution of 2,4-dibromothiazole S1 (1.97 g, 8.11 mmol, 1.0 equiv) in ethanol (10 mL) at 0° C. was added sodium thiomethoxide (1.70 g, 24.3 mmol, 3.0 equiv), and the reaction mixture was allowed to slowly warm to 25° C. After 6 h, the reaction mixture was quenched with water (30 mL) and extracted with ethyl acetate (3×50 mL). The combined organic layers were dried with anhydrous sodium sulfate and concentrated under reduced pressure. The obtained residue was purified by flash column chromatography (silica gel, 2→8% ethyl acetate in hexanes) to afford pure methylthiothiazole S2 (Nicolaou, et al., 1998; Nicolaou, et al., 1999; and Nicolaou, et al., 2002) (1.50 g, 7.14 mmol, 88% yield) as a white amorphous solid. S2: Rₜ=0.43 (silica gel, 10% ethyl ether in hexanes); FT-IR (film) vₘₐₓ 3006, 2990, 1275, 1260, 764, 750 cm⁻¹; ¹H NMR (600 MHz, CDCl₃) δ=7.07 (s, 1H), 2.70 (s, 3H) ppm; ¹³C NMR (151 MHz, CDCl₃) δ=168.1, 124.5, 115.7, 16.8 ppm.

### [2-(Methylsulfanyl)-1,3-thiazol-4-yl]methanol (S3)

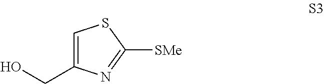

To a stirred solution of methylthiothiazole S2 (1.48 g, 7.04 mmol, 1.0 equiv) in diethyl ether (20 mL) at −78° C. was added tert-butyllithium (1.4 M in hexanes, 6.0 mL, 8.4 mmol, 1.2 equiv) dropwise. After 5 min, dimethylformamide (1.03 mL, 14.1 mmol, 2.0 equiv) was added, and stirring was continued for an additional 20 min. Then the reaction mixture was quenched with methanol (20 mL), allowed to warm to 0° C., and sodium borohydride (533 mg, 14.1 mmol, 2.0 equiv) was added. After 30 min, the reaction mixture was quenched with saturated aqueous ammonium chloride solution (20 mL), diluted with water (20 mL), and allowed to warm to 25° C. The two phases were separated, and the aqueous layer was extracted with ethyl acetate (3×20 mL). The combined organic layers were dried with anhydrous sodium sulfate and concentrated under reduced pressure. The obtained residue was purified by flash column chromatography (silica gel, 20→50% ethyl acetate in hexanes) to afford pure hydroxymethyl thiazole S3 (0.850 g, 5.27 mmol, 75% yield) as a colorless oil. S3: R_{f}=0.26 (silica gel, 50% ethyl acetate in hexanes); FT-IR (neat) vₘₐₓ 3334, 3118, 2924, 2860, 1529, 1407, 1314, 1261, 1213, 1135, 1037, 966, 944, 849, 752, 725 cm⁻¹; ¹H NMR (600 MHz, CDCl₃) δ=7.05 (s, 1H), 4.71 (s, 2H), 2.69 (s, 3H) ppm; ¹³C NMR (151 MHz, CDCl₃) δ=167.5, 156.5, 114.0, 61.2, 17.0 ppm; HRMS (ESI) calcd for C₅H₈NOS₂⁺ [M+H]⁺ 162.0042, found 162.0048.

### 4-(Bromomethyl)-2-(methylsulfanyl)-1,3-thiazole (S4)

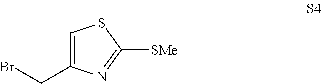

To a stirred solution of hydroxymethyl thiazole S3 (642 mg, 3.98 mmol, 1.0 equiv) in dichloromethane (6 mL) at −78° C. was added triphenylphosphine (1.10 g, 4.18 mmol, 1.05 equiv), followed by N-bromosuccinimide (708 mg, 3.98 mmol, 1.0 equiv). After 5 min, the reaction mixture was quenched with water (10 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 1→5% ethyl acetate in hexanes) to afford pure bromomethyl thiazole S4 (0.696 g, 3.10 mmol, 78% yield) as a colorless oil. S4: R_{f}=0.27 (silica gel, 10% ethyl ether in hexanes); FT-IR (neat) vₘₐₓ 3103, 2924, 2850, 1511, 1411, 1314, 1214, 1147, 1108, 1055, 1037, 966, 882, 746, 701, 672 cm⁻¹; ¹H NMR (600 MHz, CDCl₃) δ=7.16 (s, 1H), 4.51 (s, 2H), 2.69 (s, 3H) ppm; ¹³C NMR (151 MHz, CDCl₃) δ=167.7, 152.3, 117.0, 27.1, 16.9 ppm; HRMS (ESI) calcd for C₅H₇BrNS₂⁺ [M+H]⁺ 223.9198, found 223.9201.

### Diethyl {[2-(methylsulfanyl)-1,3-thiazol-4-yl]methyl}phosphonate (41)

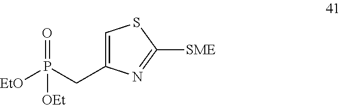

A stirred solution of bromomethyl thiazole S4 (1.02 g, 4.55 mmol, 1.0 equiv) in triethyl phosphite (5.0 mL, 29 mmol, 6.4 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 70→100% ethyl acetate in hexanes) to afford pure phosphonate 41 (1.18 g, 4.19 mmol, 92% yield) as a colorless oil. 41: R_{f}=0.20 (silica gel, ethyl acetate); FT-IR (neat) vₘₐₓ 3463, 3108, 2982, 2929, 1646, 1515 1478, 1411, 1393, 1368, 1314, 1248, 1163, 1097, 1023, 966, 947, 867, 842, 808, 781, 716, 660 cm⁻¹; ¹H NMR (600 MHz, CDCl₃) δ=7.08 (d, J=3.6 Hz, 1H), 4.08 (dq, J=8.4, 7.2 Hz, 4H), 3.32 (d, J=21.0 Hz, 2H), 2.65 (s, 3H), 1.28 (t, J=7.2 Hz, 6H) ppm; ¹³C NMR (151 MHz, CDCl₃) δ=166.1, 146.8 (d, J=8.1 Hz), 115.6 (d, J=8.0 Hz), 62.4 (d, J=6.5 Hz), 29.5 (d, J=140 Hz), 16.9, 16.5 (d, J=6.0 Hz) ppm; HRMS (ESI) calcd for C₉H₁₇NO₃PS₂⁺ [M+H]⁺ 282.0382, found 282.0378.

### (1S,3S,7S,10R,11S,12S,16R)-17-(2-{[tert-Butyl(dimethyl)silyl]oxy}ethyl)-8,8,10,12,16-pentamethyl-3-{(1E)-1-[2-(methylsulfanyl)-1,3-thiazol-4-yl]prop-1-en-2-yl}-7,11-bis[(triethylsilyl)oxy]-4-oxa-17-azabicyclo[14.1.0]heptadecane-5,9-dione (83)

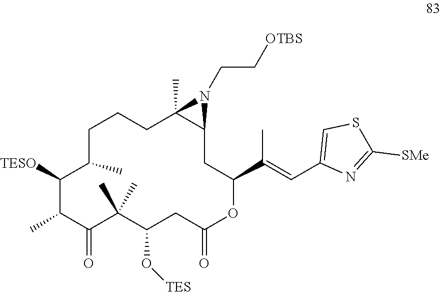

To a stirred solution of phosphonate 41 (115 mg, 0.409 mmol, 15 equiv) in tetrahydrofuran (0.5 mL) at −78° C. was added n-butyllithium (1.6 M in hexanes, 0.20 mL, 0.33 mmol, 12 equiv) dropwise. After 45 min, a solution of methyl ketone 82 (21.6 mg, 27.0 μmol, 1.0 equiv) in tetrahydrofuran (0.5 mL) was added, and the reaction mixture was allowed to slowly warm to 0° C. After 2 h, the reaction mixture was quenched with saturated aqueous ammonium chloride solution (10 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 5→15% ethyl acetate in hexanes) to afford pure protected epothilone 83 (15.0 mg, 16 μmol, 60% yield) as a colorless oil. 83: Rₜ=0.30 (silica gel, 15% ethyl acetate in hexanes); [c(]2=3.0 (c=1.2, CH₂Cl₂); FT-IR (neat) 2953, 2931, 2877, 1741, 1697, 1463, 1421, 1381, 1304, 1249, 1198, 1157, 1110, 1076, 1037, 1019, 985, 836, 779, 738, 674, 663 cm⁻¹; ¹H NMR (600 MHz, C₆D₆) δ=6.7 (s, 1H), 6.4 (s, 1H), 5.45 (dd, J=8.4, 3.6 Hz, 1H), 4.24 (dd, J=8.4, 3.6 Hz, 1H), 4.17 (d, J=9.0 Hz, 1H), 3.87-3.79 (m, 2H), 3.03 (dq, J=9.0, 7.2 Hz, 1H), 2.75 (ddd, J=12.0, 6.0, 6.0 Hz, 1H), 2.72 (dd, J=15.6, 8.4 Hz, 1H), 2.59 (dd, J=16.2, 3.0 Hz, 1H), 2.46 (ddd, J=12.0, 6.0, 6.0 Hz, 1H), 2.30 (s, 3H), 2.28-2.27 (m, 1H), 2.20 (s, 3H), 2.09-2.03 (m, 1H), 1.90-1.81 (m, 2H), 1.75-1.70 (m, 1H), 1.66-1.59 (m, 1H), 1.53-1.46 (m, 2H), 1.27 (dd, J=9.6, 3.0 Hz, 1H), 1.24-1.20 (m, 1H), 1.19 (d, J=6.6 Hz, 3H), 1.18 (s, 3H), 1.16 (s, 3H), 1.13 (d, J=7.0 Hz, 3H), 1.10-1.05 (m, 18H), 0.99 (s, 9H), 0.87 (s, 3H), 0.84-0.77 (m, 6H), 0.73-0.69 (m, 6H), 0.094 (s, 3H), 0.091 (s, 3H) ppm; ¹³C NMR (151 MHz, C₆D₆) δ=214.5, 170.7, 165.3, 153.7, 138.9, 120.5, 116.5, 80.1, 79.6, 75.9, 64.3, 54.9, 53.4, 50.1, 48.1, 43.3, 40.2, 37.4, 36.4, 35.4, 32.4, 26.2, 25.5, 23.6, 23.1, 20.1, 18.5, 17.6, 15.9, 15.6, 14.7, 7.42, 7.36, 5.95, 5.80, 5.1 ppm; HRMS (ESI) calcd for C₄₇H₈₉N₂O₆S₂Si₃⁺[M+H]⁺ 925.5464, found 925.5454.

### (1S,3S,7S,10R,11S,12S,16R)-7,11-Dihydroxy-17-(2-hydroxyethyl)-8,8,10,12,16-pentamethyl-3-{(1E)-1-[2-(methylsulfanyl)-1,3-thiazol-4-yl]prop-1-en-2-yl}-4-oxa-17-azabicyclo[14.1.0]-heptadecane-5,9-dione (12)

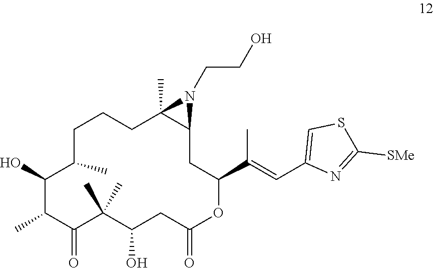

To a stirred solution of protected epothilone 83 (30.0 mg, 32.4 μmol, 1.0 equiv) in tetrahydrofuran (1.0 mL) at 0° C. was added hydrogen fluoride-pyridine complex (70% HF, 0.10 mL, 3.9 mmol, excess), and the reaction mixture was allowed to slowly warm to 25° C. After 1 h, the reaction mixture was quenched with saturated aqueous sodium bicarbonate solution (5 mL), and the two phases were separated. The aqueous layer was extracted with ethyl acetate (3×5 mL), and the combined organic layers were dried with anhydrous sodium sulfate and concentrated under reduced pressure. The obtained residue was purified by flash column chromatography (silica gel, 5→20% methanol in ethyl acetate) to afford pure epothilone 12 (15.0 mg, 25.7 mmol, 79% yield) as a white amorphous solid. 12: R_{f}=0.41 (silica gel, 20% methanol in ethyl acetate); [α]_{D}²²=−16.3 (c=0.64, CH₂Cl₂); FT-IR (film) vₘₐₓ 3373, 2927, 1729, 1685, 1654, 1559, 1460, 1452, 1424, 1259, 1149, 1037, 981, 881, 802, 735, 700 cm⁻¹; ¹H NMR (600 MHz, C₆D₆) δ=6.71 (s, 1H), 6.47 (s, 1H), 5.55 (dd, J=4.2, 4.2 Hz, 1H), 4.12 (dd, J=9.0, 3.0 Hz, 1H), 3.92-3.89 (m, 1H), 3.68-3.62 (m, 2H), 3.34 (ddd, J=13.8, 6.6, 6.6 Hz, 1H), 2.56-2.52 (m, 1H), 2.37-2.31 (m, 2H), 2.43-2.20 (m, 1H), 2.18 (s, 3H), 2.05 (s, 3H), 1.86-1.83 (m, 1H), 1.66 (ddd, J=15.6, 4.8, 4.8 Hz, 1H), 1.63-1.59 (m, 1H), 1.52-1.43 (m, 2H), 1.41-1.34 (m, 2H), 1.22 (ddd, J=13.8, 6.6, 6.6 Hz, 1H), 1.17 (s, 3H), 1.10 (d, J=7.0 Hz, 3H), 1.09-1.06 (m, 1H), 1.04 (d, J=7.0 Hz, 3H), 1.00-0.98 (m, 1H), 0.94 (s, 3H), 0.76 (s, 3H) ppm; ¹³C NMR (151 MHz, C₆D₆) δ=219.5, 170.9, 165.7, 153.6, 136.5, 118.6, 116.3, 76.8, 75.7, 63.8, 62.4, 55.7, 52.1, 47.0, 45.7, 42.3, 39.0, 35.2, 34.0, 30.8, 28.7, 25.4, 22.4, 21.8, 18.6, 16.2, 15.99, 15.98, 15.3 ppm; HRMS (ESI) calcd for C₂₉H₄₇N₂O₆S₂⁺ [M+H]⁺ 583.2870, found 583.2861.

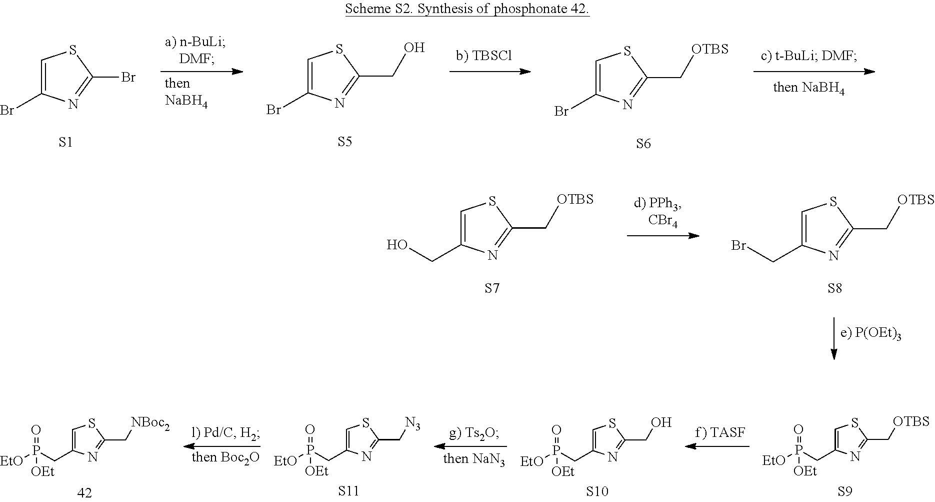

(4-Bromo-1,3-thiazol-2-yl)methanol (S5): Hydroxymethyl thiazole S5 was prepared from commercially available 2,4-dibromothiazole S1 as previously described; the physical and spectral data are consistent with those reported (Nicolaou et al., 1998).

4-Bromo-2-({[tert-butyl(dimethyl)silyl]oxy}methyl)-1,3-thiazole (S6): Silyl ether thiazole S6 was prepared from hydroxymethyl thiazole S5 as previously described; the physical and spectral data are consistent with those reported (Simeon et al., 2007).

[2-({[tert-Butyl(dimethyl)silyl]oxy}methyl)-1,3-thiazol-4-yl]methanol (S7): Prepared from silyl ether thiazole S6 (2.42 g, 7.85 mmol, 1.0 equiv) according to the procedure described above for the preparation of S3 to afford hydroxymethyl thiazole S7 (1.59 g, 6.13 mmol, 78% yield) as a colorless oil; the physical and spectral data are consistent with those reported (Lee et al., 2001).

### 4-(Bromomethyl)-2-({[tert-butyl(dimethyl)silyl]oxy}methyl)-1,3-thiazole (S8)

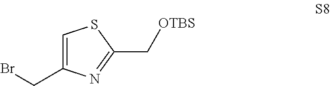 [SMILES: CC(C)(C)[Si](C)(C)OCc1nc(CBr)cs1]

To a stirred solution of hydroxymethyl thiazole S7 (1.41 g, 5.43 mmol, 1.0 equiv) in acetonitrile (45 mL) at 25° C. was added triphenylphosphine (2.42 g, 9.23 mmol, 1.7 equiv), followed by 2,6-lutidine (0.25 mL, 2.2 mmol, 0.4 equiv), and carbon tetrabromide (3.06 g, 9.23 mmol, 1.7 equiv). After 2 h, the reaction mixture was quenched with saturated aqueous sodium bicarbonate solution (20 mL), and diluted with diethyl ether (20 mL). The two phases were separated, and the aqueous layer was extracted with diethyl ether (3×15 mL). The combined organic layers were dried with anhydrous sodium sulfate and concentrated under reduced pressure. The obtained residue was purified by flash column chromatography (silica gel, 5→10% ethyl acetate in hexanes) to afford pure bromomethyl thiazole S8 (1.60 g, 4.96 mmol, 91% yield) as a colorless oil. S8: Rₜ=0.31 (silica gel, 10% ethyl acetate in hexanes); FT-IR (neat) vₘₐₓ 3106, 2954, 2929, 2885, 2857, 1519, 1492, 1471, 1463, 1426, 1390, 1355, 1255, 1197, 1145, 1111, 1006, 964, 939, 836, 778, 706, 684, 662 cm⁻¹; ¹H NMR (600 MHz, CDCl₃) δ=7.23 (s, 1H), 4.95 (s, 2H), 4.55 (s, 2H), 0.95 (s, 9H), 0.13 (s, 6H) ppm; ¹³C NMR (151 MHz, CDCl₃) δ=174.6, 151.9, 117.4, 63.3, 27.4, 25.9, 18.4, −5.3 ppm; HRMS (ESI) calcd for C₁₁H₂₁BrNOSSi⁺ [M+H]⁺ 322.0291, found 322.0285.

### Diethyl {[2-([{tert-butyl(dimethyl)silyl]oxy}methyl)-1,3-thiazol-4-yl]methyl}phosphonate (S9)

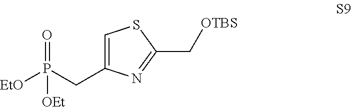 [SMILES: CCOP(=O)(Cc1csc(CO[Si](C)(C)C(C)(C)C)n1)OCC]

A stirred solution of bromomethyl thiazole S8 (0.20 g, 0.63 mmol, 1.0 equiv) in triethyl phosphite (2.2 mL, 13 mmol, 20 equiv) heated to 160° C. After 3 h, the triethyl phosphite was removed under a steady flow of nitrogen gas, and the reaction mixture was allowed to cool to 25° C. The obtained residue was purified by flash column chromatography (silica gel, 50→100% ethyl acetate in hexanes) to afford pure phosphonate S9 (190 mg, 0.51 mmol, 80% yield) as a colorless oil. S9: Rₜ=0.28 (silica gel, ethyl acetate); FT-IR (neat) vₘₐₓ 3476, 3107, 2955, 2930, 2903, 2858, 1519, 1472, 1463, 1444, 1392, 1361, 1321, 1253, 1198, 1164, 1099, 1055, 1027, 959, 837, 779, 722, 708, 674, 658 cm⁻¹; ¹H NMR (600 MHz, CDCl₃) δ=7.17 (d, J=3.5 Hz, 1H), 4.94 (s, 2H), 4.11-4.06 (m, 4H), 3.34 (d, J=21.0 Hz, 2H), 1.27 (t, J=7.1 Hz, 6H), 0.95 (s, 9H), 0.12 (s, 6H) ppm; ¹³C NMR (151 MHz, CDCl₃) δ=173.2, 146.1 (d, J=8.3 Hz), 116.1 (d, J=6.4 Hz), 63.2, 62.4 (d, J=6.6 Hz), 29.0 (d, J=141.0 Hz), 25.9, 18.4, 16.5 (d, J=6.0 Hz), 5.3 ppm; HRMS (PST) calcd for C₁₅H₃₁NO₄PSSi⁺ [M+H]⁺ 380.1475, found 380.1475.

### Diethyl {[2-(hydroxymethyl)-1,3-thiazol-4-yl]methyl}phosphonate (S10)

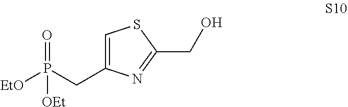 [SMILES: CCOP(=O)(Cc1csc(CO)n1)OCC]

To a stirred solution of phosphonate S9 (1.53 g, 4.03 mmol, 1.0 equiv) in dimethylformamide (25 mL) at 0° C. was added a solution of tris(dimethylamino)sulfonium difluorotrimethylsilicate (5.55 g, 20.2 mmol, 5.0 equiv) in dimethylformamide (14 mL), followed by water (0.73 mL, 40 mmol, 10.0 equiv), and the reaction mixture was allowed to slowly warm to 25° C. After 10 h, water (30 mL) was added to the reaction mixture, followed by ethyl acetate (30 mL), and the two phases were separated. The aqueous layer was extracted with ethyl acetate (3×15 mL), and the combined organic layers were dried with anhydrous sodium sulfate and concentrated under reduced pressure. The obtained residue was purified by flash column chromatography (5% methanol in dichloromethane) to afford pure hydroxymethyl phosphonate S10 (687 mg, 2.56 mmol, 64% yield) as a colorless oil. S10: R_{f}=0.33 (silica gel, 5% methanol in dichloromethane); FT-IR (neat) vₘₐₓ 3319, 2983, 2909, 1520, 1477, 1443, 1393, 1346, 1325, 1231, 1163, 1139, 1097, 1050, 1022, 957, 874, 845, 809, 784, 723, 670 cm⁻¹; ¹H NMR (600 MHz, CDCl₃) δ=7.14 (d, J=3.2 Hz, 1H), 4.81 (s, 2H), 4.08-4.04 (m, 4H), 3.32 (d, J=21.0 Hz, 2H), 1.25 (t, J=7.1 Hz, 6H) ppm; ¹³C NMR (151 MHz, CDCl₃) δ=172.5, 146.0 (d, J=8.2 Hz), 116.4 (d, J=6.8 Hz), 62.5 (d, J=6.6 Hz), 61.9, 28.9 (d, J=141.5 Hz), 16.5 (d, J=6.0 Hz) ppm; HRMS (ESI) calcd for C₉H₁₇NO₄PS⁺ [M+H]⁺ 266.0610, found 266.0601.

### Diethyl {[2-(azidomethyl)-1,3-thiazol-4-yl]methyl}phosphonate (S11)

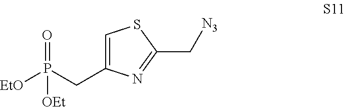 [SMILES: CCOP(=O)(Cc1csc(CN=[N+]=[N-])n1)OCC]

To a stirred solution of hydroxymethyl phosphonate S10 (687 mg, 2.59 mmol, 1.0 equiv) in dichloromethane (10.4 mL) at 25° C. was added triethylamine (0.72 mL, 5.2 mmol, 2.0 equiv), followed by 4-(dimethylamino)pyridine (32 mg, 0.26 mmol, 0.1 equiv). The reaction mixture was cooled to 20° C., and p-toluenesulfonic anhydride (1.27 g, 3.89 mmol, 1.5 equiv) was added. After 30 min, the reaction mixture was quenched with water (5 mL), and allowed to warm to 25° C. The two phases were separated, and the aqueous layer was extracted with dichloromethane (3×5 mL). The combined organic layers were dried with anhydrous sodium sulfate and concentrated under reduced pressure. The crude tosylate was then redissolved in dimethylformamide (5 mL) with stirring, and cooled to 20° C. Sodium azide (505 mg, 7.77 mmol, 3.0 equiv) was added, and after 15 min, the reaction mixture was quenched with water (5 mL) and allowed to warm to 25° C. The two phases were separated, and the aqueous layer was extracted with ethyl acetate (3×3 mL). The combined organic layers were dried with anhydrous sodium sulfate and concentrated under reduced pressure. The obtained residue was purified by flash column chromatography (5% methanol in dichloromethane) to afford pure azidomethyl phosphonate S11 (643 mg, 2.22 mmol, 86% yield) as a colorless oil. S11: R_{f}=0.44 (silica gel, 5% methanol in dichloromethane); FT-IR (neat) vₘₐₓ 3470, 3111, 2983, 2930, 2100, 1517, 1443, 1393, 1327, 1250, 1162, 1098, 1053, 1026, 965, 874, 810, 783, 724 cm⁻¹; ¹H NMR (600 MHz, CDCl₃) δ=7.25 (d, J=3.5 Hz, 1H), 4.63 (s, 2H), 4.12-4.07 (m, 4H), 3.37 (d, J=21.0 Hz, 2H), 1.27 (t, J=7.1 Hz, 6H) ppm; ¹³C NMR (151 MHz, CDCl₃) δ=164.3, 147.4 (d, J=8.1 Hz), 117.6 (d, J=7.5 Hz), 62.4 (d, J=6.6 Hz), 51.4, 29.1 (d, J=141.1 Hz), 16.5 (d, J=6.0 Hz) ppm; HRMS (ESI) calcd for C₉H₁₆N₄O₃PS⁺ [M+H]⁺ 291.0675, found 291.0675.

### Diethyl [(2-{[bis(tert-butoxycarbonyl)amino]methyl}-1,3-thiazol-4-yl)methyl]phosphonate (42)

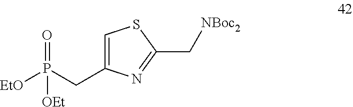

To a stirred solution of azidomethyl phosphonate S11 (0.20 g, 0.69 mmol, 1.0 equiv) in ethyl acetate (4 mL) at 25° C. was added 5% palladium on carbon (50 mg, 25% w/w), and an atmosphere of hydrogen (1 atm) was introduced. After 12 h, the hydrogen atmosphere was removed, and the reaction mixture was filtered through a pad of Celite®, rinsed thoroughly with ethyl acetate (20 mL), and concentrated under reduced pressure. The crude amine was then redissolved in tetrahydrofuran (5 mL) at 25° C. with stirring, and triethylamine (0.26 mL, 1.8 mmol, 2.6 equiv), 4-(dimethylamino)pyridine (9.0 mg, 7.0 nmol, 0.1 equiv), and di-tert-butyl dicarbonate (332 mg, 1.52 mmol, 2.2 equiv) were added sequentially. The reaction mixture was heated to 60° C. for 2.5 h, allowed to cool to 25° C., and then quenched with saturated aqueous ammonium chloride solution (3 mL). The two phases were separated, and the aqueous layer was extracted with ethyl acetate (3×2 mL). The combined organic layers were dried with anhydrous sodium sulfate and concentrated under reduced pressure. The obtained residue was purified by flash column chromatography (50→100% ethyl acetate in hexanes) to afford pure phosphonate 42 (293 mg, 0.63 mmol, 91% yield) as a colorless oil. 42: R_{f}=0.27 (silica gel, ethyl acetate); FT-IR (neat) vₘₐₓ 3459, 3109, 2980, 2934, 1793, 1753, 1699, 1519, 1479, 1458, 1422, 1393, 1367, 1341, 1254, 1228, 1129, 1054, 1026, 965, 890, 853, 783 cm¹; ¹H NMR (600 MHz, CDCl₃) δ=7.15 (d, J=3.5 Hz, 1H), 5.04 (s, 2H), 4.10-4.05 (m, 4H), 3.34 (d, J=21.0 Hz, 2H), 1.48 (s, 18H), 1.26 (t, J=7.1 Hz, 6H) ppm; ¹³C NMR (151 MHz, CDCl₃) δ=167.8, 151.9, 146.2 (d, J=7.7 Hz), 116.3 (d, J=7.2 Hz), 83.4, 62.3 (d, J=6.6 Hz), 47.8, 29.0 (d, J=140.9 Hz), 28.1, 16.5 (d, J=6.1 Hz) ppm; HRMS (ESI) calcd for Cl₉H₃₃N₂O₇PS⁺ [M+Na]+487.1638, found 487.1620.

### Di-tert-butyl ({4-[(1E)-2-{(1S,3S,7S,10R,11S,12S,16R)-17-(2-{[tert-butyl(dimethyl)silyl]oxy}ethyl)-8,8,10,12,16-pentamethyl-5,9-dioxo-7,11-bis[(triethylsilyl)oxy]-4-oxa-17-azabicyclo-[14.1.0]heptadec-3-yl}prop-1-en-1-yl]-1,3-thiazol-2-yl}methyl)imidodicarbonate (84)

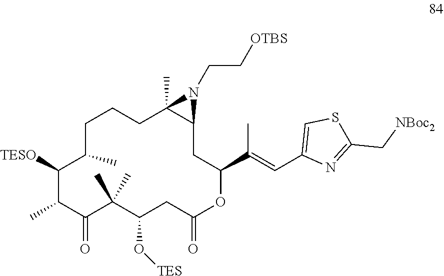

To a stirred solution of phosphonate 42 (97.0 mg, 0.209 mmol, 8.3 equiv) in tetrahydrofuran (1.0 mL) at −78° C. was added sodium bis(trimethylsilyl)amide (1.0 M in tetrahydrofuran, 0.17 mL, 0.17 mmol, 6.8 equiv) dropwise. After 35 min, a solution of methyl ketone 82 (20 mg, 25 nmol, 1.0 equiv) in tetrahydrofuran (0.4 mL) was added, and the reaction mixture was stirred for an additional 2 h. Then the reaction mixture was quenched with saturated aqueous ammonium chloride solution (10 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 5→20% ethyl acetate in hexanes) to afford pure protected epothilone 84 (18.8 mg, 17 nmol, 68% yield) as a colorless oil. 84: R_{f}=0.24 (silica gel, 15% ethyl acetate in hexanes); [α]_{D}²²=−4.4 (c=0.84, CH₂Cl₂); FT-IR (neat) vₘₐₓ 2954, 2933, 2877, 1796, 1742, 1697, 1460, 1418, 1380, 1367, 1343, 1303, 1251, 1230, 1124, 1008, 985, 836 cm⁻¹; ¹H NMR (600 MHz, C₆D₆) δ=6.68 (s, 1H), 6.54 (s, 1H), 5.44 (dd, J=9.0, 3.0 Hz, 1H), 5.08 (s, 2H), 4.23 (dd, J=9.0, 3.0 Hz, 1H), 4.19 (d, J=8.4 Hz, 1H), 3.87-3.79 (m, 2H), 3.04 (dq, J=8.4, 6.6 Hz, 1H), 2.78-2.70 (m, 2H), 2.59 (dd, J=16.2, 3.0 Hz, 1H), 2.47 (ddd, J=12.6, 6.6, 6.6 Hz, 1H), 2.33 (s, 3H), 2.27 (ddd, J=14.4, 3.0, 3.0 Hz, 1H), 2.04 (ddd, J=15.0, 9.0, 9.0 Hz, 1H), 1.90-1.80 (m, 2H), 1.76-1.71 (m, 1H), 1.66-1.59 (m, 1H), 1.52-1.48 (m, 2H), 1.37 (s, 18H), 1.26 (dd, J=15.6, 9.0 Hz, 1H), 1.22-1.17 (m, 1H), 1.20 (d, J=7.2 Hz, 3H), 1.19 (s, 3H), 1.16 (s, 3H), 1.14 (d, J=6.6 Hz, 3H), 1.09 (t, J=7.8 Hz, 9H), 1.06 (t, J=7.8 Hz, 9H), 1.00 (s, 9H), 0.85-0.78 (m, 6H), 0.74-0.70 (m, 6H), 0.101 (s, 3H), 0.097 (s, 3H) ppm; ¹³C NMR (151 MHz, C₆D₆) δ=214.4, 170.7, 167.1, 153.4, 152.4, 138.7, 120.7, 117.4, 82.4, 80.2, 79.6, 75.9, 64.3, 54.9, 53.4, 50.2, 48.1, 47.8, 43.3, 40.1, 37.4, 36.4, 35.4, 32.3, 27.9, 26.2, 25.5, 23.6, 23.3, 20.2, 18.5, 17.6, 15.6, 14.7, 7.43, 7.37, 6.0, 5.8, 5.1 ppm; HRMS (ESI) calcd for C₅₇H₁₀₆N₃O₁₀SSi₃⁺ [M+H]⁺ 1108.6901, found 1108.6892.

### (1S,3S,7S,10R,11S,12S,16R)-3-{(1E)-1-[2-(Aminomethyl)-1,3-thiazol-4-yl]prop-1-en-2-yl}-7,11-dihydroxy-17-(2-hydroxyethyl)-8,8,10,12,16-pentamethyl-4-oxa-17-azabicyclo[14.1.0]heptadecane-5,9-dione (13)

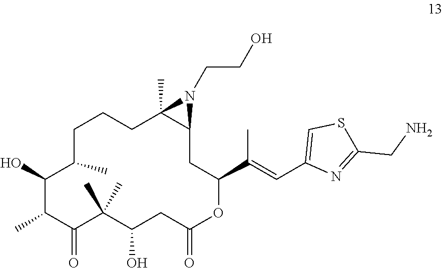

To a stirred solution of protected epothilone 84 (32 mg, 29 μmot, 1.0 equiv) in tetrahydrofuran (2.0 mL) at 0° C. was added hydrogen fluoride-pyridine complex (70% HF, 0.20 mL, 7.7 mmol, excess), and the reaction mixture was allowed to slowly warm to 25° C. After 5 h, the reaction mixture was quenched with saturated aqueous sodium bicarbonate solution (10 mL), and the two phases were separated. The aqueous layer was extracted with ethyl acetate (3×5 mL), and the combined organic layers were dried with anhydrous sodium sulfate and concentrated under reduced pressure. The crude material was redissolved in dichloromethane (2.0 mL) with stirring, and cooled to 0° C. Trifluoroacetic acid (0.50 mL, 6.5 mmol, excess) was added, and the reaction mixture was allowed to slowly warm to 25° C. After 2.5 h, the solvent was removed under reduced pressure, and the obtained residue was redissolved in ethyl acetate (15 mL) at 25° C. with stirring. Then a saturated aqueous solution of sodium bicarbonate (5 mL) was added. After 10 min, the two phases were separated, and the organic layer was dried with anhydrous sodium sulfate and concentrated under reduced pressure. The obtained residue was purified by flash column chromatography (silica gel, 0→20% methanol in acetone) to afford pure epothilone 13 (10.6 mg, 14.0 μmot, 48% yield overall) as a white amorphous solid. 13: R_{f}=0.18 (silica gel, 10% methanol in acetone); [α]_{D}²²=−0.9 (c=0.47, CH₂Cl₂); FT-IR (film) vₘₐₓ 3386, 2922, 2851, 1676, 1557, 1463, 1396, 1261, 1201, 1180, 1132, 1033, 832, 800, 721, 672 cm⁻¹; ¹H NMR (600 MHz, CD₂Cl₂) δ=7.09 (s, 1H), 6.55 (s, 1H), 5.42 (dd, J=5.4, 5.4 Hz, 1H), 4.10-4.08 (m, 1H), 3.73 (dd, J=4.8, 4.8 Hz, 1H), 3.68-3.60 (m, 2H), 3.30-3.26 (m, 1H), 2.61 (t, J=4.8 Hz, 1H), 2.50 (dd, J=13.8, 10.2 Hz, 1H), 2.38 (dd, J=13.8, 2.4 Hz, 1H), 2.09 (s, 3H), 1.96-1.87 (m, 2H), 1.70-1.65 (m, 1H), 1.56-1.49 (m, 1H), 1.46-1.26 (m, 6H), 1.35 (m, 3H), 1.15 (s, 3H), 1.12 (d, J=7.2 Hz, 3H), 1.03 (s, 3H), 0.96 (d, J=7.2 Hz, 3H) ppm; ¹³C NMR (151 MHz, CD₂Cl₂) δ=220.7, 174.2, 171.3, 152.8, 137.5, 119.4, 116.6, 77.9, 75.4, 74.4, 62.4, 55.2, 53.8, 53.0, 48.4, 44.3, 43.6, 39.6, 35.5, 35.0, 32.1, 29.8, 21.7, 20.7, 20.3, 17.5, 16.4, 15.9, 14.1 ppm; HRMS (ESI) calcd for C₂₉H₄₇N₃O₆SNa⁺ [M+Na]⁺ 588.3078, found 588.3087.

### (1S,3S,7S,10R,11S,12S,16R)-8,8,10,12,16-Pentamethyl-3-{(1E)-1-[2-(methylsulfanyl)-1,3-thiazol-4-yl]prop-1-en-2-yl}-7,11-bis[(triethylsilyl)oxy]-4-oxa-17-azabicyclo[14.1.0]heptadecane-5,9-dione (85)

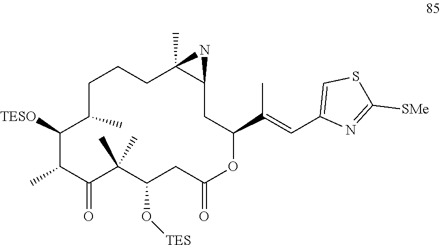

To a stirred solution of phosphonate 41 (190 mg, 0.675 mmol, 9.6 equiv) in tetrahydrofuran (1.0 mL) at −78° C. was added n-butyllithium (2.5 M in hexanes, 0.22 mL, 0.55 mmol, 7.7 equiv) dropwise. After 30 min, a solution of methyl ketone 80 (45 mg, 70 μmot, 1.0 equiv) in tetrahydrofuran (0.5 mL) was added, and the reaction mixture was allowed to slowly warm to 25° C. After 1 h, the reaction mixture was quenched with saturated aqueous ammonium chloride solution (10 mL). The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 30→100% ethyl acetate in hexanes) to afford pure protected epothilone 85 (32 mg, 42 μmol, 59% yield) as a colorless oil. 85: R_{f}=0.34 (silica gel, 30% hexanes in ethyl acetate); [α]_{D}²²=−13.3 (c=0.36, CH₂Cl₂); FT-IR (neat) vₘₐₓ 2953, 2928, 2876, 1742, 1696, 1459, 1416, 1345, 1304, 1240, 1197, 1157, 1068, 1035, 1019, 985, 915, 862, 838, 783, 737, 676 cm⁻¹; ¹H NMR (600 MHz, C₆D₆) δ=6.63 (s, 1H), 6.43 (s, 1H), 5.39 (dd, J=8.4, 3.0 Hz, 1H), 4.26 (dd, J=9.0, 3.6 Hz, 1H), 4.15 (d, J=8.4 Hz, 1H), 3.06 (dq, J=8.4, 7.2 Hz, 1H), 2.72 (dd, J=16.2, 8.4 Hz, 1H), 2.60 (dd, J=16.2, 3.6 Hz, 1H), 2.22 (s, 3H), 2.20 (s, 3H), 2.11-2.06 (m, 1H), 1.89-1.84 (m, 2H), 1.79-1.70 (m, 2H), 1.61-1.55 (m, 2H), 1.49-1.36 (m, 2H), 1.24-1.18 (m, 1H), 1.18 (d, J=7.2 Hz, 3H), 1.17 (s, 3H), 1.13 (d, J=7.2 Hz, 3H), 1.08 (t, J=7.8 Hz, 9H), 1.07 (t, J=7.8 Hz, 9H), 1.05 (s, 3H), 0.85 (s, 3H), 0.81-0.77 (m, 6H), 0.75-0.71 (m, 6H) ppm; ¹³C NMR (151 MHz, C₆D₆) δ=214.4, 170.7, 165.3, 153.6, 138.8, 120.1, 116.4, 80.2, 79.3, 75.9, 53.5, 47.9, 41.7, 40.0, 39.4, 37.2, 35.2, 34.0, 31.9, 25.8, 25.1, 23.3, 23.1, 20.0, 17.5, 15.9, 14.9, 7.4, 7.3, 6.0, 5.8 ppm; HRMS (ESI) calcd for C₃₉H₇₁N₂O₅S₂Si₂⁺ [M+H]⁺ 767.4337, found 767.4358.

### (1S,3S,7S,10R,11S,12S,16R)-7,11-Dihydroxy-8,8,10,12,16-pentamethyl-3-{(1E)-1-[2-(methyl-sulfanyl)-1,3-thiazol-4-yl]prop-1-en-2-yl}-4-oxa-17-azabicyclo[14.1.0]heptadecane-5,9-dione (14)

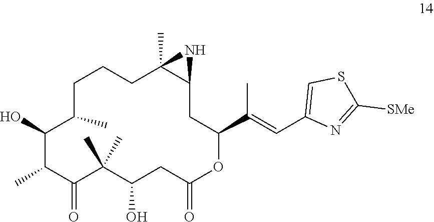

To a stirred solution of protected epothilone 85 (13.0 mg, 17.0 μmol, 1.0 equiv) in tetrahydrofuran (1.0 mL) at 0° C. was added hydrogen fluoride-pyridine complex (70% HF, 0.10 mL, 3.9 mmol, excess), and the reaction mixture was allowed to slowly warm to 25° C. After 1 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by flash column chromatography (silica gel, 5→15% methanol in ethyl acetate) to afford pure epothilone 14 (8.5 mg, 16 μmol, 93% yield) as a white amorphous solid. 14: R_{f}=0.29 (silica gel, 15% methanol in ethyl acetate); [α]2=−28.8 (c=0.85, CH₂Cl₂); FT-IR (film) vₘₐₓ 3292, 2956, 2930, 2875, 1730, 1687, 1456, 1422, 1384, 1334, 1293, 1263, 1174, 1145, 1037, 1009, 980, 881, 735, 668 cm⁻¹; ¹H NMR (600 MHz, CD₂Cl₂) δ=7.04 (s, 1H), 6.54 (s, 1H), 5.55 (dd, J=4.2, 4.2 Hz, 1H), 4.08 (ddd, J=14.4, 3.6, 3.6 Hz, 1H), 3.78 (dd, J=6.6, 3.6 Hz, 1H), 3.33 (dq, J=6.6, 6.6 Hz, 1H), 2.71 (s, 3H), 2.54 (dd, J=12.6, 10.8 Hz, 1H), 2.43 (dd, J=12.6, 4.2 Hz, 1H), 2.14 (s, 3H), 2.00 (s, 1H), 1.96 (ddd, J=15.0, 4.2, 4.2 Hz, 1H), 1.85 (dd, J=9.0, 4.8 Hz, 1H), 1.78-1.71 (m, 2H), 1.58-1.49 (m, 2H), 1.45-1.34 (m, 3H), 1.40 (s, 3H), 1.24-1.20 (m, 1H), 1.22 (s, 3H), 1.10 (d, J=7.2 Hz, 3H), 1.01 (s, 3H), 0.95 (d, J=6.6 Hz, 3H) ppm; ¹³C NMR (151 MHz, CD₂Cl₂) δ=220.6, 171.2, 165.7, 153.5, 137.2, 118.3, 116.3, 76.3, 76.2, 75.6, 60.6, 52.6, 44.9, 38.7, 38.4, 35.2, 31.1, 30.3, 28.8, 25.7, 22.6, 22.4, 18.9, 17.6, 16.9, 16.3, 14.9 ppm; HRMS (ESI) calcd for C₂₇H₄₂N₂O₅S₂Na⁺ [M+Na]⁺ 561.2427, found 561.2409.

### tert-Butyl (1S,3S,7S,10R,11S,12S,16R)-3-acetyl-8,8,10,12,16-pentamethyl-5,9-dioxo-7,11-bis[(triethylsilyl)oxy]-4-oxa-17-azabicyclo[14.1.0]heptadecane-17-carboxylate (86)

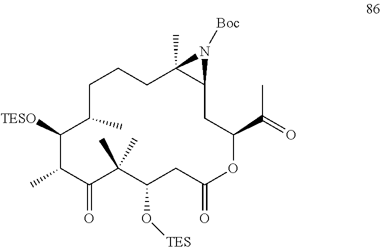

To a stirred solution of methyl ketone 80 (28 mg, 39 μmol, 1.0 equiv) in MeCN (1.0 mL) at 0° C. was added triethylamine (16 mg, 0.12 mmol, 3.0 equiv), followed by di-tert-butyl dicarbonate (26 mg, 0.12 mmol, 3.0 equiv) and 4-dimethylaminopyridine (1.0 mg, 8.2 μmol, 0.2 equiv). After 5 min, the solvent was removed under reduced pressure, and the obtained residue was purified by flash column chromatography (silica gel, 5→15% ethyl acetate in hexanes) to afford pure carbamate 86 (23 mg, 31 μmol, 78% yield) as a colorless oil. 86: R_{f}=0.36 (silica gel, 20% ethyl acetate in hexanes); [ci]2=14.1 (c=0.64, CH₂Cl₂); FT-IR (neat) vₘₐₓ 2954, 2813, 2877, 1749, 1733, 1713, 1698, 1457, 1415, 1384, 1367, 1348, 1297, 1269, 1248, 1157, 1109, 1071, 1053, 1044, 1019, 1009, 984, 941, 914, 864, 836, 811, 783, 736 cm⁻¹; ¹H NMR (600 MHz, C₆D₆) δ=4.98 (dd, J=9.6, 2.4 Hz, 1H), 4.01 (dd, J=10.2, 2.4 Hz, 1H), 3.91 (d, J=9.0 Hz, 1H), 3.00 (dq, J=9.6, 7.2 Hz, 1H), 2.91 (dd, J=16.2, 1.2 Hz, 1H), 2.74 (dd, J=16.2, 10.2 Hz, 1H), 2.39 (ddd, J=15.6, 3.0, 3.0 Hz, 1H), 2.33 (dd, J=10.8, 3.6 Hz, 1H), 2.22 (s, 3H), 1.78 (ddd, J=13.2, 13.2, 4.8 Hz, 1H), 1.62-1.57 (m, 2H), 1.50-1.40 (m, 2H), 1.46 (s, 9H), 1.36-1.32 (m, 1H), 1.25-1.18 (m, 1H), 1.22 (s, 3H), 1.21 (s, 3H), 1.15 (s, 3H), 1.07 (d, J=6.6 Hz, 3H), 1.03-0.99 (m, 1H), 0.98 (d, J=6.6 Hz, 3H), 0.97 (t, J=7.8 Hz, 9H), 0.91 (t, J=7.8 Hz, 9H), 0.64 (q, J=7.8 Hz, 6H), 0.59 (t, J=7.8 Hz, 6H) ppm; ¹³C NMR (151 MHz, C₆D₆) δ=215.3, 203.5, 171.9, 161.4, 81.4, 80.5, 76.6, 53.4, 48.7, 48.3, 46.5, 39.5, 36.6, 33.1, 31.1, 30.0, 28.3, 26.1, 24.7, 24.6, 23.8, 20.3, 19.9, 17.9, 7.3, 7.1, 5.8, 5.4 ppm; HRMS (ESI) calcd for C₃₉H₇₃NO₈Si₂Na⁺ [M+Na]⁺ 762.4767, found 762.4799.

### (1S,3S,7S,10R,11S,12S,16R)-3-Acetyl-17-(4-methoxybenzyl)-8,8,10,12,16-pentamethyl-7,11-bis-[(triethylsilyl)oxy]-4-oxa-17-azabicyclo[14.1.0]heptadecane-5,9-dione (86a)

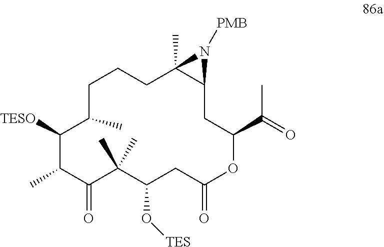

To a stirred solution of methyl ketone 80 (71.8 mg, 0.112 mmol, 1.0 equiv) in DMF (0.5 mL) at 0° C. was added p-methoxybenzyl bromide (27.1 mg, 0.135 mmol, 1.2 equiv), followed by potassium carbonate (18.7 mg, 0.135 mmol, 1.2 equiv). After 3 h, the reaction mixture was quenched with water (5 mL), allowed to warm to 25° C., and extracted with ethyl acetate (3×10 mL). The combined organic layers were dried with anhydrous sodium sulfate and concentrated under reduced pressure. The obtained residue was purified by flash column chromatography (silica gel, 5→40% ethyl acetate in hexanes) to afford p-methoxybenzyl aziridine 86a (61.7 mg, 81.1 nmol, 78% yield) as a colorless oil. 86a: R_{f}=0.29 (silica gel, 30% ethyl acetate in hexanes); [α]_{D}²²=−8.8 (c=0.42, CH₂Cl₂); FT-IR (neat) vₘₐₓ 2953, 2912, 2877, 1747, 1732, 1695, 1613, 1585, 1512, 1463, 1415, 1383, 1364, 1302, 1245, 1197, 1158, 1110, 1069, 1040, 1010, 985, 941, 914, 857, 835, 820, 783, 738, 676 cm⁻¹; ¹H NMR (600 MHz, C₆D₆) δ=7.38 (d, J=8.4 Hz, 2H), 6.88 (d, J=8.4 Hz, 2H), 4.92 (dd, J=9.6, 1.8 Hz, 1H), 4.19 (d, J=9.6 Hz, 1H), 4.05 (dd, J=6.6, 6.6 Hz, 1H), 3.62 (d, J=13.8 Hz, 1H), 3.54 (d, J=13.8 Hz, 1H), 3.35 (s, 3H), 2.85 (dq, J=9.6, 6.6 Hz 1H), 2.75-2.70 (m, 2H), 2.04 (ddd, J=15.0, 2.4, 2.4 Hz, 1H), 1.89-1.80 (m, 2H), 1.74-1.66 (m, 2H), 1.69 (s, 3H), 1.63-1.53 (m, 2H), 1.45-1.39 (m, 1H), 1.26-1.17 (m, 2H), 1.20 (d, J=7.2 Hz, 3H), 1.15 (s, 3H), 1.12 (s, 3H), 1.11-1.06 (m, 18H), 1.03 (d, J=6.6 Hz, 3H), 0.81-0.77 (m, 6H), 0.74-0.70 (m, 6H), 0.67 (s, 3H) ppm; ¹³C NMR (150 MHz, C₆D₆) δ=213.9, 202.5, 171.8, 159.2, 133.2, 129.2, 114.1, 80.8, 78.1, 76.7, 55.6, 54.8, 53.1, 49.9, 48.3, 44.1, 39.5, 37.0, 36.0, 31.7, 31.4, 25.3, 25.2, 24.9, 23.0, 20.1, 17.8, 15.3, 7.5, 7.3, 6.0, 5.8 ppm; HRMS (ESI) calcd for C₄₂H₇₄NO₇Si₂⁺ [M+H]⁺ 760.4998, found 760.5013.

### (1S,3S,7S,10R,11S,12S,16R)-3-Acetyl-8,8,10,12,16-pentamethyl-7,11-bis[(triethylsilyl)oxy]-17-{[2-(trimethylsilyl)ethoxy]methyl}-4-oxa-17-azabicyclo[14.1.0]heptadecane-5,9-dione (87)

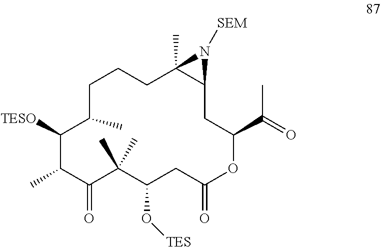

To a stirred solution of methyl ketone 80 (65.0 mg, 0.102 mmol, 1.0 equiv) in CH₂Cl₂ (0.5 mL) at 0° C. was added N,N-diisopropylethylamine (26.3 mg, 0.203 mmol, 2.0 equiv), followed by 2-(trimethylsilyl)-ethoxymethyl chloride (25.5 mg, 0.153 mmol, 1.5 equiv). After 2 h, the reaction mixture was quenched with water (10 mL), and allowed to warm to 25° C. The two phases were separated, and the aqueous layer WAC extracted with ethyl acetate (2×10 mL). The combined organic layers were dried with anhydrous sodium sulfate and concentrated under reduced pressure. The obtained residue was purified by flash column chromatography (silica gel, 5→20% ethyl acetate in hexanes) to afford pure azirdine 87 (46.0 mg, 59.7 μmol, 59% yield) as a colorless oil. 87: Rₜ=0.23 (silica gel, 20% ethyl acetate in hexanes); [α]_{D}²²=−3.1 (c=0.16, CH₂Cl₂); FT-IR (neat) vₘₐₓ 2953, 2877, 1748, 1734, 1697, 1460, 1414, 1382, 1368, 1307, 1286, 1247, 1197, 1158, 1105, 1043, 1018, 1009, 985, 940, 860, 836, 783, 736 cm⁻¹; ¹H NMR (600 MHz, C₆D₆) δ=4.93 (dd, J=9.0, 3.0 Hz, 1H), 4.18 (d, J=9.6 Hz, 1H), 4.15 (d, J=8.4 Hz, 1H), 4.07 (d, J=8.4 Hz, 1H), 4.06 (dd, J=9.0, 3.6 Hz, 1H), 3.87-3.80 (m, 2H), 2.85 (dq, J=9.0, 6.6 Hz, 1H), 2.76-2.68 (m, 2H), 2.11-2.08 (m, 1H), 1.85-1.79 (m, 2H), 1.75 (s, 3H), 1.74-1.68 (m, 1H), 1.67-1.56 (m, 2H), 1.52-1.48 (m, 1H), 1.42-1.34 (m, 1H), 1.29-1.25 (m, 1H), 1.24-1.21 (m, 1H), 1.19 (d, J=7.2 Hz, 3H), 1.15 (s, 3H), 1.11 (s, 3H), 1.10-1.06 (m, 18H), 1.08-1.02 (m, 2H), 1.04 (d, J=6.6 Hz, 3H), 0.80-0.76 (m, 6H), 0.74-0.70 (m, 6H), 0.68 (s, 3H), 0.06 (s, 9H) ppm; ¹³C NMR (151 MHz, C₆D₆) δ=213.9, 202.4, 171.7, 84.0, 80.8, 78.0, 76.7, 65.9, 53.1, 48.3, 47.7, 43.8, 39.5, 36.9, 35.5, 31.6, 31.1, 25.3, 25.0, 24.9, 22.9, 20.0, 18.4, 17.8, 15.8, 7.5, 7.3, 6.0, 5.8, 1.2 ppm; HRMS (ESI) calcd for C₄₀H₈₀NO₇Si₃⁺ [M+H]+ 770.5237, found 770.5249.

### (1S,3S,7S,10R,11S,12S,16R)-8,8,10,12,16-Pentamethyl-3-{(1E)-1-[2-(methylsulfanyl)-1,3-thiazol-4-yl]prop-1-en-2-yl}-7,11-bis[(triethylsilyl)oxy]-17-{[2-(trimethylsilyl)ethoxy]methyl}-4-oxa-17-azabicyclo[14.1.0]heptadecane-5,9-dione (89)

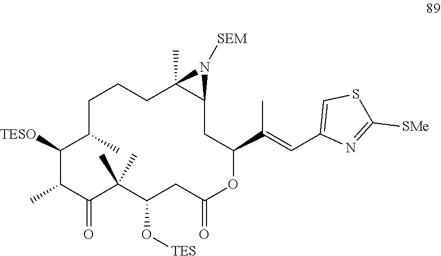

To a stirred solution of phosphonate 41 (0.120 g, 0.427 mmol, 23 equiv) in tetrahydrofuran (1.0 mL) at −78° C. was added n-butyllithium (2.5 M in hexanes, 0.14 mL, 0.34 mmol, 19 equiv) dropwise. After 30 min, the reaction mixture was transferred to a stirred solution of methyl ketone 87 (14.0 mg, 18.2 μmol, 1.0 equiv) in tetrahydrofuran (0.5 mL) at −78° C., and the reaction mixture was allowed to slowly warm to 10° C. over 2.5 h. Then the reaction mixture was quenched with saturated aqueous ammonium chloride solution (5 mL), and the two phases were separated. The aqueous layer was extracted with ethyl acetate (3×5 mL), and the combined organic layers were dried with anhydrous sodium sulfate and concentrated under reduced pressure. The obtained residue was purified by flash column chromatography (silica gel, 5→15% ethyl acetate in hexanes) to afford pure protected epothilone 89 (9.8 mg, 11 μmol, 60% yield) as a colorless oil. 89: Rₜ=0.23 (silica gel, 15% ethyl acetate in hexanes); [α]_{D}²²=5.5 (c=0.40, CH₂Cl₂); FT-IR (neat) vₘₐₓ 2952, 2912, 2876, 1742, 1696, 1459, 1417, 1380, 1345, 1303, 1281, 1247, 1197, 1181, 1157, 1095, 1069, 1036, 1018, 985, 940, 860, 836, 782, 738 cm⁻¹; ¹H NMR (600 MHz, C₆D₆) δ=6.64 (s, 1H), 6.44 (s, 1H), 5.46 (dd, J=7.8, 4.2 Hz, 1H), 4.29 (dd, J=8.4, 4.2 Hz, 1H), 4.13 (d, J=8.4 Hz, 1H), 4.12 (d, J=8, 1H), 4.08 (d, J=8.4 Hz, 1H), 3.90 (ddd, J=9.0, 7.8, 7.8 Hz, 1H), 3.78 (ddd, J=9.0, 7.8, 7.8 Hz, 1H), 3.03 (dq, J=8.4, 6.6 Hz, 1H), 2.69 (dd, J=16.2, 8.4 Hz, 1H), 2.60 (dd, J=16.2, 4.2 Hz, 1H), 2.28 (s, 3H), 2.22 (ddd, J=13.8, 4.2, 4.2 Hz, 1H), 2.20 (s, 3H), 2.10 (ddd, J=15.0, 9.0, 9.0 Hz, 1H), 1.91-1.84 (m, 2H), 1.72-1.67 (m, 1H), 1.66-1.58 (m, 1H), 1.54-1.48 (m, 2H), 1.40 (dd, J=9.0, 2.4 Hz, 1H), 1.18 (s, 3H), 1.17 (s, 3H), 1.16 (d, J=7.2 Hz, 3H), 1.13 (d, J=7.2 Hz, 3H), 1.09-1.03 (m, 18H), 0.91 (s, 3H), 0.82-0.75 (m, 6H), 0.73-0.69 (m, 6H), 0.05 (s, 9H) ppm; ¹³C NMR (150 MHz, C₆D₆) δ=214.6, 170.6, 165.3, 153.6, 138.6, 120.7, 116.5, 84.2, 80.0, 79.5, 75.6, 65.8, 53.5, 47.8, 46.8, 44.2, 40.5, 37.5, 36.3, 34.7, 32.4, 25.4, 23.5, 22.5, 20.1, 18.4, 17.5, 15.9, 15.8, 14.6, 7.4, 7.3, 5.9, 5.8, −1.1 ppm; HRMS (ESI) calcd for C₄₅H₈₄N₂O₆Si₃Na⁺ [M+Na]⁺ 919.4971, found 919.4982.

### (1S,3S,7S,10R,11S,12S,16R)-7,11-Dihydroxy-8,8,10,12,16-pentamethyl-3-{(1E)-1-[2-(methyl-sulfanyl)-1,3-thiazol-4-yl]prop-1-en-2-yl}-4-oxa-17-azabicyclo[14.1.0]heptadecane-5,9-dione (14)

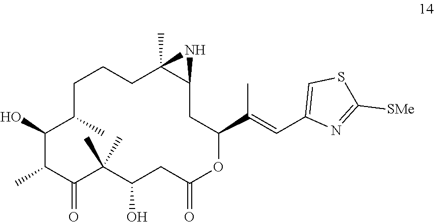

To a stirred solution of protected epothilone 89 (6.0 mg, 6.7 nmol, 1.0 equiv) in dichloromethane (1.2 mL) at 0° C. was added trifluoroacetic acid (0.3 mL, 3.9 mmol, excess), and the reaction mixture was allowed to slowly warm to 25° C. After 1.5 h, the solvent was removed under reduced pressure, and the obtained residue was purified by preparative thin layer chromatography (silica gel, 25% methanol in ethyl acetate) to afford pure epothilone 14 (2.7 mg, 5.0 nmol, 75% yield) as a white amorphous solid (for characterization data of 14, see above).

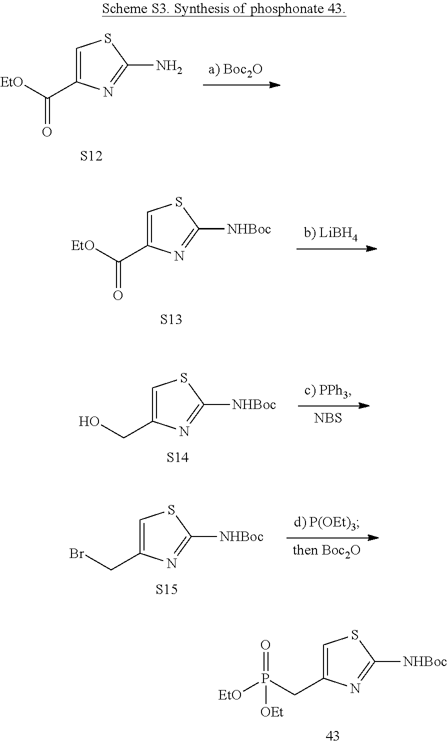

### Ethyl 2-[(tert-butoxycarbonyl)amino]-1,3-thiazole-4-carboxylate (S13)

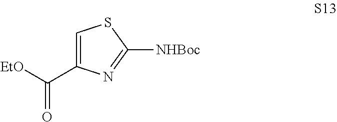 [SMILES: CCOC(=O)c1csc(NC(=O)OC(C)(C)C)n1]

To a stirred solution of aminothiazole ester S12 (0.500 g, 2.90 mmol, 1.0 equiv) in tetrahydrofuran (9.7 mL) at 25° C. was added triethylamine (0.53 mL, 3.8 mmol, 1.3 equiv), 4-(dimethylamino)pyridine (35 mg, 0.29 mmol, 0.1 equiv), and di-tert-butyl-dicarbonate (696 mg, 3.19 mmol, 1.1 equiv) sequentially, and the reaction mixture was heated to 60° C. After 1 h, the reaction mixture was allowed to cool to 25° C., and quenched with saturated aqueous ammonium chloride solution (15 mL). The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (25% ethyl acetate in hexanes) to afford pure thiazolyl carbamate S13 (569 mg, 2.10 mmol, 72% yield) as a white solid. S13: R_{f}=0.24 (silica gel, 25% ethyl acetate in hexanes); FT-IR (film) vₘₐₓ 3168, 3068, 2980, 2935, 1713, 1553, 1478, 1455, 1393, 1368, 1331, 1294, 1235, 1207, 1154, 1098, 1071, 1021, 957, 915, 875, 802, 734, 682 cm⁻¹; ¹H NMR (600 MHz, CDCl₃) δ=8.67 (br s, 1H), 7.77 (s, 1H), 4.35 (q, J=7.2 Hz, 2H), 1.52 (s, 9H), 1.36 (t, J=7.2 Hz, 3H) ppm; ¹³C NMR (151 MHz, CDCl₃) δ=161.5, 159.8, 152.3, 142.1, 121.7, 83.3, 61.4, 28.3, 14.5 ppm; HRMS (ESI) calcd for C₁₁H₁₆N₂O₄S⁺ [M+Na]⁺ 295.0723, found 295.0712.

### tert-Butyl [4-(hydroxymethyl)-1,3-thiazol-2-yl]carbamate (S14)

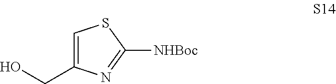 [SMILES: CC(C)(C)OC(=O)Nc1nc(CO)cs1]

To a stirred solution of thiazole carboxylate S13 (1.14 g, 4.19 mmol, 1.0 equiv) in diethyl ether (14 mL) at 25° C. was added lithium borohydride (2.0 M in tetrahydrofuran, 10.5 mL, 21.0 mmol, 5.0 equiv). After 1 h, the reaction mixture was carefully quenched with saturated aqueous ammonium chloride solution (10 mL). The two phases were separated, and the aqueous layer was extracted with ethyl acetate (3×8 mL). The combined organic layers were dried with anhydrous sodium sulfate and concentrated under reduced pressure. The obtained residue was purified by flash column chromatography (30% hexanes in ethyl acetate) to afford pure hydroxymethyl thiazole S14 (907 mg, 3.94 mmol, 94% yield) as a colorless oil. S14: R_{f}=0.57 (silica gel, 30% hexanes in ethyl acetate); FT-IR (neat) vₘₐₓ 3320, 3185, 3064, 2979, 2934, 1718, 1557, 1478, 1455, 1394, 1369, 1330, 1294, 1245, 1157, 1076, 1033, 965, 915, 868, 792, 732, 685 cm⁻¹; ¹H NMR (600 MHz, CDCl₃) δ=6.74 (s, 1H), 4.57 (s, 2H), 1.57 (s, 9H) ppm; ¹³C NMR (151 MHz, CDCl₃) δ=161.6, 152.6, 151.0, 109.2, 83.1, 60.1, 28.3 ppm; HRMS (ESI) calcd for C₉H₁₄N₂O₃SNa⁺ [M+Na]⁺ 253.0617, found 253.0616.

### tert-Butyl [4-(bromomethyl)-1,3-thiazol-2-yl]carbamate (S15)

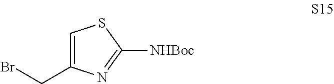 [SMILES: CC(C)(C)OC(=O)Nc1nc(CBr)cs1]

To a stirred solution of hydroxymethyl thiazole S14 (115 mg, 0.500 mmol, 1.0 equiv) in dichloromethane (5 mL) at −78° C. was added triphenylphosphine (135 mg, 0.510 mmol, 1.05 equiv), followed by N-bromosuccinimide (89 mg, 0.50 mmol, 1.0 equiv). After 15 min, the reaction mixture was quenched with water (2.5 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 20% ethyl acetate in hexanes) to afford pure bromomethyl thiazole S15 (104 mg, 0.350 mmol, 71% yield) as a colorless oil. S15: R_{f}=0.31 (silica gel, 20% ethyl acetate in hexanes); FT-IR (neat) vₘₐₓ 3164, 3056, 2978, 2933, 2803, 1713, 1553, 1478, 1454, 1432, 1393, 1368, 1332, 1289, 1243, 1215, 1151, 1068, 1033, 977, 910, 865, 791, 763, 701, 655 cm⁻¹; ¹H NMR (600 MHz, CDCl₃) δ=10.08 (br s, 1H), 6.88 (s, 1H), 4.54 (s, 2H), 1.56 (s, 9H) ppm; ¹³C NMR (151 MHz, CDCl₃) δ=161.4, 152.6, 146.8, 111.6, 83.2, 28.4, 27.8 ppm; HRMS (ESI) calcd for C₉H₁₄BrN₂O₂S⁺ [M+H]⁺ 292.9954, found 292.9950.

### Diethyl ({2-[bis(tert-butoxycarbonyl)amino]-1,3-thiazol-4-yl}methyl)phosphonate (43)

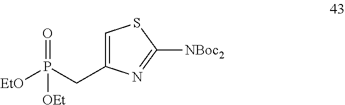

A stirred solution bromomethyl thiazole S15 (210 mg, 0.71 mmol, 1.0 equiv) in triethyl phosphite (2.4 mL, 14 mmol, 20 equiv) was heated to 160° C. After 3 h, the triethyl phosphite was removed under a steady flow of nitrogen gas, and the reaction mixture was allowed to cool to 25° C. The crude material was redissolved in tetrahydrofuran (2.4 mL) at 25° C. with stirring, and triethylamine (0.26 mL, 1.9 mmol, 2.6 equiv), 4-(dimethylamino)pyridine (9.0 mg, 70 μmot, 0.1 equiv), and di-tert-butyl-dicarbonate (340 mg, 1.6 mmol, 2.2 equiv) were added sequentially. The reaction mixture was heated to 60° C. for 3.5 h, allowed to cool to 25° C., and quenched with saturated aqueous ammonium chloride solution (10 mL). The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (35% hexanes in ethyl acetate) to afford pure phosphonate 43 (260 mg, 0.57 mmol, 80% yield) as a colorless oil. 43: R_{f}=0.28 (silica gel, 35% hexanes in ethyl acetate); FT-IR (neat) vₘₐₓ 3475, 3109, 2981, 2934, 1776, 1725, 1526, 1490, 1458, 1395, 1370, 1345, 1326, 1248, 1156, 1120, 1054, 1027, 966, 948, 846, 802, 777 cm⁻¹; ¹H NMR (600 MHz, CDCl₃) δ=7.03 (d, J=3.6 Hz, 1H), 4.10-4.05 (m, 4H), 3.28 (d, J=21.0 Hz, 2H), 1.52 (s, 18H), 1.27 (t, J=7.1 Hz, 6H) ppm; ¹³C NMR (151 MHz, CDCl₃) δ=158.0, 149.8, 142.8 (d, J=8.3 Hz), 114.5 (d, J=7.7 Hz), 84.7, 62.4 (d, J=6.2 Hz), 29.3 (d, J=140.7 Hz), 27.9, 16.5 (d, J=6.0 Hz) ppm; HRMS (ESI) calcd for C₁₈H₃₁N₂O₇PSNₐ⁺ [M+Na]⁺ 473.1482, found 473.1471.

### Di-tert-butyl {4-[(1E)-2-{(1S,3S,7S,10R,11S,12S,16R)-17-(2-{[tert-butyl(dimethyl)silyl]oxy}ethyl)-8,8,10,12,16-pentamethyl-5,9-dioxo-7,11-bis[(triethylsilyl)oxy]-4-oxa-17-azabicyclo-[14.1.0]heptadec-3-yl}prop-1-en-1-yl]-1,3-thiazol-2-yl}imidodicarbonate (15a)

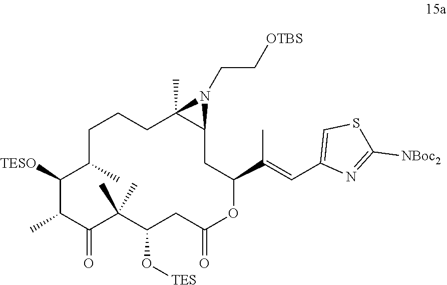

To a stirred solution of phosphonate 43 (118 mg, 0.266 mmol, 14 equiv) in tetrahydrofuran (1.2 mL) at −78° C. was added sodium bis(trimethylsilyl)amide (1.0 M in tetrahydrofuran, 0.266 mL, 0.266 mmol, 14 equiv) dropwise. After 30 min, a solution of methyl ketone 82 (15.0 mg, 19.0 μmot, 1.0 equiv) in tetrahydrofuran (1.0 mL) was added, and the reaction mixture was allowed to slowly warm to 0° C. over 3.5 h. Then the reaction mixture was quenched with saturated aqueous ammonium chloride solution (10 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 5→20% ethyl acetate in hexanes) to afford protected epothilone 15a (14.2 mg, 13.1 μmol, 69% yield) as a colorless oil. 15a: R_{f}=0.20 (silica gel, 10% ethyl acetate in hexanes); [_{α}]_{D}²²=−7.5 (c=1.0, CH₂Cl₂); FT-IR (neat) vₘₐₓ 2954, 2933, 2877, 2858, 1780, 1728, 1696, 1505, 1460, 1413, 1370, 1334, 1283, 1249, 1158, 1120, 1041, 1007, 984, 836, 806, 779, 738 cm⁻¹; ¹H NMR (600 MHz, C₆D₆) δ=6.57 (s, 1H), 6.33 (s, 1H), 5.43 (dd, J=8.6, 2.8 Hz, 1H), 4.22 (dd, J=9.2, 2.6 Hz, 1H), 4.18 (d, J=9.0 Hz, 1H), 3.87-3.79 (m, 2H), 3.01 (dq, J=7.2, 7.2 Hz, 1H), 2.73 (ddd, J=12.0, 5.9, 5.9 Hz, 1H), 2.68 (dd, J=16.1, 9.3 Hz, 1H), 2.56 (dd, J=16.1, 2.9 Hz, 1H), 2.44 (ddd, J=12.0, 6.0, 6.0 Hz, 1H), 2.34 (s, 3H), 2.26-2.24 (m, 1H), 2.08-2.02 (m, 1H), 1.87-1.80 (m, 2H), 1.75-1.69 (m, 1H), 1.64-1.57 (m, 1H), 1.51-1.48 (m, 1H), 1.37 (s, 18H), 1.34-1.21 (m, 3H), 1.19 (d, J=6.9 Hz, 3H), 1.18 (s, 3H), 1.15 (s, 3H), 1.13 (d, J=6.9 Hz, 3H), 1.10-1.05 (m, 18H), 0.99 (s, 9H), 0.85 (s, 3H), 0.83-0.77 (m, 6H), 0.74-0.70 (m, 6H), 0.100 (s, 3H), 0.097 (s, 3H) ppm; ¹³C NMR (151 MHz, C₆D₆) δ=214.6, 170.7, 157.8, 150.0, 149.2, 138.3, 121.0, 114.4, 84.2, 80.2, 79.7, 75.9, 64.3, 55.0, 53.5, 50.3, 48.2, 43.4, 40.2, 37.5, 36.4, 35.4, 32.4, 27.7, 26.2, 25.5, 23.7, 23.3, 20.2, 18.6, 17.7, 15.7, 14.6, 7.5, 7.4, 6.0, 5.9, 5.1, ppm; HRMS (ESI) calcd for C₅₆H₁₀₄N₃O₁₀SSi₃⁺ [M+H]⁺ 1094.6745, found 1094.6742.

### (1S,3S,7S,10R,11S,12S,16R)-3-[(1E)-1-(2-Amino-1,3-thiazol-4-yl)prop-1-en-2-yl]-7,11-dihydroxy-17-(2-hydroxyethyl)-8,8,10,12,16-pentamethyl-4-oxa-17-azabicyclo[14.1.0]heptadecane-5,9-dione (15)

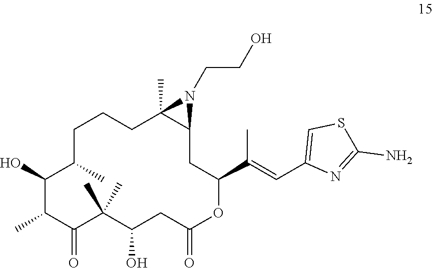

To a stirred solution of protected epothilone 15a (10 mg, 9.1 μmol, 1.0 equiv) in tetrahydrofuran (2.0 mL) at 0° C. was added hydrogen fluoride-pyridine complex (70% HF, 0.05 mL, 1.9 mmol, excess), and the reaction mixture was allowed to slowly warm to 25° C. After 5 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The crude material was redissolved in dichloromethane (1.0 mL) with stirring, and cooled to 0° C. Trifluoroacetic acid (0.10 mL, 1.3 mmol, excess) was added, and the reaction mixture was allowed to slowly warm to 25° C. After 6 h, the solvent was removed under reduced pressure, and the obtained residue was redissolved in ethyl acetate (15 mL) at 25° C. with stirring. Then a saturated aqueous solution of sodium bicarbonate (5 mL) was added. After 10 min, the two phases were separated, and the organic layer was dried with anhydrous sodium sulfate and concentrated under reduced pressure. The obtained residue was purified by flash column chromatography (silica gel, 10% methanol in dichloromethane) to afford pure epothilone 15 (4.0 mg, 7.2 μmol, 80% yield overall) as a white amorphous solid. 15: R_{f}=0.13 (silica gel, 10% methanol in dichloromethane); [α]_{D}²²=−16.7 (c=0.20, CH₂Cl₂); FT-IR (film) vₘₐₓ 3332, 2926, 2856, 1727, 1686, 1529, 1464, 1378, 1346, 1262, 1148, 1054, 1009, 982, 885, 875, 799, 735, 689 cm⁻¹; ¹H NMR (600 MHz, CD₂Cl₂) δ=6.40 (s, 1H), 6.32 (s, 1H), 5.37 (dd, J=5.2, 5.2 Hz, 1H), 5.12 (br s, 2H), 4.07 (dd, J=10.1, 2.0 Hz, 1H), 3.73-3.70 (m, 4H), 3.26 (dq, J=7.2, 7.2 Hz, 1H), 2.74-2.64 (m, 2H), 2.47 (dd, J=13.9, 10.2 Hz, 1H), 2.35 (dd, J=13.9, 2.3 Hz, 1H), 2.06 (s, 3H), 2.03-1.98 (m, 3H), 1.72-1.66 (m, 1H), 1.55-1.41 (m, 5H), 1.34 (s, 3H), 1.32-1.27 (m, 3H), 1.21 (s, 3H), 1.11 (d, J=6.9 Hz, 3H), 1.03 (s, 3H), 0.96 (d, J=6.9 Hz, 3H) ppm; ¹³C NMR (151 MHz, CD₂Cl₂) δ=220.7, 171.3, 167.0, 148.8, 136.6, 119.7, 107.6, 78.0, 75.3, 74.4, 61.7, 54.8, 53.1, 44.3, 39.6, 35.6, 34.2, 32.3, 31.5, 30.1, 27.6, 23.1, 21.7, 20.6, 17.5, 16.7, 15.7, 14.3 ppm; HRMS (ESI) calcd for C₂₈H₄₅N₃O₆SNa⁺ [M+Na]⁺ 574.2921, found 574.2899.

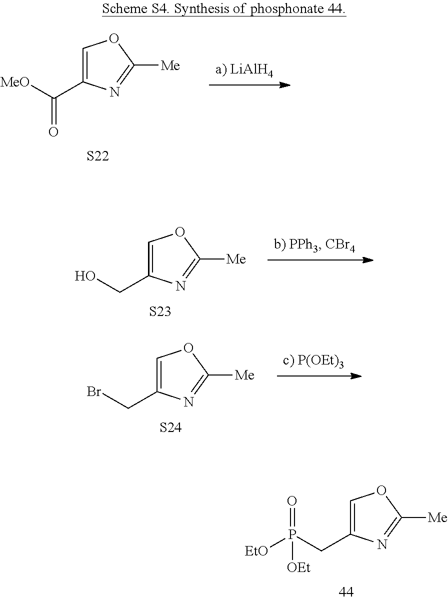

### (2-Methyl-1,3-oxazol-4-yl)methanol (S23)

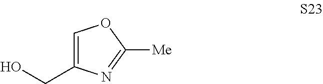

To a stirred solution of oxazole methyl ester S22 (0.500 g, 3.54 mmol, 1.0 equiv) in tetrahydrofuran (35 mL) at 0° C. was added lithium aluminum hydride (1.0 M in tetrahydrofuran, 3.54 mL, 3.54 mmol, 1.0 equiv) dropwise. After 30 min, the reaction mixture was carefully quenched with sodium sulfate decahydrate (11.4 g, 35.4 mmol, 10.0 equiv), and allowed to warm to 25° C. Then the quenched reaction mixture was filtered through a pad of Celite®, rinsed thoroughly with ethyl acetate (40 mL), and concentrated under reduced pressure. The obtained residue was purified by flash column chromatography (silica gel, ethyl acetate) to afford pure hydroxymethyl oxazole S23 (390 mg, 3.45 mmol, 97% yield) as a colorless oil. S23: R_{f}=0.39 (silica gel, ethyl acetate); FT-IR (neat) vₘₐₓ 3292, 2931, 2871, 1656, 1578, 1443, 1385, 1336, 1315, 1275, 1222, 1196, 1097, 1064, 1028, 993, 955, 929, 786, 756, 734, 655 cm⁻¹; ¹H NMR (600 MHz, CDCl₃) δ=7.47 (s, 1H), 4.54 (s, 2H), 2.90 (br s, 1H), 2.44 (s, 3H) ppm; ¹³C NMR (151 MHz, CDCl₃) δ=162.3, 140.3, 135.0, 56.4, 14.0 ppm; HRMS (CI) calcd for C₅H₈NO₂⁺ [M]⁺ 113.0477, found 113.0475.

### 4-(Bromomethyl)-2-methyl-1,3-oxazole (S24)

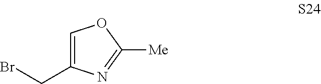

To a stirred solution of hydroxymethyl oxazole S23 (502 mg, 4.44 mmol, 1.0 equiv) in dichloromethane (37 mL) at 25° C. was added triphenylphosphine (1.98 g, 7.55 mmol, 1.7 equiv), 2,6-lutidine (0.21 mL, 1.8 mmol, 0.4 equiv) and carbon tetrabromide (2.50 g, 7.55 mmol, 1.7 equiv) sequentially. After 1 h, the reaction mixture was quenched with saturated aqueous sodium bicarbonate solution (8 mL) and a saturated aqueous solution of sodium thiosulfate (8 mL). The two phases were separated, and the aqueous layer was extracted with dichloromethane (3×5 mL). The combined organic layers were dried with anhydrous sodium sulfate and concentrated under reduced pressure. The obtained residue was purified by flash column chromatography (silica gel, 25% ethyl acetate in hexanes) to afford pure bromomethyl oxazole S24 (672 mg, 3.82 mmol, 86% yield) as a colorless oil. S24: R_{f}=0.34 (silica gel, 25% ethyl acetate in hexanes); FT-IR (neat) vₘₐₓ 3140, 2922, 2859, 1585, 1430, 1385, 1330, 1279, 1223, 1199, 1103, 1055, 1033, 989, 917, 872, 834, 772, 746, 700 cm⁻¹; ¹H NMR (600 MHz, CDCl₃) δ=7.53 (s, 1H), 4.35 (s, 2H), 2.46 (s, 3H) ppm; ¹³C NMR (151 MHz, CDCl₃) δ=162.4, 137.4, 135.9, 23.2, 14.1 ppm; HRMS (ESI) calcd for C₅H₇BrNO⁺ [M+H]⁺ 175.9711, found 175.9706.

### Diethyl [(2-methyl-1,3-oxazol-4-yl)methyl]phosphonate (44)

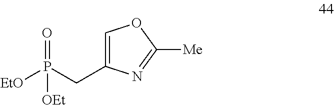 [SMILES: CCOP(=O)(Cc1coc(C)n1)OCC]

To a stirred solution of bromomethyl oxazole S24 (0.10 g, 0.57 mmol, 1.0 equiv) in benzene (1.9 mL) at 25° C. was added triethyl phosphite (0.49 mL, 2.85 mmol, 5.0 equiv). The reaction mixture was heated to 100° C. for 24 h, allowed to cool to 25° C., and concentrated under reduced pressure. The obtained residue was purified by flash column chromatography (silica gel, 0→5% methanol in ethyl acetate) to afford pure phosphonate 44 (111 mg, 0.480 mmol, 84% yield) as a colorless oil. 44: R_{f}=0.35 (silica gel, 5% methanol in ethyl acetate); FT-IR (neat) vₘₐₓ 3466, 3135, 2984, 2933, 2911, 1641, 1580, 1479, 1445, 1392, 1369, 1331, 1288, 1248, 1198, 1164, 1098, 1052, 1024, 967, 847, 811, 740, 689 cm⁻¹; ¹H NMR (600 MHz, CDCl₃) δ=7.50 (d, J=3.6 Hz, 1H), 4.14-4.08 (m, 4H), 3.06 (d, J=20.7 Hz, 2H), 2.42 (s, 3H), 1.30 (t, J=7.1 Hz, 6H) ppm; ¹³C NMR (151 MHz, CDCl₃) δ=161.4, 136.1 (d, J=7.9 Hz), 131.4 (d, J=7.8 Hz), 62.4 (d, J=6.6 Hz), 24.8 (d, J=143.2 Hz), 16.5 (d, J=6.0 Hz), 14.0 ppm; HRMS (ESI) calcd for C₉H₁₆NO₄PNa⁺ [M+Na]⁺ 256.0709, found 256.0718.

### (1S,3S,7S,10R,11S,12S,16R)-7,11-Dihydroxy-17-(2-hydroxyethyl)-8,8,10,12,16-pentamethyl-3-[(1E)-1-(2-methyl-1,3-oxazol-4-yl)prop-1-en-2-yl]-4-oxa-17-azabicyclo[14.1.0]heptadecane-5,9-dione (16)

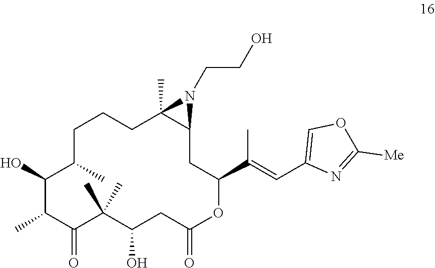

To a stirred solution of phosphonate 44 (108 mg, 0.460 mmol, 61 equiv) in tetrahydrofuran (1.0 mL) at −78° C. was added sodium bis(trimethylsilyl)amide (1.0 M in tetrahydrofuran, 0.44 mL, 0.44 mmol, 58 equiv) dropwise. After 20 min, an aliquot of the reaction mixture (0.30 mL, ca. 91 μmot, 12 equiv 44) was quickly transferred to a stirred solution of methyl ketone 82 (6.1 mg, 7.6 μmot, 1.0 equiv) in tetrahydrofuran (0.3 mL) at −78° C. The reaction mixture was allowed to slowly warm to −40° C. over 30 min, and was then quenched with saturated aqueous ammonium chloride solution (1.0 mL), and allowed to warm to 25° C. The two phases were separated, and the aqueous layer was extracted with ethyl acetate (3×1 mL). The combined organic layers were dried with anhydrous sodium sulfate and concentrated under reduced pressure. The obtained residue was purified by flash column chromatography (silica gel, 20→50% ethyl acetate in hexanes) to afford a mixture of protected epothilone (ca. 42% yield) and methyl ketone 82 (ca. 27% yield). This difficult to separate mixture was used directly in the following step.

To a stirred solution of protected epothilone (2.8 mg, 3.2 μmot, 1.0 equiv) in tetrahydrofuran (0.2 mL) at 0° C. was added hydrogen fluoride-pyridine complex (70% HF, 0.10 mL, 3.9 mmol, excess), and the reaction mixture was allowed to slowly warm to 25° C. After 3 h, the reaction mixture was quenched with saturated aqueous sodium bicarbonate solution (10 mL). The two phases were separated, and the aqueous layer was extracted with ethyl acetate (3×5 mL). The combined organic layers were dried with anhydrous sodium sulfate and concentrated under reduced pressure. The obtained residue was purified by preparative thin layer chromatography (silica gel, 5% methanol in ethyl acetate+1.5% ammonium hydroxide) to afford pure epothilone 16 [1.5 mg, 2.8 μmot, 37% yield overall, (E):(Z)=88:12] as a white amorphous solid and recovered deprotected methyl ketone 81 (0.96 mg, 2.1 μmot, 27% from 82) as a white amorphous solid. 16: R_{f}=0.40 (silica gel, 20% methanol in ethyl acetate); [α]_{D}²²=−11.5 (c=0.20, CH₂Cl₂); FT-IR (film) vₘₐₓ 3366, 2951, 2927, 2873, 1732, 1687, 1586, 1457, 1432, 1384, 1286, 1262, 1201, 1150, 1106, 1057, 1009, 981, 931, 883, 747, 666 cm⁻¹; ¹H NMR (600 MHz, CD₂Cl₂) δ=7.53 (s, 1H), 6.30 (s, 1H), 5.44 (dd, J=4.5, 4.5 Hz, 1H), 4.02 (dd, J=10.0, 2.2 Hz, 1H), 3.74 (dd, J=5.9, 4.1 Hz, 1H), 3.67-3.61 (m, 2H), 3.29 (dq, J=6.8, 6.8 Hz, 1H), 2.63 (m, 1H), 2.56 (m, 1H), 2.50 (dd, J=13.8, 10.0 Hz, 1H), 2.42 (s, 3H), 2.40 (dd, J=13.8, 2.2 Hz, 1H), 1.99 (s, 3H), 1.98-1.93 (m, 1H), 1.84-1.80 (m, 1H), 1.69-1.62 (m, 1H), 1.55-1.35 (m, 3H), 1.34 (s, 3H), 1.31-1.26 (m, 4H), 1.14 (s, 3H), 1.12 (d, J=7.0 Hz, 3H), 1.03 (s, 3H), 0.96 (d, J=7.0 Hz, 3H) ppm; ¹³C NMR (151 MHz, CD₂Cl₂) δ=220.7, 171.4, 161.4, 138.0, 136.7, 136.2, 115.7, 77.6, 76.0, 75.1, 62.5, 55.4, 52.7, 48.1, 44.9, 43.4, 39.5, 35.5, 34.7, 31.8, 29.5, 21.9, 21.6, 19.8, 17.6, 16.5, 16.0, 14.6, 14.0 ppm (¹H and ¹³C NMR were recorded as a mixture); HRMS (ESI) calcd for C₂₉H₄₇N₂O₇⁺ [M+H]⁺ 535.3378, found 535.3385.

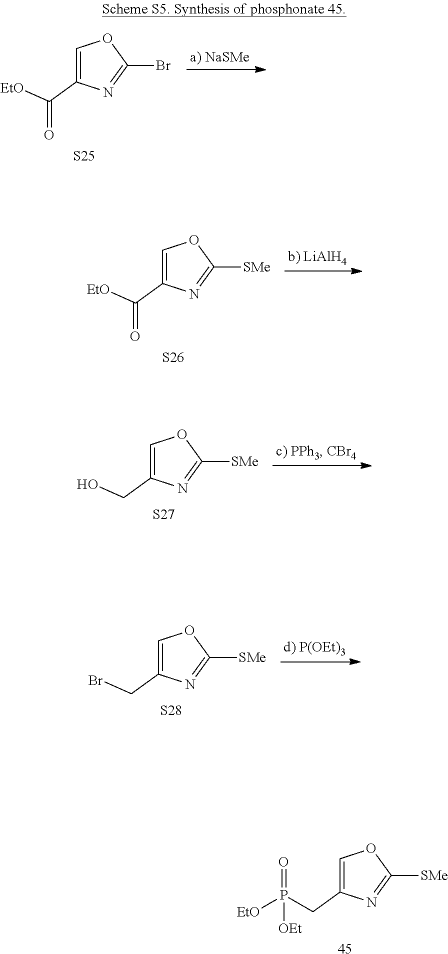

### Ethyl 2-(methylsulfanyl)-1,3-oxazole-4-carboxylate (S26)

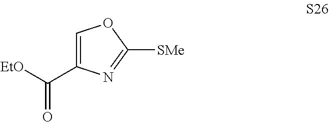 [SMILES: CCOC(=O)c1coc(SC)n1]

To a stirred solution of bromooxazole ethyl ester S25 (0.500 g, 2.27 mmol, 1.0 equiv) in ethanol (15 mL) at 25° C. was added sodium thiomethoxide (635 mg, 9.06 mmol, 4.0 equiv). After 2 h, the reaction mixture was quenched with saturated aqueous ammonium chloride solution (10 mL), and diluted with ethyl acetate (20 mL). The two phases were separated, and the aqueous layer was extracted with ethyl acetate (3×5 mL), and the combined organic layers were dried with anhydrous sodium sulfate and concentrated under reduced pressure. The obtained residue was purified by flash column chromatography (silica gel, 20% ethyl acetate in hexanes) to afford pure oxazole ethyl ester S26 (111 mg, 0.480 mmol, 84% yield) as a colorless oil. S26: R_{f}=0.35 (silica gel, 20% ethyl acetate in hexanes); FT-IR (neat) vₘₐₓ 3161, 3114, 2983, 2935, 1739, 1719, 1579, 1567, 1504, 1446, 1392, 1370, 1315, 1262, 1177, 1128, 1092, 1022, 973, 924, 863, 830, 763, 692 cm⁻¹; ¹H NMR (600 MHz, CDCl₃) δ=8.18 (s, 1H), 4.37 (q, J=7.1 Hz, 2H), 2.70 (s, 3H), 1.37 (t, J=7.1 Hz, 3H) ppm; ¹³C NMR (151 MHz, CDCl₃) δ=163.3, 161.1, 145.0, 134.8, 61.4, 14.9, 14.4 ppm; HRMS (ESI) calcd for C₇H₉NO₃SNa⁺ [M+Na]⁺ 210.0195, found 210.0195.

### [2-(Methylsulfanyl)-1,3-oxazol-4-yl]methanol (S27)

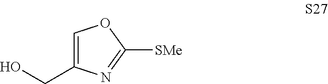

To a stirred solution of oxazole ethyl ester S26 (520 mg, 2.8 mmol, 1.0 equiv) in tetrahydrofuran (28 mL) at 0° C. was added lithium aluminum hydride (1.0 M in tetrahydrofuran, 1.4 mL, 1.4 mmol, 0.5 equiv). After 30 min, the reaction mixture was carefully quenched with sodium sulfate decahydrate (9.0 g, 28 mmol, 10 equiv), and allowed to warm to 25° C. Then the quenched reaction mixture was filtered through a pad of Celite®, rinsed thoroughly with ethyl acetate (30 mL), and the solution was concentrated under reduced pressure. The obtained residue was purified by flash column chromatography (silica gel, 25% hexanes in ethyl acetate) to afford pure hydroxymethyl oxazole S27 (333 mg, 2.29 mmol, 82% yield) as a colorless oil. S27: R_{f}=0.39 (silica gel, 25% hexanes in ethyl acetate); FT-IR (neat) vₘₐₓ 3339, 3142, 2933, 2872, 1493, 1432, 1310, 1268, 1209, 1154, 1060, 1027, 987, 935, 776, 744, 690 cm⁻¹; ¹H NMR (600 MHz, CDCl₃) δ=7.58 (s, 1H), 4.57 (d, J=1.1 Hz, 2H), 2.65 (s, 3H), 1.95 (br s, 1H) ppm; ¹³C NMR (151 MHz, CDCl₃) δ=162.2, 141.5, 136.5, 57.0, 14.8 ppm; HRMS (CI) calcd for C₅H₇NO₂S⁺[M]⁺ 144.0119, found 144.0117.

### 4-(Bromomethyl)-2-(methylsulfanyl)-1,3-oxazole (S28)

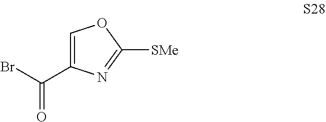

To a stirred solution of hydroxymethyl oxazole S27 (333 mg, 2.29 mmol, 1.0 equiv) in dichloromethane (23 mL) at 0° C. was added triphenylphosphine (1.02 g, 3.89 mmol, 1.7 equiv) and carbon tetrabromide (1.29 g, 3.89 mmol, 1.7 equiv) sequentially. After 1 h, the reaction mixture was quenched sequentially with a saturated aqueous solution of sodium bicarbonate (8 mL) and a saturated aqueous solution of sodium thiosulfate (8 mL), and allowed to warm to 25° C. The two phases were separated, and the aqueous layer was extracted with dichloromethane (3×5 mL). The combined organic layers were dried with anhydrous sodium sulfate and concentrated under reduced pressure. The obtained residue was purified by flash column chromatography (silica gel, 25% ethyl acetate in hexanes) to afford pure bromomethyl oxazole S28 (367 mg, 1.76 mmol, 77% yield) as a white amorphous solid. S28: R_{f}=0.32 (silica gel, 10% ethyl acetate in hexanes); FT-IR (film) vₘₐₓ 3178, 3134, 3014, 2970, 2933, 1593, 1500, 1429, 1307, 1258, 1211, 1155, 1087, 991, 981, 932, 761, 727, 694, 652 cm⁻¹; ¹H NMR (600 MHz, CDCl₃) δ=7.64 (s, 1H), 4.34 (s, 2H), 2.66 (s, 3H) ppm; ¹³C NMR (151 MHz, CDCl₃) δ=162.4, 138.8, 137.5, 22.9, 14.8 ppm; HRMS (ESI) calcd for C₅H₇BrNOS⁺ [M+H]⁺ 207.9426, found 207.9425.

### Diethyl {[2-(methylsulfanyl)-1,3-oxazol-4-yl]methyl}phosphonate (45)

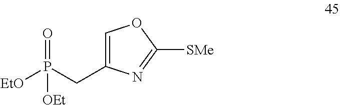 [SMILES: CCOP(=O)(Cc1coc(SC)n1)OCC]

To a stirred solution of bromomethyl oxazole S28 (416 mg, 2.00 mmol, 1.0 equiv) in benzene (6.7 mL) at 25° C. was added triethyl phosphite (1.7 mL, 10 mmol, 5.0 equiv). The reaction mixture was heated to 95° C. for 12 h, allowed to cool to 25° C., and concentrated under reduced pressure. The obtained residue was purified by flash column chromatography (silica gel, 50→100% ethyl acetate in hexanes) to afford pure phosphonate 45 (498 mg, 1.88 mmol, 94% yield) as a colorless oil. 45: R_{f}=0.28 (silica gel, ethyl acetate); FT-IR (neat) vₘₐₓ 3466, 3191, 3139, 2983, 2933, 2910, 2871, 1647, 1595, 1497, 1443, 1394, 1369, 1309, 1244, 1163, 1052, 1024, 966, 846, 808, 725, 688 cm⁻¹; ¹H NMR (600 MHz, CDCl₃) δ=7.62 (d, J=3.6 Hz, 1H), 4.16-4.09 (m, 4H), 3.08 (d, J=20.7 Hz, 2H), 2.63, (s, 3H), 1.32 (t, J=7.1 Hz, 6 Hi ppm; ¹³C NMR (151 MHz, CDCl₃) δ=161.1, 137.7 (d, J=8.0 Hz), 133.0 (d, J=7.8 Hz), 62.5 (d, J=6.4 Hz), 24.9 (d, J=143.1 Hz), 16.5 (d, J=6.1 Hz), 14.8 ppm; HRMS (ESI) calcd for C₉H₁₇NO₄PS⁺ [M+H]⁺ 266.0610, found 266.0621.

### (1S,3S,7S,10R,11S,12S,16R)-17-(2-{[tert-Butyl(dimethyl)silyl]oxy}ethyl)-8,8,10,12,16-pentamethyl-3-{(1E)-1-[2-(methylsulfanyl)-1,3-oxazol-4-yl]prop-1-en-2-yl}-7,11-bis[(triethylsilyl)oxy]-4-oxa-17-azabicyclo[14.1.0]heptadecane-5,9-dione (17a)

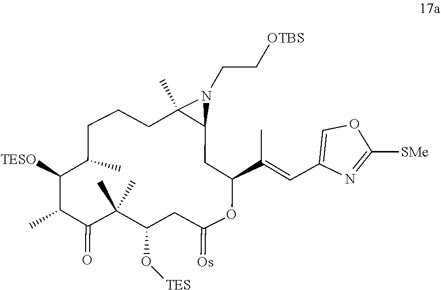

To a stirred solution of phosphonate 45 (493 mg, 1.86 mmol, 219 equiv) in tetrahydrofuran (5.0 mL) at −78° C. was added sodium bis(trimethylsilyl)amide (1.0 M in tetrahydrofuran, 1.75 mL, 1.75 mmol, 206 equiv) dropwise. After 20 min, an aliquot of the reaction mixture (0.46 mL, ca. 130 μmol, 15 equiv 45) was transferred to a stirred solution of methyl ketone 82 (6.8 mg, 8.5 μmol, 1.0 equiv) in tetrahydrofuran (0.4 mL) at −78° C. After 30 min, the reaction mixture was quenched with saturated aqueous ammonium chloride solution (0.6 mL), diluted with water (5 mL) and ethyl acetate (5 mL), and allowed to warm to 25° C. The two phases were separated, and the aqueous layer was extracted with ethyl acetate (3×3 mL). The combined organic layers were dried with anhydrous sodium sulfate and concentrated under reduced pressure. The obtained residue was purified by flash column chromatography (silica gel, 20→50% ethyl acetate in hexanes), and further purified by preparative thin layer chromatography (silica gel, 30% ethyl acetate in hexanes) to afford pure protected epothilone 17a (5.0 mg, 5.5 μmol, 65% yield) as a colorless oil. 17a: R_{f}=0.20 (silica gel, 15% ethyl acetate in hexanes); [α]2=4.6 (c=0.50, CH₂Cl₂); FT-IR (neat) vₘₐₓ 2953, 2934, 2877, 1743, 1697, 1505, 1462, 1414, 1381, 1346, 1305, 1282, 1251, 1199, 1180, 1158, 1105, 1033, 1018, 1008, 985, 939, 836, 813, 779, 738, 678, 664 cm⁻¹; NMR (600 MHz, C₆D₆) δ=7.08 (s, 1H), 6.48 (s, 1H), 5.41 (dd, J=8.2, 3.2 Hz, 1H), 4.22 (dd, J=8.8, 2.7 Hz, 1H), 4.17 (d, J=8.9 Hz, 1H), 3.86-3.78 (m, 2H), 3.02 (dq, J=8.8, 6.9 Hz, 1H), 2.76-2.68 (m, 2H), 2.58 (dd, J=16.2, 3.2 Hz, 1H), 2.45 (ddd, J=12.5, 6.5, 6.5 Hz, 1H), 2.27-2.23 (m, 1H), 2.14 (s, 3H), 2.12 (s, 3H), 2.05-1.99 (m, 1H), 1.89-1.80 (m, 2H), 1.73-1.69 (m, 1H), 1.64-1.57 (m, 1H), 1.50-1.46 (m, 2H), 1.36-1.22 m, 2H), 1.19 (d, J=6.9 Hz, 3H), 1.17 (s, 3H), 1.14 (s, 3H), 1.13 (d, J=6.9 Hz, 3H), 1.10-1.05 (m, 18H), 0.99 (s, 9H), 0.86 (s, 3H), 0.83-0.76 (m, 6H), 0.73-0.69 (m, 6H), 0.09 (s, 6H) ppm; ¹³C NMR (151 MHz, C₆D₆) δ=214.5, 170.7, 161.3, 140.0, 139.0, 137.9, 116.6, 80.2, 79.2, 75.9, 64.3, 54.9, 53.5, 50.2, 48.1, 43.4, 40.2, 37.4, 36.4, 35.3, 32.4, 26.2, 25.5, 23.6, 23.2, 20.2, 18.6, 17.7, 15.7, 14.9, 14.4, 7.5, 7.4, 6.0, 5.9, 5.1 ppm; HRMS (ESI) calcd for C₄₇H₈₉N₂O₇SSi₃⁺ [M+H]⁺ 909.5693, found 909.5703.

### (1S,3S,7S,10R,11S,12S,16R)-7,11-Dihydroxy-17-(2-hydroxyethyl)-8,8,10,12,16-pentamethyl-3-{(1E)-1-[2-(methylsulfanyl)-1,3-oxazol-4-yl]prop-1-en-2-yl}-4-oxa-17-azabicyclo[14.1.0]-heptadecane-5,9-dione (17)

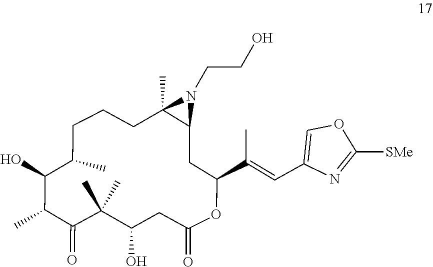

To a stirred solution of protected epothilone 17a (5.0 mg, 5.5 μmol, 1.0 equiv) in tetrahydrofuran (1.0 mL) at 0° C. was added hydrogen fluoride-pyridine complex (70% HF, 0.10 mL, 3.9 mmol, excess), and the reaction mixture was allowed to slowly warm to 25° C. After 3 h, the reaction mixture was quenched with saturated aqueous sodium bicarbonate solution (10 mL). The two phases were separated, and the aqueous layer was extracted with ethyl acetate (3×5 mL). The combined organic layers were dried with anhydrous sodium sulfate and concentrated under reduced pressure. The obtained residue was purified by preparative thin layer chromatography (silica gel, 20% methanol in ethyl acetate) to afford pure epothilone 17 (2.9 mg, 5.2 μmol, 95% yield) as a white amorphous solid. 17: R_{f}=0.32 (silica gel, 10% methanol in ethyl acetate); [α]2_{D}²²=−18.5 (c=0.20, CH₂Cl₂); FT-IR (film) vₘₐₓ 3405, 2958, 2929, 2874, 1728, 1689, 1552, 1500, 1465, 1380, 1335, 1284, 1267, 1178, 1147, 1055, 1033, 1008, 981, 938, 885, 832, 775, 741, 706, 667 cm⁻¹; ¹H NMR (600 MHz, CD₂Cl₂) δ=7.63 (s, 1H), 6.30 (s, 1H), 5.43 (dd, J=4.2, 4.2 Hz, 1H), 4.02 (dd, J=9.9, 2.0 Hz, 1H), 3.74 (dd, J=5.8, 4.1 Hz, 1H), 3.69-3.62 (m, 2H), 3.28 (dq, J=6.5, 6.5 Hz, 1H), 2.67-2.63 (m, 1H), 2.65 (s, 3H), 2.61-2.57 (m, 1H), 2.50 (dd, J=13.8, 9.9 Hz, 1H), 2.40 (dd, J=13.8, 2.2 Hz, 1H), 2.03 (br s, 1H), 2.02 (s, 3H), 2.00-1.95 (m, 1H), 1.87-1.83 (m, 1H), 1.69-1.63 (m, 1H), 1.49-1.31 (m, 7H), 1.34 (s, 3H), 1.16 (s, 3H), 1.12 (d, J=7.0 Hz, 3H), 1.03 (s, 3H), 0.96 (d, J=7.0 Hz, 3H) ppm; ¹³C NMR (151 MHz, CD₂Cl₂) δ=220.6, 171.4, 161.2, 139.3, 137.9, 137.4, 115.3, 77.6, 76.0, 75.1, 62.3, 55.3, 52.7, 48.3, 44.9, 43.7, 39.5, 35.5, 34.4, 31.6, 29.5, 21.9, 21.6, 19.9, 17.7, 16.5, 15.9, 15.0, 14.6 ppm; HRMS (ESI) calcd for C₂₉H₄₇N₂O₇S⁺ [M+H]⁺ 567.3098, found 567.3118.

### (1S,3S,7S,10R,11S,12S,16R)-17-(2-Acetoxyethyl)-8,8,10,12,16-pentamethyl-3-{(1E)-1-[2-(methylsulfanyl)-1,3-oxazol-4-yl]prop-1-en-2-yl}-5,9-dioxo-4-oxa-17-azabicyclo[14.1.0]-heptadecane-7,11-diyl diacetate (18)

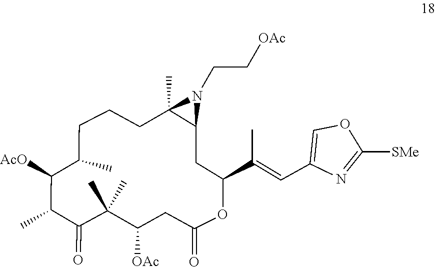

To a stirred solution of epothilone 17 (1.0 mg, 2.4 μmol, 1.0 equiv) in dichloromethane (0.25 mL) at 0° C. was added freshly distilled acetic anhydride (1.1 μL, 12 μmol, 5.0 equiv), followed by 4-dimethylaminopyridine (0.90 mg, 7.2 μmol, 3.0 equiv). The reaction mixture was allowed to slowly warm to 25° C. over 25 min, and was then quenched with saturated aqueous ammonium chloride solution (1.0 mL). The two phases were separated, and the aqueous layer was extracted with ethyl acetate (3×0.5 mL). The combined organic layers were dried with anhydrous sodium sulfate and concentrated under reduced pressure. The obtained residue was purified by preparative thin layer chromatography (silica gel, 25% hexanes in ethyl acetate) to afford pure epothilone 18 (0.90 mg, 1.8 μmol, 74% yield) as a colorless film. 18: R_{f}=0.34 (silica gel, 25% hexanes in ethyl acetate); [α]_{D}²²=+18.5 (c=0.10, CH₂Cl₂); FT-IR (film) vₘₐₓ 2923, 2855, 1736, 1661, 1631, 1467, 1377, 1260, 1054, 1033, 1016, 936, 828, 811, 793, 727, 695, 681 cm⁻¹; ¹H NMR (600 MHz, CD₂Cl₂) δ=7.63 (s, 1H), 6.23 (s, 1H), 5.54 (dd, J=10.4, 1.9 Hz, 1H), 5.28 (dd, J=5.3, 5.3 Hz, 1H), 5.17 (dd, J=8.3, 3.3 Hz, 1H), 4.18-4.14 (m, 1H), 4.12-4.08 (m, 1H), 3.46 (dq, J=6.9, 6.9 Hz, 1H), 2.81-2.75 (m, 1H), 2.65-2.57 (m, 2H), 2.64 (s, 3H), 2.56-2.48 (m, 1H), 2.062 (s, 3H), 2.058 (s, 3H), 2.01 (s, 3H), 1.99 (s, 3H), 1.95 (s, 3H), 1.74-1.44 (m, 4H), 1.42-1.29 (m, 3H), 1.19 (s, 3H), 1.16 (s, 3H), 1.03 (d, J=6.8 Hz, 3H), 1.03 (s, 3H), 0.95 (d, J=6.8 Hz, 3H) ppm; ¹³C NMR (151 MHz, CD₂Cl₂) δ=213.9, 171.11, 171.06, 170.7, 169.5, 161.2, 139.3, 138.4, 138.2, 117.1, 79.9, 77.4, 69.7, 65.1, 51.1, 49.5, 44.2, 42.7, 37.2, 37.0, 35.6, 33.7, 30.3, 30.1, 23.1, 22.4, 21.2, 21.04, 21.00, 18.1, 17.8, 16.0, 15.0, 14.2 ppm; HRMS (ESI) calcd for C₃₅H₅₃N₂O₁₀S⁺ [M+H]⁺ 693.3415, found 693.3419.

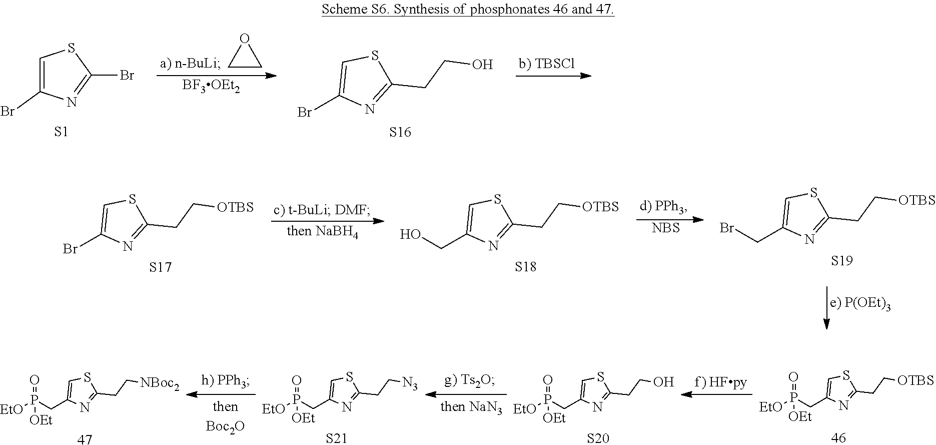

### 2-(4-Bromo-1,3-thiazol-2-yl)ethanol (S16)

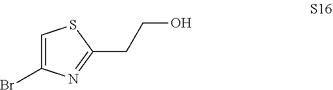

To a stirred solution of 2,4-dibromothiazole S1 (10.2 g, 42.0 mmol, 1.0 equiv) in diethyl ether (250 mL) at −78° C. was added n-butyllithium (2.5 M in hexanes, 16.8 mL, 42.0 mmol, 1.0 equiv) dropwise. After 20 min, ethylene oxide (2.5 M in tetrahydrofuran, 16.8 mL, 42.0 mmol, 1.0 equiv) was added, followed by dropwise addition of a solution of boron trifluoride diethyl etherate complex (5.18 mL, 42.0 mmol, 1.0 equiv) in diethyl ether (30 mL). After 20 min, the reaction mixture was quenched with saturated aqueous ammonium chloride solution (50 mL), and allowed to warm to 25° C. The two phases were separated, and the aqueous layer was extracted with ethyl acetate (3×80 mL). The combined organic layers were dried with anhydrous sodium sulfate and concentrated under reduced pressure. The obtained residue was purified by flash column chromatography (silica gel, 30→60% ethyl acetate in hexanes) to afford pure hydroxyethyl thiazole S16 (5.42 g, 26.0 mmol, 62% yield) as a colorless oil. S16: R_{f}=0.24 (silica gel, 50% ethyl acetate in hexanes); FT-IR (neat) vₘₐₓ 3350, 3122, 2881, 1480, 1421, 1330, 1257, 1210, 1135, 1085, 1052, 938, 887, 857, 832, 733 cm⁻¹; ¹H NMR (600 MHz, CDCl₃) δ=7.12 (s, 1H), 4.02 (td, J=6.0, 6.0 Hz, 2H), 3.22 (t, J=6.0 Hz, 2H), 2.67 (t, J=6.0 Hz, 1H) ppm; ¹³C NMR (151 MHz, CDCl₃) δ=169.7, 124.6, 116.5, 61.3, 36.2 ppm; HRMS (ESI) calcd for C₅H₇BrNOS⁺ [M+H]⁺ 207.9426, found 207.9421.

### 4-Bromo-2-(2-{[tert-butyl(dimethyl)silyl]oxy}ethyl)-1,3-thiazole (S17)

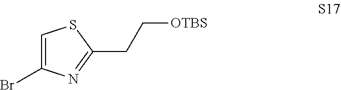 [SMILES: CC(C)(C)[Si](C)(C)OCCc1nc(Br)cs1]

To a stirred solution of hydroxyethyl thiazole S16 (5.38 g, 25.9 mmol, 1.0 equiv) in dimethylformamide (25 mL) at 25° C. was added tert-butyldimethylsilyl chloride (4.68 g, 31.0 mmol, 1.2 equiv), followed by imidazole (2.64 g, 38.9 mmol, 1.5 equiv). After 1 h, the reaction mixture was diluted with ethyl acetate (100 mL), then washed with water (20 mL) and brine (20 mL). The two phases were separated, and the organic layer was dried with anhydrous sodium sulfate and concentrated under reduced pressure. The obtained residue was purified by flash column chromatography (silica gel, 2→8% ethyl acetate in hexanes) to afford pure silyl ether S17 (8.25 g, 25.6 mmol, 99% yield) as a colorless oil. S17: R_{f}=0.24 (silica gel, 5% ethyl acetate in hexanes); FT-IR (neat) vₘₐₓ 3125, 2954, 2928, 2856, 1481, 1471, 1437, 1388, 1361, 1331, 1254, 1147, 1099, 1050, 1006, 939, 914, 884, 831, 810, 776, 728 cm⁻¹; ¹H NMR (600 MHz, CDCl₃) δ=7.09 (s, 1H), 3.93 (t, J=6.0 Hz, 2H), 3.19 (t, J=6.0 Hz, 2H), 0.87 (s, 9H), 0.02 (s, 6H) ppm; ¹³C NMR (151 MHz, CDCl₃) δ=169.6, 124.1, 116.7, 61.9, 37.2, 26.0, 18.4, 5.3 ppm; HRMS (ESI) calcd for C₁₁H₂₁BrNOSSi⁺ [M+H]⁺ 322.0291, found 322.0281.

### [2-(2-{[tert-Butyl(dimethyl)silyl]oxy}ethyl)-1,3-thiazol-4-yl]methanol (S18)

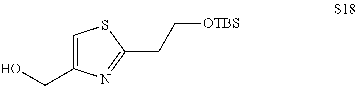 [SMILES: CC(C)(C)[Si](C)(C)OCCc1nc(CO)cs1]

To a stirred solution of silyl ether S17 (2.45 g, 7.60 mmol, 1.0 equiv) in diethyl ether (75 mL) at −78° C. was added tert-butyllithium (1.7 M in pentanes, 5.40 mL, 9.12 mmol, 1.2 equiv) dropwise. After 1 min, dimethylformamide (1.17 mL, 15.2 mmol, 2.0 equiv) was added dropwise. After 5 min, the reaction mixture was quenched with methanol (30 mL), sodium borohydride (1.44 g, 38.0 mmol, 5.0 equiv) was added, and the reaction mixture was allowed to warm to 0° C. After 5 min, the reaction mixture was quenched with water (60 mL), and allowed to warm to 25° C. The two phases were separated, and the aqueous layer was extracted with ethyl acetate (3×40 mL). The combined organic layers were dried with anhydrous sodium sulfate and concentrated under reduced pressure. The obtained residue was purified by flash column chromatography (silica gel, 30→60% ethyl acetate in hexanes) to afford pure hydroxymethyl thiazole S18 (1.70 g, 6.23 mmol, 82% yield) as a colorless oil. S18: R_{f}=0.32 (silica gel, 60% ethyl acetate in hexanes); FT-IR (neat) vₘₐₓ 3301, 2954, 2928, 2857, 1530, 1471, 1387, 1361, 1254, 1156, 1096, 969, 937, 913, 834, 810, 774, 660 cm⁻¹; ¹H NMR (600 MHz, CDCl₃) δ=7.05 (s, 1H), 4.73 (d, J=6.0 Hz, 2H), 3.94 (t, J=6.0 Hz, 2H), 3.18 (t, J=6.6 Hz, 2H), 3.09 (t, J=6.0 Hz, 1H), 0.87 (s, 9H), 0.02 (s, 6H) ppm; ¹³C NMR (151 MHz, CDCl₃) δ=169.0, 155.6, 114.7, 62.2, 60.9, 37.0, 26.0, 18.4, 5.3 ppm; HRMS (ESI) calcd for C₁₂H₂₄NO₂SSi⁺ [M+H]⁺ 296.1111, found 296.1102.

### 4-(Bromomethyl)-2-(2-{[tert-butyl(dimethyl)silyl]oxy}ethyl)-1,3-thiazole (S19)

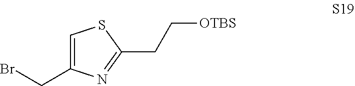 [SMILES: CC(C)(C)[Si](C)(C)OCCc1nc(CBr)cs1]

To a stirred solution of hydroxymethyl thiazole S18 (2.45 g, 8.96 mmol, 1.0 equiv) in dichloromethane (30 mL) at −78° C. was added triphenylphosphine (2.47 g, 9.41 mmol, 1.05 equiv), followed by N-bromosuccinimide (1.59 g, 8.96 mmol, 1.0 equiv). After 5 min, the reaction mixture was quenched with water (50 mL), and allowed to warm to 25° C. The two phases were separated, and the aqueous layer was extracted with ethyl acetate (3×20 mL). The combined organic layers were dried with anhydrous sodium sulfate and concentrated under reduced pressure. The obtained residue was purified by flash column chromatography (silica gel, 2→8% ethyl acetate in hexanes) to afford pure bromomethyl thiazole S19 (2.93 g, 8.71 mmol, 97% yield) as a colorless oil. S19: Rₜ=0.19 (silica gel, 5% ethyl acetate in hexanes); FT-IR (neat) vₘₐₓ 2954, 2928, 2883, 2856, 1517, 1471, 1424, 1387, 1361, 1333, 1254, 1214, 1161, 1095, 1053, 1006, 977, 937, 915, 834, 810, 775, 731, 679, 659 cm⁻¹; ¹H NMR (600 MHz, CDCl₃) δ=7.16 (s, 1H), 4.55 (s, 2H), 3.95 (t, J=6.0 Hz, 2H), 3.19 (t, J=6.0 Hz, 2H), 0.87 (s, 9H), 0.02 (s, 6H) ppm; ¹³C NMR (151 MHz, CDCl₃) δ=169.2, 151.4, 117.8, 77.4, 62.1, 37.1, 27.4, 26.0, 18.4, 5.3 ppm; HRMS (ESI) calcd for C₁₂H₂₃BrNOSSi⁺ [M+H]+ 336.0448, found 336.0441.

### Diethyl {[2-(2-{[tert-butyl(dimethyl)silyl]oxy}ethyl)-1,3-thiazol-4-yl]methyl}phosphonate (46)

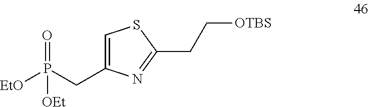 [SMILES: CCOP(=O)(Cc1csc(CCO[Si](C)(C)C(C)(C)C)n1)OCC]

A stirred solution of bromomethyl thiazole S19 (2.83 g, 8.41 mmol, 1.0 equiv) in triethyl phosphite (5.0 mL, 29 mmol, 3.5 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 50→100% ethyl acetate in hexanes) to afford pure phosphonate 46 (3.29 g, 8.36 mmol, 99% yield) as a colorless oil. 46: Rₜ=0.35 (silica gel, ethyl acetate); FT-IR (neat) vₘₐₓ 3468, 2955, 2929, 2857, 1652, 1519, 1472, 1444, 1391, 1361, 1323, 1252, 1162, 1097, 1054, 1026, 964, 917, 836, 811, 777, 723, 662 cm⁻¹; ¹H NMR (600 MHz, CDCl₃) δ=7.09 (d, J=3.6 Hz, 1H), 4.08 (dq, J=8.4, 7.2 Hz, 4H), 3.93 (t, J=6.0 Hz, 2H), 3.36 (d, J=21.0 Hz, 2H), 3.17 (t, J=6.0 Hz, 2H), 1.28 (t, J=7.2 Hz, 6H), 0.88 (s, 9H), 0.02 (s, 6H) ppm; ¹³C NMR (151 MHz, CDCl₃) δ=167.6, 145.7 (d, J=8.0 Hz), 116.2 (d, J=7.1 Hz), 62.4 (d, J=6.5 Hz), 62.3, 37.1, 29.5 (d, J=140.1 Hz), 26.0, 18.4, 16.6 (d, J=6.0 Hz), 5.3 ppm; HRMS (ESI) calcd for C₁₆H₃₂NO₄PSSiNa⁺ [M+Na]⁺ 416.1451, found 416.1441.

### (1S,3S,7S,10R,11S,12S,16R)-17-(2-{[tert-Butyl(dimethyl)silyl]oxy}ethyl)-3-{(1E)-1-[2-(2-{[tert-butyl(dimethyl)silyl]oxy}ethyl)-1,3-thiazol-4-yl]prop-1-en-2-yl}-8,8,10,12,16-pentamethyl-7,11-bis[(triethylsilyl)oxy]-4-oxa-17-azabicyclo[14.1.0]heptadecane-5,9-dione (19a)

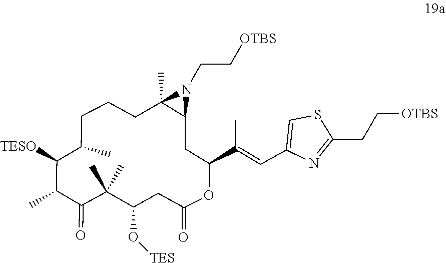

To a stirred solution of phosphonate 46 (0.200 g, 0.508 mmol, 12 equiv) in tetrahydrofuran (0.8 mL) at −78° C. was added sodium bis(trimethylsilyl)amide (1.0 m in tetrahydrofuran, 0.41 mL, 0.41 mmol, 9.7 equiv) dropwise. After 25 min, a solution of methyl ketone 82 (33.6 mg, 42.1 μmol, 1.0 equiv) in tetrahydrofuran (0.5 mL) was added, and the reaction mixture was allowed to slowly warm to 0° C. After 2 h, the reaction mixture was quenched with saturated aqueous ammonium chloride solution (10 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 5→15% ethyl acetate in hexanes) to afford pure protected epothilone 19a (21.8 mg, 21.0 μmol, 50% yield) as a colorless oil. 19a: R_{f}=0.36 (silica gel, 15% ethyl acetate in hexanes); [α]_{D}²²=−2.9 2.9 (c=0.63, CH₂Cl₂); FT-IR (neat) vₘₐₓ 2954, 2931, 2877, 2858, 1743, 1697, 1502, 1462, 1414, 1381, 1361, 1304, 1252, 1198, 1158, 1103, 1007, 984, 940, 916, 836, 812, 778, 735, 678, 662 cm⁻¹; ¹H NMR (600 MHz, CDCl₃) δ=6.78 (s, 1H), 6.60 (s, 1H), 5.48 (dd, J=8.4, 3.0 Hz, 1H), 4.23 (dd, J=8.4, 2.4 Hz, 1H), 4.18 (d, J=9.0 Hz, 1H), 3.86-3.78 (m, 2H), 3.77 (t, J=6.0 Hz, 2H), 3.06-2.99 (m, 1H), 3.02 (t, J=6.0 Hz, 2H), 2.77-2.70 (m, 2H), 2.60 (dd, J=16.2, 3.0 Hz, 1H), 2.45 (ddd, J=12.0, 6.0, 6.0 Hz, 1H), 2.36 (s, 3H), 2.30 (ddd, J=15.0, 3.0, 3.0 Hz, 1H), 2.06 (ddd, J=15.6, 9.0, 9.0 Hz, 1H), 1.90-1.81 (m, 2H), 1.76-1.71 (m, 1H), 1.66-1.58 (m, 1H), 1.52-1.48 (m, 2H), 1.28 (dd, J=9.6, 3.0 Hz, 1H), 1.24-1.20 (m, 1H), 1.20 (d, J=6.6 Hz, 3H), 1.18 (s, 3H), 1.16 (s, 3H), 1.13 (d, J=7.2 Hz, 3H), 1.09 (t, J=7.8 Hz, 9H), 1.06 (t, J=7.8 Hz, 9H), 1.00 (s, 9H), 0.94 (s, 9H), 0.86 (s, 3H), 0.85-0.77 (m, 6H), 0.74-0.70 (m, 6H), 0.096 (s, 3H), 0.094 (s, 3H), 0.02 (s, 6H) ppm; ¹³C NMR (151 MHz, CDCl₃) δ=214.5, 170.7, 166.6, 153.3, 138.3, 121.1, 117.1, 80.1, 79.7, 75.9, 64.3, 62.3, 54.9, 53.4, 50.2, 48.1, 43.3, 40.2, 37.4, 37.2, 36.4, 35.4, 32.4, 26.2, 26.0, 25.5, 23.6, 23.2, 20.2, 18.5, 18.4, 17.6, 15.6, 14.8, 7.43, 7.37, 6.0, 5.8, 5.1, 5.4 ppm; HRMS (ESI) calcd for C₅₄H₁₀₅N₂O₇SSi₄⁺ [M+H]⁺ 1037.6714, found 1037.6720.

### (1S,3S,7S,10R,11S,12S,16R)-7,11-Dihydroxy-17-(2-hydroxyethyl)-3-{(1E)-1-[2-(2-hydroxyethyl)-1,3-thiazol-4-yl]prop-1-en-2-yl}-8,8,10,12,16-pentamethyl-4-oxa-17-azabicyclo-[14.1.0]heptadecane-5,9-dione (19)

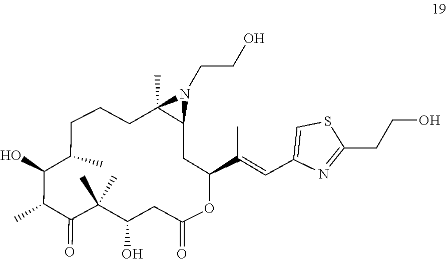

To a stirred solution of protected epothilone 19a (6.9 mg, 6.6 nmol, 1.0 equiv) in tetrahydrofuran (1.5 mL) at 0° C. was added hydrogen fluoride-pyridine complex (70% HF, 0.03 mL, 1.2 mmol, excess), and the reaction mixture was allowed to slowly warm to 25° C. After 4 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by flash column chromatography (silica gel, 0→30% methanol in ethyl acetate) to afford pure epothilone 19 (3.5 mg, 6.0 nmol, 90% yield) as a white amorphous solid. 19: R_{f}=0.35 (silica gel, 30% methanol in ethyl acetate); [α]2=−20.0 (c=0.35, 10:1 dichloromethane/methanol); FT-IR (neat) vₘₐₓ 3362, 2931, 2877, 1726, 1687, 1561, 1505, 1466, 1425, 1383, 1334, 1266, 1148, 1054, 1008, 981, 938, 883, 735, 675 cm⁻¹; ¹H NMR (600 MHz, CDCl₃) δ=7.08 (s, 1H), 6.56 (s, 1H), 5.41 (dd, J=6.0, 3.6 Hz, 1H), 4.09 (dd, J=10.2, 2.4 Hz, 1H), 3.97 (t, J=6.0 Hz, 2H), 3.71 (dd, J=4.8, 4.8 Hz, 1H), 3.69-3.62 (m, 2H), 3.29-3.25 (m, 1H), 3.20-3.18 (t, J=6.0 Hz, 2H), 2.66 (ddd, J=12.0, 4.8, 4.8 Hz, 1H), 2.59 (ddd, J=12.0, 4.8, 4.8 Hz, 1H), 2.49 (dd, J=13.8, 10.2 Hz, 1H), 2.37 (dd, J=13.8, 2.4 Hz, 1H), 2.08 (s, 3H), 2.00-1.94 (m, 1H), 1.91 (ddd, J=7.2, 7.2, 7.2 Hz, 1H), 1.68-1.64 (m, 1H), 1.67-1.35 (m, 6H), 1.35 (s, 3H), 1.30-1.22 (m, 1H), 1.26 (s, 3H), 1.17 (s, 3H), 1.13 (d, J=6.6 Hz, 3H), 1.03 (s, 3H), 0.96 (d, J=6.6 Hz, 3H) ppm; ¹³C NMR (151 MHz, CDCl₃) δ=220.6, 171.4, 168.1, 152.7, 137.2, 119.2, 116.6, 77.9, 75.9, 75.0, 62.4, 61.5, 55.3, 52.7, 48.1, 44.8, 43.4, 39.5, 36.0, 35.5, 34.7, 31.8, 29.5, 21.8, 21.4, 19.9, 17.6, 16.5, 15.9, 14.5 ppm; HRMS (ESI) calcd for C₃₀H₄₈N₂O₇SNa⁺ [M+H]⁺ 603.3074, found 603.3081.

### Diethyl {[2-(2-hydroxyethyl)-1,3-thiazol-4-yl]methyl}phosphonate (S20)

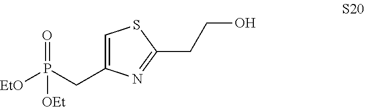 [SMILES: CCOP(=O)(Cc1csc(CCO)n1)OCC]

To a stirred solution of phosphonate 46 (2.75 g, 6.99 mmol, 1.0 equiv) in tetrahydrofuran (20 mL) at 0° C. was added hydrogen fluoride-pyridine complex (70% HF, 0.90 mL, 35 mmol, 5.0 equiv). After 1 h, the reaction mixture was quenched with saturated aqueous sodium bicarbonate solution (50 mL), and allowed to warm to 25° C. The two phases were separated, and the aqueous layer was extracted with ethyl acetate (3×20 mL). The combined organic layers were dried with anhydrous sodium sulfate and concentrated under reduced pressure. The obtained residue was purified by flash column chromatography (silica gel, 0→10% methanol in dichloromethane) to afford pure hydroxyethyl phosphonate S20 (1.94 g, 6.95 mmol, 99% yield) as a colorless oil. S20: R_{f}=0.20 (silica gel, 5% methanol in dichloromethane); FT-IR (neat) vₘₐₓ 3389, 2982, 2909, 1653, 1519, 1477, 1443, 1393, 1368, 1324, 1226, 1162, 1126, 1098, 1048, 1017, 963, 874, 842, 808, 784, 722, 668 cm⁻¹; ¹H NMR (600 MHz, CDCl₃) δ=7.07 (d, J=3.6 Hz, 1H), 4.07 (dq, J=7.8, 6.6 Hz, 4H), 3.96 (td, J=6.0, 6.0 Hz, 2H), 3.66 (t, J=6.0 Hz, 1H), 3.33 (d, J=21.0 Hz, 2H), 3.16 (t, J=6.0 Hz, 2H), 1.27 (t, J=7.2 Hz, 6H) ppm; ¹³C NMR (151 MHz, CDCl₃) δ=168.3, 146.1 (d, J=8.6 Hz), 115.8 (d, J=8.0 Hz), 62.4 (d, J=6.6 Hz), 61.3, 35.7, 29.5 (d, J=140.4 Hz), 16.5 (d, J=6.0 Hz) ppm; HRMS (ESI) calcd for C₁₀H₁₈NO₄PSNa⁺ [M+Na]⁺ 302.0586, found 302.0577.

### Diethyl {[2-(2-azidoethyl)-1,3-thiazol-4-yl]methyl}phosphonate (S21)

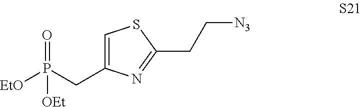 [SMILES: CCOP(=O)(Cc1csc(CCN=[N+]=[N-])n1)OCC]

To a stirred solution of hydroxyethyl phosphonate S20 (1.37 g, 4.91 mmol, 1.0 equiv) in dichloromethane (10 mL) at 25° C. was added triethylamine (2.05 mL, 14.7 mmol, 3.0 equiv) and 4-(dimethylamino)pyridine (60.0 mg, 0.491 mmol, 0.1 equiv). The reaction mixture was cooled to 20° C., and p-toluenesulfonic anhydride (3.20 g, 9.81 mmol, 2.0 equiv) was added. After 30 min, the reaction mixture was quenched with water (10 mL), and allowed to warm to 25° C. The two phases were separated, and the aqueous layer was extracted with dichloromethane (3×10 mL). The combined organic layers were dried with anhydrous sodium sulfate and concentrated under reduced pressure. The crude tosylate was then redissolved in dimethylformamide (5 mL) at 25° C. with stirring. Then sodium azide (957 mg, 14.7 mmol, 3.0 equiv) was added, and the reaction mixture was heated to 65° C. After 2 h, the reaction mixture was allowed to cool to 25° C., quenched with water (20 mL), and extracted with ethyl acetate (3×15 mL). The combined organic layers were dried with anhydrous sodium sulfate and concentrated under reduced pressure. The obtained residue was purified by flash column chromatography (silica gel, 0→4% methanol in dichloromethane) to afford pure azidoethyl phosphonate S21 (1.16 g, 3.81 mmol, 78% yield) as a colorless oil. S21: R_{f}=0.38 (silica gel, 5% methanol in dichloromethane); FT-IR (neat) vₘₐₓ 3464, 3111, 2983, 2931, 2098, 1647, 1519, 1477, 1445, 1394, 1323, 1250, 1163, 1124, 1098, 1053, 1025, 965, 873, 846, 828, 783, 725, 663 cm⁻¹; ¹H NMR (600 MHz, CDCl₃) δ=7.14 (d, J=3.6 Hz, 1H), 4.09 (dq, J=7.8, 7.2 Hz, 4H), 3.71 (t, J=6.6 Hz, 2H), 3.37 (d, J=21.0 Hz, 2H), 3.23 (t, J=6.6 Hz, 2H), 1.29 (t, J=7.2 Hz, 6H) ppm; ¹³C NMR (151 MHz, CDCl₃) δ=165.9, 146.7 (d, J=8.1 Hz), 116.5 (d, J=7.2 Hz), 62.4 (d, J=6.0 Hz), 50.7, 33.1, 29.6 (d, J=140.3 Hz), 16.6 (d, J=6.0 Hz) ppm; HRMS (ESI) calcd for C₁₀H₁₇N₄O₃PSNa⁺ [M+Na]⁺ 327.0651, found 327.0661.

### Diethyl [(2-{2-[bis(tert-butoxycarbonyl)amino]ethyl}-1,3-thiazol-4-yl)methyl]phosphonate (47)

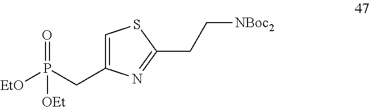

To a stirred solution of azidoethyl phosphonate S21 (1.06 g, 3.48 mmol, 1.0 equiv) in tetrahydrofuran/water (9:1, 15 mL) at 25° C. was added triphenylphosphine (2.74 g, 10.5 mmol, 3.0 equiv), and the reaction mixture was heated to 65° C. After 1.5 h, the reaction mixture was allowed to cool to 25° C., and water (6 mL), sodium bicarbonate (0.882 g, 10.5 mmol, 3.0 equiv), and di-tert-butyl dicarbonate (1.52 g, 6.96 mmol, 2.0 equiv) were added sequentially. After 2.5 h, the two phases were separated, and the aqueous layer was extracted with ethyl acetate (3×15 mL). The combined organic layers were dried with anhydrous sodium sulfate and concentrated under reduced pressure. The obtained residue was purified by flash column chromatography (silica gel, 0→7.5% methanol in dichloromethane) to afford phosphonate 47 (1.65 g, 3.44 mmol, 99% yield) as a colorless oil. 47: R_{f}=0.37 (silica gel, 5% methanol in dichloromethane); FT-IR (neat) vₘₐₓ 3471, 2980, 2933, 1791, 1748, 1697, 1519, 1478, 1444, 1393, 1367, 1353, 1254, 1220, 1166, 1126, 1054, 1026, 962, 892, 854, 806, 779, 722 cm⁻¹; ¹H NMR (600 MHz, CDCl₃) δ=7.10 (d, J=3.6 Hz, 1H), 4.08 (dq, J=7.8, 7.2 Hz, 4H), 3.96-3.94 (m, 2H), 3.35 (d, J=21.0 Hz, 2H), 3.25-3.23 (m, 2H), 1.49 (s, 18H), 1.28 (t, J=7.2 Hz, 6H) ppm; ¹³C NMR (151 MHz, CDCl₃) δ=166.7, 152.3, 146.5 (d, J=7.8 Hz), 116.1 (d, J=7.2 Hz), 82.8, 62.4 (d, J=6.0 Hz), 46.1, 32.9, 29.5 (d, J=140.0 Hz), 28.2, 16.6 (d, J=6.0 Hz) ppm; HRMS (ESI) calcd for C₂₀H₃₅N₂O₇PSNa⁺ [M+Na]⁺ 501.1795, found 501.1803.

### Di-tert-butyl (2-{4-[(1E)-2-{(1S,3S,7S,10R,11S,12S,16R)-17-(2-{[tert-butyl(dimethyl)silyl]oxy}ethyl)-8,8,10,12,16-pentamethyl-5,9-dioxo-7,11-bis[(triethylsilyl)oxy]-4-oxa-17-azabicyclo-[14.1.0]heptadec-3-yl}prop-1-en-1-yl]-1,3-thiazol-2-yl}ethyl)imidodicarbonate (20a)

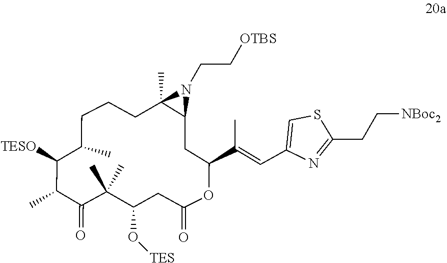

To a stirred solution of phosphonate 47 (330 mg, 0.690 mmol, 12 equiv) in tetrahydrofuran (1.0 mL) at −78° C. was added sodium bis(trimethylsilyl)amide (1.0 M in tetrahydrofuran, 0.41 mL, 0.41 mmol, 9.7 equiv) dropwise. After 25 min, a solution of methyl ketone 82 (45.0 mg, 56.4 μmot, 1.0 equiv) in tetrahydrofuran (0.5 mL) was added, and the reaction mixture was allowed to slowly warm to 0° C. After 2 h, the reaction mixture was quenched with saturated aqueous ammonium chloride solution (10 mL) and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by flash column chromatography (silica gel, 5→20% ethyl acetate in hexanes) to afford pure protected epothilone 20a (28.2 mg, 25.1 mmol, 45% yield) as a colorless oil. 20a: R_{f}=0.30 (silica gel, 20% ethyl acetate in hexanes); [α]_{D}22=4.0 (c=1.0, CH₂Cl₂); FT-IR (neat) vₘₐₓ 2954, 2935, 2877, 1794, 1744, 1697, 1500, 1459, 1390, 1367, 1353, 1306, 1278, 1251, 1220, 1158, 1118, 1040, 1008, 984, 858, 835, 779, 738, 668 cm⁻¹; ¹H NMR (600 MHz, CDCl₃) δ=6.71 (s, 1H), 6.53 (s, 1H), 5.43 (dd, J=8.4, 3.0 Hz, 1H), 4.20 (dd, J=9.0, 3.0 Hz, 1H), 4.18 (d, J=8.4 Hz, 1H), 4.08 (t, J=7.2 Hz, 2H), 3.86-3.79 (m, 2H), 3.28-3.20 (m, 2H), 3.03 (dq, J=9.0, 7.2 Hz, 1H), 2.78-2.70 (m, 2H), 2.61 (dd, J=16.2, 3.0 Hz, 1H), 2.46 (ddd, J=13.2, 6.6, 6.6 Hz, 1H), 2.34 (s, 3H), 2.29 (ddd, J=15.0, 3.0, 3.0 Hz, 1H), 2.04 (ddd, J=15.6, 9.0, 9.0 Hz, 1H), 1.91-1.81 (m, 2H), 1.75-1.71 (m, 1H), 1.67-1.59 (m, 1H), 1.52-1.48 (m, 2H), 1.39 (s, 18H), 1.26 (dd, J=10.2, 3.6 Hz, 1H), 1.22-1.8 (m, 1H), 1.20 (d, J=7.2 Hz, 3H), 1.19 (s, 3H), 1.16 (s, 3H), 1.13 (d, J=6.6 Hz, 3H), 1.09 (t, J=7.8 Hz, 9H), 1.06 (t, J=7.8 Hz, 9H), 0.99 (s, 9H), 0.87 (s, 3H), 0.85-0.77 (m, 6H), 0.74-0.70 (m, 6H), 0.097 (s, 3H), 0.093 (s, 3H) ppm; ¹³C NMR (151 MHz, CDCl₃) δ=214.4, 170.7, 165.9, 153.8, 152.6, 138.6, 120.8, 117.0, 81.8, 80.2, 79.6, 75.9, 64.3, 54.9, 53.4, 50.2, 48.1, 46.1, 43.3, 40.1, 37.3, 36.4, 35.4, 32.9, 32.3, 28.0, 26.2, 25.5, 23.6, 23.3, 20.1, 15.5, 17.6, 15.6, 14.8, 7.43, 7.37, 6.0, 5.8, 5.1 ppm; HRMS (ESI) calcd for C₅₈H₁₀₈N₃O₁₀SSi₃⁺ [M+H]⁺ 1122.7058, found 1122.7033.

### (1S,3S,7S,10R,11S,12S,16R)-3-{(1E)-1-[2-(2-Aminoethyl)-1,3-thiazol-4-yl]prop-1-en-2-yl}-7,11-dihydroxy-17-(2-hydroxyethyl)-8,8,10,12,16-pentamethyl-4-oxa-17-azabicyclo[14.1.0]heptadecane-5,9-dione (20)

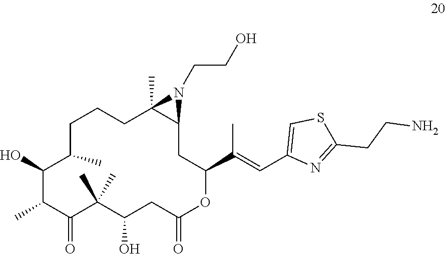

To a stirred solution of protected epothilone 20a (14.9 mg, 13.3 μmol, 1.0 equiv) in tetrahydrofuran (2.0 mL) at 0° C. was added hydrogen fluoride-pyridine complex (70% HF, 0.06 mL, 2.31 mmol, excess), and the reaction mixture was allowed to slowly warm to 25° C. After 5 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The crude material was redissolved in dichloromethane (1.0 mL) with stirring, and cooled to 0° C. Trifluoroacetic acid (0.10 mL, 1.3 mmol, excess) was added, and the reaction mixture was allowed to slowly warm to 25° C. and stirred for an additional 3 h. The solvent was removed under reduced pressure, and the obtained residue was redissolved in ethyl acetate (15 mL) at 25° C. with stirring. Then a saturated aqueous solution of sodium bicarbonate (5 mL) was added. After 10 min, the two phases were separated, and the organic layer was dried with anhydrous sodium sulfate and concentrated under reduced pressure. The obtained residue was purified by flash column chromatography (silica gel, 40% methanol in acetone) to afford pure epothilone 20 (5.5 mg, 10 μmol, 71% yield overall) as a white amorphous solid. 20: R_{f}=0.39 (silica gel, 40% methanol in acetone); [α]_{D}²²=−27.2 (c=0.50, 10:1 dichloromethane/methanol); FT-IR (neat) vₘₐₓ 3360, 2925, 2855, 1727, 1686, 1559, 1505, 1464, 1425, 1382, 1336, 1265, 1147, 1053, 1008, 980, 937, 883, 826, 733, 701, 669 cm⁻¹; ¹H NMR (600 MHz, CDCl₃) δ=7.05 (s, 1H), 6.56 (s, 1H), 5.43 (dd, J=4.8, 4.8 Hz, 1H), 4.09 (dd, J=10.2, 2.4 Hz, 1H), 3.73 (t, J=4.8 Hz, 1H), 3.68-3.60 (m, 2H), 3.28 (dq, J=6.6, 6.6 Hz, 1H), 3.09 (s, 4H), 2.61 (t, J=5.4 Hz, 2H), 2.50 (dd, J=13.8, 10.2 Hz, 1H), 2.38 (dd, J=13.8, 2.4 Hz, 1H), 2.10 (s, 3H), 1.96-1.86 (m, 2H), 1.70-1.65 (m, 1H), 1.54-1.24 (m, 9H), 1.35 (s, 3H), 1.26 (s, 2H), 1.15 (s, 3H), 1.12 (d, J=6.6 Hz, 3H), 1.03 (s, 3H), 0.96 (d, J=6.6 Hz, 3H) ppm; ¹³C NMR (151 MHz, CDCl₃) δ=220.7, 171.3, 168.4, 152.7, 137.5, 119.3, 116.5, 78.0, 75.4, 74.4, 62.4, 55.3, 53.0, 48.3, 44.3, 43.5, 42.0, 39.6, 37.5, 35.5, 35.1, 32.1, 29.8, 21.7, 20.7, 20.3, 17.5, 16.4, 15.9, 14.1 ppm; HRMS (ESI) calcd for C₃₀H₄₉N₃O₆SNa⁺ [M+Na]⁺ 602.3234, found 602.3217.

### Scheme S7. Synthesis of Phosphonate 48

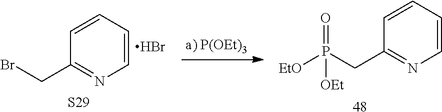

### Diethyl(pyridin-2-ylmethyl)phosphonate (48)

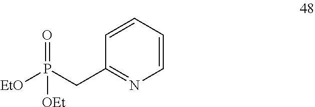 [SMILES: CCOP(=O)(Cc1ccccn1)OCC]

A stirred solution of bromomethyl pyridinium hydrobromide salt S29 (0.410 g, 2.38 mmol, 1.0 equiv) in triethyl phosphite (1.5 mL, 8.8 mmol, 3.7 equiv) was heated to 160° C. After 2.5 h, the triethyl phosphite was removed under a steady flow of nitrogen gas, and the reaction mixture was allowed to cool to 25° C. The obtained residue was purified by flash column chromatography (silica gel, 50→80% ethyl acetate in hexanes) to afford pure phosphonate 48 (355 mg, 2.87 mmol, 65% yield) as a colorless oil. 48: R_{f}=0.33 (silica gel, ethyl acetate); FT-IR (neat) vₘₐₓ 3467, 2983, 2931, 2908, 1588, 1570, 1474, 1435, 1392, 1368, 1238, 1199, 1162, 1097, 1048, 1018, 957, 839, 809, 748, 704 cm⁻¹; ¹H NMR (600 MHz, CDCl₃) δ=8.54 (dd, J=4.8, 1.8 Hz, 1H), 7.64 (ddd, J=7.8, 7.8, 1.8 Hz, 1H), 7.39 (ddd, J=7.8, 2.4, 1.2 Hz, 1H), 7.19-7.16 (m, 1H), 4.08 (dq, J=7.8, 7.2 Hz, 4H), 3.42 (d, J=22.2 Hz, 2H), 1.27 (t, J=7.2 Hz, 6H) ppm; ¹³C NMR (151 MHz, CDCl₃) δ=152.9 (d, J=8.3 Hz), 149.7 (d, J=2.5 Hz), 136.7 (d, J=2.6 Hz), 124.5 (d, J=5.0 Hz), 122.0 (d, J=3.3 Hz), 62.4 (d, J=6.5 Hz), 36.9 (d, J=134.6 Hz), 16.5 (d, J=6.0 Hz) ppm; HRMS (ESI) calcd for C₁₀H₁₇NO₃P⁺ [M+H]⁺ 230.0941, found 230.0948.

### (1S,3S,7S,10R,11S,12S,16R)-17-(2-{[tert-Butyl(dimethyl)silyl]oxy}ethyl)-8,8,10,12,16-pentamethyl-3-[(1E)-1-(pyridin-2-yl)prop-1-en-2-yl]-7,11-bis[(triethylsilyl)oxy]-4-oxa-17-azabicyclo[14.1.0]heptadecane-5,9-dione (21a)

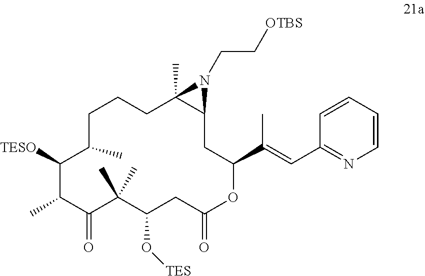

To a stirred solution of phosphonate 48 (317 mg, 1.38 mmol, 28 equiv) in tetrahydrofuran (1 mL) at −78° C. was added n-butyllithium (2.5 M in hexanes, 0.44 mL, 1.1 mmol, 22 equiv) dropwise. After 30 min, a solution of methyl ketone 82 (40.0 mg, 50 μmol, 1.0 equiv) in tetrahydrofuran (0.5 mL) was added, and the reaction mixture was allowed to slowly warm to 25° C. After 1.5 h, the reaction mixture was quenched with saturated aqueous ammonium chloride solution (10 mL), and the two phases were separated. The aqueous layer was extracted with ethyl acetate (3×5 mL), and the combined organic layers were dried with anhydrous sodium sulfate and concentrated under reduced pressure. The obtained residue was purified by flash column chromatography (silica gel, 10→40% ethyl acetate in hexanes) to afford pure protected epothilone 21a (41 mg, 47 μmol, 94% yield) as a colorless oil. 21a: R_{f}=0.23 (silica gel, 30% ethyl acetate in hexanes); [α]2=4.5 (c=1.0, CH₂Cl₂); FT-IR (neat) vₘₐₓ 2953, 2935, 2877, 1743, 1696, 1655, 1586, 1561, 1464, 1430, 1381, 1304, 1250, 1198, 1158, 1108, 1069, 1018, 1007, 985, 835, 777, 739, 676 cm⁻¹; ¹H NMR (600 MHz, C₆D₆) δ=8.51 (d, J=5.0 Hz, 1H), 7.02 (ddd, J=7.2, 7.2, 1.8 Hz, 1H), 6.89 (d, J=7.8 Hz, 1H), 6.75 (s, 1H), 6.53 (dd, J=7.2, 5.4 Hz, 1H), 5.48 (dd, J=8.4, 3.0 Hz, 1H), 4.22 (dd, J=9.0, 3.0 Hz, 1H), 4.18 (d, J=9.0 Hz, 1H), 3.86-3.78 (m, 2H), 3.04 (dq, J=9.0, 6.6 Hz, 1H), 2.77-2.71 (m, 2H), 2.59 (dd, J=16.2, 3.0 Hz, 1H), 2.48-2.43 (m, 1H), 2.45 (s, 3H), 2.31 (ddd, J=15.0, 3.0, 3.0 Hz, 1H), 2.07 (ddd, J=15.0, 9.0, 9.0 Hz, 1H), 1.90-1.81 (m, 2H), 1.76-1.71 (m, 1H), 1.66-1.59 (m, 1H), 1.52-1.48 (m, 2H), 1.29 (dd, J=9.6, 3.6 Hz, 1H), 1.25-1.21 (m, 1H), 1.20 (d, J=7.2 Hz, 3H), 1.18 (s, 3H), 1.16 (s, 3H), 1.13 (d, J=6.6 Hz, 3H), 1.09 (t, J=7.8 Hz, 9H), 1.06 (t, J=7.8 Hz, 9H), 0.99 (s, 9H), 0.84 (s, 3H), 0.83-0.78 (m, 6H), 0.73-0.69 (m, 6H), 0.09 (s, 3H), 0.08 (s, 3H) ppm; ¹³C NMR (151 MHz, C₆D₆) δ=214.5, 170.7, 157.0, 149.4, 142.5, 135.6, 126.6, 124.7, 121.0, 80.2, 79.8, 75.9, 64.3, 54.9, 53.4, 50.3, 48.1, 43.3, 40.2, 37.4, 36.4, 35.4, 32.3, 26.2 (3C), 25.4, 23.6, 23.2, 20.2, 18.5, 17.6, 15.6, 14.7, 7.43, 7.37, 6.0, 5.8, 5.1 ppm; HRMS (ESI) calcd for C₄₈H₈₉N₂O₆Si₃⁺ [M+H]⁺ 873.6023, found 873.6044.

### (1S,3S,7S,10R,11S,12S,16R)-7,11-Dihydroxy-17-(2-hydroxyethyl)-8,8,10,12,16-pentamethyl-3-[(1E)-1-(pyridin-2-yl)prop-1-en-2-yl]-4-oxa-17-azabicyclo[14.1.0]heptadecane-5,9-dione (21)

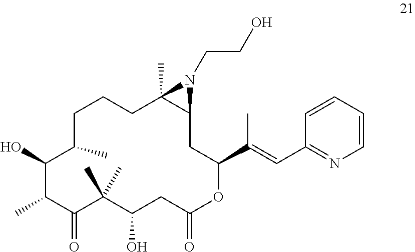

To a stirred solution of protected epothilone 21a (39.0 mg, 44.5 μmol, 1.0 equiv) in tetrahydrofuran (2.0 mL) at 0° C. was added hydrogen fluoride-pyridine complex (70% HF, 0.20 mL, 7.7 mmol, excess), and the reaction mixture was allowed to slowly warm to 25° C. After 5 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by flash column chromatography (silica gel, 5→40% methanol in ethyl acetate) to afford pure epothilone 21 (22 mg, 42 μmol, 93% yield) as a white amorphous solid. 21: R_{f}=0.40 (silica gel, 30% methanol in ethyl acetate); [α]_{D}22=34.4 (c=1.0, CH₂Cl₂); FT-IR (neat) vₘₐₓ 3340, 2959, 2927, 2875, 1731, 1686, 1589, 1562, 1469, 1434, 1383, 1334, 1261, 1150, 1049, 1010, 982, 885, 800, 771, 745, 704 cm⁻¹; ¹H NMR (600 MHz, CD₂Cl₂) δ=8.54 (d, J=4.8 Hz, 1H), 7.70 (ddd, J=7.8, 7.8, 1.8 Hz, 1H), 7.28 (d, J=7.8 Hz, 1H), 7.15 (ddd, J=7.8, 4.8, 1.2 Hz, 1H), 6.60 (s, 1H), 5.40 (dd, J=7.2, 3.0 Hz, 1H), 4.22 (dd, J=11.2, 2.4 Hz, 1H), 3.65 (t, J=4.8 Hz, 1H), 3.62-3.55 (m, 2H), 3.21 (qd, J=6.6, 5.2 Hz, 1H), 2.70-2.66 (m, 1H), 2.62-2.59 (m, 1H), 2.48 (dd, J=13.2, 10.2 Hz, 1H), 2.34 (dd, J=13.8, 2.4 Hz, 1H), 2.08 (s, 3H), 2.01-1.96 (m, 1H), 1.95-1.90 (m, 1H), 1.73-1.67 (m, 1H), 1.54-1.43 (m, 4H), 1.41-1.34 (m, 3H), 1.37 (s, 3H), 1.17 (s, 3H), 1.11 (d, J=6.6 Hz, 3H), 1.03 (s, 3H), 0.95 (d, J=7.2 Hz, 3H) ppm; ¹³C NMR (151 MHz, CD₂Cl₂) δ=220.8, 171.3, 156.2, 149.4, 141.4, 136.8, 125.0, 124.4, 121.8, 77.7, 74.8, 73.6, 62.2, 60.6, 55.0, 48.9, 44.2, 43.7, 39.8, 35.6, 35.2, 32.2, 30.1, 21.4, 20.8, 19.6, 17.3, 16.5, 15.8, 13.6 ppm; HRMS (ESI) calcd for C₃₀H₄₆N₂O₆Na⁺ [M+Na]⁺ 553.3248, found 553.3255.

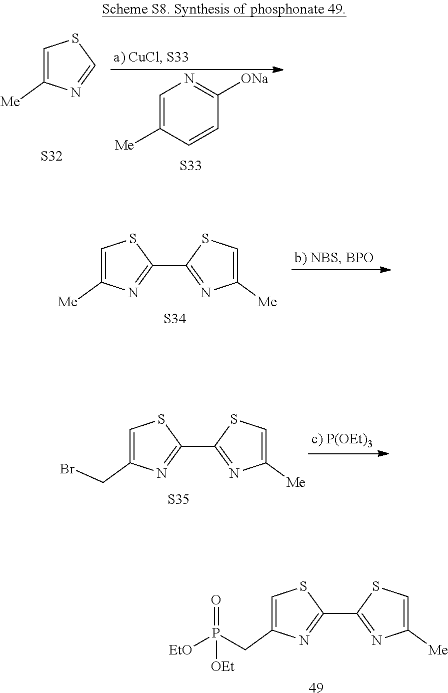

### 4,4′-Dimethyl-2,2′-bi-1,3-thiazole (S34)

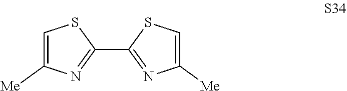 [SMILES: Cc1csc(-c2nc(C)cs2)n1]

To a stirred solution of 4-methylthiazole S32 (2.00 g, 20.2 mmol, 1.0 equiv) in m-xylene (40 mL) at 25° C. was added sodium 2-pyridonate S33 (53.0 mg, 0.404 mmol, 0.02 equiv), followed by CuCl (20.0 mg, 0.202 mmol, 0.01 equiv). The reaction mixture was heated to 150° C. for 5 min under an atmosphere of argon. Then the atmosphere of argon was removed, and refluxing was continued under open air. After 60 h, the reaction mixture was allowed to cool to 25° C., filtered through Celite®, and rinsed thoroughly with ethyl acetate (50 mL). The filtrate was washed with water (20 mL) and brine (20 mL), and the organic layer was dried with anhydrous sodium sulfate and concentrated under reduced pressure. The obtained residue was purified by flash column chromatography (silica gel, 10→30% ethyl acetate in hexanes) to afford pure thiazole S34⁵ (0.720 g, 3.67 mmol, 36% yield) as a pale yellow solid. S34: R_{f}=0.44 (silica gel, 30% ethyl acetate in hexanes); m.p. 134-135° C.; FT-IR (film) vₘₐₓ 3075, 3030, 2989, 2955, 2917, 2848, 1535, 1509, 1432, 1398, 1363, 1304, 1161, 1038, 999, 980, 907, 873, 765, 732, 673 cm′; ¹H NMR (600 MHz, CDCl₃) δ=6.93 (d, J=1.2 Hz, 2H), 2.47 (d, J=1.2 Hz, 6H) ppm; ¹³C NMR (151 MHz, CDCl₃) δ=160.9, 154.2, 115.5, 17.3 ppm.

### 4-(Bromomethyl)-4′-methyl-2,2′-bi-1,3-thiazole (S35)

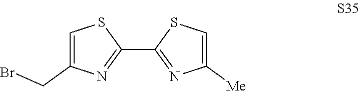 [SMILES: Cc1csc(-c2nc(CBr)cs2)n1]

To a stirred solution of thiazole S34 (557 mg, 2.84 mmol, 1.0 equiv) in carbon tetrachloride (8 mL) at 25° C. was added N-bromosuccinimide (556 mg, 3.12 mmol, 1.1 equiv). The reaction mixture was heated to 80° C. for 5 min, and then benzoyl peroxide (68.8 mg, 0.284 mmol, 0.1 equiv) was added. After 1.5 h, the reaction mixture was allowed to cool to 25° C., diluted with ethyl acetate (100 mL), and washed with a saturated aqueous solution of sodium bicarbonate (30 mL) and brine (30 mL). The two phases were separated, and the organic layer was dried over anhydrous sodium sulfate and concentrated under reduced pressure. The obtained residue was purified by flash column chromatography (silica gel, 2.5→15% ethyl acetate in hexanes) to afford pure bromomethyl thiazole S35⁶ (0.704 g, 2.56 mmol, 90% yield) as a pale yellow amorphous solid. S35: R_{f}=0.34 (silica gel, 20% ethyl acetate in hexanes); FT-IR (film) vₘₐₓ 3102, 3028, 2958, 2921, 2853, 1505, 1442, 1426, 1404, 1373, 1326, 1304, 1260, 1214, 1147, 1109, 1036, 983, 880, 743, 700, 672 cm⁻¹; ¹H NMR (600 MHz, CDCl₃) δ=7.37 (s, 1H), 7.00 (d, J=1.2 Hz, 1H), 4.61 (s, 2H), 2.50 (d, J=1.2 Hz, 3H) ppm; ¹³C NMR (151 MHz, CDCl₃) δ=162.2, 160.2, 154.5, 153.5, 119.6, 116.3, 26.8, 17.3 ppm.

### Diethyl [(4′-methyl-2,2′-bi-1,3-thiazol-4-yl)methyl]phosphonate (49)

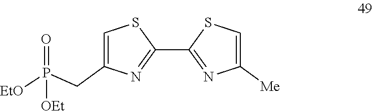 [SMILES: CCOP(=O)(Cc1csc(-c2nc(C)cs2)n1)OCC]

A stirred solution of bromomethyl thiazole S35 (0.670 g, 2.44 mmol, 1.0 equiv) in triethyl phosphite (2.02 g, 12.2 mmol, 5.0 equiv) was heated to 160° C. After 1.5 h, the triethyl phosphite was removed under a steady flow of nitrogen gas, and the reaction mixture was allowed to cool to 25° C. The obtained residue was purified by flash column chromatography (silica gel, 50→100% ethyl acetate in hexanes) to afford pure phosphonate 49 (0.423 g, 1.27 mmol, 52% yield) as a colorless oil. 49: R_{f}=0.21 (silica gel, ethyl acetate); FT-IR (neat) vₘₐₓ 3457, 3102, 2982, 2925, 1643, 1505, 1443, 1394, 1374, 1324, 1247, 1162, 1097, 1051, 1022, 981, 933, 881, 82, 826, 809, 782, 747, 699, 660 cm⁻¹; NMR (600 MHz, CDCl₃) δ=7.29 (d, J=3.0 Hz, 1H), 6.95 (d, J=1.2 Hz, 1H), 4.15-4.06 (m, 4H), 3.42 (d, J=21.0 Hz, 2H), 2.48 (d, J=1.2 Hz, 3H), 1.28 (t, J=7.2 Hz, 6H) ppm; ¹³C NMR (151 MHz, CDCl₃) δ=161.1, 160.6, 154.4, 148.1, 148.0 (d, J=7.8 Hz), 118.3 (d, J=7.4 Hz), 62.5 (d, J=6.6 Hz), 30.0 (d, J=139.8 Hz), 17.3, 16.6 (d, J=6.0 Hz) ppm; HRMS (ESI) calcd for C₁₂H₁₇N₂O₃PS₂Na⁺ [M+Na]⁺ 355.0310, found 355.0317.

### (1S,3S,7S,10R,11S,12S,16R)-17-(2-{[tert-Butyl(dimethyl)silyl]oxy}ethyl)-8,8,10,12,16-pentamethyl-3-[(1E)-1-(4′-methyl-2,2′-bi-1,3-thiazol-4-yl)prop-1-en-2-yl]-7,11-bis [(triethylsilyl)oxy]-4-oxa-17-azabicyclo[14.1.0]heptadecane-5,9-dione (22a)

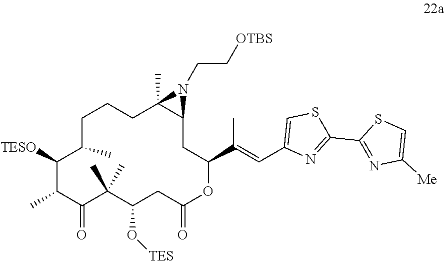

To a stirred solution of phosphonate 49 (130 mg, 0.391 mmol, 21 equiv) in tetrahydrofuran (1.0 mL) at −78° C. was added n-butyllithium (2.5 M in hexanes, 0.13 mL, 0.33 mmol, 18 equiv). After 10 min, the reaction mixture was transferred to a stirred solution of methyl ketone 82 (14.5 mg, 18.2 μmot, 1.0 equiv) in tetrahydrofuran (0.5 mL) at −78° C., and the reaction mixture was allowed to slowly warm to 25° C. over 4 h. Then the reaction mixture was quenched with saturated aqueous ammonium chloride solution (5 mL), and the two phases were separated. The organic layer was extracted with ethyl acetate (3×5 mL), and the combined organic layers were dried with anhydrous sodium sulfate and concentrated under reduced pressure. The obtained residue was purified by flash column chromatography (silica gel, 5→15% ethyl acetate in hexanes) to afford pure protected epothilone 22a (11.8 mg, 12.1 μmot, 67%, plus 28% recovered methyl ketone 82) as a colorless oil. 22a: Rₜ=0.27 (silica gel, 20% ethyl acetate in hexanes); [α]_{D}²²+2.0 (c=0.40, CH₂C₁₂); FT-IR (neat) vₘₐₓ 2953, 2931, 2877, 1742, 1696, 1503, 1462, 1413, 1380, 1304, 1248, 1199, 1158, 1107, 1008, 980, 883, 835, 780, 733 cm⁻¹; ¹H NMR (600 MHz, C₆D₆) δ=6.73 (s, 1H), 6.53 (s, 1H), 6.19 (s, 1H), 5.45 (dd, J=7.8, 3.0 Hz, 1H), 4.24 (dd, J=9.0, 3.0 Hz, 1H), 4.19 (d, J=9.0 Hz, 1H), 3.88-3.80 (m, 2H), 3.04 (dq, J=8.4, 6.6 Hz, 1H), 2.77 (ddd, J=12.0, 6.0, 6.0 Hz, 1H), 2.73 (dd, J=16.2, 9.0 Hz, 1H), 2.60 (dd, J=16.2, 3.0 Hz, 1H), 2.47 (ddd, J=12.0, 6.6, 6.6 Hz, 1H), 2.30 (s, 3H), 2.30-2.28 (m, 1H), 2.20 (s, 3H), 2.06 (ddd, J=15.6, 9.0, 9.0 Hz, 1H), 1.91-1.82 (m, 2H), 1.76-1.71 (m, 1H), 1.67-1.59 (m, 1H), 1.53-1.48 (m, 2H), 1.27 (dd, J=10.2, 3.6 Hz, 1H), 1.24-1.20 (m, 1H), 1.20 (d, J=7.2 Hz, 3H), 1.18 (s, 3H), 1.17 (s, 3H), 1.15 (d, J=6.6 Hz, 3H), 1.11-1.06 (m, 18H), 0.99 (s, 9H), 0.87 (s, 3H), 0.85-0.78 (m, 6H), 0.74-0.71 (m, 6H), 0.097 (s, 3H), 0.094 (s, 3H) ppm; ¹³C NMR (151 MHz, C₆D₆) δ=214.5, 170.7, 160.93, 160.90, 154.5, 154.4, 139.9, 120.3, 119.0, 115.8, 80.2, 79.4, 75.9, 64.3, 54.9, 53.4, 50.2, 48.1, 43.3, 40.2, 37.4, 36.4, 35.4, 32.4, 26.2, 25.5, 23.6, 23.2, 20.1, 18.5, 17.7, 16.9, 15.6, 14.9, 7.44, 7.37, 6.0, 5.8, 5.1 ppm; HRMS (ESI) calcd for C₅₀H₀₉N₃O₆S₂Si₃⁺ [M+H]⁺ 976.5573, found 976.5566.

### (1S,3S,7S,10R,11S,12S,16R)-7,11-Dihydroxy-17-(2-hydroxyethyl)-8,8,10,12,16-pentamethyl-3-[(1E)-1-(4′-methyl-2,2′-bi-1,3-thiazol-4-yl)prop-1-en-2-yl]-4-oxa-17-azabicyclo[14.1.0]-heptadecane-5,9-dione (22)

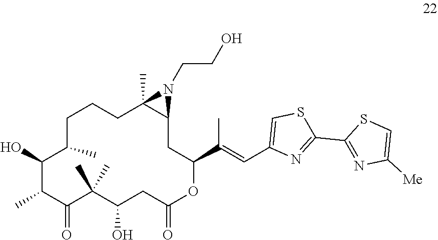

To a stirred solution of protected epothilone 22a (11 mg, 11 μmot, 1.0 equiv) in tetrahydrofuran (2.0 mL) at 0° C. was added hydrogen fluoride-pyridine complex (70%, 0.20 mL, 7.6 mmol, excess), and the reaction mixture was allowed to slowly warm to 25° C. After 5 h, the reaction mixture was quenched with saturated aqueous sodium bicarbonate solution (20 mL), and the two phases were separated. The aqueous layer was extracted with ethyl acetate (3×15 mL), and the combined organic layers were dried with anhydrous sodium sulfate and concentrated under reduced pressure. The obtained residue was purified by flash column chromatography (silica gel, 5→20% methanol in ethyl acetate) to afford pure epothilone 22 (5.2 mg, 8.2 μmol, 74% yield) as a white amorphous solid. 22: R_{f}=0.32 (silica gel, 15% methanol in ethyl acetate); [α]2=27.2 (c=0.25, CH₂Cl₂); FT-IR (film) vₘₐₓ 3387, 3105, 2931, 2876, 1731, 1687, 1568, 1525, 1501, 1468, 1408, 1376, 1334, 1251, 1147, 1052, 1008, 980, 883, 736 cm⁻¹; ¹H NMR (600 MHz, CD₂Cl₂) δ=7.25 (s, 1H), 7.03 (s, 1H), 6.61 (s, 1H), 5.48 (dd, J=4.2, 4.2 Hz, 1H), 4.10-4.04 (m, 1H), 4.06 (dd, J=9.6, 1.8 Hz, 1H), 3.75 (dd, J=5.4, 4.2 Hz, 1H), 3.70-3.62 (m, 2H), 3.30 (ddd, J=13.2, 6.6, 6.6 Hz, 1H), 2.69-2.65 (m, 1H), 2.61-2.57 (m, 1H), 2.53 (dd, J=13.8, 10.2 Hz, 1H), 2.49 (s, 3H), 2.43 (dd, J=13.8, 1.8 Hz, 1H), 2.18 (s, 3H), 2.03-1.99 (m, 3H), 1.90-1.85 (m, 1H), 1.70-1.64 (m, 1H), 1.57-1.51 (m, 1H), 1.50-1.44 (m, 2H), 1.41-1.32 (m, 2H), 1.37 (s, 3H), 1.16 (s, 3H), 1.13 (d, J=7.2 Hz, 3H), 1.04 (s, 3H), 0.97 (d, J=6.6 Hz, 3H) ppm; ¹³C NMR (151 MHz, CD₂Cl₂) δ=220.6, 171.4, 160.9, 160.7, 154.8, 154.2, 138.4, 118.9, 118.7, 116.2, 77.8, 76.0, 75.1, 62.4, 55.4, 52.7, 48.1, 44.9, 43.4, 39.5, 35.5, 34.6, 31.8, 29.5, 21.8, 21.5, 19.9, 17.6, 17.2, 16.5, 15.9, 14.5 ppm; HRMS (ESI) calcd for C₃₂H₄₈N₃O₆S₂⁺ [M+H]+ 634.2979, found 634.2996.

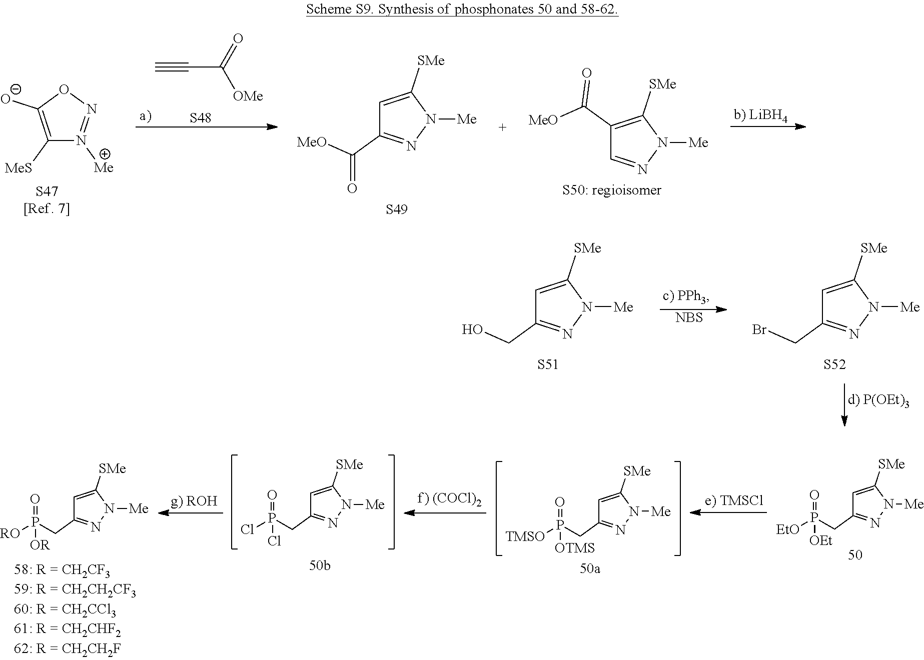

### Methyl 1-methyl-5-(methylsulfanyl)-1H-pyrazole-3-carboxylate (S49)

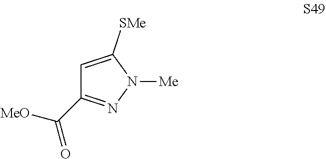 [SMILES: COC(=O)c1cc(SC)n(C)n1]

To a stirred suspension of sydnone S47 (Hammick & Voaden, 1961 and Masuda & Okutani, 1974) (3.09 g, 21.1 mmol, 1.0 equiv) in xylenes (10 mL) at 25° C. was added methyl propiolate (3.76 mL, 42.3 mmol, 2.0 equiv), and the reaction mixture was heated to 130° C. After 12 h, the reaction mixture was allowed to cool to 25° C., and concentrated under reduced pressure. The obtained residue was purified by flash column chromatography (silica gel, 5→30% ethyl acetate in hexanes) to afford pure pyrazolyl ester S49 (2.44 g, 13.1 mmol, 62% yield) as a colorless oil and its regioisomer S50 (0.943 g, 5.06 mmol, 24% yield) as a colorless oil. S49: R_{f}=0.26 (silica gel, 40% ethyl acetate in hexanes); FT-IR (neat) vₘₐₓ 3137, 2996, 2951, 1718, 1503, 1458, 1441, 1397, 1366, 1320, 1290, 1217, 1126, 1076, 1044, 1010, 977, 946, 808, 776, 721 cm⁻¹; ¹H NMR (600 MHz, CDCl₃) δ=6.77 (s, 1H), 3.92 (s, 3H), 3.90 (s, 3H), 2.43 (s, 3H) ppm; ¹³C NMR (151 MHz, CDCl₃) δ=162.6, 142.7, 138.8, 111.2, 52.2, 37.5, 18.6 ppm; HRMS (ESI) calcd for C₇H₁₁N₂O₂S⁺ [M+H]⁺ 187.0536, found 187.0531. S50: R_{f}=0.37 (silica gel, 40% ethyl acetate in hexanes); FT-IR (neat) vₘₐₓ 2996, 2949, 1713, 1519, 1435, 1405, 1389, 1365, 1314, 1275, 1221, 1169, 1108, 1045, 983, 945, 871, 805, 777, 728 cm⁻¹; ¹H NMR (600 MHz, CDCl₃) δ=7.93 (s, 1H), 3.97 (s, 3H), 3.85 (s, 3H), 2.48 (s, 3H) ppm; ¹³C NMR (151 MHz, CDCl₃) δ=163.0, 141.9, 139.8, 116.4, 51.5, 37.4, 18.8 ppm; HRMS (ESI) calcd for C₇H₁₁N₂O₂S⁺ [M+H]⁺ 187.0536, found 187.0529.

### [1-Methyl-5-(methylsulfanyl)-1H-pyrazol-3-yl]methanol (S51)

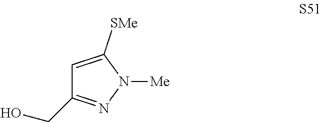

To a stirred solution of pyrazolyl ester S50 (2.44 g, 13.1 mmol, 1.0 equiv) in dichloromethane (36 mL) at −78° C. was added diisobutylaluminum hydride (1.0 M in dichloromethane, 40.0 mL, 40.0 mmol, 3.0 equiv) dropwise. After 10 min, the reaction mixture was quenched with aqueous hydrochloric acid solution (2.0 M, 30 mL), and allowed to warm to 25° C. After 2 h, the two phases were separated, and the aqueous layer was extracted with ethyl acetate (3×20 mL). The combined organic layers were dried with anhydrous sodium sulfate and concentrated under reduced pressure. The obtained residue was purified by flash column chromatography (silica gel, 50→100% ethyl acetate in hexanes) to afford pure hydroxymethyl pyrazole S51 (1.70 g, 10.7 mmol, 82% yield) as a colorless oil. S51: R_{f}=0.22 (silica gel, ethyl acetate); FT-IR (neat) vₘₐₓ 3327, 2925, 2869, 1508, 1423, 1318, 1379, 1279, 1216, 1147, 1057, 1020, 1001, 976, 771 cm¹; ¹H NMR (600 MHz, CDCl₃) δ=6.25 (s, 1H), 4.62 (d, J=6.0 Hz, 2H), 3.83 (s, 3H), 2.40 (s, 3H), 2.27 (t, J=6.0 Hz, 1H) ppm; ¹³C NMR (151 MHz, CDCl₃) δ=151.7, 137.5, 107.1, 59.2, 36.5, 18.8 ppm; HRMS (ESI) calcd for C₆H11N₂OS⁺ [M+H]+ 159.0587, found 159.0581.

### 3-(Bromomethyl)-1-methyl-5-(methylsulfanyl)-1H-pyrazole (S52)

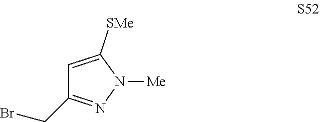

To a stirred solution of hydroxymethyl pyrazole S51 (1.70 g, 10.7 mmol, 1.0 equiv) in dichloromethane (20 mL) at −78° C. was added triphenylphosphine (2.96 g, 11.3 mmol, 1.05 equiv), followed by N-bromosuccinimide (1.90 g, 10.7 mmol, 1.0 equiv). After 5 min, the reaction mixture was quenched with water (20 mL), and allowed to warm to 25° C. The two phases were separated, and the aqueous layer was extracted with ethyl acetate (3×15 mL). The combined organic layers were dried with anhydrous sodium sulfate and concentrated under reduced pressure. The obtained residue was purified by flash column chromatography (silica gel, 5→20% ethyl acetate in hexanes) to afford pure bromomethyl pyrazole S52 (2.03 g, 9.06 mmol, 85% yield) as a colorless oil. S52: R_{f}=0.30 (silica gel, 20% ethyl acetate in hexanes); FT-IR (neat) vₘₐₓ 3122, 2924, 1503, 1425, 1317, 1285, 1213, 1159, 1111, 1082, 1043, 1007, 974, 801, 767, 711 cm⁻¹; ¹H NMR (600 MHz, CDCl₃) δ=6.30 (s, 1H), 4.43 (s, 2H), 3.84 (s, 3H), 2.41 (s, 3H) ppm; ¹³C NMR (151 MHz, CDCl₃) δ=148.4, 138.1, 108.4, 36.7, 25.3, 18.7 ppm; HRMS (ESI) calcd for C₆H₁₀BrN₂S⁺ [M+H]⁺ 220.9743, found 220.9749.

### Diethyl {[1-methyl-5-(methylsulfanyl)-1H-pyrazol-3-yl]methyl}phosphonate (50)

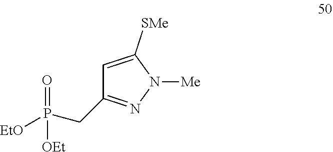 [SMILES: CCOP(=O)(Cc1cc(SC)n(C)n1)OCC]

A stirred solution of bromomethyl pyrazole S52 (2.03 g, 9.06 mmol, 1.0 equiv) in triethyl phosphite (4.0 mL, 23 mmol, 2.6 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 50→90% acetone in hexanes) to afford pure phosphonate 50 (2.49 g, 8.97 mmol, 99% yield) as a colorless oil. 50: R_{f}=0.43 (silica gel, acetone); FT-IR (neat) vₘₐₓ 3471, 2982, 2927, 1505, 1441, 1425, 1392, 1368, 1253, 1163, 1097, 1054, 1025, 963, 848, 815, 757, 727, 696 cm⁻¹; ¹H NMR (600 MHz, CDCl₃) δ=6.27 (d, J=1.8 Hz, 1H), 4.08 (dq, J=7.2, 7.2 Hz, 4H), 3.81 (s, 3H), 3.15 (d, J=20.4 Hz, 2H), 2.39 (s, 3H), 1.28 (t, J=7.2 Hz, 6H) ppm; ¹³C NMR (151 MHz, CDCl₃) δ=142.6 (d, J=7.1 Hz), 137.4 (d, J=2.3 Hz), 108.7 (d, J=3.3 Hz), 62.3 (d, J=6.3 Hz), 36.5, 26.9 (d, J=141.0 Hz), 18.7, 16.6 (d, J=6.0 Hz) ppm; HRMS (ESI) calcd for C₁₀H₂₀N₂O₃PS⁺ [M+H]⁺ 279.0927, found 279.0930.

### Bis(2,2,2-trifluoroethyl) {[1-methyl-5-(methylsulfanyl)-1H-pyrazol-3-yl]methyl}phosphonate (58)

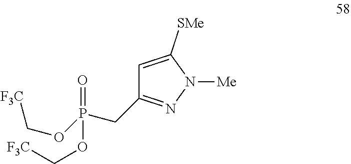 [SMILES: CSc1cc(CP(=O)(OCC(F)(F)F)OCC(F)(F)F)nn1C]

A stirred solution of phosphonate 50 (2.49 g, 8.97 mmol, 1.0 equiv) in trimethylsilyl chloride (5.75 mL, 45.3 mmol, 5.1 equiv) was heated to 80° C. After 72 h, the reaction mixture was allowed to cool to 25° C., and the trimethylsilyl chloride was removed under reduced pressure to afford crude 50a. The crude residue was redissolved in dichloromethane (30 mL) with stirring, and cooled to 0° C. Then a solution of oxalyl chloride (3.05 g, 24.0 mmol, 2.5 equiv) in dichloromethane (5 mL) was added dropwise, and the reaction mixture was allowed to slowly warm to 25° C. After 4 h, the solvent was removed under reduced pressure to afford 50b. The crude residue was redissolved in dichloromethane (30 mL) with stirring, and cooled to 0° C. Then triethylamine (7.58 mL, 54.4 mmol, 6.0 equiv), 2,2,2-trifluoroethanol (2.72 mL, 36.2 mmol, 4.0 equiv) and 4-(dimethylamino)pyridine (22.1 mg, 0.181 mmol, 0.02 equiv) were added sequentially, and the reaction mixture was allowed to slowly warm to 25° C. After 12 h, the reaction mixture was diluted with ethyl acetate (100 mL), and washed with water (20 mL) and brine (20 mL). The two phases were separated, and the organic layer was dried with anhydrous sodium sulfate and concentrated under reduced pressure. The obtained residue was purified by flash column chromatography (silica gel, 20→60% ethyl acetate in hexanes) to afford pure trifluoroethyl phosphonate 58 (3.00 g, 7.77 mmol, 87% yield overall) as a colorless oil. 58: R_{f}=0.17 (silica gel, 50% ethyl acetate in hexanes); FT-IR (neat) vₘₐₓ 2971, 1504, 1422, 1291, 1260, 1168, 1103, 1070, 1007, 963, 879, 845, 780, 704 cm⁻¹; ¹H NMR (600 MHz, CDCl₃) δ=6.21 (d, J=1.8 Hz, 1H), 4.42-4.31 (m, 4H), 3.81 (d, J=1.2 Hz, 3H), 3.33 (d, J=21.0 Hz, 2H), 2.39 (s, 3H) ppm; ¹³C NMR (151 MHz, CDCl₃) δ=140.3 (d, J=8.1 Hz), 138.2 (d, J=2.1 Hz), 122.7 (qd, J=275.9, 8.0 Hz), 108.6 (d, J=5.3 Hz), 62.5 (qd, J=37.7, 6.0 Hz), 36.6, 26.8 (d, J=143.3 Hz), 18.6 ppm; HRMS (ESI) calcd for C₁₀H₁₄N₂O₃PS⁺ [M+H]⁺ 387.0361, found 387.0346.

### Bis(3,3,3-trifluoropropyl) {[1-methyl-5-(methylsulfanyl)-1H-pyrazol-3-yl]methyl}phosphonate (59)

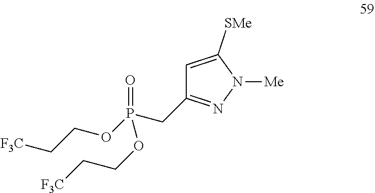 [SMILES: CSc1cc(CP(=O)(OCCC(F)(F)F)OCCC(F)(F)F)nn1C]

To a stirred solution of crude 506 (ca. 172 mg, 0.662 mmol, 1.0 equiv) in dichloromethane (10 mL) at 0° C. was added triethylamine (0.29 mL, 4.0 mmol, 6.0 equiv), followed by 3,3,3-trifluoropropanol (0.300 g, 2.65 mmol, 4.0 equiv) and 4-dimethylaminopyridine (1.6 mg, 13 μmol, 0.02 equiv), and the reaction mixture was allowed to slowly warm to 25° C. After 12 h, the reaction mixture was diluted with ethyl acetate (50 mL), and washed with water (20 mL) and brine (20 mL). The two phases were separated, and the organic layer was dried with anhydrous sodium sulfate and concentrated under reduced pressure. The obtained residue was purified by flash column chromatography (silica gel, 50→100% ethyl acetate in hexanes) to afford pure trifluoropropyl phosphonate 59 (0.137 g, 0.330 mmol, 50% yield overall) as a colorless oil. 59: R_{f}=0.34 (silica gel, ethyl acetate); FT-IR (neat) vₘₐₓ 3476, 2928, 1505, 1434, 1391, 1351, 1297, 1254, 1153, 1132, 1067, 1006, 974, 929, 878, 846, 823, 762, 700 cm¹; ¹H NMR (600 MHz, CDCl₃) δ=6.22 (d, J=1.2 Hz, 1H), 4.26-4.19 (m, 4H), 3.80 (d, J=0.6 Hz, 3H), 3.20 (d, J=21.0 Hz, 2H), 2.50-2.42 (m, 4H), 2.38 (s, 3H) ppm; ¹³C NMR (151 MHz, CDCl₃) δ=141.4 (d, J=7.7 Hz), 138.0 (d, J=2.1 Hz), 125.6 (q, J=275.1 Hz), 108.6 (d, J=4.1 Hz), 59.2 (m), 36.5, 35.1 (qd, J=28.8, 6.5 Hz), 26.8 (d, J=140.7 Hz), 18.6 ppm; HRMS (ESI) calcd for C₁₂H₁₈F₆N₂O₃PS⁺ [M+H]⁺ 415.0674, found 415.0673.

### Bis(2,2,2-trichloroethyl) {[1-methyl-5-(methylsulfanyl)-1H-pyrazol-3-yl]methyl}phosphonate (60)

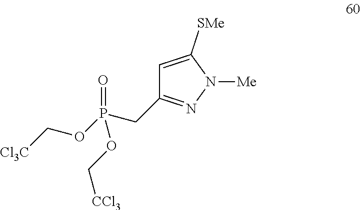

To a stirred solution of crude 506 (ca. 172 mg, 0.662 mmol, 1.0 equiv) in dichloromethane (10 mL) at 0° C. was added triethylamine (0.29 mL, 4.0 mmol, 6.0 equiv), followed by 2,2,2-trichloroethanol (0.396 g, 2.65 mmol, 4.0 equiv) and 4-dimethylaminopyridine (1.6 mg, 13 μmol, 0.02 equiv), and the reaction mixture was allowed to slowly warm to 25° C. After 12 h, the reaction mixture was diluted with ethyl acetate (50 mL), and washed with water (20 mL) and brine (20 mL). The two phases were separated, and the organic layer was dried with anhydrous sodium sulfate and concentrated under reduced pressure. The obtained residue was purified by flash column chromatography (silica gel, 40→70% ethyl acetate in hexanes) to afford pure trichloroethyl phosphonate 60 (0.167 g, 0.344 mmol, 52% yield overall) as a colorless oil. 60: R_{f}=0.45 (silica gel, 20% hexanes in ethyl acetate); FT-IR (neat) vₘₐₓ 3480, 2990, 2947, 2926, 1504, 1447, 1425, 1391, 1376, 1317, 1262, 1111, 1095, 1045, 1023, 974, 874, 833, 814, 790, 765, 747, 719 cm⁻¹; ¹H NMR (600 MHz, CDCl₃) δ=6.29 (d, J=1.8 Hz, 1H), 4.63-4.58 (m, 4H), 3.81 (s, 3H), 3.43 (d, J=21.0 Hz, 2H), 2.38 (s, 3H) ppm; ¹³C NMR (151 MHz, CDCl₃) δ=140.6 (d, J=8.1 Hz), 138.0 (d, J=2.1 Hz), 108.9 (d, J=4.8 Hz), 95.3 (d, J=8.9 Hz), 76.0 (d, J=6.0 Hz), 36.6, 27.3 (d, J=142.8 Hz), 18.7 ppm; HRMS (ESI) calcd for C₁₀H₁₄C₁₆N₂O₃PS [M+H]⁺ 482.8588, found 482.8608.

### Bis(2,2-difluoroethyl) {[1-methyl-5-(methylsulfanyl)-1H-pyrazol-3-yl]methyl}phosphonate (61)

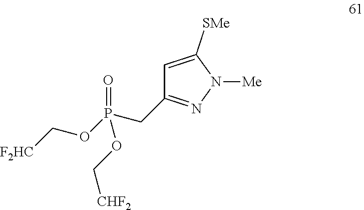 [SMILES: CSc1cc(CP(=O)(OCC(F)F)OCC(F)F)nn1C]

To a stirred solution of crude 50b (ca. 191 mg, 0.737 mmol, 1.0 equiv) in dichloromethane (10 mL) at 0° C. was added triethylamine (0.62 mL, 4.4 mmol, 6.0 equiv), followed by 2,2,-difluoroethanol (0.242 g, 2.95 mmol, 4.0 equiv) and 4-dimethylaminopyridine (2.0 mg, 16 μmol, 0.02 equiv), and the reaction mixture was allowed to slowly warm to 25° C. After 3 h, the reaction mixture was diluted with ethyl acetate (60 mL), and washed with water (20 mL) and brine (20 mL). The two phases were separated, and the organic layer was dried with anhydrous sodium sulfate and concentrated under reduced pressure. The obtained residue was purified by flash column chromatography (silica gel, 50→90% ethyl acetate in hexanes) to afford pure difluoroethyl phosphonate 61 (0.205 g, 0.585 mmol, 79% yield overall) as a colorless oil. 61: R_{f}=0.33 (silica gel, 10% hexanes in ethyl acetate); FT-IR (neat) vₘₐₓ 3465, 2962, 2930, 1504, 1426, 1395, 1375, 1324, 1260, 1143, 1080, 1048, 1007, 946, 903, 870, 780, 703 cm⁻¹; ¹H NMR (600 MHz, CDCl₃) δ=6.23 (d, J=1.2 Hz, 1H), 5.91 (tt, J=55.2, 4.2 Hz, 2H), 4.25-4.15 (m, 4H), 3.81 (s, 3H), 3.28 (d, J=21.0 Hz, 2H), 2.40 (s, 3H) ppm; ¹³C NMR (151 MHz, CDCl₃) δ=140.9 (d, J=8.0 Hz), 138.2 (d, J=2.1 Hz), 113.0 (td, J=240.6, 7.1 Hz), 108.6 (d, J=4.7 Hz), 64.3 (td, J=29.9, 6.3 Hz), 36.6, 26.8 (d, J=142.5 Hz), 18.6 ppm; HRMS (ESI) calcd for C₁₀H₁₅F₄N₂O₃PSNa⁺ [M+Na]⁺ 373.0369, found 373.0377.

### Bis(2-fluoroethyl) {[1-methyl-5-(methylsulfanyl)-1H-pyrazol-3-yl]methyl}phosphonate (62)

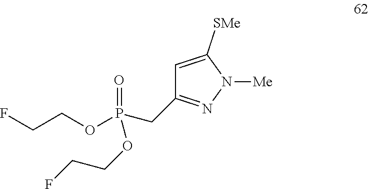 [SMILES: CSc1cc(CP(=O)(OCCF)OCCF)nn1C]

To a stirred solution of crude 50b (ca. 191 mg, 0.737 mmol, 1.0 equiv) in dichloromethane (10 mL) at 0° C. was added triethylamine (0.62 mL, 4.4 mmol, 6.0 equiv), followed by 2-fluoroethanol (0.189 g, 2.95 mmol, 4.0 equiv) and 4-dimethylaminopyridine (2.0 mg, 16 μmot, 0.02 equiv), and the reaction mixture was allowed to slowly warm to 25° C. After 3 h, the reaction mixture was diluted with ethyl acetate (60 mL), and washed with water (20 mL) and brine (20 mL). The two phases were separated, and the organic layer was dried with anhydrous sodium sulfate and concentrated under reduced pressure. The obtained residue was purified by flash column chromatography (silica gel, 40→80% ethyl acetate in hexanes) to afford pure fluoroethyl phosphonate 62 (0.180 g, 0.573 mmol, 78% yield overall) as a colorless oil. 62: R_{f}=0.28 (silica gel, 40% hexanes in acetone); FT-IR (neat) vₘₐₓ 3455, 2954, 1654, 1504, 1452, 1426, 1394, 1373, 1252, 1119, 1072, 1028, 958, 868, 848, 814, 758, 728 cm⁻¹; ¹H NMR (600 MHz, CDCl₃) δ=6.26 (d, J=1.8 Hz, 1H), 4.55 (dt, J=47.4, 4.2 Hz, 4H), 4.33-4.20 (m, 4H), 3.80 (d, J=0.6 Hz, 3H), 3.24 (d, J=20.4 Hz, 2H), 2.38 (s, 3H) ppm; ¹³C NMR (151 MHz, CDCl₃) δ=141.7 (d, J=7.5 Hz), 137.8 (d, J=2.3 Hz), 108.6 (d, J=3.8 Hz), 82.4 (dd, J=170.9, 6.0 Hz), 65.1 (dd, J=20.4, 6.5 Hz), 36.5, 26.8 (d, J=142.2 Hz), 18.6 ppm; HRMS (ESI) calcd for C¹⁰H₁₇F₂N₂O₃PSNa⁺ [M+Na]⁺ 337.0558, found 337.0549.

### (1S,3S,7S,10R,11S,12S,16R)-17-(2-{[tert-Butyl(dimethyl)silyl]oxy}ethyl)-8,8,10,12,16-pentamethyl-3-{(1E)-1-[1-methyl-5-(methylsulfanyl)-1H-pyrazol-3-yl]prop-1-en-2-yl}-7,11-bis[(tri-ethylsilyl)oxy]-4-oxa-17-azabicyclo[14.1.0]heptadecane-5,9-dione 103 [(E) only]

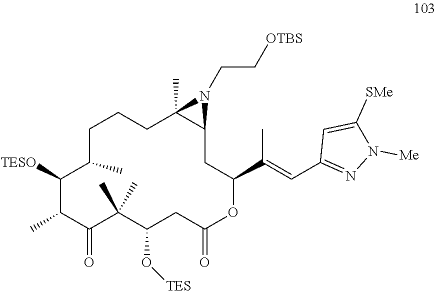

To a stirred solution of phosphonate 62 (124 mg, 0.395 mmol, 16 equiv) in tetrahydrofuran (1 mL) at −78° C. was added n-butyllithium (2.5 M in hexanes, 0.13 mL, 0.32 mmol, 13 equiv) dropwise. After 20 min, an aliquot (0.4 mL, ca. 5.7 equiv 62) of the reaction mixture was added to a solution of methyl ketone 82 (19.6 mg, 24.6 μmot, 1.0 equiv) in tetrahydrofuran (0.5 mL) at −78° C., and the reaction mixture was allowed to slowly warm to 40° C. over 30 min. Then the reaction mixture was quenched with saturated aqueous ammonium chloride solution (10 mL), and allowed to warm to 25° C. The two phases were separated, and the aqueous layer was extracted with ethyl acetate (3×15 mL). The combined organic layers were dried with anhydrous sodium sulfate and concentrated under reduced pressure. The obtained residue was purified by flash column chromatography (silica gel, 10→30% ethyl acetate in hexanes) to afford pure protected epothilone 103 (14.0 mg, 15.2 μmot, 62% yield) as a colorless oil. 103: R_{f}=0.32 (silica gel, 30% ethyl acetate in hexanes); [α]_{D}²²=−5.3 5.3 (c=0.34, CH₂Cl₂); FT-IR (neat) vₘₐₓ 2952, 2931, 2877, 1742, 1696, 1462, 1415, 1381, 1284, 1250, 1198, 1158, 1107, 1018, 1007, 985, 940, 916, 859, 836, 811, 779, 737 cm⁻¹; ¹H NMR (600 MHz, C₆D₆) δ=6.80 (s, 1H), 6.30 (s, 1H), 5.50 (dd, J=7.8, 3.0 Hz, 1H), 4.23 (dd, J=9.0, 3.6 Hz, 1H), 4.19 (d, J=9.0 Hz, 1H), 3.86-3.78 (m, 2H), 3.42 (s, 3H), 3.04 (dq, J=8.4, 6.6 Hz, 1H), 2.74 (dq, J=6.0, 6.0 Hz, 1H), 2.71 (dd, J=16.2, 9.0 Hz, 1H), 2.60 (dd, J=16.2, 1.6 Hz, 1H), 2.45 (ddd, J=12.0, 6.0, 6.0 Hz, 1H), 2.30 (ddd, J=16.0, 3.6, 3.6 Hz, 1H), 2.26 (s, 3H), 2.06 (ddd, J=15.0, 9.0, 9.0 Hz, 1H), 1.89-1.80 (m, 2H), 1.76 (s, 3H), 1.74-1.71 (m, 1H), 1.65-1.58 (m, 1H), 1.52-1.47 (m, 2H), 1.27 (dd, J=9.6, 3.0 Hz, 1H), 1.20 (d, J=6.6 Hz, 3H), 1.18 (s, 3H), 1.15 (s, 3H), 1.13 (d, J=6.6 Hz, 3H), 1.10-1.05 (m, 18H), 0.99 (s, 9H), 0.85 (s, 3H), 0.84-0.77 (m, 12H), 0.09 (s, 6H) ppm; ¹³C NMR (151 MHz, C₆D₆) δ=214.5, 170.7, 148.8, 137.3, 135.9, 119.7, 110.2, 80.1, 79.5, 75.8, 64.3, 54.9, 53.4, 50.2, 48.1, 43.2, 40.2, 37.4, 36.4, 36.1, 35.4, 32.3, 26.2, 25.5, 23.5, 23.2, 20.1, 18.5, 17.6, 15.6, 14.9, 7.4, 7.3, 6.0, 5.8, −5.1 ppm; HRMS (ESI) calcd for C₄₈H₉₂N₃O₆SSi₃⁺ [M+H]+ 922.6009, found 922.6010.

### Protected Epothilone 103 [(E):(Z) ca. 1:1]

To a stirred solution of phosphonate 58 (350 mg, 906 μmot, 16 equiv) in tetrahydrofuran (1 mL) at −78° C. was added n-butyllithium (2.5 M in hexanes, 0.29 mL, 0.73 mmol, 13 equiv) dropwise. After 45 min, a solution of methyl ketone 82 (45.0 mg, 56.4 μmot, 1.0 equiv) in tetrahydrofuran (0.6 mL) was added, and the reaction mixture was allowed to slowly warm to 25° C., and stirred for an additional 2 h. Then the reaction mixture was quenched with saturated aqueous ammonium chloride solution (10 mL), and the two phases were separated. The aqueous layer was extracted with ethyl acetate (3×15 mL), and the combined organic layers were dried with anhydrous sodium sulfate and concentrated under reduced pressure. The obtained residue was purified by flash column chromatography (silica gel, 10→60% ethyl acetate in hexanes) to afford pure protected epothilone 103 [36.5 mg, 39.6 μmot, 70%, (E):(Z) ca. 1:1] as a colorless oil (for characterization data of 103, see above).

### Protected Epothilone 103 [(E):(Z) ca. 30:70]

To a stirred solution of phosphonate adduct 102 (2.5 mg, 2.3 μmot, 1.0 equiv) in tetrahydrofuran (0.6 mL) at −20° C. was added potassium tert-butoxide (1.3 mg, 12 μmot, 5.0 equiv). After 5 min, the reaction mixture was quenched with water (3 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was purified by preparative thin layer chromatography (silica gel, 30% ethyl acetate in hexanes) to afford pure protected epothilone 103 [1.6 mg, 1.7 μmot, 75%, (E):(Z) ca. 30:70] as a colorless oil (for characterization data of 103, see above).

### Diethyl (21(1S,7S,10R,11S,12S,16R)-17-(2-{[tert-butyl(dimethyl)silyl]oxy}ethyl)-8,8,10,12,16-pentamethyl-5,9-dioxo-7,11-bis[(triethylsilyl)oxy]-4-oxa-17-azabicyclo[14.1.0]heptadec-3-yl)-2-hydroxy-1-[1-methyl-5-(methylsulfanyl)-1H-pyrazol-3-yl]propyl)phosphonate (102)

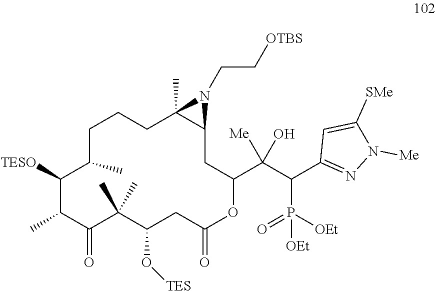

To a stirred solution of phosphonate 50 (120 mg, 0.431 mmol, 11 equiv) in tetrahydrofuran (0.5 mL) at −78° C. was added sodium bis(trimethylsilyl)amide (1.0 M in tetrahydrofuran, 0.21 mL, 0.21 mmol, 4.9 equiv) dropwise. After 30 min, the reaction mixture was transferred to a stirred solution of methyl ketone 82 (32.8 mg, 41.0 μmol, 1.0 equiv) in tetrahydrofuran (0.4 mL) at −78° C., and stirred for an additional 30 min. Then the reaction mixture was quenched with saturated aqueous ammonium chloride solution (3 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, ethyl acetate) to afford phosphonate adduct 102 (16.5 mg, 15.3 μmol, 37%, dr ca. 3:7) as a colorless oil. 102: Rₜ=0.24 (silica gel, 80% ethyl acetate in hexanes); [α]_{D}²²=−11.3 (c=1.0, CH₂Cl₂); FT-IR (neat) vₘₐₓ 3399, 2953, 2934, 2877, 1742, 1695, 1555, 1462, 1418, 1383, 1285, 1236, 1202, 1158, 1098, 1055, 1019, 983, 966, 835, 815, 777, 726, 678, 665 cm⁻¹; ¹H NMR (600 MHz, C₆D₆) δ=6.86 (s, 1H), 5.58-5.54 (m, 1H), 5.24-5.22 (m, 1H), 4.59-4.54 (m, 1H), 4.15-3.80 (m, 6H), 3.63-3.58 (m, 1H), 3.57 (s, 3H), 3.17-3.12 (m, 1H), 2.81-2.66 (m, 5H), 1.99 (s, 3H), 1.91 (s, 3H), 1.68-1.58 (m, 1H), 1.55-1.44 (m, 1H), 1.35-1.27 (m, 1H), 1.25-1.10 (m, 24H), 1.08-1.02 (m, 19H), 1.03 (s, 9H), 0.88-0.82 (m, 9H), 0.72-0.66 (m, 6H), 0.15 (s, 6H) ppm; ¹³C NMR (151 MHz, C₆D₆) δ=215.0, 170.9, 169.6, 145.0, 144.9, 137.6, 136.7, 110.5, 79.8, 79.4, 76.5, 76.4, 76.0, 75.9, 75.5, 74.7, 74.3, 64.5, 64.2, 63.5, 63.4, 61.1, 61.0, 55.7, 55.2, 54.7, 54.3, 54.1, 50.1, 48.9, 48.3, 46.6, 46.0, 45.7, 43.9, 43.5, 43.1, 41.5, 41.3, 38.4, 38.0, 37.1, 36.3, 36.1, 36.0, 32.4, 32.1, 31.9, 31.2, 26.3, 25.2, 24.9, 22.9, 22.1, 20.6, 19.1, 18.6, 18.5, 17.4, 16.2, 16.1, 15.7, 7.4, 5.9, 5.0 ppm; HRMS (ESI) calcd for C₅₂H₁₀₃N₃O₁₀PSSi₃⁺ [M+H]⁺ 1076.6404, found 1076.6405.

### (1S,3S,7S,10R,11S,12S,16R)-7,11-Dihydroxy-17-(2-hydroxyethyl)-8,8,10,12,16-pentamethyl-3-{(1E)-1-[1-methyl-5-(methylsulfanyl)-1H-pyrazol-3-yl]prop-1-en-2-yl}-4-oxa-17-azabicyclo[14.1.0]heptadecane-5,9-dione (23)

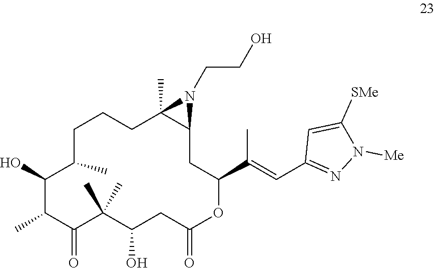

To a stirred solution of protected epothilone 103 (8.0 mg, 8.7 μmol, 1.0 equiv) in tetrahydrofuran (1.0 mL) at 0° C. was added hydrogen fluoride-pyridine complex (70%, 0.10 mL, 3.9 mmol, excess), and the reaction mixture was allowed to slowly warm to 25° C. After 5 h, the reaction mixture was quenched with saturated aqueous sodium bicarbonate solution (10 mL), and the two phases were separated. The aqueous layer was extracted with ethyl acetate (3×20 mL), and the combined organic layers were dried with anhydrous sodium sulfate and concentrated under reduced pressure. The obtained residue was purified by flash column chromatography (silica gel, 5→20% methanol in ethyl acetate) to afford pure epothilone 23 (22.0 mg, 41.5 μmol, 82% yield) as a white amorphous solid. 23: R_{f}=0.28 (silica gel, 20% methanol in ethyl acetate); [α]_{D}²²=−19.2 (c=0.38, CH₂Cl₂); FT-IR (film) vₘₐₓ 3367, 2922, 2852, 1727, 1687, 1555, 1462, 1378, 1334, 1274, 1261, 1148, 1057, 980, 885, 802, 764, 749, 671 cm⁻¹; ¹H NMR (600 MHz, CD₂Cl₂) δ=6.40 (s, 1H), 6.33 (s, 1H), 5.43 (dd, J=4.2, 4.2 Hz, 1H), 4.03 (dd, J=9.6, 1.8 Hz, 1H), 3.84 (s, 3H), 3.74 (dd, J=4.8, 4.8 Hz, 1H), 3.67-3.60 (m, 2H), 3.29 (dq, J=6.6, 6.6 Hz, 1H), 2.65-2.60 (m, 1H), 2.58-2.54 (m, 1H), 2.49 (dd, J=13.8, 10.2 Hz, 1H), 2.41 (s, 3H), 2.39 (dd, J=13.8, 1.8 Hz, 1H), 2.04-2.03 (m, 1H), 2.00 (s, 3H), 1.97-1.93 (m, 1H), 1.85-1.80 (m, 1H), 1.50-1.25 (m, 7H), 1.34 (s, 3H), 1.14 (s, 3H), 1.12 (d, J=6.6 Hz, 3H), 1.03 (s, 3H), 0.96 (d, J=6.6 Hz, 3H) ppm; ¹³C NMR (151 MHz, CD₂Cl₂) δ=220.7, 171.4, 148.4, 137.0, 135.8, 118.1, 109.3, 77.8, 75.9, 75.1, 62.5, 55.4, 52.7, 48.0, 44.8, 43.2, 39.4, 36.8, 35.5, 34.7, 31.8, 29.4, 21.8, 21.5, 19.8, 19.1, 17.6, 16.4, 15.7, 14.5 ppm; HRMS (ESI) calcd for C₃₀H₄₉N₃O₆SNa⁺ [M+Na]⁺ 602.3234, found 602.3235.

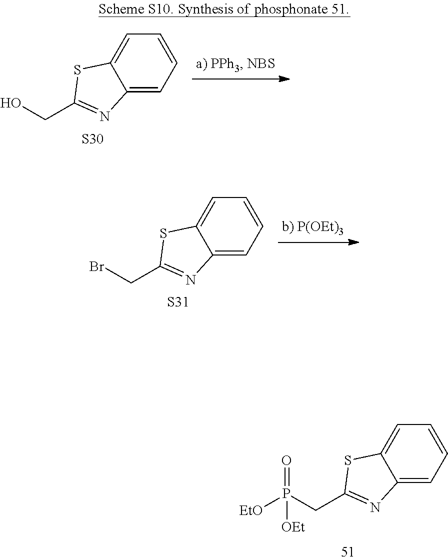 [SMILES: OCc1nc2ccccc2s1 ; "S30" ; BrCc1nc2ccccc2s1 ; "S31" ; CCOP(=O)(Cc1nc2ccccc2s1)OCC ; "51"]

### 2-(Bromomethyl)-1,3-benzothiazole (S31)

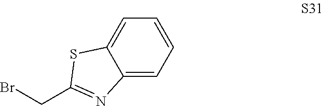 [SMILES: BrCc1nc2ccccc2s1]

To a stirred solution of hydroxymethyl benzothiazole S30 (1.00 g, 6.05 mmol, 1.0 equiv) in dichloro-methane/tetrahydrofuran (1:1, 40 mL) at −78° C. was added triphenylphosphine (1.59 g, 6.05 mmol, 1.0 equiv), followed by N-bromosuccinimide (1.08 g, 6.05 mmol, 1.0 equiv). After 5 min, the reaction mixture was quenched with water (20 mL), and allowed to warm to 25° C. The two phases were separated, and the aqueous layer was extracted with ethyl acetate (3×30 mL). The combined organic layers were dried with anhydrous sodium sulfate and concentrated under reduced pressure. The obtained residue was purified by flash column chromatography (silica gel, 2→8% ethyl acetate in hexanes) to afford pure bromomethyl benzothiazole S31 (780 mg, 3.42 mmol, 57% yield) as a white amorphous solid. S31: R_{f}=0.48 (silica gel, 10% ethyl acetate in hexanes); FT-IR (film) vₘₐₓ 3059, 3028, 1594, 1557, 1505, 1456, 1430, 1313, 1278, 1242, 1190, 1157, 1125, 1090, 1061, 1013, 938, 901, 851, 756, 727, 706 cm⁻¹; ¹H NMR (600 MHz, CDCl₃) δ=8.03 (d, J=8.4 Hz, 1H), 7.88 (d, J=7.8 Hz, 1H), 7.51 (ddd, J=7.8, 7.8, 1.2 Hz, 1H), 7.43 (ddd, J=7.8, 7.8, 1.2 Hz, 1H), 4.82 (s, 2H) ppm; ¹³C NMR (151 MHz, CDCl₃) δ=166.2, 152.8, 136.2, 126.5, 125.9, 123.5, 121.8, 27.1 ppm; HRMS (ESI) calcd for C₈H7BrNS₂⁺ [M+H]⁺ 227.9477, found 227.9466.

### Diethyl (1,3-benzothiazol-2-ylmethyl)phosphonate (51)

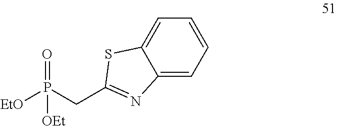 [SMILES: CCOP(=O)(Cc1nc2ccccc2s1)OCC]

A stirred solution of bromomethyl benzothiazole S31 (775 mg, 3.40 mmol, 1.0 equiv) in triethyl phosphite (2.0 mL, 12 mmol, 3.4 equiv) was heated to 160° C. After 2 h, the excess triethyl phosphite was removed under a steady flow of nitrogen gas, and the reaction mixture was allowed to cool to 25° C. The obtained residue was purified by flash column chromatography (silica gel, 60→90% ethyl acetate in hexanes) to afford pure phosphonate 51 (820 mg, 2.87 mmol, 84% yield) as a colorless oil. 51: R_{f}=0.32 (silica gel, ethyl acetate); FT-IR (neat) vₘₐₓ 3470, 3060, 2982, 2907, 1639, 1593, 1539, 1511, 1475, 1456, 1436, 1392, 1368, 1313, 1244, 1195, 1162, 1093, 1045, 1015, 963, 891, 842, 761, 731, 708, 677 cm¹; ¹H NMR (600 MHz, CDCl₃) δ=7.80 (d, J=7.8 Hz, 1H), 7.86 (d, J=7.8 Hz, 1H), 7.47 (ddd, J=8.4, 7.2, 1.2 Hz, 1H), 7.38 (ddd, J=8.4, 7.2, 1.2 Hz, 1H), 4.19-4.13 (m, 4H), 3.73 (d, J=21.6 Hz, 2H), 1.32 (t, J=7.2 Hz, 6H) ppm; ¹³C NMR (151 MHz, CDCl₃) δ=161.2 (d, J=9.3 Hz), 153.1 (d, J=2.4 Hz), 136.1, 126.3, 125.3, 123.1, 121.7, 63.0 (d, J=6.6 Hz), 33.3 (d, J=139 Hz), 16.5 (d, J=6.0 Hz) ppm; HRMS (ESI) calcd for C₁₂H₁₆NO₃PSNa⁺ [M+Na]⁺ 308.0481, found 308.0482.

### (1S,3R,7R,10S,11S,12R,16S)-3-[(1E)-1-(1,3-Benzothiazol-2-yl)prop-1-en-2-yl]-17-(2-{[tert-butyl(dimethyl)silyl]oxy}ethyl)-8,8,10,12,16-pentamethyl-7,11-bis[(triethylsilyl)oxy]-4-oxa-17-azabicyclo[14.1.0]heptadecane-5,9-dione (24a)

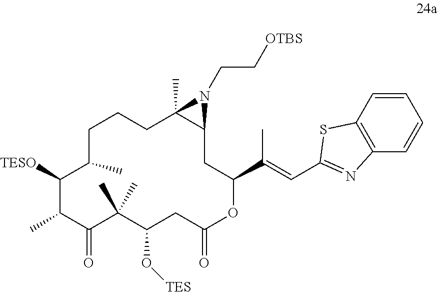

To a stirred solution of phosphonate 51 (150 mg, 0.533 mmol, 13 equiv) in tetrahydrofuran (0.5 mL) at −78° C. was added n-butyllithium (2.5 M in hexanes, 0.17 mL, 0.43 mmol, 10.0 equiv) dropwise. After 20 min, a solution of methyl ketone 82 (28.7 mg, 35.9 μmol, 1.0 equiv) in tetrahydrofuran (0.5 mL) was added, and the reaction mixture was allowed to slowly warm to 10° C., and stirred for an additional 1 h. Then the reaction mixture was quenched with saturated aqueous ammonium chloride solution (10 mL), and the two phases were separated. The aqueous layer was extracted with ethyl acetate (3×5 mL), and the combined organic layers were dried with anhydrous sodium sulfate and concentrated under reduced pressure. The obtained residue was purified by flash column chromatography (silica gel, 5→30% ethyl acetate in hexanes) to afford pure protected epothilone 24a (22 mg, 27 μmol, 65% yield) as a colorless oil. 24a: R_{f}=0.30 (silica gel, 15% ethyl acetate in hexanes); [α]2=−4.8 (c=1.0, CH₂Cl₂); FT-IR (neat) vₘₐₓ 2952, 2876, 1745, 1696, 1643, 1460, 1434, 1414, 1381, 1306, 1283, 1248, 1198, 1157, 1107, 1008, 985, 835 cm⁻¹; ¹H NMR (600 MHz, CD₂Cl₂) δ=7.99 (dd, J=8.4, 1.2 Hz, 1H), 7.91 (dd, J=8.4, 1.2 Hz, 1H), 7.48 (ddd, J=7.8, 6.6, 1.2 Hz, 1H), 7.38 (ddd, J=7.8, 6.6, 1.2 Hz, 1H), 6.80 (s, 1H), 5.22 (dd, J=7.8, 3.6 Hz, 1H), 4.15 (dd, J=7.8, 3.6 Hz, 1H), 3.88 (d, J=8.4 Hz, 1H), 3.71-3.65 (m, 2H), 3.05 (dq, J=8.4, 6.6 Hz, 1H), 2.72-2.62 (m, 3H), 2.39 (ddd, J=13.2, 6.6, 6.6 Hz, 1H), 2.29 (s, 3H), 2.16 (ddd, J=15.0, 3.0, 3.0 Hz, 1H), 1.78 (ddd, J=15.0, 9.0, 9.0 Hz, 1H), 1.66-1.58 (m, 4H), 1.48-1.43 (m, 2H), 1.31 (dd, J=10.2, 3.0 Hz, 1H), 1.28-1.24 (m, 1H), 1.19 (s, 3H), 1.16 (s, 3H), 1.12 (s, 3H), 1.09 (d, J=7.2 Hz, 3H), 0.99 (t, J=8.4 Hz, 9H), 0.98 (d, J=6.6 Hz, 3H), 0.94 (t, J=7.8 Hz, 9H), 0.86 (s, 9H), 0.69-0.62 (m, 12H), 0.03 (s, 3H), 0.02 (s, 3H) ppm; ¹³C NMR (151 MHz, CD₂Cl₂) δ=215.8, 171.2, 164.7, 153.9, 146.9, 135.4, 126.5, 125.3, 123.3, 121.7, 120.5, 80.1, 79.0, 75.8, 64.1, 54.8, 49.9, 48.2, 43.7, 40.6, 37.1, 36.1, 34.9, 32.2, 26.1, 25.3, 24.1, 22.6, 20.0, 18.6, 17.7, 15.52, 15.50, 7.3, 7.1, 5.8, 5.6, 5.23, 5.25 ppm; HRMS (ESI) calcd for C₅₀H₈₉N₂O₆SSi₃⁺ [M+H]⁺ 929.5744, found 929.5768.

### (1S,3S,7S,10R,11S,12S,16R)-3-[(1E)-1-(1,3-Benzothiazol-2-yl)prop-1-en-2-yl]-7,11-dihydroxy-17-(2-hydroxyethyl)-8,8,10,12,16-pentamethyl-4-oxa-17-azabicyclo[14.1.0]heptadecane-5,9-dione (24)

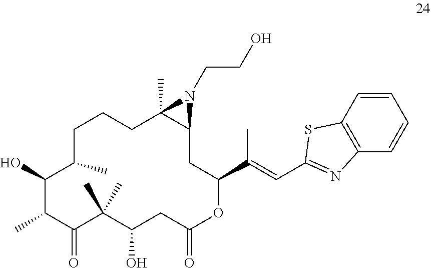

To a stirred solution of protected epothilone 24a (22.0 mg, 23.6 mmol, 1.0 equiv) in tetrahydrofuran (2.0 mL) at 0° C. was added hydrogen fluoride-pyridine complex (70% HF, 0.10 mL, 3.9 mmol, excess), and the reaction mixture was allowed to slowly warm to 25° C. After 9 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by flash column chromatography (silica gel, 5→20% methanol in ethyl acetate) to afford pure epothilone 24 (11.3 mg, 19.2 μmol, 81% yield) as a white amorphous solid. 24: Rₜ=0.39 (silica gel, 20% methanol in ethyl acetate); [α]_{D}²²=−15.7 (c=1.1, CH₂Cl₂); FT-IR (film) vₘₐₓ 3366, 2927, 2855, 1735, 1688, 1647, 1467, 1434, 1380, 1261, 1148, 1052, 1010, 980, 937, 876, 761, 730, 709 cm⁻¹; ¹H NMR (600 MHz, CD₂Cl₂) δ=7.99 (dd, J=7.8, 1.2 Hz, 1H), 7.91 (dd, J=7.8, 1.2 Hz, 1H), 7.49 (ddd, J=8.4, 7.2, 1.2 Hz, 1H), 7.39 (ddd, J=8.4, 7.2, 1.2 Hz, 1H), 6.88 (s, 1H), 5.56 (dd, J=3.6, 3.6 Hz, 1H), 4.05 (dd, J=10.2, 2.4 Hz, 1H), 3.75 (dd, J=6.0, 3.6 Hz, 1H), 3.71-3.61 (m, 2H), 3.31 (dq, J=7.2, 7.2 Hz, 1H), 2.70 (ddd, J=13.2, 7.2, 4.2 Hz, 1H), 2.58-2.53 (m, 2H), 2.45 (dd, J=13.8, 1.8 Hz, 1H), 2.32 (s, 3H), 2.06 (ddd, J=15.0, 6.0, 6.0 Hz, 1H), 1.86 (ddd, J=15.6, 7.8, 3.6 Hz, 1H), 1.70-1.64 (m, 1H), 1.60-1.48 (m, 2H), 1.47-1.38 (m, 2H), 1.37 (s, 3H), 1.38-1.28 (m, 2H), 1.27-1.25 (m, 1H), 1.15 (s, 3H), 1.13 (d, J=7.2 Hz, 3H), 1.04 (s, 3H), 0.97 (d, J=7.2 Hz, 3H) ppm; ¹³C NMR (151 MHz, CD₂Cl₂) δ=220.5, 171.4, 164.8, 153.8, 144.8, 153.3, 126.6, 125.4, 123.2, 121.8, 119.2, 77.1, 76.4, 75.4, 62.5, 55.5, 52.6, 47.7, 45.2, 43.2, 39.4, 35.3, 34.3, 31.5, 30.1, 29.1, 21.8, 19.5, 17.7, 16.9, 16.5, 14.8 ppm; HRMS (ESI) calcd for C₃₂H₄₇N₂O₆S⁺ [M+H]⁺ 587.3149, found 587.3153.

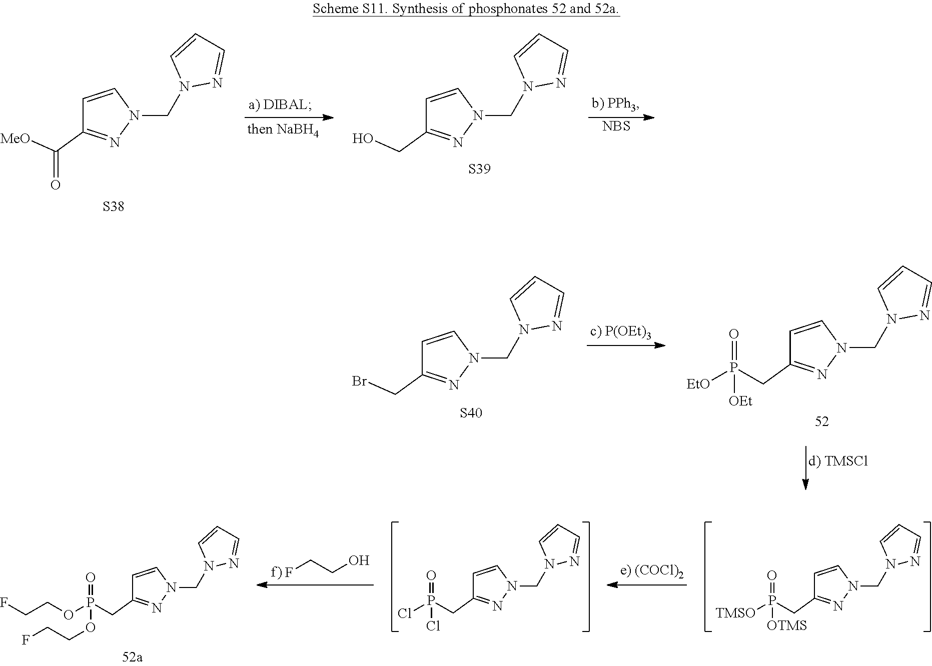

### [1-(1H-Pyrazol-1-ylmethyl)-1H-pyrazol-3-yl]methanol (S39)

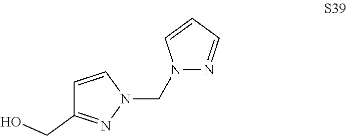 [SMILES: OCc1ccn(Cn2cccn2)n1]

To a stirred solution of pyrazolyl ester S38 (523 mg, 2.54 mmol, 1.0 equiv) in dichloromethane (11 mL) at −78° C. was added diisobutylaluminum hydride (1.0 min dichloromethane, 3.80 mL, 3.80 mmol, 1.5 equiv) dropwise. After 10 min, the reaction mixture was quenched with methanol (12 mL), and allowed to warm to 0° C. Sodium borohydride (0.288 g, 7.62 mmol, 3.0 equiv) was added, and after 10 min the reaction mixture was quenched with saturated aqueous potassium sodium tartrate solution (40 mL), and allowed to warm to 25° C. After 4 h, the quenched reaction mixture was filtered through a pad of Celite®. The two phases were separated, and the aqueous layer was extracted with ethyl acetate (3×30 mL). The combined organic layers were dried with anhydrous sodium sulfate and concentrated under reduced pressure. The obtained residue was purified by flash column chromatography (silica gel, 1→5% methanol in ethyl acetate) to afford pure hydroxymethyl pyrazole S39 (0.370 g, 2.08 mmol, 82% yield) as a colorless oil. S39: R_{f}=0.26 (silica gel, 5% methanol in ethyl acetate); FT-IR (neat) vₘₐₓ 3344, 3118, 2929, 2871, 1646, 1520, 1447, 1392, 1371, 1310, 1278, 1203, 1090, 1052, 997, 961, 917, 759 cm⁻¹; ¹H NMR (600 MHz, CDCl₃) δ=7.64 (d, J=2.4 Hz, 1H), 7.61 (d, J=2.4 Hz, 1H), 7.54 (d, J=1.8 Hz, 1H), 6.29 (dd, J=2.4, 1.8 Hz, 1H), 6.28 (d, J=2.4 Hz, 1H), 6.24 (s, 2H), 4.68 (s, 2H), 2.06 (br s, 1H) ppm; ¹³C NMR (151 MHz, CDCl₃) δ=153.8, 141.1, 131.0, 129.9, 107.4, 105.8, 65.4, 59.2 ppm; HRMS (CI) calcd for C₈₁H₁₁N₄O⁺ [M+H]⁺ 179.0927, found 179.0932.

### 3-(Bromomethyl)-1-(1H-pyrazol-1-ylmethyl)-1H-pyrazole (S40)

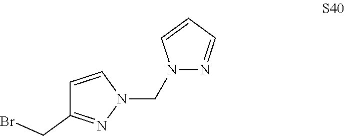 [SMILES: BrCc1ccn(Cn2cccn2)n1]

To a stirred solution of hydroxymethyl pyrazole S39 (0.358 g, 2.01 mmol, 1.0 equiv) in dichloromethane (12 mL) at −78° C. was added triphenylphosphine (580 mg, 2.21 mmol, 1.1 equiv), followed by N-bromosuccinimide (0.393 g, 2.21 mmol, 1.1 equiv). After 5 min, the reaction mixture was quenched with water (20 mL), and allowed to warm to 25° C. The two phases were separated, and the aqueous layer was extracted with ethyl acetate (3×15 mL). The combined organic layers were dried with anhydrous sodium sulfate and concentrated under reduced pressure. The obtained residue was purified by flash column chromatography (silica gel, 20→50% ethyl acetate in hexanes) to afford pure bromomethyl pyrazole S40 (0.330 g, 1.37 mmol, 68% yield) as a white amorphous solid. S40: R_{f}=0.22 (silica gel, 50% ethyl acetate in hexanes); FT-IR (film) vₘₐₓ 3122, 3096, 3022, 2972, 1718, 1627, 1520, 1474, 1447, 1430, 1392, 1366, 1312, 1278, 1213, 1206, 1154, 1115, 1089, 1055, 1009, 998, 973, 960, 917, 904, 867, 758, 737, 719 cm⁻¹; ¹H NMR (600 MHz, CDCl₃) δ=7.65 (d, J=2.4 Hz, 1H), 7.59 (d, J=2.4 Hz, 1H), 7.55 (d, J=1.8 Hz, 1H), 6.34 (d, J=2.4 Hz, 1H), 6.30 (dd, J=2.4, 1.8 Hz, 1H), 6.25 (s, 2H), 4.45 (s, 2H) ppm; ¹³C NMR (151 MHz, CDCl₃) δ=150.5, 141.2, 131.2, 129.9, 107.5, 65.5, 24.8 ppm; HRMS (CI) calcd for C₈₁H₁₀BrN₄⁺ [M+H]⁺ 241.0083, found 241.0089.

### Diethyl {[1-(1H-pyrazol-1-ylmethyl)-1H-pyrazol-3-yl]methyl}phosphonate (52)

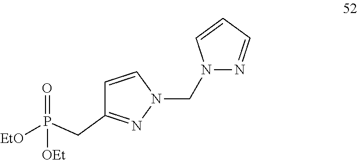 [SMILES: CCOP(=O)(Cc1ccn(Cn2cccn2)n1)OCC]

A stirred solution of bromomethyl pyrazole S40 (0.301 g, 1.25 mmol, 1.0 equiv) in triethyl phosphite (0.312 g, 1.88 mmol, 1.5 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 0→10% methanol in ethyl acetate) to afford pure phosphonate 52 (2.49 g, 8.97 mmol, 89% yield) as a colorless oil. 52: R_{f}=0.23 (silica gel, 10% methanol in ethyl acetate); FT-IR (neat) vₘₐₓ 3455, 3115, 2984, 2908, 1647, 1522, 1475, 1446, 1391, 1367, 1313, 1279, 1215, 1162, 1089, 1051, 1025, 963, 918, 848, 763 cm⁻¹; ¹H NMR (600 MHz, CDCl₃) δ=7.62 (d, J=2.4 Hz, 1H), 7.57 (d, J=2.4 Hz, 1H), 7.52 (d, J=1.8 Hz, 1H), 6.30 (dd, J=1.8, 1.8 Hz, 1H), 6.26 (dd, J=2.4, 1.8 Hz, 1H), 6.22 (s, 2H), 4.06-3.97 (m, 4H), 3.19 (d, J=20.4 Hz, 2H), 1.22 (t, J=7.2 Hz, 6H) ppm; ¹³C NMR (151 MHz, CDCl₃) δ=144.9 (d, J=7.2 Hz), 140.9, 131.0 (d, J=2.0 Hz), 129.7, 107.9 (d, J=3.3 Hz), 107.2, 65.4, 62.3 (d, J=6.5 Hz), 26.9 (d, J=140.6 Hz), 16.5 (d, J=6.0 Hz) ppm; HRMS (ESI) calcd for C₁₂H₂₀N₄O₃P⁺ [M+H]⁺ 299.1268, found 299.1264.

### Bis(2-fluoroethyl) {[1-(1H-pyrazol-1-ylmethyl)-1H-pyrazol-3-yl]methyl}phosphonate (52a)

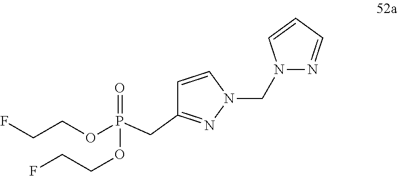 [SMILES: O=P(Cc1ccn(Cn2cccn2)n1)(OCCF)OCCF]

A stirred solution of phosphonate 52 (0.230 g, 0.771 mmol, 1.0 equiv) in trimethylsilyl chloride (0.490 mL, 3.86 mmol, 5.0 equiv) was heated to 80° C. After 72 h, the reaction mixture was allowed to cool to 25° C., and the trimethylsilyl chloride was removed under reduced pressure. The crude residue was redissolved in dichloromethane (10 mL) with stirring, and cooled to 0° C. Then a solution of oxalyl chloride (0.244 g, 1.93 mmol, 2.5 equiv) in dichloromethane (5 mL) was added dropwise, and the reaction mixture was allowed to slowly warm to 25° C. After 4 h, the solvent was removed under reduced pressure. The crude residue was redissolved in dichloromethane (10 mL) with stirring, and cooled to 0° C. Then triethylamine (0.644 mL, 4.63 mmol, 6.0 equiv), 2-fluoroethanol (197 mg, 3.08 mmol, 4.0 equiv) and 4-(dimethylamino)pyridine (5.0 mg, 41 nmol, 0.05 equiv) were added sequentially, and the reaction mixture was allowed to slowly warm to 25° C. After 1 h, the reaction mixture was diluted with ethyl acetate (20 mL), and washed with water (10 mL) and brine (10 mL). The two phases were separated, and the organic layer was dried with anhydrous sodium sulfate and concentrated under reduced pressure. The obtained residue was purified by flash column chromatography (silica gel, 20→60% ethyl acetate in hexanes) to afford pure phosphonate 52a (185 mg, 0.553 mmol, 72% yield overall) as a colorless oil. 52a: R_{f}=0.27 (silica gel, 10% methanol in ethyl acetate); FT-IR (neat) vₘₐₓ 3443, 3117, 2957, 2921, 2851, 1644, 1520, 1449, 1391, 1367, 1313, 1279, 1251, 1120, 1075, 1031, 962, 870, 847, 811, 764 cm⁻¹; ¹H NMR (600 MHz, CDCl₃) δ=7.64 (d, J=2.4 Hz, 1H), 7.59 (d, J=1.8 Hz, 1H), 7.53 (d, J=1.8 Hz, 1H), 6.32 (dd, J=1.8, 1.8 Hz, 1H), 6.28 (dd, J=1.8, 1.8 Hz, 1H), 6.23 (s, 2H), 4.56-4.51 (m, 2H), 4.49-4.43 (m, 2H), 4.27-4.11 (m, 4H), 3.30 (d, J=21.6 Hz, 2H) ppm; ¹³C NMR (151 MHz, CDCl₃) δ=144.2 (d, J=7.8 Hz), 141.0, 131.2 (d, J=2.0 Hz), 129.8, 108.0 (d, J=3.6 Hz), 107.3, 82.4 (dd, J=170.9, 6.0 Hz), 65.4, 65.2 (d, J=20.6, 6.6 Hz), 26.8 (d, J=141.9 Hz) ppm; HRMS (ESI) calcd for Cl₂H₁₇F₂N₄O₃PNa⁺ [M+Na]⁺ 357.0899, found 357.0909.

### (1S,3S,7S,10R,11S,12S,16R)-17-(2-{[tert-Butyl(dimethyl)silyl]oxy}ethyl)-8,8,10,12,16-pentamethyl-3-{(1E)-1-[1-(1H-pyrazol-1-ylmethyl)-1H-pyrazol-3-yl]prop-1-en-2-yl}-7,11-bis[(triethylsilyl)oxy]-4-oxa-17-azabicyclo[14.1.0]heptadecane-5,9-dione (100)

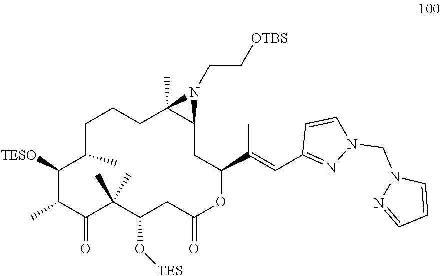

To a stirred solution of phosphonate 52 (45.0 mg, 0.151 mmol, 11 equiv) in tetrahydrofuran (0.6 mL) at −78° C. was added n-butyllithium (2.5 M in hexanes, 55 μL 0.14 mmol, 10.0 equiv) dropwise. After 40 min, the reaction mixture was transferred to a stirred solution of methyl ketone 82 (10.5 mg, 13.2 μmol, 1.0 equiv) in tetrahydrofuran (0.5 mL) at −78° C., and stirred for an additional 1 h. Then the reaction mixture was quenched with saturated aqueous ammonium chloride solution (5 mL), and allowed to warm to 25° C. The two phases were separated, and the aqueous layer was extracted with ethyl acetate (3×10 mL). The combined organic layers were dried with anhydrous sodium sulfate and concentrated under reduced pressure to afford a crude mixture of adduct 98 {confirmed by HRMS (ESI): calcd for C₅₄H₁₀₃N₅O₁₀PSi₃⁺ [M+H]⁺ 1096.6745, found 1096.6749} and protected epothilone 100.

The obtained crude residue was redissolved in tetrahydrofuran (1.0 mL) with stirring, and cooled to 20° C. Potassium tert-butoxide (7.3 mg, 66 μmol, 5.0 equiv) was added, and after 5 min the reaction mixture was quenched with water (3 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by flash column chromatography (silica gel, 20→50% ethyl acetate in hexanes) to afford pure protected epothilone 100 (9.1 mg, 9.7 μmol, 73% yield overall, plus 10% recovered methyl ketone 82) as a colorless oil. 100: R_{f}=0.19 (silica gel, 40% ethyl acetate in hexanes); [α]2_{D}²²=−1.5 (c=0.20, CH₂Cl₂); FT-IR (neat) vₘₐₓ 2954, 2877, 1741, 1696, 1517, 1460, 1414, 1381, 1305, 1280, 1250, 1202, 1159, 1108, 1068, 1007, 985, 957, 860, 836, 779, 741 cm⁻¹; ¹H NMR (600 MHz, C₆D₆) δ=7.42 (d, J=1.8 Hz, 1H), 7.13 (d, J=2.4 Hz, 1H), 7.10 (d, J=2.4 Hz, 1H), 6.75 (s, 1H), 6.05 (d, J=2.4 Hz, 1H), 5.89 (dd, J=2.4, 1.8 Hz, 1H), 5.56 (s, 2H), 5.45 (dd, J=8.4, 4.2 Hz, 1H), 4.21-4.17 (m, 2H), 3.86-3.78 (m, 2H), 3.03 (dq, J=9.0, 6.6 Hz, 1H), 2.74 (ddd, J=12.0, 6.0, 6.0 Hz, 1H), 2.70 (dd, J=16.2, 9.0 Hz, 1H), 2.57 (dd, J=16.2, 2.4 Hz, 1H), 2.44 (ddd, J=12.0, 6.0, 6.0 Hz, 1H), 2.26 (ddd, J=15.0, 3.0, 3.0 Hz, 1H), 2.18 (s, 3H), 2.02 (ddd, J=15.0, 9.0, 9.0 Hz, 1H), 1.88-1.80 (m, 2H), 1.76-1.70 (m, 1H), 1.65-1.57 (m, 1H), 1.51-1.43 (m, 2H), 1.24 (dd, J=9.6, 3.0 Hz, 1H), 1.21-1.18 (m, 1H), 1.20 (d, J=6.6 Hz, 3H), 1.18 (s, 3H), 1.14 (s, 3H), 1.13 (d, J=6.6 Hz, 3H), 1.08 (t, J=7.8 Hz, 9H), 1.07 (t, J=7.8 Hz, 9H), 0.99 (s, 9H), 0.84-0.77 (m, 6H), 0.82 (s, 3H), 0.74-0.70 (m, 6H), 0.09 (s, 6H) ppm; ¹³C NMR (151 MHz, C₆D₆) δ=214.4, 170.7, 150.5, 140.6, 138.4, 129.9, 129.3, 119.4, 107.8, 107.0, 80.2, 79.3, 75.9, 65.1, 64.3, 54.9, 53.4, 50.2, 48.1, 43.3, 40.1, 37.4, 36.4, 35.5, 32.3, 26.2, 25.4, 23.5, 23.3, 20.2, 18.5, 17.6, 15.6, 14.9, 7.4, 7.3, 6.0, 5.8, 5.1 ppm; HRMS (ESI) calcd for C₅₀H₉₂N₅O₆Si₃⁺ [M+H]⁺ 942.6350, found 942.6362.

### (1S,3S,7S,10R,11S,12S,16R)-7,11-Dihydroxy-17-(2-hydroxyethyl)-8,8,10,12,16-pentamethyl-3-{(1E)-1-[1-(1H-pyrazol-1-ylmethyl)-1H-pyrazol-3-yl]prop-1-en-2-yl}-4-oxa-17-azabicyclo-[14.1.0]heptadecane-5,9-dione (25)

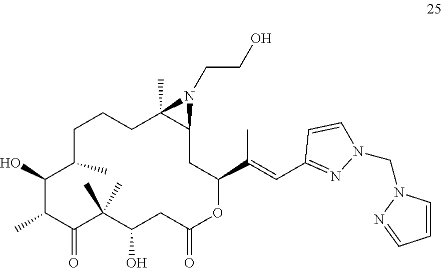

To a stirred solution of protected epothilone 100 (8.1 mg, 8.6 μmol, 1.0 equiv) in tetrahydrofuran (1.8 mL) at 0° C. was added hydrogen fluoride-pyridine complex (70%, 0.30 mL, 11 mmol, excess), and the reaction mixture was allowed to slowly warm to 25° C. After 3 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by flash column chromatography (silica gel, 5→30% methanol in ethyl acetate) to afford pure epothilone 25 (4.9 mg, 8.2 μmol, 95% yield) as a white amorphous solid. 25: R_{f}=0.27 (silica gel, 25% methanol in ethyl acetate); [α]2=22.0 (c=0.10, CH₂Cl₂); FT-IR (film) vma, 3372, 2932, 2877, 1730, 1687, 1511, 1466, 1424, 1382, 1280, 1215, 1174, 1149, 1087, 1054, 1007, 980, 939, 883, 760, 734 cm⁻¹; ¹H NMR (600 MHz, CD₂Cl₂) δ=7.68 (d, J=2.4 Hz, 1H), 7.63 (d, J=2.4 Hz, 1H), 7.52 (d, J=1.8 Hz, 1H), 6.45 (s, 1H), 6.33 (d, J=2.4 Hz, 1H), 6.30 (dd, J=2.4, 1.8 Hz, 1H), 6.25 (s, 2H), 5.45 (dd, J=4.8, 4.8 Hz, 1H), 4.01 (dd, J=10.2, 2.4 Hz, 1H), 3.74 (dd, J=6.0, 4.2 Hz, 1H), 3.67-3.60 (m, 2H), 3.28 (dq, J=6.6, 6.6 Hz, 1H), 2.64 (ddd, J=12.6, 6.6, 3.6 Hz, 1H), 2.60-2.53 (m, 1H), 2.51 (dd, J=14.4, 10.2 Hz, 1H), 2.41 (dd, J=14.4, 1.8 Hz, 1H), 2.01 (s, 3H), 1.97 (ddd, J=15.0, 6.0, 6.0 Hz, 1H), 1.83-1.79 (m, 1H), 1.67-1.63 (m, 3H), 1.56-1.52 (m, 1H), 1.50-1.45 (m, 1H), 1.44-1.39 (m, 1H), 1.40-1.35 (m, 1H), 1.34 (s, 3H), 1.32-1.30 (m, 1H), 1.14 (s, 3H), 1.12 (d, J=7.2 Hz, 3H), 1.03 (s, 3H), 0.96 (d, J=6.6 Hz, 3H) ppm; ¹³C NMR (151 MHz, CD₂Cl₂) δ=220.6, 171.4, 150.5, 141.0, 136.9, 130.6, 130.0, 117.9, 107.6, 107.2, 77.7, 76.1, 75.3, 65.7, 62.5, 55.4, 52.6, 47.9, 45.0, 43.2, 39.4, 35.5, 34.6, 31.7, 29.4, 21.9, 21.7, 19.7, 17.7, 16.5, 15.7, 14.6 ppm; HRMS (ESI) calcd for C₃₂H₅₀N₅O₆⁺ [M+H]⁺ 600.3756, found 600.3760.

### 2-[(1S,3S,7S,10R,11S,12S,16R)-7,11-Dihydroxy-8,8,10,12,16-pentamethyl-5,9-dioxo-3-{(1E)-1-[1-(1H-pyrazol-1-ylmethyl)-1H-pyrazol-3-yl]prop-1-en-2-yl}-4-oxa-17-azabicyclo[14.1.0]heptadec-17-yl]ethyl acetate (26)

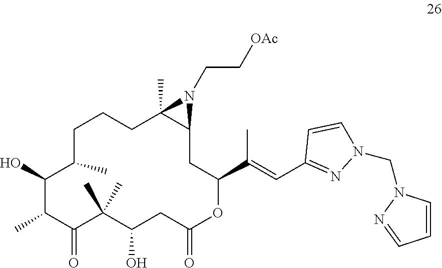

To a stirred solution of epothilone 25 (3.7 mg, 6.2 μmol, 1.0 equiv) in dichloromethane (1 mL) at 0° C. was added N,N-diisopropylethylamine (1.6 mg, 12 μmol, 2.0 equiv), followed by acetic anhydride (1.3 mg, 12 μmol, 2.0 equiv) and 4-dimethylaminopyridine (0.4 mg, 3 μmol, 0.05 equiv). After 1.5 h, the reaction mixture was quenched with methanol (1 mL), and allowed to warm to 25° C. The solvent was removed under reduced pressure, and the obtained residue was purified by preparative thin layer chromatography (silica gel, 5% methanol in ethyl acetate) to afford epothilone 26 (2.8 mg, 4.4 μmol, 71% yield) as a white amorphous solid. 26: R_{f}=0.33 (silica gel, 5% methanol in ethyl acetate); [α]_{D}²²=−22.0 (c=0.10, CH₂Cl₂); FT-IR (film) vₘₐₓ 3362, 2928, 1734, 1687, 1511, 1453, 1382, 1368, 1279, 1249, 1150, 1087, 1048, 1008, 980, 918, 884, 759, 733 cm⁻¹; ¹H NMR (600 MHz, CD₂Cl₂) δ=7.67 (d, J=2.4 Hz, 1H), 7.63 (d, J=2.4 Hz, 1H), 7.52 (d, J=1.8 Hz, 1H), 6.43 (s, 1H), 6.33 (d, J=2.4 Hz, 1H), 6.30 (dd, J=2.4, 1.8 Hz, 1H), 6.25 (s, 2H), 5.42 (dd, J=4.8, 4.8 Hz, 1H), 4.26 (ddd, J=12.0, 6.0, 6.0 Hz, 1H), 4.14 (ddd, J=12.0, 6.0, 6.0 Hz, 1H), 4.07-4.04 (m, 1H), 3.73 (ddd, J=4.8, 4.8, 4.8 Hz, 1H), 3.30 (dq, J=6.6, 6.6 Hz, 1H), 2.73 (ddd, J=12.0, 6.0, 6.0 Hz, 1H), 2.64 (ddd, J=12.0, 6.0, 6.0 Hz, 1H), 2.47 (dd, J=14.4, 10.2 Hz, 1H), 2.40 (dd, J=14.4, 2.4 Hz, 1H), 2.02 (s, 3H), 2.01 (s, 3H), 1.90-1.88 (m, 1H), 1.84 (ddd, J=13.2, 6.6, 6.6 Hz, 1H), 1.65-1.62 (m, 1H), 1.57-1.51 (m, 2H), 1.50-1.38 (m, 4H), 1.35 (s, 3H), 1.28-1.25 (m, 1H), 1.15 (s, 3H), 1.12 (d, J=7.2 Hz, 3H), 1.04 (s, 3H), 0.95 (d, J=7.2 Hz, 3H) ppm; ¹³C NMR (151 MHz, CD₂Cl₂) δ=220.6, 171.3, 171.2, 150.6, 141.0, 137.5, 130.6, 130.0, 118.0, 107.6, 107.3, 78.3, 75.1, 74.5, 65.7, 64.7, 52.9, 51.1, 48.2, 44.4, 43.4, 39.6, 35.8, 35.4, 32.2, 30.4, 22.1, 21.2, 20.9, 20.6, 17.7, 16.5, 15.5, 14.0 ppm; HRMS (ESI) calcd for C₃₄H₅₂N₅O₇⁺ [M+H]+ 642.3861, found 642.3863.

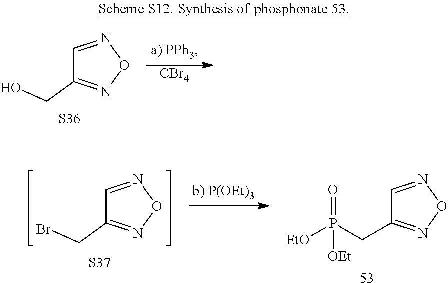

### Diethyl (1,2,5-oxadiazol-3-ylmethyl)phosphonate (53)

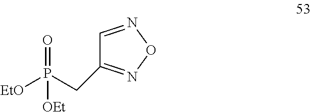 [SMILES: CCOP(=O)(Cc1cnon1)OCC]

To a stirred solution of hydroxymethyl oxadiazole S36 (0.245 g, 2.45 mmol, 1.0 equiv) in dichloromethane (6 mL) at 0° C. was added triphenylphosphine (0.706 g, 2.69 mmol, 1.1 equiv), followed by carbon tetrabromide (0.892 g, 2.69 mmol, 1.1 equiv). After 10 min, the reaction mixture was quenched with water (10 mL), and allowed to warm to 25° C. The two phases were separated, and the aqueous layer was extracted with ethyl acetate (3×5 mL). The combined organic layers were dried with anhydrous sodium sulfate and concentrated under reduced pressure. The obtained residue was filtered through a pad of silica gel and rinsed thoroughly with hexanes (20 mL) to afford crude bromomethyl oxadiazole S37 as a volatile oil (crude NMR provided). S37: R_{f}=0.27 (silica gel, 20% ethyl acetate in hexanes); ¹H NMR (600 MHz, CDCl₃) δ=8.35 (s, 1H), 4.56 (s, 2H) ppm; ¹³C NMR (150 MHz, CDCl₃) δ=152.5, 141.7, 17.0 ppm.

A stirred solution of crude bromomethyl oxadiazole S37 in triethyl phosphite (0.814 g, 4.90 mmol, 2.0 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 50→80% ethyl acetate in hexanes) to afford pure phosphonate 53 (0.205 g, 0.93 mmol, 38% yield overall) as a colorless oil. 53: R_{f}=0.30 (silica gel, ethyl acetate); FT-IR (neat) vₘₐₓ 3465, 2986, 2913, 1568, 1479, 1445, 1388, 1253, 1164, 1098, 1051, 1019, 987, 971, 887, 849, 827, 804 cm⁻¹; ¹H NMR (600 MHz, CDCl₃) δ=8.34 (s, 1H), 4.16-4.10 (m, 4H), 3.38 (d, J=21.0 Hz, 2H), 1.13 (t, J=7.2 Hz, 6H) ppm; ¹³C NMR (151 MHz, CDCl₃) δ=147.7 (d, J=7.2 Hz), 142.1 (d, J=1.8 Hz), 63.1 (d, J=6.6 Hz), 22.6 (d, J=142.2 Hz), 16.5 (d, J=5.9 Hz) ppm; HRMS (ESI) calcd for C₇H₁₃N₂O₄PNa⁺ [M+Na]⁺ 243.0505, found 243.0511.

### (1S,3S,7S,10R,11S,12S,16R)-17-(2-{[tert-Butyl(dimethyl)silyl]oxy}ethyl)-8,8,10,12,16-pentamethyl-3-[(1E)-1-(1,2,5-oxadiazol-3-yl)prop-1-en-2-yl]-7,11-bis[(triethylsilyl)oxy]-4-oxa-17-azabicyclo[14.1.0]heptadecane-5,9-dione (27a)

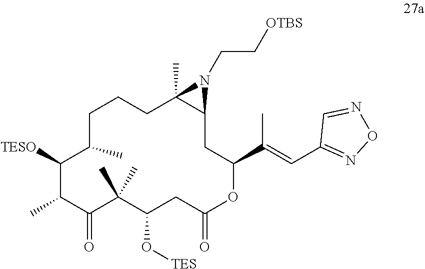

To a stirred solution of phosphonate 53 (90.0 mg, 0.409 mmol, 31 equiv) in tetrahydrofuran (0.5 mL) at −78° C. was added n-butyllithium (2.5 M in hexanes, 0.15 mL, 0.37 mmol, 29 equiv) dropwise. After 10 min, the reaction mixture was added to a stirred solution of methyl ketone 82 (10.5 mg, 13.2 μmot, 1.0 equiv) in tetrahydrofuran (0.5 mL) at −78° C., and was allowed to slowly warm to 0° C. over 3 h. The reaction mixture was then quenched with water (5 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, 20% ethyl acetate in hexanes) to afford pure protected epothilone 27a (3.6 mg, 4.2 μmot, 32% yield) as a colorless oil. 27a: R_{f}=0.31 (silica gel, 20% ethyl acetate in hexanes); [α]_{D}²²=−5.0 (c=0.10, CH₂Cl₂); FT-IR (neat) vₘₐₓ 2954, 2929, 2877, 2856, 1731, 1697, 1463, 1414, 1381, 1367, 1285, 1256, 1157, 1112, 1073, 1041, 1019, 986, 836, 779, 741 cm⁻¹; ¹H NMR (600 MHz, C₆D₆) δ=7.38 (s, 1H), 6.24 (s, 1H), 5.31 (dd, J=7.2, 3.6 Hz, 1H), 4.21 (dd, J=7.8, 3.0 Hz, 1H), 4.15 (d, J=8.4 Hz, 1H), 3.84 (ddd, J=10.2, 6.6, 6.6 Hz, 1H), 3.80 (ddd, J=10.2, 6.0, 6.0 Hz, 1H), 3.00 (dq, J=7.2, 6.6 Hz, 1H), 2.74 (ddd, J=12.0, 6.0, 6.0 Hz, 1H), 2.70 (dd, J=16.2, 8.4 Hz, 1H), 2.56 (dd, J=16.2, 3.6 Hz, 1H), 2.44 (ddd, J=12.0, 6.0, 6.0 Hz, 1H), 2.17 (ddd, J=15.0, 4.2, 4.2 Hz, 1H), 2.00-1.93 (m, 1H), 1.97 (s, 3H), 1.90-1.79 (m, 2H), 1.70-1.59 (m, 2H), 1.50-1.42 (m, 2H), 1.38-1.27 (m, 2H), 1.19 (d, J=6.6 Hz, 3H), 1.17 (s, 3H), 1.15 (s, 3H), 1.13 (d, J=7.2 Hz, 3H), 1.08-1.04 (m, 18H), 0.98 (s, 9H), 0.89 (s, 3H), 0.80-0.75 (m, 6H), 0.72-0.69 (m, 6H), 0.08 (s, 6H) ppm; ¹³C NMR (151 MHz, C₆D₆) δ=214.4, 170.7, 151.4, 148.3, 141.5, 110.8, 80.2, 78.3, 75.8, 64.3, 54.9, 53.4, 49.8, 48.0, 43.3, 40.1, 37.3, 36.4, 35.0, 32.5, 30.2, 26.1, 25.5, 23.6, 22.9, 20.0, 18.5, 17.3, 15.6, 7.4, 7.3, 5.9, 5.7, 5.1 ppm; HRMS (ESI) calcd for C₄₅H₈₆N₃O₇Si₃⁺ [M+H]⁺ 864.5768, found 864.5771.

### (1S,3S,7S,10R,11S,12S,16R)-7,11-Dihydroxy-17-(2-hydroxyethyl)-8,8,10,12,16-pentamethyl-3-[(1E)-1-(1,2,5-oxadiazol-3-yl)prop-1-en-2-yl]-4-oxa-17-azabicyclo[14.1.0]heptadecane-5,9-dione (27)

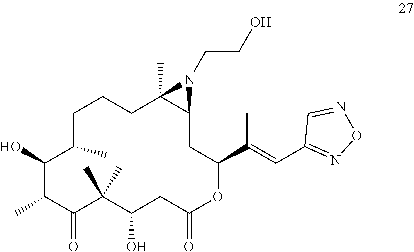

To a stirred solution of protected epothilone 27a (1.5 mg, 1.7 nmol, 1.0 equiv) in tetrahydrofuran (0.9 mL) at 0° C. was added hydrogen fluoride-pyridine complex (70%, 0.10 mL, 3.9 mmol, excess), and the reaction mixture was allowed to slowly warm to 25° C. After 3 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by preparative thin layer chromatography (silica gel, 20% methanol in ethyl acetate) to afford pure epothilone 27 (0.8 mg, 1.5 μmol, 80% yield) as a white amorphous solid. 27: R_{f}=0.25 (silica gel, 15% methanol in ethyl acetate); [α]2_{D}²²=−6.3 (c=0.10, CH₂Cl₂); FT-IR (film) vₘₐₓ 3360, 2923, 2852, 1733, 1660, 1633, 1468, 1411, 1260, 1147, 1056, 981, 886, 800 cm⁻¹; ¹H NMR (600 MHz, C₆D₆) δ=7.40 (s, 1H), 6.35 (s, 1H), 5.38 (dd, J=4.2, 4.2 Hz, 1H), 4.07 (dd, J=6.6, 5.4 Hz, 1H), 3.86 (dd, J=7.2, 3.6 Hz, 1H), 3.61-3.54 (m, 2H), 3.31 (dq, J=7.2, 7.2 Hz, 1H), 2.42-2.38 (m, 1H), 2.37-2.36 (m, 1H), 2.13-2.09 (m, 1H), 1.85-1.80 (m, 1H), 1.71 (s, 3H), 1.63-1.57 (m, 1H), 1.54-1.49 (m, 1H), 1.48-1.44 (m, 1H), 1.40-1.26 (m, 4H), 1.23-1.18 (m, 1H), 1.15 (s, 3H), 1.09 (d, J=6.6 Hz, 3H), 1.03 (d, J=7.2 Hz, 3H), 1.02-0.95 (m, 1H), 0.91 (s, 3H), 0.81-0.77 (m, 1H), 0.74 (s, 3H) ppm; ¹³C NMR (151 MHz, C₆D₆) δ=219.2, 170.8, 151.3, 145.9, 141.7, 109.4, 77.1, 75.9, 75.8, 62.2, 55.6, 52.0, 46.7, 45.9, 42.5, 38.8, 35.0, 33.6, 30.3, 30.0, 28.4, 22.7, 21.8, 18.3, 17.8, 16.8, 16.2 ppm; HRMS (ESI) calcd for C₂₇H₄₄N₃O₇⁺ [M+H]⁺ 522.3174, found 522.3182.

### Scheme S13. Synthesis of Phosphonate 54

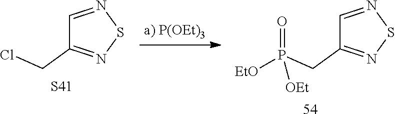

### Diethyl (1,2,5-thiadiazol-3-ylmethyl)phosphonate (54)

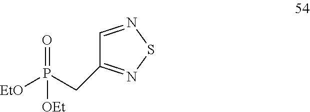 [SMILES: CCOP(=O)(Cc1cnsn1)OCC]

A stirred solution of chloromethyl thiadiazole S41 (0.150 g, 1.11 mmol, 1.0 equiv) in triethyl phosphite (0.484 g, 2.92 mmol, 2.6 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 40→70% ethyl acetate in hexanes) to afford pure phosphonate 54 (0.210 g, 0.889 mmol, 80% yield) as a colorless oil. 54: R_{f}=0.30 (silica gel, ethyl acetate); FT-IR (neat) vₘₐₓ 3466, 2983, 2910, 1645, 1487, 1444, 1393, 1357, 1320, 1249, 1163, 1097, 1048, 1017, 961, 885, 960, 828, 777, 735 cm⁻¹; ¹H NMR (600 MHz, CDCl₃) δ=8.57 (s, 1H), 4.14-1.08 (m, 4H), 3.57 (d, J=21.0 Hz, 2H), 1.29 (t, J=6.6 Hz, 6H) ppm; ¹³C NMR (151 MHz, CDCl₃) δ=155.0 (d, J=7.2 Hz), 150.5 (d, J=3.3 Hz), 62.8 (d, J=6.5 Hz), 29.1 (d, J=139.1 Hz), 16.5 (d, J=6.0 Hz) ppm; HRMS (ESI) calcd for C₇H₁₃N₂O₃PS⁺ [M+Na]⁺ 259.0277, found 259.0278.

### (1S,3S,7S,10R,11S,12S,16R)-17-(2-{[tert-Butyl(dimethyl)silyl]oxy}ethyl)-8,8,10,12,16-pentamethyl-3-[(1E)-1-(1,2,5-thiadiazol-3-yl)prop-1-en-2-yl]-7,11-bis [(triethylsilyl)oxy]-4-oxa-17-azabicyclo[14.1.0]heptadecane-5,9-dione (28a)

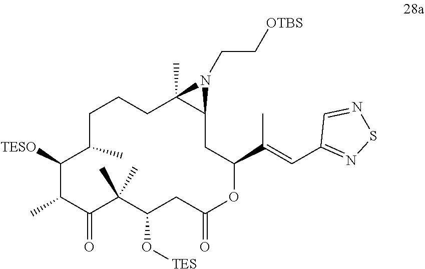

To a stirred solution of phosphonate 54 (90.0 mg, 0.381 mmol, 60 equiv) in tetrahydrofuran (0.5 mL) at −78° C. was added n-butyllithium (2.5 M in hexanes, 0.12 mL, 0.31 mmol, 48 equiv) dropwise. After 10 min, the reaction mixture was transferred to a stirred solution of methyl ketone 82 (5.0 mg, 6.3 μmol, 1.0 equiv) in tetrahydrofuran (0.5 mL) at −78° C., and was allowed to slowly warm to 0° C. over 3 h. Then the reaction mixture was quenched with water (5 mL), and allowed to warm to 25° C. The two phases were separated, the aqueous layer was extracted with ethyl acetate (3×10 mL), and the combined organic layers were dried with anhydrous sodium sulfate and concentrated under reduced pressure. The obtained residue was purified by preparative thin layer chromatography (silica gel, 20% ethyl acetate in hexanes) to afford pure protected epothilone 28a (3.9 mg, 4.4 μmol, 71% yield) as a colorless oil. 28a: R_{f}=0.37 (silica gel, 20% ethyl acetate in hexanes); [α]_{D}²²=−7.8 (c=0.32, CH₂Cl₂); FT-IR (neat) vₘₐₓ 2953, 2935, 2877, 1746, 1697, 1655, 1462, 1413, 1381, 1308, 1250, 1197, 1158, 1108, 1007, 985, 885, 835, 801, 779, 738 cm⁻¹; ¹H NMR (600 MHz, C₆D₆) δ=8.01 (s, 1H), 6.64 (s, 1H), 5.39 (dd, J=8.4, 4.2 Hz, 1H), 4.23 (dd, J=8.4, 3.6 Hz, 1H), 4.16 (d, J=8.4 Hz, 1H), 3.84 (ddd, J=10.2, 6.6, 6.6 Hz, 1H), 3.80 (ddd, J=9.6, 5.4, 5.4 Hz, 1H), 3.02 (dq, J=8.4, 6.6 Hz, 1H), 2.75 (ddd, J=12.0, 6.0, 6.0 Hz, 1H), 2.72 (dd, J=15.6, 8.4 Hz, 1H), 2.58 (dd, J=16.2, 3.0 Hz, 1H), 2.45 (ddd, J=12.0, 6.0, 6.0 Hz, 1H), 2.26 (s, 3H), 2.25-2.23 (m, 1H), 2.02 (ddd, J=15.0, 9.0, 9.0 Hz, 1H), 1.92-1.82 (m, 2H), 1.73-1.59 (m, 2H), 1.53-1.45 (m, 2H), 1.26-1.24 (m, 1H), 1.19 (d, J=6.6, 3H), 1.17 (s, 3H), 1.16 (s, 3H), 1.14 (d, J=6.6 Hz, 3H), 1.09-1.04 (m, 19H), 0.97 (s, 9H), 0.88 (s, 3H), 0.81-0.76 (m, 6H), 0.73-0.69 (m, 6H), 0.08 (s, 3H), 0.07 (s, 3H) ppm; ¹³C NMR (151 MHz, C₆D₆) δ=214.4, 170.7, 159.6, 151.1, 145.6, 117.0, 80.1, 79.0, 75.8, 64.3, 54.9, 53.4, 49.9, 48.0, 43.3, 40.2, 37.3, 36.4, 35.2, 32.5, 26.2, 25.5, 23.6, 22.9, 20.1, 18.5, 17.6, 15.6, 15.2, 7.4, 7.3, 6.0, 5.8, 5.1 ppm; HRMS (ESI) calcd for C₄₅H₈₆N₃O₆SSi₃⁺ [M+H]⁺ 880.5540, found 880.5549.

### (1S,3S,7S,10R,11S,12S,16R)-7,11-Dihydroxy-17-(2-hydroxyethyl)-8,8,10,12,16-pentamethyl-3-[(1E)-1-(1,2,5-thiadiazol-3-yl)prop-1-en-2-yl]-4-oxa-17-azabicyclo[14.1.0]heptadecane-5,9-dione (28)

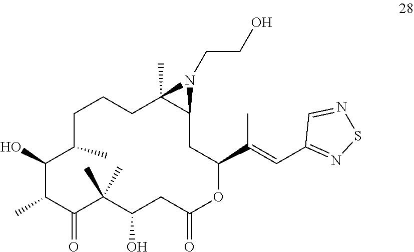

To a stirred solution of protected epothilone 28a (2.8 mg, 3.2 μmol, 1.0 equiv) in tetrahydrofuran (0.9 mL) at 0° C. was added hydrogen fluoride-pyridine complex (70%, 0.10 mL, excess), and the reaction mixture was allowed to slowly warm to 25° C. After 3 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by preparative thin layer chromatography (silica gel, 20% methanol in ethyl acetate) to afford pure epothilone 28 (1.3 mg, 2.4 μmol, 76% yield) as a white amorphous solid. 28: R_{f}=0.29 (silica gel, 15% methanol in ethyl acetate); [α]_{D}²²=−10.9 (c=0.11, CH₂Cl₂); FT-IR (film) vₘₐₓ 3404, 2924, 2851, 1732, 1687, 1463, 1374, 1261, 1147, 1059, 980, 886, 800, 750 cm⁻¹; ¹H NMR (600 MHz, C₆D₆) δ=8.06 (s, 1H), 6.75 (s, 1H), 5.48 (dd, J=4.2, 4.2 Hz, 1H), 4.08 (dd, J=7.2, 4.2 Hz, 1H), 3.88 (dd, J=6.6, 3.6 Hz, 1H), 3.61-3.59 (m, 2H), 3.32 (dq, J=6.6, 6.6 Hz, 1H), 2.47-2.43 (m, 1H), 2.38-2.36 (m, 2H), 2.13 (ddd, J=13.2, 4.8, 4.8 Hz, 1H), 1.99 (s, 3H), 1.86-1.81 (m, 1H), 1.63-1.57 (m, 2H), 1.51-1.44 (m, 1H), 1.43-1.32 (m, 4H), 1.24-1.20 (m, 1H), 1.16 (s, 3H), 1.09 (d, J=6.6 Hz, 3H), 1.03 (d, J=7.2 Hz, 3H), 0.92 (s, 3H), 0.89-0.87 (m, 1H), 0.73 (s, 3H) ppm; ¹³C NMR (151 MHz, C₆D₆) δ=219.3, 170.9, 159.5, 151.3, 143.1, 115.4, 77.0, 76.4, 75.9, 62.3, 55.6, 52.0, 46.9, 45.8, 38.9, 35.1, 33.6, 30.5, 30.2, 28.5, 22.7, 21.8, 18.4, 17.9, 16.4, 16.2, 15.4 ppm; HRMS (ESI) calcd for C₂₇H₄₄N₃O₆S⁺ [M+H]+ 538.2945, found 538.2955.

### Scheme S14. Synthesis of Phosphonate 55

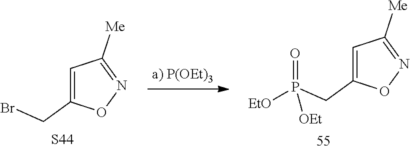

### Diethyl [(3-methyl-1,2-oxazol-5-yl)methyl]phosphonate (55)

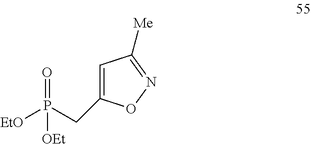 [SMILES: CCOP(=O)(Cc1cc(C)no1)OCC]

To a stirred solution of bromomethyl isoxazole S44 (250 mg, 1.42 mmol, 1.0 equiv) in benzene (4.7 mL) at 25° C. was added triethyl phosphite (1.2 mL, 7.1 mmol, 5.0 equiv). The reaction mixture was heated to 90° C. for 6 h, allowed to cool to 25° C., and concentrated under reduced pressure. The obtained residue was purified by flash column chromatography (silica gel, 0→5% methanol in ethyl acetate) to afford pure phosphonate 55 (77 mg, 0.33 mmol, 23% yield) as a colorless oil. 55: R_{f}=0.37 (silica gel, 5% methanol in ethyl acetate); FT-IR (neat) vₘₐₓ 3480, 3129, 2985, 2935, 2913, 1606, 1481, 1445, 1422, 1395, 1371, 1253, 1163, 1098, 1050, 1022, 1002, 970, 903, 832, 808, 743, 670 cm⁻¹; ¹H NMR (600 MHz, CDCl₃) δ=6.10 (d, J=2.9 Hz, 1H), 4.12 (dq, J=8.2, 7.0 Hz, 4H), 3.30 (d, J=21.3 Hz, 2H), 2.28 (s, 3H), 1.31 (t, J=7.1 Hz, 6H) ppm; ¹³C NMR (151 MHz, CDCl₃) δ=163.5 (d, J=7.1 Hz), 160.4 (d, J=3.1 Hz), 104.3 (d, J=5.4 Hz), 62.9 (d, J=6.6 Hz), 25.7 (d, J=142.8 Hz), 16.5 (d, J=6.0 Hz), 11.6 ppm; HRMS (ESI) calcd for C₉H₁₆NO₄PNa⁺ [M+Na]⁺ 256.0709, found 256.0713.

### (1S,3S,7S,10R,11S,12S,16R)-17-(2-{[tert-Butyl(dimethyl)silyl]oxy}ethyl)-8,8,10,12,16-pentamethyl-3-[(1E)-1-(3-methyl-1,2-oxazol-5-yl)prop-1-en-2-yl]-7,11-bis[(triethylsilyl)oxy]-4-oxa-17-azabicyclol[4.1.0]heptadecane-5,9-dione (29a)

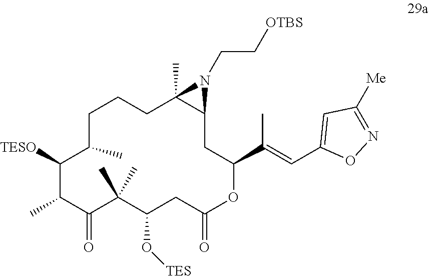

To a stirred solution of phosphonate 55 (73 mg, 0.31 mmol, 43 equiv) in tetrahydrofuran (0.7 mL) at 50° C. was added sodium bis(trimethylsilyl)amide (1.0 M in tetrahydrofuran, 0.29 mL, 0.29 mmol, 41 equiv) dropwise. After 20 min, an aliquot of the reaction mixture (0.25 mL, ca. 79 μmol, 11 equiv 55) was transferred to a stirred solution of methyl ketone 82 (5.7 mg, 7.2 μmol, 1.0 equiv) in tetrahydrofuran (0.25 mL) at −78° C., and the reaction mixture was allowed to slowly warm to 60° C. over 1 h. Then the reaction mixture was quenched with saturated aqueous ammonium chloride solution (0.8 mL), and allowed to warm to 25° C. The two phases were separated, and the aqueous layer was extracted with ethyl acetate (3×1 mL). The combined organic layers were dried with anhydrous sodium sulfate and concentrated under reduced pressure. The obtained residue was purified by flash column chromatography (silica gel, 15→50% ethyl acetate in hexanes), and further purified by preparative thin layer chromatography (silica gel, 30% ethyl acetate in hexanes) to afford pure protected epothilone 29a [3.6 mg, 4.1 μmol, 57%, (E):(Z) ca. 75:25] as a colorless oil. 29a: R_{f}=0.33 (silica gel, 20% ethyl acetate in hexanes); [α]2=1.0 (c=0.30, CH₂Cl₂); FT-IR (neat) vₘₐₓ 2953, 2925, 2875, 2855, 1746, 1696, 1513, 1462, 1414, 1378, 1249, 1157, 1104, 1040, 1019, 985, 836, 780, 739, 675 cm⁻¹; ¹H NMR (600 MHz, C₆D₆) δ=6.50 (s, 1H), 5.58 (s, 1H), 5.30 (dd, J=8.1, 2.9 Hz, 1H), 4.19-4.16 (m, 2H), 3.89-3.84 (m, 1H), 3.82-3.77 (m, 1H), 3.00 (dq, J=6.9, 6.9 Hz, 1H), 2.77-2.65 (m, 2H), 2.56-2.52 (m, 2H), 2.49-2.42 (m, 1H), 2.23-2.18 (m, 1H), 2.00 (s, 3H), 1.93 (s, 3H), 1.89-1.83 (m, 2H), 1.71-1.63 (m, 2H), 1.58-1.47 (m, 3H), 1.44-1.34 (m, 1H), 1.34-1.21 (m, 3H), 1.19 (d, J=6.9 Hz, 3H), 1.16-1.11 (m, 9H), 1.09-1.03 (m, 18H), 0.99 (s, 9H), 0.81-0.76 (m, 6H), 0.73-0.69 (m, 6H), 0.095 (s, 6H) ppm; ¹³C NMR (151 MHz, C₆D₆) δ=214.5, 173.9, 167.8, 159.4, 143.7, 113.2, 104.2, 80.2, 78.5, 75.9, 63.4, 54.5, 53.4, 50.2, 48.2, 43.7, 40.1, 37.3, 35.8, 34.7, 32.7, 26.2, 25.5, 23.6, 20.2, 20.1, 18.6, 17.7, 15.7, 15.3, 11.2, 7.5, 7.4, 6.0, 5.8, 5.1 ppm (¹H and ¹³C NMR were recorded as a mixture); HRMS (ESI) calcd for C₄₇H₈₉N₂O₇Si₃⁺ [M+H]⁺ 877.5972, found 877.5979.

### (1S,3S,7S,10R,11S,12S,16R)-7,11-Dihydroxy-17-(2-hydroxyethyl)-8,8,10,12,16-pentamethyl-3-[(1E)-1-(3-methyl-1,2-oxazol-5-yl)prop-1-en-2-yl]-4-oxa-17-azabicyclo[14.1.0]heptadecane-5,9-dione (29)

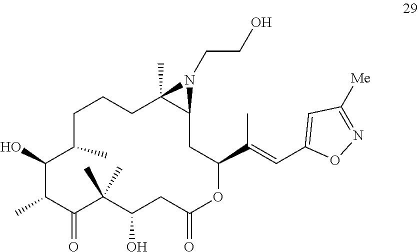

To a stirred solution of protected epothilone 29a (3.6 mg, 4.1 μmol, 1.0 equiv) in tetrahydrofuran (0.7 mL) at 0° C. was added hydrogen fluoride-pyridine complex (70% HF, 0.10 mL, 3.9 mmol, excess), and the reaction mixture was allowed to slowly warm to 25° C. After 3 h, the reaction mixture was quenched with saturated aqueous sodium bicarbonate solution (10 mL). The two phases were separated, and the aqueous layer was extracted with ethyl acetate (3×5 mL). The combined organic layers were dried with anhydrous sodium sulfate and concentrated under reduced pressure. The obtained residue was purified by preparative thin layer chromatography (silica gel, 20% methanol in ethyl acetate) to afford pure epothilone 29 [2.0 mg, 3.7 μmol, 90%, (E):(Z) ca. 75:25] as a white amorphous solid. Geometrically pure (E)-29 was obtained by repeated preparative thin layer chromatography (silica gel, 20% methanol in ethyl acetate, ran 4 times). 29: R_{f}=0.25 (silica gel, 20% methanol in ethyl acetate); [α]2=−13.0 (c=0.10, CH₂Cl₂); FT-IR (film) vₘₐₓ 3361, 2956, 2922, 2852, 1735, 1655, 1632, 1467, 1411, 1377, 1261, 1055, 1033, 1012, 807, 706, 668 cm⁻¹; ¹H NMR (600 MHz, CD₂Cl₂) δ=6.46 (s, 1H), 6.08 (s, 1H), 5.50 (dd, J=3.8, 3.8 Hz, 1H), 3.97 (dd, J=9.8, 2.0 Hz, 1H), 3.74 (dd, J=6.5, 3.6 Hz, 1H), 3.70-3.62 (m, 2H), 3.29 (dq, J=6.8, 6.8 Hz, 1H), 2.72-2.68 (m, 1H), 2.55-2.48 (m, 2H), 2.52 (dd, J=13.7, 9.8 Hz, 1H), 2.45 (dd, J=13.7, 2.2 Hz, 1H), 2.28 (s, 3H), 2.16-2.13 (m, 1H), 2.13-2.02 (m, 2H), 2.06 (s, 3H), 2.05-2.02 (m, 1H), 1.70-1.48 (m, 5H), 1.46-1.36 (m, 2H), 1.35 (s, 3H), 1.16 (s, 3H), 1.12 (d, J=7.0 Hz, 3H), 1.03 (s, 3H), 0.96 (d, J=7.0 Hz, 3H) ppm; ¹³C NMR (151 MHz, CD₂Cl₂) δ=220.6, 171.4, 167.8, 160.2, 141.8, 112.2, 104.4, 76.9, 76.6, 75.8, 62.4, 55.5, 52.4, 47.7, 45.5, 39.3, 38.0, 35.3, 34.0, 31.2, 29.0, 22.3, 21.9, 19.2, 17.7, 16.6, 16.3, 15.0, 11.5 ppm; HRMS (ESI) calcd for C₂₉H₄₆N₂O₇Na⁺ [M+Na]⁺ 557.3197, found 557.3204.

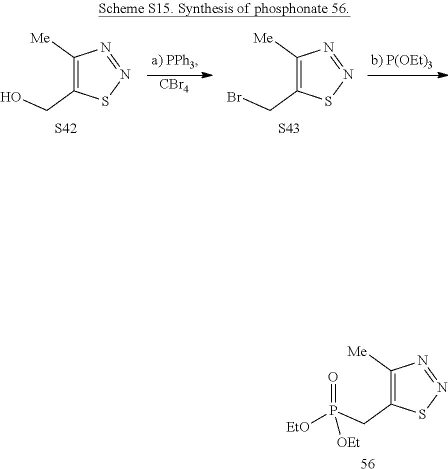

### 5-(Bromomethyl)-4-methyl-1,2,3-thiadiazole (S43)

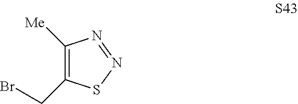

To a stirred solution of hydroxymethyl thiadiazole S42 (0.600 g, 4.61 mmol, 1.0 equiv) in dichloromethane (46 mL) at 25° C. was added triphenylphosphine (1.81 g, 6.92 mmol, 1.5 equiv), followed by carbon tetrabromide (2.29 g, 6.92 mmol, 1.5 equiv). After 15 min, the reaction mixture was quenched with saturated aqueous sodium bicarbonate solution (10 mL) and a saturated aqueous solution of sodium thiosulfate (10 mL). The two phases were separated, and the aqueous layer was extracted with dichloromethane (3×8 mL). The combined organic layers were dried with anhydrous sodium sulfate and concentrated under reduced pressure. The obtained residue was purified by flash column chromatography (silica gel, 5→15% ethyl acetate in hexanes) to afford pure bromomethyl thiadiazole S43 (0.870 g, 4.51 mmol, 98% yield) as a colorless oil. S43: R_{f}=0.27 (silica gel, 15% ethyl acetate in hexanes); FT-IR (neat) vₘₐₓ 3025, 2975, 2928, 2862, 1514, 1440, 1382, 1320, 1233, 1217, 1182, 1033, 997, 969, 864, 799, 689, 666 cm⁻¹; ¹H NMR (600 MHz, CDCl₃) δ=4.69 (s, 2H), 2.70 (s, 3H) ppm; ¹³C NMR (151 MHz, CDCl₃) δ=157.7, 146.9, 18.1, 12.5 ppm; HRMS (ESI) calcd for C₄H₆BrN₂S⁺ [M+H]⁺ 192.9430, found 192.9428.

### Diethyl [(4-methyl-1,2,3-thiadiazol-5-yl)methyl]phosphonate (56)

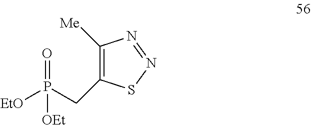 [SMILES: CCOP(=O)(Cc1snnc1C)OCC]

To a stirred solution of bromomethyl thiadiazole S43 (0.870 g, 4.51 mmol, 1.0 equiv) in benzene (9.0 mL) at 25° C. was added triethyl phosphite (3.9 mL, 23 mmol, 5.0 equiv). The reaction mixture was heated to 95° C. for 16 h, allowed to cool to 25° C., and concentrated under reduced pressure. The obtained residue was purified by flash column chromatography (silica gel, 50→100% ethyl acetate in hexanes) to afford pure phosphonate 56 (1.02 g, 4.08 mmol, 90% yield) as a colorless oil. 56: R_{f}=0.29 (silica gel, ethyl acetate); FT-IR (neat) vₘₐₓ 3484, 2983, 2933, 2911, 1649, 1513, 1479, 1444, 1392, 1369, 1247, 1163, 1097, 1049, 1023, 973, 807, 724 cm⁻¹; ¹H NMR (600 MHz, CDCl₃) δ=4.10 (dq, J=8.3, 7.1 Hz, 4H), 3.40 (d, J=21.3 Hz, 2H), 2.69 (d, J=2.3 Hz, 3H), 1.30 (t, J=7.1 Hz, 6H) ppm; ¹³C NMR (151 MHz, CDCl₃) δ=157.9 (d, J=8.7 Hz), 140.8 (d, J=10.4 Hz), 63.1 (d, J=6.8 Hz), 24.0 (d, J=145.0 Hz), 16.5 (d, J=5.9 Hz), 12.6 ppm; HRMS (ESI) calcd for C₈H₁₆N₂O₃PS⁺ [M+H]⁺ 251.0614, found 251.0612.

### (1S,3S,7S,10R,11S,12S,16R)-17-(2-{[tert-Butyl(dimethyl)silyl]oxy}ethyl)-8,8,10,12,16-pentamethyl-3-[(1E)-1-(4-methyl-1,2,3 thiadiazol-5-yl) prop-1-en-2-yl]-7,11-bis [(triethylsilyl) oxy]-4-oxa-17-azabicyclo[14.1.0]heptadecane-5,9-dione (30a)

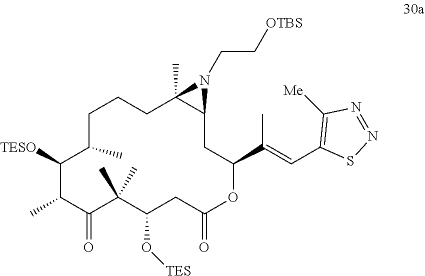

To a stirred solution of phosphonate 56 (133 mg, 0.530 mmol, 98 equiv) in tetrahydrofuran (1.3 mL) at 30° C. was added sodium bis(trimethylsilyl)amide (1.0 M in tetrahydrofuran, 0.50 mL, 0.50 mmol, 93 equiv) dropwise. After 20 min, an aliquot of the reaction mixture (0.28 mL, ca. 81 μmot, 15 equiv 56) was transferred to a stirred solution of methyl ketone 82 (4.3 mg, 5.4 μmot, 1.0 equiv) in tetrahydrofuran (0.25 mL) at −78° C., and was allowed to slowly warm to 15° C. over 2 h. Then the reaction mixture was quenched with saturated aqueous ammonium chloride solution (0.5 mL), and diluted with water (5 mL) and ethyl acetate (5 mL). The two phases were separated, and the aqueous layer was extracted with ethyl acetate (3×3 mL). The combined organic layers were dried with anhydrous sodium sulfate and concentrated under reduced pressure. The obtained residue was purified by flash column chromatography (silica gel, 15→50% ethyl acetate in hexanes), and further purified by preparative thin layer chromatography (silica gel, 20% ethyl acetate in hexanes) to afford pure protected epothilone 30a (3.2 mg, 3.6 μmot, 33% yield) as a colorless oil. 30a: R_{f}=0.34 (silica gel, 20% ethyl acetate in hexanes); [c]2=_4.7 (c=0.30, CH₂Cl₂); FT-IR (neat) vₘₐₓ 2955, 2927, 2876, 2855, 1746, 1697, 1461, 1380, 1246, 1157, 1110, 1010, 985, 837, 777, 741 cm⁻¹; ¹H NMR (600 MHz, C₆D₆) δ=6.55 (s, 1H), 5.43 (dd, J=7.3, 4.6 Hz, 1H), 4.25 (dd, J=7.7, 3.8 Hz, 1H), 4.12 (d, J=8.5 Hz, 1H), 3.85-3.81 (m, 1H), 3.79-3.76 (m, 1H), 3.01 (dq, J=8.4, 7.0 Hz, 1H), 2.75-2.68 (m, 2H), 2.58 (dd, J=16.1, 4.0 Hz, 1H), 2.42 (ddd, J=12.5, 6.5, 6.5 Hz, 1H), 2.36 (s, 3H), 2.17 (dt, J=15.1, 3.7 Hz, 1H), 2.03-1.97 (m, 1H), 1.92-1.81 (m, 2H), 1.71 (s, 3H), 1.67-1.59 (m, 2H), 1.53-1.19 (m, 5H), 1.17 (d, J=6.9 Hz, 3H), 1.16 (s, 3H), 1.155 (s, 3H), 1.12 (d, J=6.9 Hz, 3H), 1.07-1.03 (m, 18H), 0.96 (s, 9H), 0.95 (s, 3H), 0.77-0.73 (m, 6H), 0.71-0.67 (m, 6H), 0.070 (s, 3H), 0.068 (s, 3H) ppm; ¹³C NMR (151 MHz, C₆D₆) δ=214.6, 170.6, 157.1, 144.4, 144.3, 115.4, 80.0, 78.9, 75.7, 64.3, 55.0, 53.5, 49.6, 47.9, 43.4, 40.5, 37.4, 36.5, 35.0, 32.7, 26.2, 25.6, 23.8, 22.3, 20.0, 18.6, 17.6, 15.7, 14.9, 12.6, 7.4, 7.3, 5.9, 5.8, 5.1 ppm; HRMS (ESI) calcd for C₄₆H₈₈N₃O₆SSi₃⁺ [M+H]⁺ 894.5696, found 894.5713.

### (1S,3S,7S,10R,11S,12S,16R)-7,11-Dihydroxy-17-(2-hydroxyethyl)-8,8,10,12,16-pentamethyl-3-[(1E)-1-(4-methyl-1,2,3-thiadiazol-5-yl)prop-1-en-2-yl]-4-oxa-17-azabicyclo[14.1.0]heptadecane-5,9-dione (30)

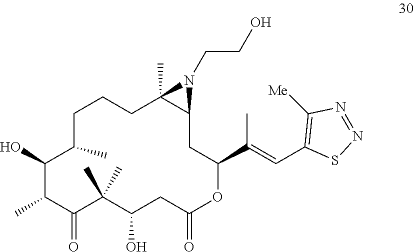

To a stirred solution of protected epothilone 30a (3.2 mg, 3.6 μmol, 1.0 equiv) in tetrahydrofuran (0.6 mL) at 0° C. was added hydrogen fluoride-pyridine complex (70% HF, 0.10 mL, 3.9 mmol, excess), and the reaction mixture was allowed to slowly warm to 25° C. After 3 h, the reaction mixture was quenched with saturated aqueous sodium bicarbonate solution (10 mL), and diluted with ethyl acetate (5 mL). The two phases were separated, and the aqueous layer was extracted with ethyl acetate (3×5 mL). The combined organic layers were dried with anhydrous sodium sulfate and concentrated under reduced pressure. The obtained residue was purified by preparative thin layer chromatography (silica gel, 20% methanol in ethyl acetate) to afford pure epothilone 30 (1.9 mg, 3.4 μmol, 95% yield) as a white amorphous solid. 30: R_{f}=0.21 (silica gel, 10% methanol in ethyl acetate); [α]_{D}²²=−27.0 (c=0.20, CH₂Cl₂); FT-IR (film) voax 3402, 2927, 1738, 1686, 1555, 1465, 1380, 1334, 1245, 1204, 1148, 1038, 1009, 980, 874, 847, 833, 816, 797, 715, 692, 662 cm⁻¹; ¹H NMR (600 MHz, CD₂Cl₂) δ=6.71 (s, 1H), 5.55 (dd, J=4.3, 4.3 Hz, 1H), 4.01 (dd, J=9.7, 2.5 Hz, 1H), 3.75 (dd, J=6.0, 4.0 Hz, 1H), 3.69-3.67 (m, 2H), 3.28 (dq, J=6.5, 6.5 Hz, 1H), 2.70 (s, 3H), 2.66-2.63 (m, 2H), 2.53 (dd, J=14.0, 9.7 Hz, 1H), 2.47 (dd, J=14.0, 2.6 Hz, 1H), 2.10-1.99 (m, 3H), 1.95 (s, 3H), 1.70-1.63 (m, 1H), 1.57-1.37 (m, 5H), 1.35 (s, 3H), 1.26 (br s, 1H), 1.19 (s, 3H), 1.13 (d, J=7.0 Hz, 3H), 1.05 (s, 3H), 0.97 (d, J=7.0 Hz, 3H) ppm; ¹³C NMR (151 MHz, CD₂Cl₂) δ=220.4, 171.3, 157.5, 145.0, 143.2, 114.1, 77.5, 75.9, 75.2, 62.1, 55.2, 52.6, 48.5, 45.0, 39.5, 35.5, 34.1, 31.6, 30.1, 29.5, 21.9, 21.7, 20.0, 17.7, 16.6, 16.5, 14.6, 13.0 ppm; HRMS (ESI) calcd for C₂₈H₄₆N₃O₆S⁺ [M+H]⁺ 552.3102, found 552.3114.

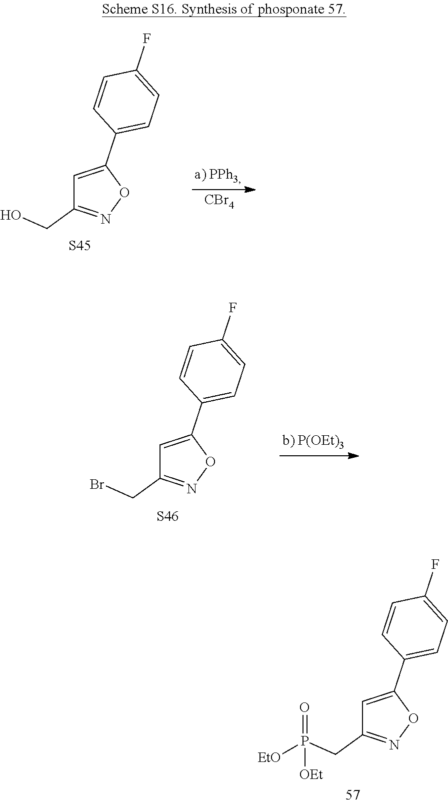

### 3-(Bromomethyl)-5-(4-fluorophenyl)-1,2-oxazole (S46)

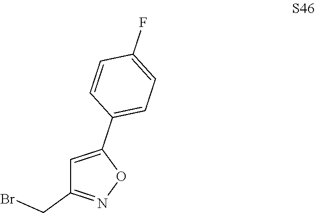 [SMILES: Fc1ccc(-c2cc(CBr)no2)cc1]

To a stirred solution of hydroxymethyl isoxazole S45 (630 mg, 3.26 mmol, 1.0 equiv) in dichloromethane (26 mL) at 0° C. was added triphenylphosphine (1.63 g, 4.90 mmol, 1.5 equiv), followed by carbon tetrabromide (2.04 g, 4.90 mmol, 1.5 equiv). After 25 min, the reaction mixture was quenched with saturated aqueous sodium bicarbonate solution (10 mL) and a saturated aqueous solution of sodium thiosulfate (10 mL), and allowed to warm to 25° C. The two phases were separated, and the aqueous layer was extracted with dichloromethane (3×5 mL). The combined organic layers were dried with anhydrous sodium sulfate and concentrated under reduced pressure. The obtained residue was purified by flash column chromatography (silica gel, 5→10% ethyl acetate in hexanes) to afford pure bromomethyl isoxazole S46 (810 mg, 3.16 mmol, 97% yield) as a white amorphous solid. S46: R_{f}=0.33 (silica gel, 10% ethyl acetate in hexanes); FT-IR (film) vₘₐₓ 3133, 2974, 1617, 1510, 1468, 1447, 1305, 1236, 1159, 1123, 1100, 1047, 1033, 1013, 949, 925, 840, 813, 796, 725, 696, 665 cm⁻¹; ¹H NMR (600 MHz, CDCl₃) δ=7.80-7.75 (m, 2H), 7.20-7.14 (m, 2H), 6.57 (s, 1H), 4.46 (s, 2H) ppm; ¹³C NMR (151 MHz, CDCl₃) δ=170.0, 164.1 (d, J=251.7 Hz), 161.7, 128.1 (d, J=8.5 Hz), 123.6 (d, J=8.5 Hz), 116.5 (d, J=22.2 Hz), 99.4, 20.8 ppm; HRMS (ESI) calcd for C₁₀H₈BrFNO⁺ [M+H]+ 255.9768, found 255.9776.

### Diethyl {[5-(4-fluorophenyl)-1,2-oxazol-3-yl]methyl}phosphonate (57)

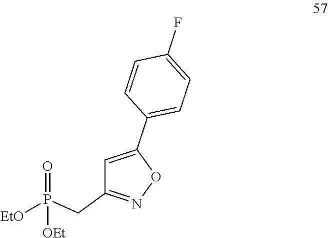 [SMILES: CCOP(=O)(Cc1cc(-c2ccc(F)cc2)on1)OCC]

To a stirred solution of bromomethyl isoxazole S46 (800 mg, 3.1 mmol, 1.0 equiv) in benzene (10 mL) at 25° C. was added triethyl phosphite (2.7 mL, 16 mmol, 5.0 equiv). The reaction mixture was heated to 95° C. for 18 h, allowed to cool to 25° C., and concentrated under reduced pressure. The obtained residue was purified by flash column chromatography (silica gel, 50→100% ethyl acetate in hexanes 5% methanol in ethyl acetate) to afford pure phosphonate 57 (880 mg, 4.0 mmol, 90% yield) as a colorless oil. 57: R_{f}=0.39 (silica gel, ethyl acetate); FT-IR (neat) vₘₐₓ 3476, 3126, 2985, 2911, 1617, 1602, 1511, 1463, 1433, 1394, 1369, 1236, 1161, 1100, 1051, 1025, 969, 949, 842, 814, 723, 676 cm⁻¹; ¹H NMR (600 MHz, CDCl₃) δ=7.79-7.74 (m, 2H), 7.19-7.12 (m, 2H), 6.60 (d, J=1.0 Hz, 1H), 4.14 (dq, J=8.5, 7.1 Hz, 4H), 3.28 (d, J=21.0 Hz, 2H), 1.32 (t, J=7.1 Hz, 6H) ppm; ¹³C NMR (151 MHz, CDCl₃) δ=169.5, 163.9 (d, J=251.4 Hz), 156.7 (d, J=7.0 Hz), 128.1 (d, J=8.6 Hz), 123.8 (d, J=3.4 Hz), 116.3 (d, J=22.1 Hz), 100.2, 62.8 (d, J=6.6 Hz), 24.9 (d, J=142.1 Hz), 16.5 (d, J=5.9 Hz) ppm; HRMS (ESI) calcd for C₁₄H₁₇FNO₄PNa⁺ [M+Na]⁺ 336.0771, found 336.0775.

### (1S,3S,7S,10R,11S,12S,16R)-17-(2-{[tert-Butyl(dimethyl)silyl]oxy}ethyl)-3-{(1E)-1-[5-(4-fluorophenyl)-1,2-oxazol-3-yl]prop-1-en-2-yl}-8,8,10,12,16-pentamethyl-7,11-bis[(triethyl-silyl)oxy]-4-oxa-17-azabicyclo[14.1.0]heptadecane-5,9-dione (31a)

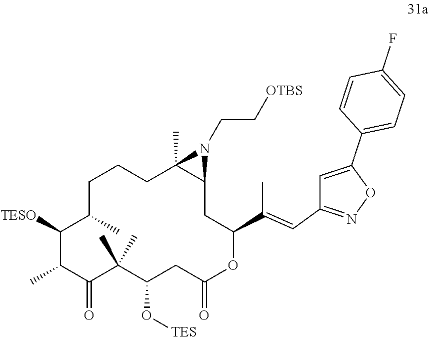

To a stirred solution of phosphonate 57 (312 mg, 1.00 mmol, 145 equiv) in tetrahydrofuran (3.0 mL) at 50° C. was added sodium bis(trimethylsilyl)amide (1.0 M in tetrahydrofuran, 0.95 mL, 0.95 mmol, 138 equiv) dropwise. After 20 min, an aliquot of the reaction mixture (0.40 mL, ca. 0.10 mmol, 15 equiv 57) was transferred to a stirred solution of methyl ketone 82 (5.5 mg, 6.9 μmol, 1.0 equiv) in tetrahydrofuran (0.35 mL) at 60° C. After 1 h, the reaction mixture was quenched with saturated aqueous ammonium chloride solution (1.2 mL), and allowed to warm to 25° C. The two phases were separated, and the aqueous layer was extracted with ethyl acetate (3×1 mL). The combined organic layers were dried with anhydrous sodium sulfate and concentrated under reduced pressure. The obtained residue was purified by flash column chromatography (silica gel, 20→40% ethyl acetate in hexanes), and further purified by preparative thin layer chromatography (silica gel, 20% ethyl acetate in hexanes) to afford pure protected epothilone 31a [2.3 mg, 2.4 μmol, 35%, (E):(Z) ca. 86:14, plus 34% recovered methyl ketone 82] as a colorless oil. 31a: R_{f}=0.44 (silica gel, 20% ethyl acetate in hexanes); [α]2=1.5 (c=0.20, CH₂Cl₂); FT-IR (neat) vₘₐₓ 2954, 2929, 2877, 2857, 1745, 1697, 1615, 1508, 1461, 1380, 1237, 1158, 1110, 1018, 985, 838, 812, 779, 739, 680 cm⁻¹; ¹H NMR (600 MHz, C₆D₆) δ=7.31-7.28 (m, 2H), 6.66-6.64 (m, 3H), 6.02 (s, 1H), 5.44 (d, J=7.9, 2.8 Hz, 1H), 4.23 (dd, J=8.5, 2.6 Hz, 1H), 4.18 (d, J=8.9 Hz, 1H), 3.89-3.85 (m, 1H), 3.84-3.79 (m, 1H), 3.03 (dq, J=8.8, 7.0 Hz, 1H), 2.78-2.71 (m, 2H), 2.60 (dd, J=16.2, 3.1 Hz, 1H), 2.50-2.45 (m, 1H), 2.31-2.29 (m, 1H), 2.22 (s, 3H), 2.13-2.01 (m, 1H), 1.91-1.81 (m, 2H), 1.52-1.44 (m, 2H), 1.38-1.24 (m, 4H), 1.20 (d, 6.8 Hz, 3H), 1.18 (s, 3H), 1.17 (s, 3H), 1.14 (d, J=6.8 Hz, 3H), 1.11-1.05 (m, 18H), 1.00 (s, 9H), 0.86 (s, 3H), 0.84-0.79 (m, 6H), 0.74-0.68 (m, 6H), 0.10 (s, 6H) ppm; ¹³C NMR (151 MHz, C₆D₆) δ=214.5, 170.9, 168.6, 163.9 (d, J=250.1 Hz), 160.7, 145.0, 124.2 (d, J=3.3 Hz), 116.2 (d, J=22.0 Hz), 115.0, 100.2, 80.3, 78.8, 76.0, 64.3, 54.9, 53.5, 50.2, 48.2, 43.4, 40.2, 37.3, 36.4, 35.3, 32.4, 26.2, 25.5, 23.6, 23.2, 20.1, 18.6, 17.7, 15.7, 15.6, 7.5, 7.4, 6.0, 5.9, 5.1 ppm (¹H and ¹³C NMR were recorded as a mixture); HRMS (ESI) calcd for C₅₂H₉₀FN₂O₇Si₃⁺ [M+H]⁺ 957.6034, found 957.6050.

### (1S,3S,7S,10R,11S,12S,16R)-3-{(1E)-1-[5-(4-Fluorophenyl)-1,2-oxazol-3-yl]prop-1-en-2-yl}-7,11-dihydroxy-17-(2-hydroxyethyl)-8,8,10,12,16-pentamethyl-4-oxa-17-azabicyclo[14.1.0]heptadecane-5,9-dione (31)

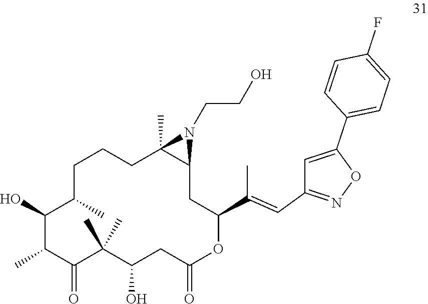

To a stirred solution of protected epothilone 31a (2.3 mg, 2.4 μmol, 1.0 equiv) in tetrahydrofuran (0.5 mL) at 0° C. was added hydrogen fluoride-pyridine complex (70% HF, 0.10 mL, 3.9 mmol, excess), and the reaction mixture was allowed to slowly warm to 25° C. After 3 h, the reaction mixture was quenched with saturated aqueous sodium bicarbonate solution (10 mL), and diluted with ethyl acetate (10 mL). The two phases were separated, and the aqueous layer was extracted with ethyl acetate (3×5 mL). The combined organic layers were dried with anhydrous sodium sulfate and concentrated under reduced pressure. The obtained residue was purified by preparative thin layer chromatography (silica gel, 20% methanol in ethyl acetate) to afford pure epothilone 31 [1.4 mg, 2.3 μmol, 97%, (E):(Z)=86:14] as a white amorphous solid. 31: R_{f}=0.34 (silica gel, 10% methanol in ethyl acetate); [α]_{D}²² 10.0 (c=0.20, CH₂Cl₂); FT-IR (film) vₘₐₓ 3357, 2923, 2852, 1737, 1665, 1633, 1615, 1532, 1507, 1466, 1379, 1260, 1233, 1158, 1055, 1014, 980, 842, 811, 778, 738, 653 cm⁻¹; ¹H NMR (600 MHz, CD₂Cl₂) δ=7.82-7.79 (m, 2H), 7.22-7.18 (m, 2H), 6.56 (s, 1H), 6.48 (s, 1H), 5.52 (dd, J=4.2, 4.2 Hz, 1H), 4.01 (dd, J=9.8, 2.0 Hz, 1H), 3.75 (dd, J=6.2, 3.8 Hz, 1H), 3.72-3.64 (m, 2H), 3.30 (dq, J=6.7, 6.7 Hz, 1H), 2.75-2.71 (m, 1H), 2.62-2.56 (m, 1H), 2.54 (dd, J=13.7, 9.9 Hz, 1H), 2.45 (dd, J=13.7, 2.2 Hz, 1H), 2.13-2.02 (m, 2H), 2.11 (d, J=0.7 Hz, 3H), 1.90-1.83 (m, 1H), 1.72-1.39 (m, 7H), 1.36 (s, 3H), 1.18 (s, 3H), 1.13 (d, J=7.0 Hz, 3H), 1.04 (s, 3H), 0.96 (d, J=7.0 Hz, 3H) ppm; ¹³C NMR (151 MHz, CD₂Cl₂) δ=220.6, 171.4, 168.9, 164.2 (d, J=250.2 Hz), 160.8, 143.0, 128.3 (d, J=8.8 Hz), 124.3 (d, J=3.0 Hz), 116.6 (d, J=22.2 Hz), 113.8, 100.4, 77.1, 76.4, 75.5, 62.3, 55.4, 52.6, 47.9, 45.3, 39.4, 35.4, 34.1, 31.3, 30.1, 29.2, 22.0, 21.9, 19.4, 17.7, 16.6, 16.5, 14.8 ppm (¹H and ¹³C NMR were recorded as a mixture); HRMS (ESI) calcd for C₃₄H₄₈FN₂O₇S⁺ [M+H]+ 615.3440, found 615.3447.

### (1S,3S,7S,10R,11S,12S,16R)-8,8,10,12,16-Pentamethyl-3-[(1E)-1-(4′-methyl-2,2′-bi-1,3-thiazol-4-yl)prop-1-en-2-yl]-7,11-bis[(triethylsilyl)oxy]-17-{[2-(trimethylsilyl)ethoxy]methyl}-4-oxa-17-azabicyclo[14.1.0]heptadecane-5,9-dione (32a)

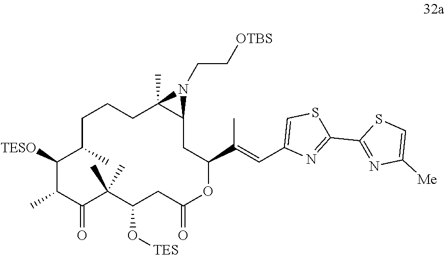

To a stirred solution of phosphonate 49 (40.0 mg, 120 μmol, 18 equiv) in tetrahydrofuran (5.0 mL) at −78° C. was added n-butyllithium (2.5 M in hexanes, 39 μL, 96 μmol, 15 equiv) dropwise. After 10 min, the reaction mixture was transferred to a stirred solution of methyl ketone 87 (5.0 mg, 6.5 μmol, 1.0 equiv) in tetrahydrofuran (0.2 mL) at −78° C. After 1 h, the reaction mixture was allowed to slowly warm to 25° C., and stirred for an additional 3 h. Then the reaction mixture was quenched with saturated aqueous ammonium chloride solution (3 mL), and the two phases were separated. The aqueous layer was extracted with ethyl acetate (3×10 mL), and the combined organic layers were dried with anhydrous sodium sulfate and concentrated under reduced pressure. The obtained residue was purified by preparative thin layer chromatography (silica gel, 30% ethyl acetate in hexanes) to afford pure protected epothilone 32a (2.1 mg, 2.2 μmol, 34%, plus 50% recovered methyl ketone 87) as a colorless oil. 32a: R_{f}=0.33 (silica gel, 20% ethyl acetate in hexanes); [α]2=2.5 (c=0.10, CH₂Cl₂); FT-IR (neat) vₘₐₓ 2953, 2922, 2876, 2854, 1741, 1695, 1503, 1459, 1412, 1379, 1247, 1198, 1157, 1093, 1069, 1017, 979, 940, 882, 859, 835, 803, 783, 732, 668 cm⁻¹; NMR (600 MHz, C₆D₆) δ=6.72 (s, 1H), 6.53 (s, 1H), 6.19 (s, 1H), 5.47 (dd, J=7.8, 4.2 Hz, 1H), 4.30 (dd, J=8.4, 3.6 Hz, 1H), 4.15 (d, J=9.0 Hz, 1H), 4.13 (d, J=9.0 Hz, 1H), 4.09 (d, J=9.0 Hz, 1H), 3.90 (ddd, J=9.0, 7.8, 7.8 Hz, 1H), 3.79 (ddd, J=9.0, 7.8, 7.8 Hz, 1H), 3.05 (dq, J=8.4, 7.2 Hz, 1H), 2.70 (dd, J=10.2, 8.4 Hz, 1H), 2.60 (dd, J=10.2, 4.2 Hz, 1H), 2.29 (s, 3H), 2.23 (ddd, J=15.0, 4.2, 4.2 Hz, 1H), 2.18 (s, 3H), 2.14-2.09 (m, 1H), 1.92-1.84 (m, 2H), 1.75-1.68 (m, 1H), 1.67-1.59 (m, 1H), 1.55-1.45 (m, 2H), 1.42-1.40 (m, 1H), 1.24-1.20 (m, 1H), 1.18 (s, 3H), 1.175 (s, 3H), 1.17 (d, J=7.2 Hz, 3H), 1.15 (d, J=6.6 Hz, 3H), 1.09-1.06 (m, 18H), 1.07-1.00 (m, 2H), 0.91 (s, 3H), 0.81-0.76 (m, 6H), 0.74-0.70 (m, 6H), 0.05 (s, 9H) ppm; ¹³C NMR (151 MHz, C₆D₆) δ=214.6, 170.6, 160.93, 160.88, 154.5, 154.4, 139.6, 120.5, 119.0, 115.8, 84.2, 80.0, 79.3, 75.6, 65.9, 53.5, 47.9, 45.8, 44.2, 40.5, 37.5, 36.3, 34.7, 32.4, 25.4, 23.5, 22.6, 20.1, 18.4, 17.6, 16.9, 15.8, 14.8, 7.4, 7.3, 5.9, 5.8, 1.1 ppm; HRMS (ESI) calcd for C₄₈H₈₆N₃O₆S₂Si₃⁺ [M+H]⁺ 948.5260, found 948.5284.

### (1S,3S,7S,10R,11S,12S,16R)-7,11-Dihydroxy-8,8,10,12,16-pentamethyl-3-[(1E)-1-(4′-methyl-2,2′-bi-1,3-thiazol-4-yl)prop-1-en-2-yl]-4-oxa-17-azabicyclo[14.1.0]heptadecane-5,9-dione (32)

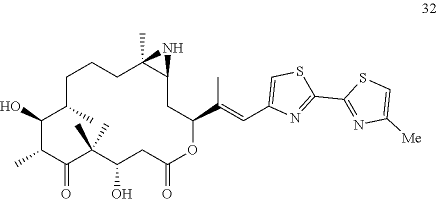

To a stirred solution of protected epothilone 32a (1.9 mg, 2.0 μmol, 1.0 equiv) in dichloromethane (0.4 mL) at 0° C. was added trifluoroacetic acid (0.10 mL, 1.3 mmol, excess), and the reaction mixture was allowed to slowly warm to 25° C. After 2 h, the solvent was removed under reduced pressure, and the obtained residue was purified by preparative thin layer chromatography (silica gel, 25% methanol in ethyl acetate) to afford pure epothilone 32 (1.0 mg, 1.7 μmol, 85% yield) as a white amorphous solid. 32: R_{f}=0.37 (silica gel, 20% methanol in ethyl acetate); [α]_{D}²²=−17.0 (c=0.10, CH₂Cl₂); FT-IR (film) vₘₐₓ 3353, 3102, 2955, 2923, 2853, 1734, 1686, 1501, 1463, 1408, 1377, 1293, 1259, 1174, 1146, 1082, 1039, 1009, 980, 883, 799, 736, 670 cm⁻¹; ¹H NMR (600 MHz, CD₂Cl₂) δ=7.26 (s, 1H), 7.03 (s, 1H), 6.66 (s, 1H), 5.58 (dd, J=3.6, 3.6 Hz, 1H), 4.10-4.06 (m, 2H), 3.78 (dd, J=6.0, 3.6 Hz, 1H), 3.38-3.36 (m, 1H), 3.34 (dq, J=6.6, 6.6 Hz, 1H), 2.57 (dd, J=12.6, 4.8 Hz, 1H), 2.49 (s, 3H), 2.44 (dd, J=12.6, 3.0 Hz, 1H), 2.17 (s, 3H), 2.00-1.96 (m, 1H), 1.89-1.84 (m, 1H), 1.79-1.74 (m, 2H), 1.64-1.50 (m, 5H), 1.42 (s, 3H), 1.23 (s, 3H), 1.10 (d, J=7.2 Hz, 3H), 1.01 (s, 3H), 0.95 (d, J=6.6 Hz, 3H) ppm; ¹³C NMR (151 MHz, CD₂Cl₂) δ=220.6, 171.2, 160.8, 160.7, 154.8, 154.5, 118.6, 118.2, 116.2, 76.3, 76.2, 75.8, 60.6, 52.5, 45.0, 39.0, 38.6, 38.3, 35.2, 31.0, 30.1, 28.7, 25.8, 22.7, 22.4, 18.7, 17.6, 17.2, 16.5, 14.9 ppm; HRMS (ESI) calcd for C₃₀H₄₄N₃O₅S₂⁺ [M+H]⁺ 590.2717, found 590.2722.

### (1S,3S,7S,10R,11S,12S,16R)-8,8,10,12,16-Pentamethyl-3-{(1E)-1-[1-(1H-pyrazol-1-ylmethyl)-1H-pyrazol-3-yl]prop-1-en-2-yl}-7,11-bis[(triethylsilyl)oxy]-17-{[2-(trimethylsilyl)ethoxy]methyl}-4-oxa-17-azabicyclo[14.1.0]heptadecane-5,9-dione (101)

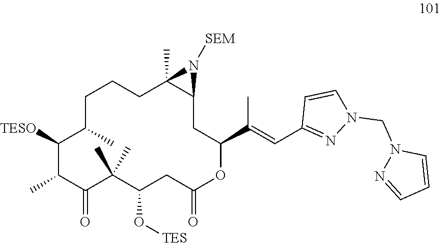

To a stirred solution of phosphonate 52 (68.0 mg, 0.228 mmol, 15 equiv) in tetrahydrofuran (0.7 mL) at −78° C. was added of n-butyllithium (2.5 M in hexanes, 82 μL 0.21 mmol, 14 equiv) dropwise. After 10 min, the reaction mixture was added to a stirred solution of methyl ketone 87 (11.5 mg, 14.9 μmol, 1.0 equiv) in tetrahydrofuran (0.5 mL) at −78° C. After 30 min, the reaction mixture was quenched with saturated aqueous ammonium chloride solution (3 mL), and the two phases were separated. The aqueous layer was extracted with ethyl acetate (3×10 mL), and the combined organic layers were dried with anhydrous sodium sulfate and concentrated under reduced pressure to afford crude phosphonate adduct 99 {confirmed by HRMS (ESI): calcd for C₅₂H₉₉N₅O₁₀PSi₃⁺ [M+H]⁺ 1068.6432, found 1068.6462}.

The obtained crude residue was dissolved in tetrahydrofuran (1.0 mL) with stirring, and cooled to 20° C. Potassium tert-butoxide (5.0 mg, 45 μmol, 3.0 equiv) was added, and after 5 min the reaction mixture was quenched with water (3 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, 45% ethyl acetate in hexanes) to afford pure protected epothilone 101 (7.2 mg, 7.9 nmol, 53% yield overall, plus 28% recovered methyl ketone 87) as a colorless oil. 101: Rₜ=0.32 (silica gel, 40% ethyl acetate in hexanes); [α]_{D}²²=−2.8 (c=0.25, CH₂Cl₂); FT-IR (neat) vₘₐₓ 2953, 2877, 1742, 1696, 1518, 1459, 1413, 1380, 1305, 1280, 1247, 1020, 1181, 1158, 1089, 1069, 1050, 1019, 985, 940, 917, 960, 836, 740 cm⁻¹; ¹H NMR (600 MHz, C₆D₆) δ=7.42 (d, J=1.8 Hz, 1H), 7.13 (d, J=2.4 Hz, 1H), 7.10 (d, J=1.8 Hz, 1H), 6.74 (s, 1H), 6.05 (d, J=2.4 Hz, 1H), 5.89 (dd, J=2.4, 1.8 Hz, 1H), 5.57 (s, 2H), 5.46 (dd, J=8.4, 4.2 Hz, 1H), 4.25 (dd, J=8.4, 3.6 Hz, 1H), 4.15 (d, J=8.4 Hz, 1H), 4.10 (d, J=8.4 Hz, 1H), 4.07 (d, J=8.4 Hz, 1H), 3.90 (ddd, J=9.0, 7.2, 7.2 Hz, 1H), 3.77 (ddd, J=9.0, 7.2, 7.2 Hz, 1H), 3.03 (dq, J=7.8, 7.2 Hz, 1H), 2.68 (dd, J=16.2, 8.4 Hz, 1H), 2.57 (dd, J=16.2, 3.6 Hz, 1H), 2.18 (ddd, J=15.6, 3.6, 3.6 Hz, 1H), 2.16 (s, 3H), 2.07 (ddd, J=15.6, 15.6, 9.0 Hz, 1H), 1.90-1.82 (m, 2H), 1.74-1.68 (m, 1H), 1.65-1.57 (m, 1H), 1.53-1.43 (m, 2H), 1.36 (dd, J=9.0, 2.4 Hz, 1H), 1.20-1.12 (m, 2H), 1.17 (d, J=7.2 Hz, 3H), 1.16 (s, 3H), 1.15 (s, 3H), 1.13 (d, J=6.6 Hz, 3H), 1.08-1.05 (m, 18H), 1.04-1.02 (m, 1H), 0.86 (s, 3H), 0.80-0.76 (m, 6H), 0.74-0.70 (m, 6H), 0.05 (s, 9H) ppm; ¹³C NMR (151 MHz, C₆D₆) δ=214.6, 170.5, 150.5, 140.6, 138.1, 129.9, 129.3, 119.6, 107.8, 107.0, 84.2, 80.0, 79.2, 75.7, 65.8, 65.1, 53.5, 47.9, 46.9, 44.2, 40.4, 37.5, 36.2, 34.8, 32.3, 25.4, 23.5, 22.7, 20.1, 18.4, 17.5, 15.8, 14.8, 7.4, 7.3, 5.9, 5.8, 1.1 ppm; HRMS (ESI) calcd for C₄₈H₈₈N₅O₆Si₃⁺ [M+H]⁺ 914.6037, found 914.6041.

### (1S,3S,7S,10R,11S,12S,16R)-7,11-Dihydroxy-8,8,10,12,16-pentamethyl-3-{(1E)-1-[1-(1H-pyrazol-1-ylmethyl)-1H-pyrazol-3-yl]prop-1-en-2-yl}-4-oxa-17-azabicyclo[14.1.0]heptadecane-5,9-dione (33)

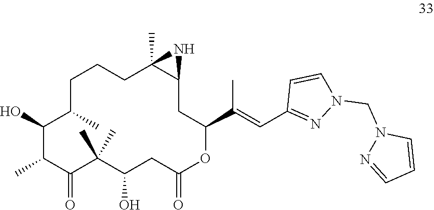

To a stirred solution of protected epothilone 101 (6.2 mg, 6.8 μmol, 1.0 equiv) in dichloromethane (1.2 mL) at 0° C. was added trifluoroacetic acid (0.30 mL, 3.9 mmol, excess), and the reaction mixture was allowed to warm to 25° C. After 2 h, the solvent was removed under reduced pressure, and the obtained residue was purified by preparative thin layer chromatography (silica gel, 20% methanol in ethyl acetate) to afford pure epothilone 33 (2.0 mg, 3.6 μmol, 52% yield) as a white amorphous solid. 33: Rₜ=0.44 (silica gel, 20% methanol in ethyl acetate); [α]_{D}²²=−10.0 (c=0.10, CH₂Cl₂); FT-IR (film) vₘₐₓ 3356, 2956, 2921, 2852, 1731, 1687, 1633, 1511, 1467, 1415, 1384, 1343, 1299, 1280, 1261, 1214, 1175, 1147, 1088, 1050, 1010, 980, 962, 918, 885, 798, 759, 733 cm⁻¹; ¹H NMR (600 MHz, CD₂Cl₂) δ=7.68 (d, J=2.4 Hz, 1H), 7.64 (d, J=2.4 Hz, 1H), 7.52 (d, J=1.8 Hz, 1H), 6.50 (s, 1H), 6.35 (d, J=2.4 Hz, 1H), 6.30 (dd, J=2.4, 1.8 Hz, 1H), 6.25 (s, 2H), 5.55 (dd, J=3.0, 3.0 Hz, 1H), 4.06 (dd, J=10.2, 3.6 Hz, 1H), 3.79 (dd, J=6.6, 3.6 Hz, 1H), 3.32 (dq, J=6.6, 6.6 Hz, 1H), 2.53 (dd, J=12.6, 4.8 Hz, 1H), 2.42 (dd, J=12.6, 3.6 Hz, 1H), 2.01 (s, 3H), 1.94 (ddd, J=14.4, 3.6, 3.6 Hz, 1H), 1.83-1.81 (m, 1H), 1.77-1.74 (m, 1H), 1.71-1.66 (m, 1H), 1.63-1.45 (m, 4H), 1.44-1.41 (m, 1H), 1.40 (s, 3H), 1.36-1.30 (m, 1H), 1.20 (s, 3H), 1.09 (d, J=6.6 Hz, 3H), 1.00 (s, 3H), 0.94 (d, J=6.6 Hz, 3H) ppm; ¹³C NMR (151 MHz, CD₂Cl₂) δ=220.7, 171.2, 150.8, 141.0, 136.6, 130.6, 130.0, 117.2, 107.5, 107.2, 76.3, 76.1, 75.8, 65.7, 52.5, 45.0, 38.4, 38.2, 37.3, 35.2, 30.9, 30.1, 28.6, 25.8, 22.9, 22.4, 18.6, 17.6, 16.3, 14.9 ppm; HRMS (ESI) calcd for C₃₀H₄₆N₅O₅⁺[M+H]⁺ 556.3493, found 556.3488.

### Bis(2-fluoroethyl) {2-hydroxy-1-[1-methyl-5-(methylsulfanyl)-1H-pyrazol-3-yl]-2-[(1S,3S,7S,10R,11S,12S,16R)-8,8,10,12,16-pentamethyl-5,9-dioxo-7,11-bis[(triethylsilyl)oxy]-17-{[2-(trimethylsilyl)ethoxy]methyl}-4-oxa-17-azabicyclo[14.1.0]heptadec-3-yl]propyl}phosphonate (104)

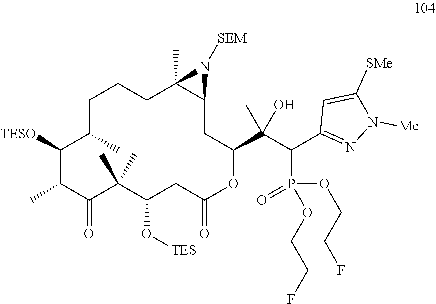

To a stirred solution of phosphonate 62 (49.0 mg, 0.156 mmol, 22 equiv) in tetrahydrofuran (0.5 mL) at −78° C. was added n-butyllithium (2.5 M in hexanes, 50 μL, 0.13 mmol, 18 equiv) dropwise. After 10 min, the reaction mixture was transferred to a stirred solution of methyl ketone 87 (5.5 mg, 7.1 μmol, 1.0 equiv) in tetrahydrofuran (0.4 mL) at −78° C., and stirred for an additional 1 h. Then, the reaction mixture was quenched with saturated aqueous ammonium chloride solution (3 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, ethyl acetate) to afford pure phosphonate adduct 104 (3.2 mg, 3.0 μmol, 28%, dr ca. 3:7, plus 42% recovered methyl ketone 87) as a colorless oil. 104: R_{f}=0.33 (silica gel, 80% ethyl acetate in hexanes); [α]_{D}²²=−15.0 (c=0.20, CH₂Cl₂); FT-IR (neat) vₘₐₓ 3396, 2953, 2877, 1742, 1697, 1498, 1458, 1417, 1382, 1289, 1269, 1246, 1201, 1156, 1082, 1047, 1019, 971, 861, 837, 781, 759, 728 cm⁻¹; ¹H NMR (600 MHz, C₆D₆) δ=6.95 (s, 1H), 5.21-5.18 (m, 1H), 5.17 (dd, J=9.6, 5.4 Hz, 1H), 4.62 (d, J=7.8 Hz, 1H), 4.38-4.29 (m, 1H), 4.23-3.82 (m, 13H), 3.65 (ddd, J=9.6, 9.6, 6.0 Hz, 1H), 3.54 (s, 3H), 3.04 (dq, J=6.6, 6.6 Hz, 1H), 2.86-2.75 (m, 2H), 2.60 (dd, J=15.0, 7.8 Hz, 1H), 2.30 (s, 3H), 1.98 (s, 3H), 1.91-1.79 (m, 3H), 1.50-1.27 (m, 5H), 1.27 (s, 3H), 1.22-1.12 (m, 17H), 1.05-1.00 (m, 12H), 0.94 (s, 3H), 0.93-0.86 (m, 6H), 0.67-0.63 (m, 6H), 0.12 (s, 9H) ppm; ¹³C NMR (151 MHz, C₆D₆) δ=215.4, 171.5, 169.1, 145.0, 136.3, 111.5, 85.9, 83.1, 82.7, 82.0, 81.6, 75.9, 75.5, 73.9, 65.5, 65.9, 65.8, 65.7, 63.9, 63.7, 54.5, 47.1, 45.6, 42.9, 35.9, 33.4, 25.8, 23.0, 19.9, 18.7, 18.5, 18.3, 17.9, 17.2, 15.9, 7.4, 7.3, 5.7, 5.6, 1.2 ppm; HRMS (ESI) calcd for C₅₀H₉₇F₂N₃O₁₀PSSi₃⁺ [M+H]+ 1084.5903, found 1084.5928.

### (1S,3S,7S,10R,11S,12S,16R)-8,8,10,12,16-Pentamethyl-3-{(1E)-1-[1-methyl-5-(methylsulfanyl)-1H-pyrazol-3-yl]prop-1-en-2-yl}-7,11-bis[(triethylsilyl)oxy]-17-{[2-(trimethylsilyl)ethoxy]methyl}-4-oxa-17-azabicyclo[14.1.0]heptadecane-5,9-dione (105)

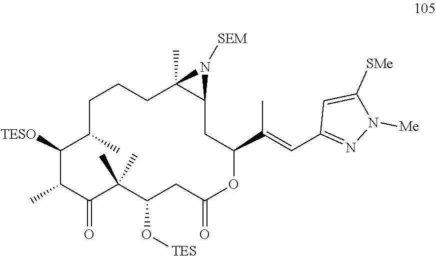

To a stirred solution of phosphonate adduct 104 (4.5 mg, 4.1 μmol, 1.0 equiv) in tetrahydrofuran (0.8 mL) at 20° C. was added potassium tert-butoxide (2.5 mg, 22 μmol, 5.0 equiv). After 5 min, the reaction mixture was quenched with water (3 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, 30% ethyl acetate in hexanes) to afford pure protected epothilone 105 (2.6 mg, 2.9 μmol, 70% yield) as a colorless oil. 105: Rₜ=0.31 (silica gel, 25% ethyl acetate in hexanes); [α]_{D}²²=−8.0 (c=0.15, CH₂Cl₂); FT-IR (neat) vₘₐₓ 2953, 2877, 1742, 1696, 1459, 1415, 1380, 1343, 1283, 1247, 1198, 1157, 1103, 1018, 985, 941, 860, 836, 783, 739 cm⁻¹; ¹H NMR (600 MHz, C₆D₆) δ=6.79 (s, 1H), 6.30 (s, 1H), 5.51 (dd, J=8.4, 4.2 Hz, 1H), 4.28 (dd, J=7.8, 4.2 Hz, 1H), 4.15 (d, J=8.4 Hz, 1H), 4.12-4.08 (m, 2H), 3.91 (ddd, J=9.0, 8.4, 8.4 Hz, 1H), 3.78 (ddd, J=9.0, 8.4, 8.4 Hz, 1H), 3.43 (s, 3H), 3.05 (dq, J=8.4, 7.2 Hz, 1H), 2.69 (dd, J=16.2, 8.4 Hz, 1H), 2.60 (dd, J=16.2, 3.6 Hz, 1H), 2.24 (s, 3H), 2.22 (ddd, J=15.6, 4.2, 4.2 Hz, 1H), 2.11 (ddd, J=15.6, 8.4, 8.4 Hz, 1H), 1.90-1.83 (m, 2H), 1.77 (s, 3H), 1.74-1.68 (m, 1H), 1.65-1.58 (m, 1H), 1.54-1.44 (m, 2H), 1.38 (dd, J=6.0, 3.0 Hz, 1H), 1.22-1.14 (3H), 1.17 (s, 6H), 1.16 (d, J=6.6 Hz, 3H), 1.14 (d, J=6.6 Hz, 3H), 1.09-1.05 (m, 18H), 0.89 (s, 3H), 0.83-0.76 (m, 6H), 0.74-0.69 (m, 6H), 0.05 (m, 9H) ppm; ¹³C NMR (151 MHz, C₆D₆) δ=214.6, 170.6, 148.8, 137.0, 135.9, 119.9, 110.2, 84.2, 80.0, 79.4, 75.6, 65.8, 53.5, 47.8, 46.8, 44.2, 40.5, 37.5, 36.3, 36.1, 34.7, 32.3, 25.4, 23.4, 22.6, 20.1, 18.5, 18.4, 17.5, 15.8, 14.9, 7.4, 7.3, 5.9, 5.8, 1.1 ppm; HRMS (ESI) calcd for C₄₆H₈₈N₃O₆SSi₃⁺ [M+H]⁺ 894.5696, found 894.5708.

### Protected Epothilone 105 (One Pot Procedure)

To a stirred solution of phosphonate 62 (47.7 mg, 0.152 mmol, 22 equiv) in tetrahydrofuran (1.0 mL) at −78° C. was added a solution of n-butyllithium (2.5 M in hexanes, 48 μL, 0.120 mmol, 18 equiv) in hexanes. After 10 min, the reaction mixture was transferred to a stirred solution of methyl ketone 87 (5.3 mg, 6.9 μmol, 1.0 equiv) in tetrahydrofuran (0.5 mL). After 30 min, the reaction mixture was allowed to slowly warm to 0° C. After 4 h, the reaction mixture was quenched by adding saturated aqueous ammonium chloride (3 mL), extracted with ethyl acetate (3×10 mL), and the combined organic layers were dried with anhydrous sodium sulfate and concentrated under reduced pressure. The obtained residue was purified by preparative thin layer chromatography (silica gel, 30% ethyl acetate in hexanes) to afford pure protected epothilone 105 (3.9 mg, 4.4 μmol, 63% yield) as a colorless oil (for characterization data of 105, see above).

### (1S,3S,7S,10R,11S,12S,16R)-7,11-Dihydroxy-8,8,10,12,16-pentamethyl-3-{(1E)-1-[1-methyl-5-(methylsulfanyl)-1H-pyrazol-3-yl]prop-1-en-2-yl}-4-oxa-17-azabicyclo[14.1.0]heptadecane-5,9-dione (40)

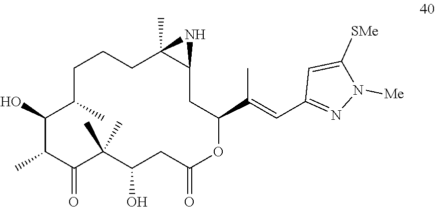

To a stirred solution of protected epothilone 105 (3.7 mg, 4.1 μmol, 1.0 equiv) in dichloromethane (0.8 mL) at 0° C. was added trifluoroacetic acid (0.20 mL, 2.6 mmol, excess), and the reaction mixture was allowed to slowly warm to 25° C. After 2 h, the solvent was removed under reduced pressure, and the obtained residue was purified by preparative thin layer chromatography (silica gel, 20% methanol in ethyl acetate) to afford pure epothilone 40 (1.8 mg, 3.4 μmol, 82% yield) as a white amorphous solid. 40: R_{f}=0.34 (silica gel, 20% methanol in ethyl acetate); [α]2=27.0 (c=0.10, CH₂Cl₂); FT-IR (film) vₘₐₓ 3368, 2954, 2926, 2856, 1732, 1687, 1492, 1457, 1378, 1335, 1283, 1258, 1200, 1174, 1144, 1084, 1038, 1007, 980, 886, 830, 798, 765, 750, 720 cm⁻¹; ¹H NMR (600 MHz, CD₂Cl₂) δ=6.46 (s, 1H), 6.35 (s, 1H), 5.54 (dd, J=4.2, 4.2 Hz, 1H), 4.06 (dd, J=10.8, 3.6 Hz, 1H), 3.84 (s, 3H), 3.79 (dd, J=6.0, 3.0 Hz, 1H), 3.32 (dq, J=6.6, 6.6 Hz, 1H), 2.53 (dd, J=12.6, 10.8 Hz, 1H), 2.42 (dd, J=12.6, 3.6 Hz, 1H), 2.41 (s, 3H), 2.02 (s, 3H), 1.96-1.91 (m, 1H), 1.85-1.82 (m, 1H), 1.78-1.74 (m, 1H), 1.73-1.68 (m, 1H), 1.45-1.21 (m, 7H), 1.40 (s, 3H), 1.21 (s, 3H), 1.09 (d, J=6.6 Hz, 3H), 1.00 (s, 3H), 0.94 (d, J=6.6 Hz, 3H) ppm; ¹³C NMR (151 MHz, CD₂Cl₂) δ=220.7, 171.2, 148.6, 136.9, 135.3, 117.6, 109.3, 76.2, 75.7, 52.6, 45.0, 38.7, 38.3, 36.7, 35.3, 31.0, 30.1, 28.8, 27.5, 25.6, 23.0, 22.6, 22.4, 19.2, 18.7, 17.6, 16.2, 14.9 ppm; HRMS (ESI) calcd for C₂₈H₄₅N₃O₅SNa⁺ [M+Na]⁺ 558.2972, found 558.2971.

### tert-Butyl (2-{(1S,3S,7S,10R,11S,12S,16R)-8,8,10,12,16-pentamethyl-3-{(1E)-1-[2-(methylsulfanyl)-1,3-thiazol-4-yl]prop-1-en-2-yl}-5,9-dioxo-7,11-bis[(triethylsilyl)oxy]-4-oxa-17-azabicyclo[14.1.0]heptadec-17-yl}ethyl)carbamate (91)

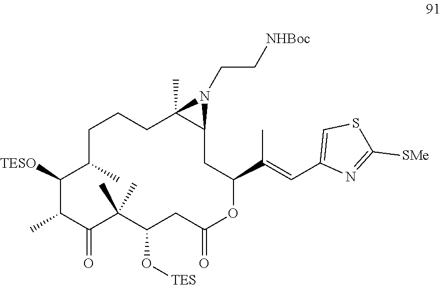

To a stirred solution of protected epothilone 85 (20.0 mg, 26.1 μmol, 1.0 equiv) in dimethylformamide (0.3 mL) at 25° C. was added tert-butyl-N-(2-bromoethyl) carbamate 90 (35.0 mg, 157 μmol, 6.0 equiv), followed by potassium carbonate (18.0 mg, 0.130 mmol, 5.0 equiv), and the reaction mixture was heated to 75° C. After 12 h, the reaction mixture was allowed to cool to 25° C., and quenched with water (5 mL). The mixture was extracted with ethyl acetate (3×10 mL), and the combined organic layers were dried with anhydrous sodium sulfate and concentrated under reduced pressure. The obtained residue was purified by flash column chromatography (silica gel, 10→40% ethyl acetate in hexanes) to afford pure protected epothilone 91 (7.5 mg, 8.2 μmol, 32%, plus 35% recovered protected epothilone 85) as a colorless oil. 91: R_{f}=0.34 (silica gel, 40% ethyl acetate in hexanes); [α]_{D}²²=5.1 (c=0.75, CH₂Cl₂); FT-IR (neat) vₘₐₓ 3373, 2955, 2934, 2876, 1741, 1697, 1500, 1458, 1424, 1383, 1365, 1248, 1159, 1111, 1069, 1036, 1019, 985, 863, 837, 782, 739, 677 cm⁻¹; ¹H NMR (600 MHz, C₆D₆) δ=6.64 (s, 1H), 6.47 (s, 1H), 5.37 (dd, J=8.4, 3.6 Hz, 1H), 5.02 (br s, 1H), 4.24 (dd, J=9.0, 3.6 Hz, 1H), 4.15 (d, J=9.0 Hz, 1H), 3.31 (dddd, J=12.6, 6.6, 6.6, 6.6 Hz, 1H), 3.21 (dddd, J=12.6, 6.0, 6.0, 6.0, Hz, 1H), 3.00 (ddd, J=14.4, 6.6, 6.6 Hz, 1H), 2.68 (dd, J=16.2, 8.4 Hz, 1H), 2.57 (dd, J=16.2, 3.6 Hz, 1H), 2.44-2.40 (m, 1H), 2.27 (s, 3H), 2.21 (s, 3H). 2.11-2.07 (m, 2H), 1.97-1.92 (m, 1H), 1.87-1.83 (m, 1H), 1.70-1.63 (m, 2H), 1.57-1.51 (m, 1H), 1.47 (s, 9H), 1.44-1.38 (m, 1H), 1.36-1.29 (m, 1H), 1.18 (d, J=6.6 Hz, 3H), 1.17 (s, 3H), 1.17-1.15 (m, 1H), 1.12 (d, J=6.6 Hz, 3H), 1.073 (t, J=8.4 Hz, 9H), 1.070 (t, J=8.4 Hz, 9H), 1.03-1.00 (m, 1H), 0.97 (s, 3H), 0.87 (s, 3H), 6.54 (m, 6H), 0.74-0.70 (m, 6H) ppm; ¹³C NMR (150 MHz, C₆D₆) δ=214.5, 170.6, 165.4, 155.9, 153.6, 138.7, 120.7, 116.6, 80.1, 79.4, 78.5, 75.8, 53.4, 51.7, 49.6, 48.0, 44.0, 41.5, 40.3, 37.3, 36.1, 34.9, 32.3, 28.5, 25.4, 23.6, 22.9, 20.2, 17.6, 15.9, 15.4, 14.7, 7.4, 7.3, 6.0, 5.8 ppm; HRMS (ESI) calcd for C₄₆H₈₄N₃O₇S₂Si₂ [M+H]⁺ 910.5284, found 910.5293.

### (1S,3S,7S,10R,11S,12S,16R)-17-(2-Aminoethyl)-7,11-dihydroxy-8,8,10,12,16-pentamethyl-3-{(1E)-1-[2-(methylsulfanyl)-1,3-thiazol-4-yl]prop-1-en-2-yl}-4-oxa-17-azabicyclo[14.1.0]heptadecane-5,9-dione (37)

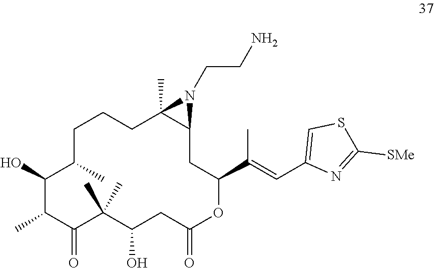

To a stirred solution of protected epothilone 91 (6.0 mg, 6.6 μmot, 1.0 equiv) in tetrahydrofuran (1.0 mL) at 0° C. was added hydrogen fluoride-pyridine complex (70%, 0.10 mL, 3.9 mmol, excess), and the reaction mixture was allowed to slowly warm to 25° C. After 2 h, the reaction mixture was quenched with saturated aqueous sodium bicarbonate solution (5 mL), and the two phases were separated. The aqueous layer was extracted with ethyl acetate (3×10 mL), and the combined organic layers were dried with anhydrous sodium sulfate and concentrated under reduced pressure. The obtained residue was dissolved in dichloromethane (1 mL) at 0° C. with stirring, and trifluoroacetic acid (0.10 mL, 1.3 mmol, excess) was added. After 1 h, the reaction mixture was concentrated under reduced pressure. The obtained residue was redissolved in ethyl acetate (50 mL) at 25° C. with stirring, and a saturated aqueous solution of sodium bicarbonate (5 mL) was added. After 5 min, the two phases were separated, and the organic layer was dried with anhydrous sodium sulfate and concentrated under reduced pressure. The obtained residue was purified by flash column chromatography (silica gel, 10→30% methanol in acetone) to afford pure epothilone 37 (2.5 mg, 4.3 μmot, 65% yield overall) as a white amorphous solid. 37: R_{f}=0.30 (silica gel, 30% methanol in acetone); [α]_{D}²²=−10.8 (c=0.25, CH₂Cl₂); FT-IR (film) vₘₐₓ 3366, 2929, 1729, 1686, 1565, 1421, 1370, 1338, 1252, 1149, 1037, 1008, 981, 881, 715 cm⁻¹; ¹H NMR (600 MHz, CD₂Cl₂) δ=7.02 (s, 1H), 6.49 (s, 1H), 5.41 (dd, J=5.4, 5.4 Hz, 1H), 4.09 (dd, J=9.6, 3.0 Hz, 1H), 3.74 (dd, J=4.8, 4.8 Hz, 1H), 3.41 (s, 1H), 3.27 (ddd, J=12.0, 6.6, 6.6 Hz, 1H), 2.84-2.75 (m, 2H), 2.70 (s, 3H), 2.54-2.38 (m, 5H), 2.11 (s, 3H), 1.95-1.93 (m, 1H), 1.90-1.85 (m, 2H), 1.83-1.82 (m, 1H), 1.71-1.65 (m, 1H), 1.60-1.36 (m, 4H), 1.35 (s, 3H), 1.30-1.22 (m, 2H), 1.28 (s, 3H), 1.11 (d, J=6.6 Hz, 3H), 1.05 (s, 3H), 0.96 (d, J=6.6 Hz, 3H) ppm; ¹³C NMR (151 MHz, CD₂Cl₂) δ=206.7, 171.4, 165.9, 153.3, 138.0, 119.0, 116.4, 78.6, 75.4, 74.6, 70.4, 53.0, 50.8, 48.4, 44.3, 43.1, 39.6, 35.6, 32.3, 30.2, 28.2, 21.9, 20.9, 20.6, 17.6, 16.9, 16.1, 15.6, 14.0 ppm; HRMS (ESI) calcd for C₂₉H₄₈N₃O₅S₂⁺ [M+H]⁺ 604.2849, found 604.2854.

### (1S,3S,7S,10R,11S,12S,16R)-3-Acetyl-17-(cyclopropylmethyl)-8,8,10,12,16-pentamethyl-7,11-bis[(triethylsilyl)oxy]-4-oxa-17-azabicyclo[14.1.0]heptadecane-5,9-dione (93)

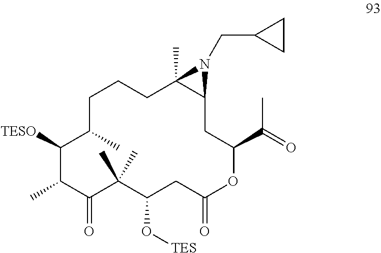

To a stirred solution of aziridine 80 (40.0 mg, 62.5 μmot, 1.0 equiv) in dimethylformamide (0.4 mL) at 25° C. was added (bromomethyl)cyclopropane 92 (50.6 mg, 0.375 mmol, 6.0 equiv), followed by potassium carbonate (43.0 mg, 0.312 mmol, 5.0 equiv), and the reaction mixture was heated to 75° C. After 16 h, the reaction mixture was allowed to cool to 25° C., and was quenched with water (3 mL). The mixture was extracted with ethyl acetate (3×15 mL), and the combined organic layers were dried with anhydrous sodium sulfate and concentrated under reduced pressure. The obtained residue was purified by flash column chromatography (silica gel, 10→40% ethyl acetate in hexanes) to afford pure cyclopropylmethyl aziridine 93 (40.0 mg, 57.6 μmol, 92% yield) as a pale yellow oil. 93: R_{f}=0.23 (silica gel, 30% ethyl acetate in hexanes); [α]_{D}²²=−6.5 (c=1.0, CH₂Cl₂); FT-IR (neat) vₘₐₓ 2952, 2918, 2877, 1747, 1732, 1696, 1460, 1414, 1381, 1308, 1284, 1239, 1197, 1158, 1109, 1070, 1042, 1010, 984, 941, 862, 835, 783, 725, 676 cm⁻¹; ¹H NMR (600 MHz, C₆D₆) δ=4.93 (dd, J=9.0, 3.6 Hz, 1H), 4.19 (d, J=9.6 Hz, 1H), 4.06 (dd, J=7.8, 4.8 Hz, 1H), 2.85 (dq, J=9.6, 6.6 Hz, 1H), 2.72-2.71 (m, 2H), 2.60 (dd, J=12.0, 5.4 Hz, 1H), 2.11 (ddd, J=15.6, 3.0, 3.0 Hz, 1H), 1.98 (dd, J=12.0, 7.2 Hz, 1H), 1.85-1.76 (m, 2H), 1.77 (s, 3H), 1.73-1.56 (m, 3H), 1.48-1.35 (m, 2H), 1.26-1.22 (m, 1H), 1.21 (d, J=6.6 Hz, 3H), 1.16 (s, 3H), 1.10 (t, J=7.8 Hz, 9H), 1.08 (t, J=7.8 Hz, 9H), 1.04 (d, J=6.6 Hz, 3H), 1.03 (s, 3H), 0.99-0.92 (m, 2H), 0.86-0.77 (m, 6H), 0.74-0.70 (m, 6H), 0.68 (s, 3H), 0.45-0.42 (m, 2H), 0.22-0.16 (m, 2H) ppm; ¹³C NMR (151 MHz, C₆D₆) δ=213.9, 202.5, 171.8, 80.8, 78.2, 76.7, 56.7, 53.1, 50.1, 48.3, 43.2, 39.4, 36.9, 36.0, 31.6, 25.3, 25.1, 24.9, 23.0, 20.1, 17.8, 15.3, 12.0, 7.5, 7.3, 6.0, 5.8, 3.6, 3.4 ppm; HRMS (ESI) calcd for C₃₈H₇₂NO₆Si₂⁺ [M+H]⁺ 694.4893, found 694.4895.

### (1S,3S,7S,10R,11S,12S,16R)-17-(Cyclopropylmethyl)-8,8,10,12,16-pentamethyl-3-{(1E)-1-[2-(methylsulfanyl)-1,3-thiazol-4-yl]prop-1-en-2-yl}-7,11-bis[(triethylsilyl)oxy]-4-oxa-17-azabicyclo[14.1.0]heptadecane-5,9-dione (94)

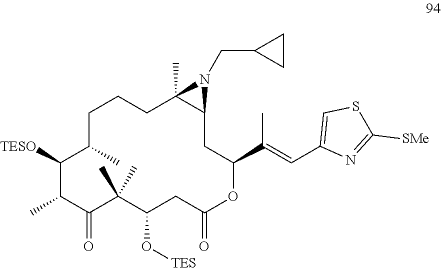

To a stirred solution of phosphonate 41 (150 mg, 0.533 mmol, 13 equiv) in tetrahydrofuran (0.5 mL) at −78° C. was added n-butyllithium (2.5 M in hexanes, 0.17 mL, 0.425 mmol, 10.3 equiv) dropwise. After 20 min, a solution of cyclopropylmethyl aziridine 93 (28.7 mg, 41.3 μmol, 1.0 equiv) in tetrahydrofuran (0.5 mL) was added, and the reaction mixture was allowed to slowly warm to 10° C., and stirred for an additional 1 h. Then the reaction mixture was quenched with saturated aqueous ammonium chloride solution (10 mL), and the two phases were separated. The aqueous layer was extracted with ethyl acetate (3×15 mL), and the combined organic layers were dried with anhydrous sodium sulfate and concentrated under reduced pressure. The obtained residue was purified by flash column chromatography (silica gel, 5→30% ethyl acetate in hexanes) to afford pure protected epothilone 94 (22.1 mg, 26.9 μmol, 65% yield) as a colorless oil. 94: R_{f}=0.22 (silica gel, 20% ethyl acetate in hexanes); [α]2=+4.2 (c=1.0, CH₂Cl₂); FT-IR (neat) vₘₐₓ 2953, 2926, 2876, 1741, 1696, 1461, 1423, 1380, 1240, 1180, 1158, 1110, 1069, 1036, 1017, 985, 915, 863, 836, 782, 738, 674 cm⁻¹; ¹H NMR (600 MHz, C₆D₆) δ=6.66 (s, 1H), 6.42 (s, 1H), 5.47 (dd, J=7.8, 3.6 Hz, 1H), 4.28 (dd, J=8.4, 3.6 Hz, 1H), 4.16 (d, J=9.0 Hz, 1H), 3.03 (dq, J=8.4, 6.6 Hz, 1H), 2.71 (dd, J=16.2, 8.4 Hz, 1H), 2.62-2.58 (m, 2H), 2.31-2.27 (m, 1H), 2.30 (s, 3H), 2.20 (s, 3H), 2.10 (ddd, J=15.0, 9.0, 9.0 Hz, 1H), 1.99 (dd, J=12.0, 7.2 Hz, 1H), 1.90-1.82 (m, 2H), 1.74-1.68 (m, 1H), 1.66-1.59 (m, 1H), 1.54-1.47 (m, 2H), 1.24-1.18 (m, 1H), 1.19 (d, J=6.6 Hz, 3H), 1.18 (s, 3H), 1.16-1.13 (m, 1H), 1.14 (d, J=6.6 Hz, 3H), 1.10 (s, 3H), 1.08 (t, J=7.8 Hz, 9H), 1.06 (t, J=7.8 Hz, 9H), 1.00-0.96 (m, 1H), 0.90 (s, 3H), 0.83-0.76 (m, 6H), 0.73-0.69 (m, 6H), 0.45-0.38 (m, 2H), 0.23-0.15 (m, 2H) ppm; ¹³C NMR (151 MHz, C₆D₆) δ=214.6, 170.7, 165.2, 153.7, 138.8, 120.6, 116.5, 79.7, 75.7, 56.9, 53.5, 49.5, 47.9, 43.5, 40.4, 37.5, 36.6, 35.2, 32.4, 30.2, 25.5, 23.6, 22.7, 20.1, 17.6, 15.9, 15.4, 14.6, 12.0, 7.43, 7.36, 5.9, 5.8, 3.6, 3.4 ppm; HRMS (ESI) calcd for C₄₃H₇₇N₂O₅S₂Si₂⁺ [M+H]⁺ 821.4807, found 821.4789.

### (1S,3S,7S,10R,11S,12S,16R)-17-(Cyclopropylmethyl)-7,11-dihydroxy-8,8,10,12,16-pentamethyl-3-{(1E)-1-[2-(methylsulfanyl)-1,3-thiazol-4-yl]prop-1-en-2-yl}-4-oxa-17-azabicyclo-[14.1.0]heptadecane-5,9-dione (38)

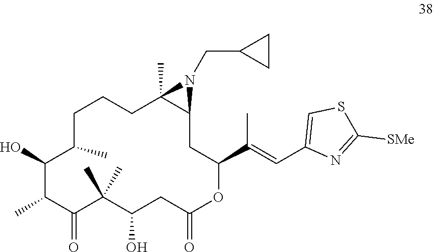

To a stirred solution of protected epothilone 94 (18.0 mg, 21.9 μmol, 1.0 equiv) in tetrahydrofuran (2.0 mL) at 0° C. was added hydrogen fluoride-pyridine complex (70%, 0.20 mL, 7.7 mmol, excess), and the reaction mixture was allowed to slowly warm to 25° C. After 3.5 h, the reaction mixture was quenched with saturated aqueous sodium bicarbonate solution (20 mL), and the two phases were separated. The aqueous layer was extracted with ethyl acetate (3×20 mL), and the combined organic layers were dried with anhydrous sodium sulfate and concentrated under reduced pressure. The obtained residue was purified by flash column chromatography (silica gel, 1→5% methanol in ethyl acetate) to afford pure epothilone 38 (12.0 mg, 20.2 μmol, 92% yield) as a white amorphous solid. 38: R_{f}=0.39 (silica gel, 5% methanol in ethyl acetate); [α]_{D}²²=−31.2 31.2 (c=1.0, CH₂Cl₂); FT-IR (film) vₘₐₓ 3375, 2957, 2924, 2853, 1729, 1687, 1555, 1464, 1424, 1378, 1251, 1148, 1036, 1009, 981, 939, 882, 832, 734 cm⁻¹; ¹H NMR (600 MHz, CD₂Cl₂) δ=7.01 (s, 1H), 6.48 (s, 1H), 5.44 (dd, J=4.8, 4.8 Hz, 1H), 4.09 (dd, J=10.2, 3.0 Hz, 1H), 3.73 (dd, J=4.8, 4.8 Hz, 1H), 3.32 (dq, J=6.6, 6.6 Hz, 1H), 2.70 (s, 3H), 2.48 (dd, J=13.8, 4.2 Hz, 1H), 2.41-2.37 (m, 1H), 2.40 (dd, J=13.8, 3.0 Hz, 1H), 2.30-2.26 (m, 1H), 2.12 (s, 3H), 1.92-1.90 (m, 2H), 1.76-1.66 (m, 2H), 1.56-1.38 (m, 4H), 1.37 (s, 3H), 1.33-1.27 (m, 2H), 1.24-1.22 (m, 1H), 1.13 (s, 3H), 1.11 (d, J=7.2 Hz, 3H), 1.04 (s, 3H), 0.96 (d, J=7.2 Hz, 3H), 0.53-0.45 (m, 2H), 0.20-0.16 (m, 1H), 0.11-0.08 (m, 1H) ppm; ¹³C NMR (151 MHz, CD₂Cl₂) δ=220.8, 171.6, 165.8, 153.3, 137.9, 118.9, 116.3, 78.1, 75.5, 57.9, 52.9, 48.2, 44.5, 43.2, 39.6, 35.5, 35.2, 32.0, 30.3, 23.0, 22.0, 21.3, 20.4, 17.7, 16.9, 16.3, 15.7, 14.1, 11.5, 4.2, 4.0 ppm; HRMS (ESI) calcd for C₃₁H₄₉N₂O₅S₂⁺ [M+H]⁺ 593.3077, found 593.3063.

### (1S,3S,7S,10R,11S,12S,16R)-3-Acetyl-8,8,10,12,16-pentamethyl-7,11-bis[(triethylsilyl)oxy]-17-[4-(trimethylsilyl)but-3-yn-1-yl]-4-oxa-17-azabicyclo[14.1.0]heptadecane-5,9-dione (96)

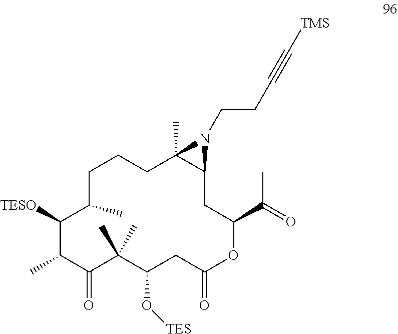

To a stirred solution of aziridine 80 (26.4 mg, 41.2 μmol, 1.0 equiv) in dimethylformamide (0.5 mL) at 25° C. was added homopropargyl bromide 95 (42.3 mg, 0.206 mmol, 5.0 equiv), followed by potassium carbonate (22.8 mg, 0.165 mmol, 4.0 equiv), and the reaction mixture was heated to 80° C. After 18 h, the reaction mixture was allowed to cool to 25° C., and quenched with water (3 mL). The two phases were separated, and the aqueous layer was extracted with ethyl acetate (3×10 mL). The combined organic layers were dried with anhydrous sodium sulfate and concentrated under reduced pressure. The obtained residue was purified by flash column chromatography (silica gel, 10→30% ethyl acetate in hexanes) to afford pure alkyne 96 (28.4 mg, 37.2 μmol, 90% yield) as a pale yellow oil. 96: R_{f}=0.31 (silica gel, 30% ethyl acetate in hexanes); [α]_{D}²²=−10.8 (c=0.25, CH₂Cl₂); FT-IR (neat) vₘₐₓ 2954, 2877, 2176, 1747, 1733, 1697, 1459, 1415, 1382, 1364, 1307, 1248, 1197, 1158, 1111, 1073, 1042, 1018, 1010, 985, 903, 842, 758, 739 cm⁻¹; ¹H NMR (600 MHz, C₆D₆) δ=4.89 (dd, J=9.6, 1.8 Hz, 1H), 4.19 (d, J=9.6 Hz, 1H), 4.04 (dd, J=6.0, 6.0 Hz, 1H), 2.83 (dq, J=9.6, 6.6 Hz, 1H), 2.70 (d, J=6.0 Hz, 2H), 2.62-2.58 (m, 1H), 2.52-2.46 (m, 1H), 2.43-2.35 (m, 2H), 2.04 (d, J=15.0 Hz, 1H), 1.84-1.77 (m, 2H), 1.80 (s, 3H), 1.67-1.55 (m, 3H), 1.45-1.32 (m, 2H), 1.23-1.18 (m, 1H), 1.20 (d, J=6.6 Hz, 3H), 1.16 (s, 3H), 1.10-1.07 (m, 18H), 1.04 (d, J=6.6 Hz, 3H), 1.03 (s, 3H), 0.98 (dd, J=9.6, 3.6 Hz, 1H), 0.80-0.76 (m, 6H), 0.75-0.71 (m, 6H), 0.67 (s, 3H), 0.26 (s, 9H) ppm; ¹³C NMR (151 MHz, C₆D₆) δ=213.8, 202.5, 171.9, 106.8, 85.2, 80.8, 78.1, 76.8, 53.1, 50.9, 50.1, 48.3, 44.0, 39.3, 36.9, 35.7, 31.6, 31.5, 25.5, 25.1, 23.0, 22.6, 20.0, 17.8, 15.4, 7.5, 7.3, 6.0, 5.8, 0.3 ppm; HRMS (ESI) calcd for C₄₁H₇₈NO₆Si₃⁺ [M+H]⁺ 764.5131, found 764.5133.

### (1S,3S,7S,10R,11S,12S,16R)-8,8,10,12,16-Pentamethyl-3-{(1E)-1-[2-(methylsulfanyl)-1,3-thiazol-4-yl]prop-1-en-2-yl}-7,11-bis[(triethylsilyl)oxy]-17-[4-(trimethylsilyl)but-3-yn-1-yl]-4-oxa-17-azabicyclo[14.1.0]heptadecane-5,9-dione (97)

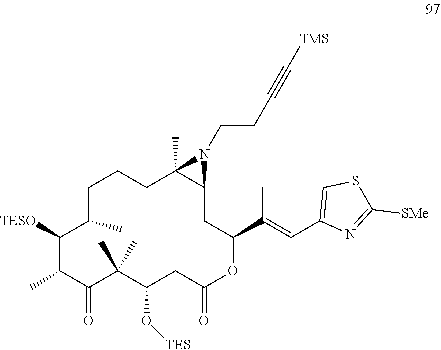

To a stirred solution of phosphonate 41 (82.0 mg, 0.291 mmol, 20 equiv) in tetrahydrofuran (0.5 mL) at −78° C. was added n-butyllithium (2.5 M in hexanes, 0.10 mL, 0.25 mmol, 17 equiv) dropwise. After 10 min, the reaction mixture was added to a stirred solution of alkyne 96 (11.0 mg, 14.4 μmot, 1.0 equiv) in tetrahydrofuran (0.5 mL) at −78° C., and the reaction mixture was allowed to slowly warm to 0° C. over 3 h. Then the reaction mixture was quenched with water (5 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, 20% ethyl acetate in hexanes) to afford pure protected epothilone 97 (8.1 mg, 9.1 μmot, 63% yield) as a colorless oil. 97: R_{f}=0.43 (silica gel, 20% ethyl acetate in hexanes); [c(]2=4.1 (c=0.32, CH₂Cl₂); FT-IR (neat) vₘₐₓ 2954, 2911, 2876, 2175, 1742, 1696, 1459, 1420, 1381, 1346, 1304, 1283, 1248, 1198, 1158, 1111, 1069, 1036, 1018, 984, 903, 841, 782, 758, 739 cm⁻¹; ¹H NMR (600 MHz, C₆D₆) δ=6.66 (s, 1H), 6.44 (s, 1H), 5.41 (dd, J=8.4, 3.0 Hz, 1H), 4.21-4.13 (m, 2H), 3.01 (dq, J=9.6, 7.2 Hz, 1H), 2.72 (dd, J=16.2, 9.0 Hz, 1H), 2.63 (ddd, J=10.8, 6.6, 6.6 Hz, 1H), 2.58 (dd, J=16.2, 3.0 Hz, 1H), 2.50-2.36 (m, 3H), 2.27 (s, 3H), 2.24-2.22 (m, 1H), 2.21 (s, 3H), 2.02 (ddd, J=15.6, 9.6, 9.6 Hz, 1H), 1.89-1.79 (m, 2H), 1.74-1.69 (m, 1H), 1.64-1.56 (m, 1H), 1.49-1.41 (m, 2H), 1.24-1.15 (m, 2H), 1.20 (d, J=6.6 Hz, 3H), 1.18 (s, 3H), 1.12 (d, J=6.6 Hz, 3H), 1.10-1.05 (m, 18H), 1.09 (s, 3H), 0.83 (s, 3H), 0.82-0.79 (m, 6H), 0.74-0.70 (m, 6H), 0.23 (s, 9H) ppm; ¹³C NMR (151 MHz, C₆D₆) δ=214.3, 170.8, 165.3, 153.6, 138.8, 120.7, 116.5, 107.0, 85.0, 80.3, 79.6, 76.0, 53.4, 51.4, 50.0, 48.2, 44.1, 40.0, 37.2, 36.2, 35.4, 32.2, 25.4, 23.5, 23.4, 22.6, 20.1, 17.7, 15.9, 15.5, 14.6, 7.5, 7.4, 6.0, 5.8, 0.3 ppm; HRMS (ESI) calcd for C₄₆H₈₂N₂O₅S₂Si₃Na⁺ [M+Na]⁺913.4865, found 913.4883.

### (1S,3S,7S,10R,11S,12S,16R)-17-(But-3-yn-1-yl)-7,11-dihydroxy-8,8,10,12,16-pentamethyl-3-{(1E)-1-[2-(methylsulfanyl)-1,3-thiazol-4-yl]prop-1-en-2-yl}-4-oxa-17-azabicyclo[14.1.0]heptadecane-5,9-dione (39)

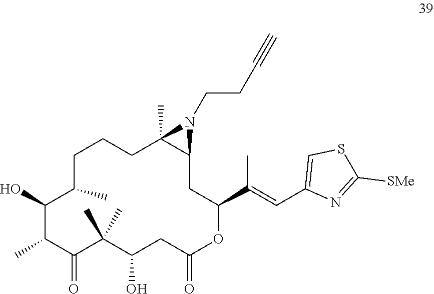

To a stirred solution of protected epothilone 97 (5.6 mg, 6.3 μmot, 1.0 equiv) in tetrahydrofuran (0.9 mL) at 0° C. was added hydrogen fluoride-pyridine complex (70%, 0.10 mL, 3.9 mmol, excess). After 2 h, the reaction mixture was quenched with saturated aqueous sodium bicarbonate solution (20 mL), and allowed to warm to 25° C. The two phases were separated, the aqueous layer was extracted with ethyl acetate (3×10 mL), and the combined organic layers were dried with anhydrous sodium sulfate and concentrated under reduced pressure. The obtained residue was redissolved in tetrahydrofuran (0.5 mL) at 25° C. with stirring, and a premixed solution of acetic acid (90.0 mg, 1.50 mmol, excess) and n-tetrabutyl ammonium fluoride (1.0 M in THF, 1.50 mL, 1.50 mmol, excess) in tetrahydrofuran (0.5 mL) was added. After 8 h, the reaction mixture was quenched with water (10 mL), and the two phases were separated. The aqueous layer was extracted with ethyl acetate (3×10 mL), and the combined organic layers were dried with anhydrous sodium sulfate and concentrated under reduced pressure. The obtained residue was purified by preparative thin layer chromatography (silica gel, ethyl acetate) to afford pure epothilone 39 (2.5 mg, 4.2 μmol, 68% yield overall) as a white amorphous solid. 39: R_{f}=0.24 (silica gel, 20% ethyl acetate in hexanes); [α]_{D}²²=−48.2 (c=0.11, CH₂Cl₂); FT-IR (film) vₘₐₓ 3494, 3299, 2280, 1735, 1686, 1553, 1466, 1424, 1385, 1330, 1291, 1262, 1150, 1036, 1009, 979, 880, 813, 748 cm⁻¹; ¹H NMR (600 MHz, C₆D₆) δ=6.68 (s, 1H), 6.46 (s, 1H), 5.58 (dd, J=4.8, 4.8 Hz, 1H), 4.69 (br s, 1H), 4.19-4.18 (m, 1H), 3.91-3.88 (m, 1H), 3.23 (dq, J=7.2, 6.6 Hz, 1H), 2.62-2.56 (m, 2H), 2.45-2.36 (m, 2H), 2.29 (dd, J=13.2, 3.0 Hz, 1H), 2.24-2.18 (m, 2H), 2.19 (s, 3H), 2.05 (s, 3H), 1.87-1.83 (m, 1H), 1.81-1.76 (m, 2H), 1.70-1.66 (m, 1H), 1.49-1.36 (m, 3H), 1.35-1.28 (m, 1H), 1.24-1.20 (m, 1H), 1.09 (s, 3H), 1.08 (d, J=7.2 Hz, 3H), 1.03 (dd, J=6.6, 6.6 Hz, 1H), 0.98 (d, J=7.2 Hz, 3H), 0.95 (s, 3H), 0.81 (s, 3H) ppm; ¹³C NMR (151 MHz, C₆D₆) δ=219.4, 170.9, 165.6, 153.5, 137.4, 118.9, 116.1, 82.8, 77.4, 75.6, 75.2, 69.6, 52.4, 52.0, 47.8, 44.7, 42.8, 39.1, 35.2, 34.7, 31.5, 29.8, 21.9, 21.6, 20.4, 19.6, 17.8, 16.0, 15.78, 15.75, 14.4 ppm; HRMS (ESI) calcd for C₃₁H₄₆N₂O₅S₂Na⁺ [M+Na]⁺ 613.2740, found 613.2746.

### (1S,3S,7S,10R,11S,12S,16R)-17-(2-Azidoethyl)-7,11-dihydroxy-8,8,10,12,16-pentamethyl-3-{(1E)-1-[2-(methylsulfanyl)-1,3-thiazol-4-yl]prop-1-en-2-yl}-4-oxa-17-azabicyclo[14.1.0]heptadecane-5,9-dione (34)

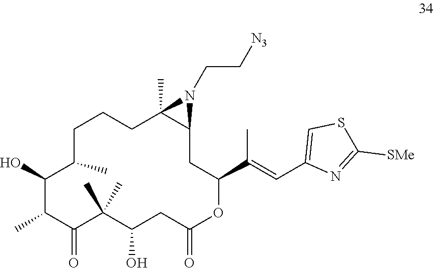

To a stirred solution of epothilone 12 (12.8 mg, 22.0 μmol, 1.0 equiv) in dichloromethane (1 mL) at 0° C. was added p-toluenesulfonic anhydride (35.8 mg, 0.110 mmol, 5.0 equiv), followed by triethylamine (12.3 μL, 88.0 μmol, 4.0 equiv). The reaction mixture was allowed to slowly warm to 25° C. over 30 min, and stirred for an additional 15 min. Then the reaction mixture was quenched with methanol (0.5 mL) and water (10 mL), and the two phases were separated. The aqueous layer was extracted with ethyl acetate (3×15 mL), and the combined organic layers were dried with anhydrous sodium sulfate and concentrated under reduced pressure. The obtained residue was filtered through a pad of silica gel, rinsed thoroughly with ethyl acetate (15 mL), and concentrated under reduced pressure. Then the obtained crude tosylate was dissolved in dimethylformamide (0.5 mL) at 25° C. with stirring, and sodium azide (5.7 mg, 88.0 μmol, 4.0 equiv) was added. After 17 h, the reaction mixture was quenched with water (5 mL), and extracted with ethyl acetate (3×15 mL). The combined organic layers were dried with anhydrous sodium sulfate and concentrated under reduced pressure. The obtained residue was purified by flash column chromatography (silica gel, 50→90% methanol in ethyl acetate) to afford pure epothilone 34 (5.3 mg, 8.7 μmol, 40% yield overall) as a white amorphous solid. 34: R_{f}=0.35 (silica gel, ethyl acetate); [α]2=34.2 (c=0.55, CH₂Cl₂); FT-IR (film) vₘₐₓ 3432, 2929, 2101, 1731, 1687, 1554, 1423, 1384, 1263, 1148, 1036, 1009, 979, 881, 735 cm⁻¹; ¹H NMR (600 MHz, CD₂Cl₂) δ=7.03 (s, 1H), 6.48 (s, 1H), 5.38 (dd, J=7.8, 3.6 Hz, 1H), 4.12-4.09 (m, 1H), 3.95 (br s, 1H), 3.74 (ddd, J=4.8, 4.8, 4.8 Hz, 1H), 3.44-3.37 (m, 2H), 3.26 (dq, J=6.6, 4.8 Hz, 1H), 2.70 (s, 3H), 2.67 (ddd, J=12.6, 6.0, 6.0 Hz, 1H), 2.59 (ddd, J=12.6, 6.6, 6.6 Hz, 1H), 2.49-2.45 (m, 2H), 2.39 (dd, J=15.0, 3.0 Hz, 1H), 2.13 (s, 3H), 2.01 (ddd, J=15.0, 4.2, 4.2 Hz, 1H), 1.82 (ddd, J=16.2, 7.8, 7.8 Hz, 1H), 1.71-1.65 (m, 1H), 1.51-1.41 (m, 4H), 1.35 (s, 3H), 1.30-1.28 (m, 1H), 1.26-1.22 (m, 1H), 1.16 (s, 3H), 1.12 (d, J=7.2 Hz, 3H), 1.05 (s, 3H), 0.97 (d, J=6.6 Hz, 3H) ppm; ¹³C NMR (151 MHz, CD₂Cl₂) δ=220.7, 171.2, 166.0, 153.1, 138.3, 119.3, 116.5, 78.8, 74.5, 74.2, 53.1, 52.3, 51.9, 49.0, 43.9, 43.8, 39.7, 35.9, 35.8, 32.7, 30.7, 22.0, 21.3, 20.3, 17.5, 16.9, 16.1, 15.4, 13.6 ppm; HRMS (ESI) calcd for C₂₉H₄₆N₅O₅S₂⁺ [M+H]⁺ 608.2935, found 608.2933.

### (1S,3S,7S,10R,11S,12S,16R)-7,11-Dihydroxy-8,8,10,12,16-pentamethyl-3-{(1E)-1-[2-(methyl-sulfanyl)-1,3-thiazol-4-yl]prop-1-en-2-yl}-17-(2-sulfanylethyl)-4-oxa-17-azabicyclo[14.1.0]-heptadecane-5,9-dione (35)

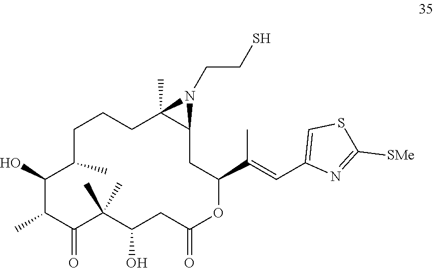

To a stirred solution of epothilone 12 (5.0 mg, 8.6 μmol, 1.0 equiv) in dichloromethane (0.5 mL) at 0° C. was added p-toluenesulfonic anhydride (5.6 mg, 17 μmol, 2.0 equiv), followed by triethylamine (2.4 μL, 17 μmol, 2.0 equiv). After 5 min, the reaction mixture was quenched with methanol (0.5 mL) and water (10 mL), and the two phases were separated. The aqueous layer was extracted with ethyl acetate (3×10 mL), and the combined organic layers were dried with anhydrous sodium sulfate and concentrated under reduced pressure. The obtained residue was filtered through a pad of silica gel, rinsed thoroughly with ethyl acetate (15 mL), and concentrated under reduced pressure. The obtained crude tosylate was dissolved in dimethylformamide (0.5 mL) at 0° C. with stirring, sodium hydrosulfide (1.0 mg, 17 μmol, 2.0 equiv) was added, and the reaction mixture was allowed to slowly warm to 15° C. over 1.5 h. Then the reaction mixture was quenched with water (5 mL), and extracted with ethyl acetate (3×10 mL). The combined organic layers were dried with anhydrous sodium sulfate and concentrated under reduced pressure. The obtained residue was purified by preparative thin layer chromatography (silica gel, 10% methanol in ethyl acetate) to afford pure epothilone 35 (2.6 mg, 4.3 μmol, 54% yield overall) as a white amorphous solid. 35: R_{f}=0.18 (silica gel, 10% methanol in ethyl acetate); [α]_{D}²²=−10.9 (c=0.11, CH₂Cl₂); FT-IR (film) vₘₐₓ 3470, 2928, 1730, 1688, 1466, 1422, 1384, 1262, 1144, 1036, 1009, 979, 882, 764, 749 cm⁻¹; ¹H NMR (600 MHz, C₆D₆) δ=6.70 (s, 1H), 6.55 (s, 1H), 5.60 (dd, J=4.8, 4.8 Hz, 1H), 4.22-4.20 (m, 1H), 3.90-3.88 (m, 1H), 3.32-3.28 (m, 1H), 3.12-3.07 (m, 1H), 2.98-2.93 (m, 1H), 2.88-2.84 (m, 1H), 2.73-2.68 (m, 1H), 2.36-2.35 (m, 2H), 2.20 (s, 3H), 2.10 (s, 3H), 1.88-1.83 (m, 1H), 1.77-1.72 (m, 1H), 1.55-1.35 (m, 4H), 1.31-1.26 (m, 1H), 1.16 (s, 3H), 1.14-1.04 (m, 2H), 1.12 (d, J=6.6 Hz, 3H), 1.02-0.88 (m, 1H), 1.01 (d, J=7.2 Hz, 3H), 0.98 (s, 3H), 0.94 (s, 3H) ppm; ¹³C NMR (151 MHz, C₆D₆) δ=219.5, 170.9, 165.7, 153.4, 137.4, 119.1, 116.4, 77.6, 76.0, 74.9, 60.0, 52.8, 52.5, 48.0, 44.9, 43.1, 39.4, 39.3, 35.4, 34.9, 31.7, 30.1, 22.2, 21.6, 20.1, 17.9, 16.02, 16.01, 14.7 ppm; HRMS (ESI) calcd for C₅₈H90O₄O₁₀S₆Na⁺ [2M−2H+Na]⁺ 1217.4873, found 1217.4873.

### 2-[(1S,3S,7S,10R,11S,12S,16R)-7,11-Dihydroxy-8,8,10,12,16-pentamethyl-3-{(1E)-1-[2-(methylsulfanyl)-1,3-thiazol-4-yl]prop-1-en-2-yl}-5,9-dioxo-4-oxa-17-azabicyclo[14.1.0]-heptadec-17-yl]ethyl acetate (36)

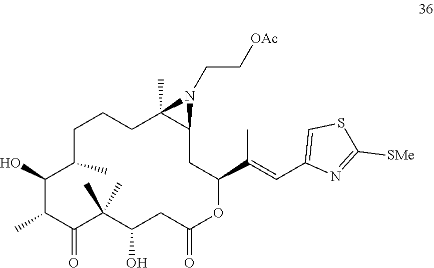

To a stirred solution of epothilone 12 (3.5 mg, 6.0 μmol, 1.0 equiv) in dichloromethane (1 mL) at 0° C. was added N,N-diisopropylethylamine (2.1 mg, 12 μmol, 2.0 equiv), followed by acetyl chloride (1.1 mg, 12 μmol, 2.0 equiv). After 1 h, the reaction mixture was quenched with methanol (1 mL) and water (5 mL), and allowed to warm to 25° C. The two phases were separated, and the aqueous layer was extracted with ethyl acetate (3×10 mL). The combined organic layers were dried with anhydrous sodium sulfate and concentrated under reduced pressure. The obtained residue was purified by preparative thin layer chromatography (silica gel, 2.5% methanol in ethyl acetate) to afford pure epothilone 36 (3.3 mg, 5.3 μmol, 88% yield) as a white amorphous solid. 36: R_{f}=0.19 (silica gel, ethyl acetate); [α]_{D}²²=−31.7 (c=0.12, CH₂Cl₂); FT-IR (film) vₘₐₓ 3458, 2928, 1736, 1688, 1457, 1425, 1382, 1367, 1248, 1150, 1039, 979, 885, 800 720 cm⁻¹; ¹H NMR (600 MHz, C₆D₆) δ=6.69 (s, 1H), 6.51 (s, 1H), 5.60 (dd, J=4.2, 4.2 Hz, 1H), 4.86 (br s, 1H), 4.42 (ddd, J=11.4, 6.6, 6.6 Hz, 1H), 4.23 (ddd, J=12.0, 6.0, 6.0 Hz, 1H), 4.18 (ddd, J=8.4, 4.2, 4.2 Hz, 1H), 3.92-3.89 (m, 1H), 3.35 (dq, J=6.6, 6.6 Hz, 1H), 2.61 (ddd, J=12.0, 6.0, 6.0 Hz, 1H), 2.41 (ddd, J=12.0, 6.0, 6.0 Hz, 1H), 2.33-2.31 (m, 2H), 2.28-2.23 (m, 1H), 2.17 (s, 3H), 2.06 (s, 3H), 1.85-1.81 (m, 1H), 1.73-1.70 (m, 2H), 1.72 (s, 3H), 1.56-1.51 (m, 1H), 1.50-1.45 (m, 1H), 1.40-1.32 (m, 2H), 1.26-1.22 (m, 1H), 1.16 (s, 3H), 1.14 (d, J=6.6 Hz, 3H), 1.04-0.99 (m, 1H), 1.03 (d, J=7.2 Hz, 3H), 0.96 (s, 3H), 0.87 (s, 3H) ppm; ¹³C NMR (151 MHz, C₆D₆) δ=219.4, 170.9, 170.3, 165.6, 153.5, 137.2, 118.8, 116.3, 77.2, 76.2, 75.1, 64.3, 52.4, 51.1, 47.2, 45.2, 42.5, 39.2, 35.5, 34.8, 31.2, 29.9, 22.2, 21.8, 20.6, 19.5, 18.0, 16.5, 16.0, 15.9, 14.8 ppm; HRMS (ESI) calcd for C₃₁H₁₄N₂O₇S₂⁺ [M+H]⁺ 625.2976, found 625.2982.

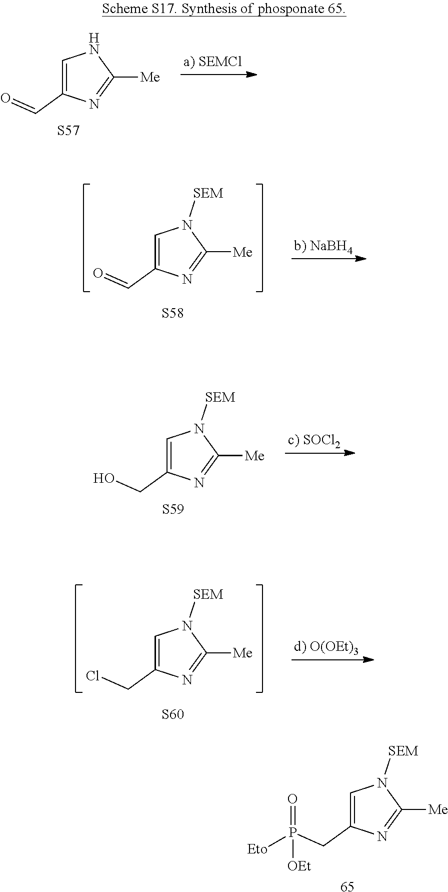

### (2-Methyl-1-{[2-(trimethylsilyl)ethoxy]methyl}-1H-imidazol-4-yl)methanol (S59)

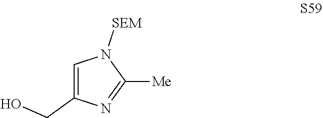 [SMILES: Cc1nc(CO)cn1COCC[Si](C)(C)C]

To a stirred solution of aldehyde S57 (0.485 g, 4.40 mmol, 1.0 equiv) in dichloromethane (12 mL) at 0° C. was added 2-(trimethylsilyl)ethoxymethyl chloride (0.953 g, 5.72 mmol, 1.3 equiv), followed by N,N-diisopropylethylamine (0.996 mL, 5.72 mmol, 1.3 equiv), and 4-dimethylaminopyridine (27 mg, 0.22 mmol, 0.05 equiv). After 20 min, the reaction mixture was diluted with methanol (12 mL), and sodium borohydride (0.168 g, 4.40 mmol, 1.0 equiv) was added. After 10 min, the reaction mixture was quenched with acetone (10 mL) and water (40 mL), and the two phases were separated. The aqueous layer was extracted with ethyl acetate (3×10 mL), and the combined organic layers were dried with anhydrous sodium sulfate and concentrated under reduced pressure. The obtained residue was purified by flash column chromatography (silica gel, 0→15% methanol in ethyl acetate) to afford pure hydroxymethyl imidazole S59 (0.820 g, 3.38 μmot, 77% yield) as a colorless oil. S59: R_{f}=0.41 (silica gel, 20% methanol in ethyl acetate); FT-IR (neat) vₘₐₓ 3156, 2952, 2894, 1578, 1509, 1421, 1362, 1262, 1248, 1202, 1142, 1124, 1083, 1023, 971, 915, 857, 832, 754 cm⁻¹; ¹H NMR (600 MHz, CDCl₃) δ=6.81 (s, 1H), 5.30 (s, 2H), 4.60 (s, 2H), 3.54-3.52 (m, 2H), 2.42 (s, 3H), 0.92-0.90 (m, 2H), 0.02 (s, 9H) ppm; ¹³C NMR (151 MHz, CDCl₃) δ=146.9, 131.5, 127.0, 73.0, 66.3, 54.6, 18.1, 13.6, 1.3 ppm; HRMS (ESI) calcd for C₁₁H₂₃N₂O₂Si⁺ [M+H]⁺ 243.1523, found 243.1524.

### Diethyl [(2-methyl-1-{[2-(trimethylsilyl)ethoxy]methyl}-1H-imidazol-4-yl)methyl]-phosphonate (65)

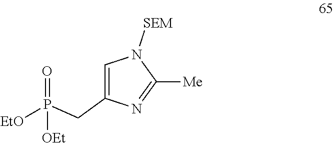 [SMILES: CCOP(=O)(Cc1cn(COCC[Si](C)(C)C)c(C)n1)OCC]

To a flask with hydroxymethyl imidazole S59 (0.604 g, 2.49 mmol, 1.0 equiv) was added thionyl chloride (1.8 mL, 25 mmol, 10 equiv) at 0° C. with stirring, and the reaction mixture was allowed to slowly warm to 25° C. After 2 h, the thionyl chloride was removed under reduced pressure. To the obtained residue was added triethyl phosphite (830 mg, 5.0 mmol, 2.0 equiv) with stirring, and the reaction mixture was heated 160° C. After 2 h, the triethyl phosphite was removed under a steady flow of nitrogen gas, and the reaction mixture was allowed to cool to 25° C. The obtained residue was purified by flash column chromatography (silica gel, 0→15% methanol in acetone) to afford pure phosphonate 65 (550 mg, 1.5 mmol, 61% yield) as a colorless oil. 65: R_{f}=0.36 (silica gel, 20% methanol in ethyl acetate); FT-IR (neat) vₘₐₓ 3444, 2981, 2954, 2901, 1642, 1569, 1519, 1478, 1443, 1420, 1395, 1363, 1312, 1247, 1162, 1084, 1053, 1024, 963, 918, 857, 835, 777, 725 cm⁻¹; ¹H NMR (600 MHz, CDCl₃) δ=6.94 (d, J=3.0 Hz, 1H), 5.14 (s, 2H), 4.11-4.06 (m, 4H), 3.50-3.47 (m, 2H), 3.14 (d, J=20.4 Hz, 2H), 2.41 (s, 3H), 1.29 (t, J=7.2 Hz, 6H), 0.91-0.88 (m, 2H), 0.02 (s, 9H) ppm; ¹³C NMR (151 MHz, CDCl₃) δ=144.9, 130.0 (d, J=7.1 Hz), 118.3 (d, J=5.9 Hz), 75.3, 66.3, 62.2 (d, J=6.6 Hz), 26.4 (d, J=140.7 Hz), 17.9, 16.6 (d, J=6.0 Hz), 13.0 ppm; HRMS (ESI) calcd for C₁₅H₃₂N₂O₄PSi⁺ [M+H]⁺ 363.1863, found 363.1870.

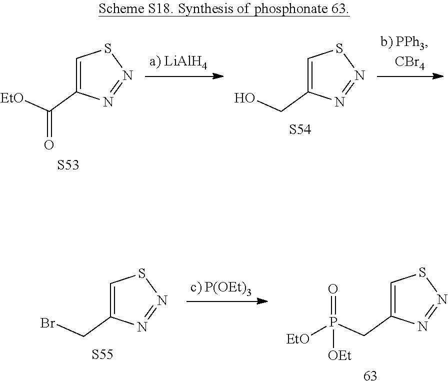

### 1,2,3-Thiadiazol-4-ylmethanol (S54)

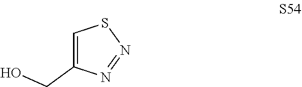

To a stirred solution of thiadiazole ethyl ester S53 (650 mg, 4.11 mmol, 1.0 equiv) in tetrahydrofuran (41 mL) at 0° C. was added lithium aluminum hydride (1.0 M in tetrahydrofuran, 1.39 mL, 1.39 mmol, 0.5 equiv) dropwise. After 45 min, the reaction mixture was quenched with the careful addition of sodium sulfate decahydrate (13.2 g, 41.0 mmol, 10.0 equiv), and allowed to warm to 25° C. The reaction mixture was then filtered through a pad of Celite®, rinsed thoroughly with ethyl acetate (50 mL), and concentrated under reduced pressure. The obtained residue was purified by flash column chromatography (silica gel, 50→100% ethyl acetate in hexanes) to afford pure hydroxymethyl thiadiazole S54 (267 mg, 2.30 mmol, 56% yield) as a colorless oil. S54: R_{f}=0.21 (silica gel, 50% ethyl acetate in hexanes); FT-IR (neat) vₘₐₓ 3368, 3108, 2938, 2878, 1719, 1641, 1541, 1452, 1420, 1231, 1117, 1054, 1032, 983, 886, 811, 727 cm⁻¹; ¹H NMR (600 MHz, CDCl₃) δ=8.52 (s, 1H), 5.20 (s, 2H), 3.12 (br s, 1H) ppm; ¹³C NMR (151 MHz, CDCl₃) δ=163.6, 133.4, 58.6 ppm; HRMS (CI) calcd for C₃H₅N₂OS⁺ [M+H]⁺ 117.0117, found 117.0120.

### 4-(Bromomethyl)-1,2,3-thiadiazole (S55)

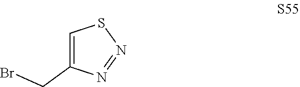

To a stirred solution of hydroxymethyl thiadiazole S54 (251 mg, 2.16 mmol, 1.0 equiv) in dichloromethane (22 mL) at 0° C. was added triphenylphosphine (850 mg, 3.24 mmol, 1.5 equiv), followed by carbon tetrabromide (1.07 g, 3.24 mmol, 1.5 equiv). After 30 min, the reaction mixture was quenched sequentially with a saturated aqueous solution of sodium bicarbonate (8 mL) and a saturated aqueous solution of sodium thiosulfate (8 mL), and allowed to warm to 25° C. The two phases were separated, the aqueous layer was extracted with dichloromethane (3×5 mL), and the combined organic layers were dried with anhydrous sodium sulfate and concentrated under reduced pressure. The obtained residue was purified by flash column chromatography (silica gel, 5→15% ethyl acetate in hexanes) to afford pure bromomethyl thiadiazole S55 (248 mg, 1.39 mmol, 64% yield) as a white amorphous solid. S55: R_{f}=0.26 (silica gel, 15% ethyl acetate in hexanes); FT-IR (film) vₘₐₓ 3105, 3036, 2978, 1489, 1427, 1245, 1215, 1121, 984, 889, 805, 705 cm⁻¹; ¹H NMR (600 MHz, CDCl₃) δ=8.56 (s, 1H), 4.94 (s, 2H) ppm; ¹³C NMR (151 MHz, CDCl₃) δ=159.8, 135.2, 22.2 ppm; HRMS (ESI) calcd for C₃H₄BrN₂S⁺ [M+H]⁺ 178.9273, found 178.9272.

### Diethyl (1,2,3-thiadiazol-4-ylmethyl)phosphonate (63)

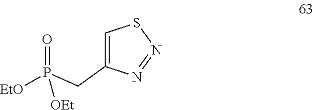 [SMILES: CCOP(=O)(Cc1csnn1)OCC]

To a stirred solution of bromomethyl thiadiazole S55 (248 mg, 1.39 mmol, 1.0 equiv) in benzene (4.6 mL) at 25° C. was added triethyl phosphite (1.2 mL, 7.0 mmol, 5.0 equiv). The reaction mixture was heated to 100° C. for 12 h, allowed to cool to 25° C., and concentrated under reduced pressure. The obtained residue was purified by flash column chromatography (silica gel, 0→5% methanol in ethyl acetate) to afford pure phosphonate 63 (305 mg, 1.29 mmol, 93% yield) as a colorless oil. 63: R_{f}=0.32 (silica gel, 5% methanol in ethyl acetate); FT-IR (neat) vₘₐₓ 3470, 3109, 2984, 2931, 2910, 1646, 1485, 1444, 1393, 1369, 1323, 1247, 1234, 1163, 1097, 1050, 1023, 969, 890, 837, 811, 778, 723, 700 cm⁻¹; ¹H NMR (600 MHz, CDCl₃) δ=8.56 (d, J=2.7 Hz, 1H), 4.11 (dq, J=8.2, 7.1 Hz, 4H), 3.77 (d, J=20.8 Hz, 2H), 1.29 (t. J=7.1 Hz, 6H) ppm; ¹³C NMR (151 MHz, CDCl₃) δ=154.1 (d, J=7.0 Hz), 134.3 (d, J=4.9 Hz), 62.8 (d, J=6.6 Hz), 26.6 (d, J=142.3 Hz), 16.5 (d, J=6.0 Hz) ppm; HRMS (ESI) calcd for C₇H₁₃N₂O₃PSNa⁺ [M+Na]⁺ 259.0277, found 259.0278.

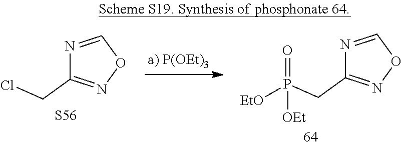

### Diethyl (1,2,4-oxadiazol-3-ylmethyl)phosphonate (64)

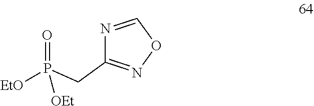 [SMILES: CCOP(=O)(Cc1ncon1)OCC]

A stirred solution of chloromethyl oxadiazole S56 (0.230 g, 1.94 mmol, 1.0 equiv) in triethyl phosphite (678 mg, 4.08 mmol, 2.1 equiv) was heated to 160° C. After 2 h, the triethyl phosphite was removed under a steady flow of nitrogen gas, and the reaction mixture was allowed to cool to 25° C. The obtained residue was purified by flash column chromatography (silica gel, 40→90% ethyl acetate in hexanes) to afford pure phosphonate 64 (375 mg, 1.70 mmol, 89% yield) as a colorless oil. 64: R_{f}=0.25 (silica gel, ethyl acetate); FT-IR (neat) vₘₐₓ 3476, 3075, 2985, 2934, 1646, 1551, 1479, 1445, 1395, 1370, 1343, 1255, 1163, 1139, 1107, 1051, 1021, 972, 959, 888, 831, 807, 762, 727 cm⁻¹; ¹H NMR (600 MHz, CDCl₃) δ=8.70 (s, 1H), 4.21-4.15 (m, 4H), 3.41 (d, J=21.6 Hz, 2H), 1.34 (t, J=7.2 Hz, 6H) ppm; ¹³C NMR (151 MHz, CDCl₃) δ=165.2, 163.3 (d, J=9.2 Hz), 63.0 (d, J=6.6 Hz), 24.9 (d, J=140.3 Hz), 16.5 (d, J=6.0 Hz) ppm; HRMS (ESI) calcd for C₇H₁₃N₂O₄PNa⁺ [M+Na]⁺ 243.0505, found 243.0510.

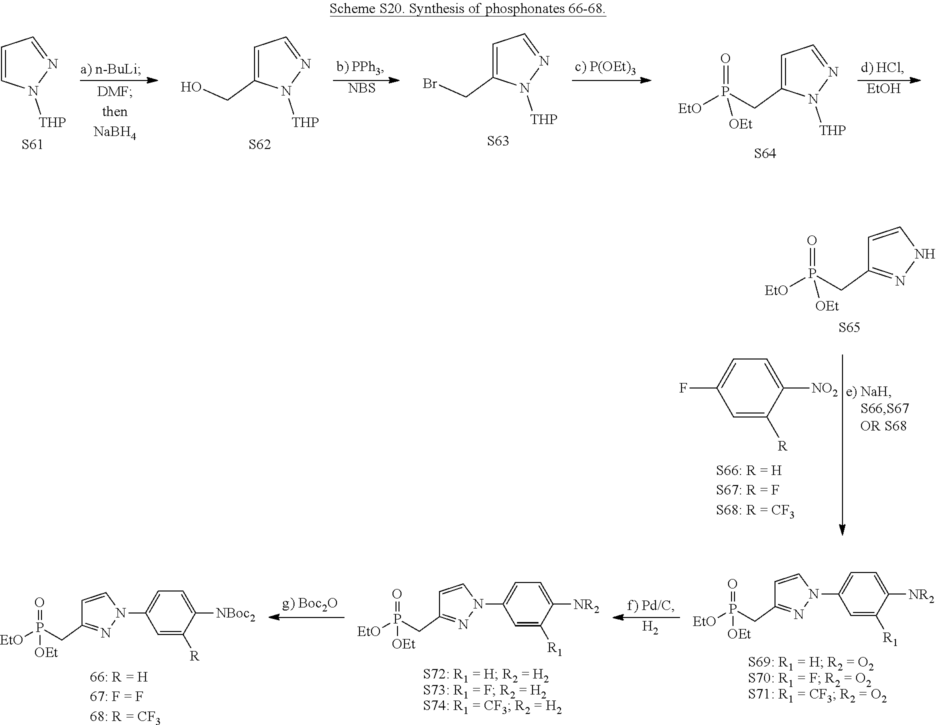

### [1-(Tetrahydro-2H-pyran-2-yl)-1H-pyrazol-5-yl]methanol (S62)

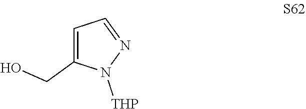

To a stirred solution of pyrazole S61 (15.0 g, 98.6 mmol, 1.0 equiv) in tetrahydrofuran (274 mL) at −78° C. was added n-butyllithium (2.5 M in hexanes, 47.3 mL, 118 mmol, 1.2 equiv) dropwise. After 20 min, dimethylformamide (9.10 mL, 118 mmol, 1.2 equiv) was added, and stirring was continued for an additional 20 min. Then the reaction mixture was quenched with methanol (60 mL), allowed to warm to 0° C., and sodium borohydride (7.46 g, 197 mmol, 2.0 equiv) was added in portions. After 30 min, the reaction mixture was quenched with saturated aqueous ammonium chloride solution (100 mL), and diluted with water (100 mL). The two phases were separated, and the aqueous layer was extracted with ethyl acetate (3×50 mL). The combined organic layers were dried with anhydrous sodium sulfate and concentrated under reduced pressure. The obtained residue was purified by flash column chromatography (silica gel, 50% ethyl acetate in hexanes) to afford pure hydroxymethyl pyrazole S62 (17.4 g, 95.6 mmol, 97% yield) as a colorless oil. S62: R_{f}=0.22 (silica gel, 50% ethyl acetate in hexanes); FT-IR (neat) vₘₐₓ 3344, 2941, 2859, 2738, 1642, 1544, 1469, 1442, 1415, 1379, 1352, 1316, 1288, 1260, 1205, 1180, 1143, 1083, 1041, 1021, 1003, 936, 913, 879, 845, 825, 788, 739, 664 cm⁻¹; ¹H NMR (600 MHz, CDCl₃) δ=7.45 (d, J=1.7 Hz, 1H), 6.26 (d, J=1.7 Hz, 1H), 5.50 (dd, J=9.6, 2.6 Hz, 1H), 4.67 (d, J=1.2 Hz, 2H), 4.03-4.00 (m, 1H), 3.72-3.68 (m, 1H), 2.41-2.35 (m, 1H), 2.13-2.05 (m, 3H), 1.74-1.66 (m, 2H), 1.64-1.60 (m, 1H) ppm; ¹³C NMR (151 MHz, CDCl₃) δ=142.3, 138.8, 107.2, 85.8, 67.8, 55.3, 29.5, 25.0, 22.5 ppm; HRMS (CI) calcd for C₉H₁₄N₂O₂⁺[M]⁺ 182.1055, found 182.1055.

### 5-(Bromomethyl)-1-(tetrahydro-2H-pyran-2-yl)-1H-pyrazole (S63)

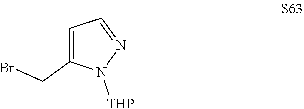

To a stirred solution of hydroxymethyl pyrazole S62 (9.0 g, 49 mmol, 1.0 equiv) in dichloromethane (490 mL) at −78° C. was added triphenylphosphine (13.7 g, 51.9 mmol, 1.05 equiv), followed by N-bromosuccinimide (8.88 g, 49.4 mmol, 1.0 equiv). After 40 min, the reaction mixture was quenched with saturated aqueous sodium bicarbonate solution (250 mL), and allowed to warm to 25° C. The two phases were separated, and the aqueous layer was extracted with dichloromethane (3×50 mL). The combined organic layers were dried with anhydrous sodium sulfate and concentrated under reduced pressure. The obtained residue was purified by flash column chromatography (silica gel, 20→40% diethyl ether in hexanes) to afford pure bromomethyl pyrazole S63 (8.0 g, 33 mmol, 66% yield) as a colorless oil. S63: R_{f}=0.34 (silica gel, 40% diethyl ether in hexanes); FT-IR (neat) vₘₐₓ 3105, 3033, 2942, 2858, 1544, 1466, 1440, 1406, 1376, 1349, 1319, 1286, 1260, 1222, 1208, 1184, 1156, 1112, 1084, 1057, 1043, 1005, 931, 914, 880, 844, 824, 789, 706, 674 cm⁻¹; ¹H NMR (600 MHz, CDCl₃) δ=7.48 (d, J=1.7 Hz, 1H), 6.33 (d, J=1.7 Hz, 1H), 5.51 (dd, J=9.3, 2.7 Hz, 1H), 4.64 (d, J=11.6 Hz, 1H), 4.57 (d, J=11.6 Hz, 1H), 4.05-3.97 (m, 1H), 3.76-3.66 (m, 1H), 2.51-2.40 (m, 1H), 2.18-2.09 (m, 1H), 2.10-2.02 (m, 1H), 1.79-1.67 (m, 2H), 1.67-1.56 (m, 1H) ppm; ¹³C NMR (151 MHz, CDCl₃) δ=139.0, 138.5, 108.2, 85.1, 67.7, 29.4, 25.1, 22.6, 20.5 ppm; HRMS (CI) calcd for C₉H₁₃BrN₂O⁺[M]⁺ 244.0211, found 244.0214.

### Diethyl {[1-(tetrahydro-2H-pyran-2-yl)-1H-pyrazol-5-yl]methyl}phosphonate (S64)

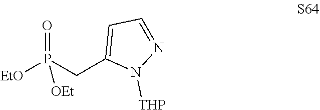

A stirred solution of bromomethyl pyrazole S63 (3.61 g, 14.7 mmol, 1.0 equiv) in triethyl phosphite (17.6 mL, 103 mmol, 7.0 equiv) was heated to 100° C. After 18 h, the triethyl phosphite was removed under a steady flow of nitrogen gas, and the reaction mixture was allowed to cool to 25° C. The obtained residue was purified by flash column chromatography (silica gel, 50→100% ethyl acetate in hexanes) to afford pure phosphonate S64 (3.55 g, 11.8 mmol, 80% yield) as a colorless oil. S64: R_{f}=0.33 (silica gel, ethyl acetate); FT-IR (neat) vₘₐₓ 3466, 3102, 2980, 2940, 2863, 1652, 1542, 1469, 1443, 1407, 1394, 1319, 1250, 1210, 1184, 1163, 1083, 1042, 1023, 967, 914, 880, 841, 827, 788, 714, 659 cm⁻¹; ¹H NMR (600 MHz, CDCl₃) δ=7.47 (d, J=1.4 Hz, 1H), 6.25 (dd, J=1.4, 1.4 Hz, 1H), 5.48 (dd, J=9.5, 2.7 Hz, 1H), 4.12-4.04 (m, 2H), 4.04-3.95 (m, 3H), 3.70-3.62 (m, 1H), 3.33 (dd, J=20.2, 16.0 Hz, 1H), 3.29 (dd, J=21.7, 16.0 Hz, 1H), 2.50-2.40 (m, 1H), 2.14-2.05 (m, 1H), 2.04-1.97 (m, 1H), 1.74-1.64 (m, 2H), 1.62-1.54 (m, 1H), 1.28 (t, J=7.1 Hz, 3H), 1.24 (t, J=7.1 Hz, 3H) ppm; ¹³C NMR (151 MHz, CDCl₃) δ=139.3 (d, J=2.8 Hz), 133.4 (d, J=8.0 Hz), 107.7 (d, J=5.2 Hz), 84.5, 67.7, 62.8 (d, J=6.8 Hz), 62.5 (d, J=6.7 Hz), 29.4, 25.1, 24.2 (d, J=143.6 Hz), 22.7, 16.5 (d, J=5.9 Hz) ppm; HRMS (CI) calcd for C₁₃H₂₃N₂O₄P⁺ [M]⁺ 302.1395, found 302.1366.

### Diethyl (1H-pyrazol-3-ylmethyl)phosphonate (S65)

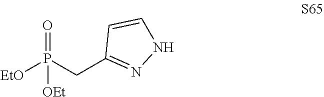 [SMILES: CCOP(=O)(Cc1cc[nH]n1)OCC]

To a stirred solution of phosphonate S64 (3.55 g, 11.7 mmol, 1.0 equiv) in ethanol (117 mL) at 25° C. was added hydrochloric acid (conc., 0.11 mL, 3.5 mmol, 0.3 equiv). After 18 h, the reaction mixture was concentrated under reduced pressure and purified directly by flash column chromatography (silica gel, ethyl acetate 0→5% methanol in ethyl acetate) to afford pure phosphonate S65 (2.51 g, 11.5 mmol, 98% yield) as a colorless oil. S65: R_{f}=0.35 (silica gel, 5% methanol in ethyl acetate); FT-IR (neat) vₘₐₓ 3406, 3197, 3062, 2983, 2931, 2912, 1647, 1572, 1537, 1466, 1444, 1393, 1368, 1290, 1225, 1163, 1128, 1097, 1049, 1023, 967, 843, 808, 792, 723 cm⁻¹; ¹H NMR (600 MHz, CDCl₃) δ=7.50 (d, J=1.3 Hz, 1H), 6.26 (dd, J=1.3, 1.3 Hz, 1H), 4.09-4.00 (m, 4H), 3.27 (d, J=20.7 Hz, 2H), 1.25 (t, J=7.1 Hz, 6H) ppm; ¹³C NMR (151 MHz, CDCl₃) δ=139.2, 133.4, 105.6, 62.5, (d, J=6.7 Hz), 25.8 (d, J=142.1 Hz), 16.4 (d, J=6.0 Hz) ppm; HRMS (ESI) calcd for C₈H₁₅N₂O₃PNa⁺ [M+Na]⁺ 241.0713, found 241.0716.

### Diethyl {[1-(4-nitrophenyl)-1H-pyrazol-3-yl]methyl}phosphonate (S69)

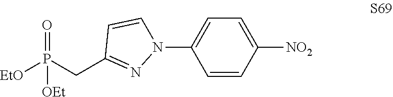 [SMILES: CCOP(=O)(Cc1ccn(-c2ccc([N+](=O)[O-])cc2)n1)OCC]

To a stirred solution of phosphonate S65 (544 mg, 2.49 mmol, 1.0 equiv) in tetrahydrofuran (25 mL) at 25° C. was added sodium hydride (60% w/w in mineral oil, 398 mg, 9.96 mmol, 4.0 equiv). After 30 min, fluorobenzene S66 (0.28 mL, 2.61 mmol, 1.05 equiv) was added, and the reaction mixture was heated to 70° C. After 1 h, the reaction mixture was allowed to cool to 25° C., and then quenched with saturated aqueous ammonium chloride solution (15 mL). The two phases were separated, and the aqueous layer was extracted with ethyl acetate (3×7 mL). The combined organic layers were dried with anhydrous sodium sulfate and concentrated under reduced pressure. The obtained residue was purified by flash column chromatography (silica gel, 50% ethyl acetate in hexanes) to afford pure phosphonate S69 (255 mg, 0.750 mmol, 30% yield) as a white amorphous solid. S69: R_{f}=0.30 (silica gel, 50% ethyl acetate in hexanes); FT-IR (film) vₘₐₓ 3457, 3119, 2983, 2930, 2909, 1611, 1596, 1537, 1518, 1506, 1479, 1455, 1443, 1381, 1335, 1311, 1249, 1163, 1111, 1099, 1043, 1024, 964, 944, 853, 808, 792, 765, 750, 687, 654 cm⁻¹; ¹H NMR (600 MHz, CDCl₃) δ=8.32 (d, J=9.1 Hz, 2H), 7.97 (d, J=2.5 Hz, 1H), 7.84 (d, J=9.1 Hz, 2H), 6.59 (d, J=2.5 Hz, 1H), 4.15-4.10 (m, 4H), 3.32 (d, J=21.0 Hz, 2H), 1.31 (t, J=7.1 Hz, 6H) ppm; ¹³C NMR (151 MHz, CDCl₃) δ=147.4 (d, J=7.4 Hz), 144.9 (d, J=161.9 Hz), 128.1 (d, J=1.9 Hz), 125.5, 118.4, 110.1 (d, J=3.0 Hz), 62.5 (d, J=6.6 Hz), 27.1 (d, J=141.5 Hz), 16.5 (d, J=6.1 Hz) ppm; HRMS (ESI) calcd for C₁₄H₁₈N₃O₅PNa⁺ [M+Na]⁺ 362.0876, found 362.0876.

### Diethyl {[1-(3-fluoro-4-nitrophenyl)-1H-pyrazol-3-yl]methyl}phosphonate (S70)

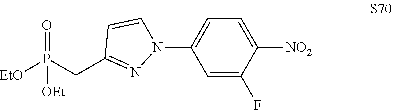 [SMILES: CCOP(=O)(Cc1ccn(-c2ccc([N+](=O)[O-])c(F)c2)n1)OCC]

To a stirred solution of phosphonate S65 (823 mg, 3.77 mmol, 1.0 equiv) in tetrahydrofuran (38 mL) at 25° C. was added sodium hydride (60% w/w in mineral oil, 604 mg, 15.1 mmol, 4.0 equiv). After 30 min, fluorobenzene S67 (0.43 mL, 3.96 mmol, 1.05 equiv) was added, and the reaction mixture was heated to 70° C. After 1 h, the reaction mixture was allowed to cool to 25° C., and then quenched with saturated aqueous ammonium chloride solution (20 mL). The two phases were separated, and the aqueous layer was extracted with ethyl acetate (3×8 mL). The combined organic layers were dried with anhydrous sodium sulfate and concentrated under reduced pressure. The obtained residue was purified by flash column chromatography (silica gel, 25% hexanes in ethyl acetate) to afford pure phosphonate S70 (700 mg, 1.96 mmol, 52% yield) as a white amorphous solid. S70: R_{f}=0.28 (silica gel, 25% hexanes in ethyl acetate); FT-IR (film) vₘₐₓ 3447, 3092, 2985, 2931, 2909, 1620, 1597, 1535, 1507, 1459, 1440, 1382, 1361, 1288, 1246, 1196, 1163, 1138, 1107, 1047, 1025, 972, 882, 847, 831, 808, 790, 767, 678 cm⁻¹; ¹H NMR (600 MHz, CDCl₃) δ=7.91 (dd, J=9.0, 5.2 Hz, 1H), 7.62 (d, J=2.5 Hz, 1H), 7.31 (dd, J=8.5, 2.7 Hz, 1H), 7.17 (ddd, J=9.4, 7.2, 2.7 Hz, 1H), 6.57 (d, J=2.5 Hz, 1H), 4.08-4.13 (m, 4H), 3.26 (d, J=20.9 Hz, 2H), 1.31 (t, J=7.1 Hz, 6H) ppm; ¹³C NMR (151 MHz, CDCl₃) δ=164.2 (d, J=257.6 Hz), 147.3 (d, J=7.1 Hz), 140.6 (d, J=2.4 Hz), 135.5 (d, J=11.3 Hz), 131.0 (d, J=2.0 Hz), 127.7 (d, J=10.2 Hz), 115.2 (d, J=23.2 Hz), 113.8 (d, J=26.2 Hz), 109.5 (d, J=3.1 Hz), 62.5 (d, J=6.5 Hz), 26.8 (d, J=141.3 Hz), 16.5 (d, J=6.1 Hz) ppm; HRMS (ESI) calcd for C₁₄H₁₇FN₃O₅PNa⁺ [M+Na]⁺ 380.0782, found 380.0787.

### Diethyl ({1-[4-nitro-3-(trifluoromethyl)phenyl]-1H-pyrazol-3-yl}methyl)phosphonate (S71)

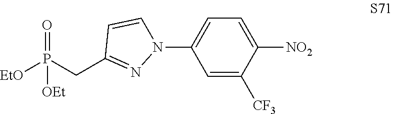 [SMILES: CCOP(=O)(Cc1ccn(-c2ccc([N+](=O)[O-])c(C(F)(F)F)c2)n1)OCC]

To a stirred solution of phosphonate S65 (809 mg, 3.71 mmol, 1.0 equiv) in tetrahydrofuran (37 mL) at 25° C. was added sodium hydride (60% w/w in mineral oil, 592 mg, 14.8 mmol, 4.0 equiv). After 30 min, fluorobenzene S68 (0.54 mL, 3.90 mmol, 1.05 equiv) was added, and the reaction mixture was heated to 70° C. After 1 h, the reaction mixture was allowed to cool to 25° C., and then quenched with saturated aqueous ammonium chloride solution (20 mL). The two phases were separated, and the aqueous layer was extracted with ethyl acetate (3×8 mL). The combined organic layers were dried with anhydrous sodium sulfate and concentrated under reduced pressure. The obtained residue was purified by flash column chromatography (silica gel, ethyl acetate) to afford pure phosphonate S71 (664 mg, 1.63 mmol, 44% yield) as a white amorphous solid. S71: R_{f}=0.39 (silica gel, ethyl acetate); FT-IR (film) vₘₐₓ 3455, 3109, 2986, 2911, 1597, 1536, 1499, 1464, 1444, 1386, 1372, 1349, 1312, 1291, 1242, 1148, 1098, 1044, 1027, 959, 904, 859, 849, 793, 759, 722, 660 cm⁻¹; ¹H NMR (600 MHz, CDCl₃) δ=8.19 (d, J=2.4 Hz, 1H), 8.06 (d, J=8.9 Hz, 1H), 7.98 (d, J=2.5 Hz, 1H), 7.96 (dd, J=8.9, 2.4 Hz, 1H), 6.63 (dd, J=2.5, 2.5 Hz, 1H), 4.16-4.11 (m, 4H), 3.32 (d, J=21.0 Hz, 2H), 1.32 (t, J=7.1 Hz, 6H) ppm; ¹³C NMR (151 MHz, CDCl₃) δ=148.1 (d, J=7.2 Hz), 144.9, 142.8, 128.1 (d, J=1.9 Hz), 127.5, 126.0 (q, J=34.3 Hz), 121.8 (q, J=273.9 Hz), 120.9, 117.6 (q, J=5.7 Hz), 110.8 (d, J=3.1 Hz), 62.5 (d, J=6.5 Hz), 27.1 (d, J=141.7 Hz), 16.5 (d, J=6.0 Hz) ppm; HRMS (ESI) calcd for C₁₅H₁₇F₃N₃O₅PNa⁺ [M+Na]⁺ 430.0750, found 430.0756.

### Diethyl {[1-(4-aminophenyl)-1H-pyrazol-3-yl]methyl}phosphonate (S72)

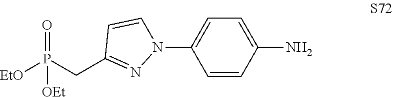 [SMILES: CCOP(=O)(Cc1ccn(-c2ccc(N)cc2)n1)OCC]

To a stirred solution of phosphonate S69 (350 mg, 1.03 mmol, 1.0 equiv) in ethyl acetate (21 mL) at 25° C. was added 10% Pd/C (25% w/w, 88 mg), and then an atmosphere of hydrogen was introduced (1 atm). After 2 h, the hydrogen atmosphere was removed, and the reaction mixture was filtered through a short pad of Celite®, rinsed thoroughly with ethyl acetate (50 mL), and concentrated under reduced pressure. The obtained phosphonate S72 (pale yellow amorphous solid, 316 mg, 1.02 mmol, 99% yield) was sufficiently pure for direct use in the following step. S72: R_{f}=0.27 (silica gel, 5% methanol in ethyl acetate); FT-IR (film) vₘₐₓ 3430, 3344, 3232, 3139, 2982, 2928, 2908, 1716, 1631, 1525, 1467, 1446, 1390, 1369, 1290, 1233, 1175, 1163, 1134, 1097, 1047, 1022, 965, 952, 831, 790, 762, 732, 661 cm⁻¹; ¹H NMR (600 MHz, CDCl₃) δ=7.70 (d, J=2.3 Hz, 1H), 7.41-7.39 (m, 2H), 6.74-6.71 (m, 2H), 6.45 (dd, J=2.3, 2.3 Hz, 1H), 4.13-4.08 (m, 4H), 3.71 (br s, 2H), 3.31 (d, J=20.7 Hz, 2H), 1.29 (t, J=7.1 Hz, 6H) ppm; ¹³C NMR (151 MHz, CDCl₃) δ=145.2, 144.2 (d, J=7.1 Hz), 132.5, 127.9 (d, J=2.1 Hz), 121.1, 115.6, 107.4 (d, J=3.2 Hz), 62.3 (d, J=6.5 Hz), 26.9 (d, J=141.5 Hz), 16.5 (d, J=6.0 Hz) ppm; HRMS (ESI) calcd for C₁₄H₂₀N₃O₃PNa⁺ [M+Na]⁺ 332.1134, found 332.1138.

### Diethyl {[1-(4-amino-3-fluorophenyl)-1H-pyrazol-3-yl]methyl}phosphonate (S73)

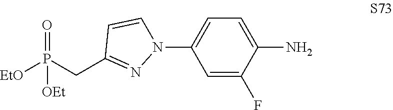 [SMILES: CCOP(=O)(Cc1ccn(-c2ccc(N)c(F)c2)n1)OCC]

To a stirred solution of phosphonate S70 (700 mg, 1.96 mmol, 1.0 equiv) in ethyl acetate (39 mL) at 25° C. was added 10% Pd/C (25% w/w, 175 mg), and then an atmosphere of hydrogen was introduced (1 atm). After 2 h, the hydrogen atmosphere was removed, and the reaction mixture was filtered through a short pad of Celite®, rinsed thoroughly with ethyl acetate (75 mL), and concentrated under reduced pressure.

The obtained phosphonate S73 (white amorphous solid, 628 mg, 1.92 mmol, 99% yield) was sufficiently pure for direct use in the following step. S73: R_{f}=0.39 (silica gel, ethyl acetate); FT-IR (film) vₘₐₓ 3439, 3333, 3119, 2983, 2928, 2909, 1628, 1602, 1516, 1465, 1443, 1390, 1322, 1239, 1207, 1183, 1163, 1144, 1097, 1050, 1025, 967, 876, 848, 813, 785, 767, 734, 681, 656 cm⁻¹; ¹H NMR (600 MHz, CDCl₃) δ=7.64 (d, J=1.9 Hz, 1H), 6.92 (dd, J=8.8, 2.8 Hz, 1H), 6.88 (td, J=8.8, 2.8 Hz, 1H), 6.75 (dd, J=8.8, 5.1 Hz, 1H), 6.47 (dd, J=1.9, 1.9 Hz, 1H), 4.13-4.08 (m, 4H), 3.30 (d, J=20.8 Hz, 2H), 1.29 (t, J=7.1 Hz, 6H) ppm; ¹³C NMR (151 MHz, CDCl₃) δ=155.4 (d, J=237.2 Hz), 145.0 (d, J=7.6 Hz), 137.2 (d, J=1.8 Hz), 130.9, 126.3 (d, J=9.3 Hz), 118.1 (d, J=8.2 Hz), 115.2 (d, J=22.0 Hz), 110.8 (d, J=25.4 Hz), 107.4 (d, J=3.5 Hz), 62.4 (d, J=6.5 Hz), 27.0 (d, J=141.8), 16.6 (d, J=6.0 Hz) ppm; HRMS (ESI) calcd for C₁₄H₁₉FN₃O₃PNa⁺ [M+Na]+350.1040, found 350.1034.

### Diethyl ({1-[4-amino-3-(trifluoromethyl)phenyl]-1H-pyrazol-3-yl}methyl)phosphonate (S74)

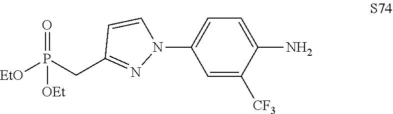 [SMILES: CCOP(=O)(Cc1ccn(-c2ccc(N)c(C(F)(F)F)c2)n1)OCC]

To a stirred solution of phosphonate S71 (667 mg, 1.64 mmol, 1.0 equiv) in ethyl acetate (33 mL) at 25° C. was added 10% Pd/C (25% w/w, 167 mg), and then an atmosphere of hydrogen was introduced (1 atm). After 2 h, the hydrogen atmosphere was removed, and the reaction mixture was filtered through a short pad of Celite®, rinsed thoroughly with ethyl acetate (70 mL), and concentrated under reduced pressure. The obtained phosphonate S74 (white amorphous solid, 611 mg, 1.62 mmol, 99% yield) was sufficiently pure for direct use in the following step. S74: R_{f}=0.39 (silica gel, ethyl acetate); FT-IR (film) vₘₐₓ 3494, 3352, 3240, 3118, 2985, 2933, 2909, 1647, 1587, 1527, 1509, 1453, 1393, 1376, 1332, 1316, 1301, 1234, 1163, 1142, 1108, 1048, 1026, 966, 897, 827, 792, 765, 733, 686, 666, 660 cm⁻¹; ¹H NMR (600 MHz, CDCl₃) δ=7.71 (d, J=2.1 Hz, 1H), 7.68 (d, J=2.5 Hz, 1H), 7.54 (dd, J=8.7, 2.5 Hz, 1H), 6.79 (d, J=8.7 Hz, 1H), 6.46 (dd, J=2.1, 2.1 Hz, 1H), 6.25 (br s, 2H), 4.13-4.08 (m, 4H), 3.30 (d, J=20.7 Hz, 2H), 1.30 (t, J=7.1 Hz, 6H) ppm; ¹³C NMR (151 MHz, CDCl₃) δ=144.8 (d, J=7.3 Hz), 143.2 (q, J=1.6 Hz), 131.5, 127.8 (d, J=2.0 Hz), 124.5 (q, J=272.0 Hz), 124.4, 118.1 (q, J=5.5 Hz), 118.1, 114.1 (q, J=30.7 Hz), 108.0 (d, J=3.2 Hz), 62.4 (d, J=6.5 Hz), 26.9 (d, J=141.4 Hz), 16.5 (d, J=6.0 Hz) ppm; HRMS (ESI) calcd for C₁₅H₁₉F₃N₃O₃PNa⁺ [M+Na]⁺ 400.1008, found 400.1008.

### Diethyl [(1-{4-[bis(tert-butoxycarbonyl)amino]phenyl}-1H-pyrazol-3-yl)methyl]phosphonate (66)

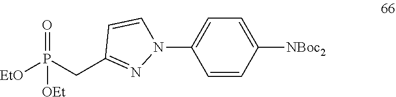

To a stirred solution of phosphonate S72 (251 mg, 0.810 mmol, 1.0 equiv) in acetonitrile (8.1 mL) at 25° C. was added triethylamine (0.34 mL, 2.4 mmol, 3.0 equiv), followed by di-tert-butyl dicarbonate (0.56 mL, 2.4 mmol, 3.0 equiv), and 4-dimethylaminopyridine (29 mg, 0.24 mmol, 0.3 equiv), and the reaction mixture was heated to 70° C. After 12 h, the reaction mixture was allowed to cool to 25° C., and then quenched with saturated aqueous ammonium chloride solution (5 mL). The two phases were separated, and the aqueous layer was extracted with ethyl acetate (3×3 mL). The combined organic phases were dried with anhydrous sodium sulfate and concentrated under reduced pressure. The obtained residue was purified by column chromatography (silica gel, ethyl acetate) to afford pure phosphonate 66 (220 mg, 0.43 mmol, 53% yield) as a colorless oil. 66: R_{f}=0.30 (silica gel, ethyl acetate); FT-IR (neat) vₘₐₓ 3450, 3120, 3055, 2980, 2933, 1789, 1749, 1709, 1609, 1531, 1478, 1459, 1431, 1388, 1368, 1354, 1314, 1272, 1247, 1152, 1115, 1100, 1051, 1026, 1005, 965, 950, 844, 801, 779, 765, 730, 670 cm⁻¹; ¹H NMR (600 MHz, CDCl₃) 43=7.86 (d, J=2.2 Hz, 1H), 7.65 (d, J=8.7 Hz, 2H), 7.20 (d, J=8.7 Hz, 2H), 6.51 (s, 1H), 4.14-4.09 (m, 4H), 3.32 (d, J=20.9 Hz, 2H), 1.42 (s, 18H), 1.30 (t, J=7.1 Hz, 6H) ppm; ¹³C NMR (151 MHz, CDCl₃) 43=151.8, 145.5 (d, J=7.1 Hz), 139.1, 137.5, 129.1, 127.9 (d, J=2.1 Hz), 119.1, 108.5 (d, J=3.3 Hz), 83.1, 62.4 (d, J=6.5 Hz), 28.1, 27.0 (d, J=141.4 Hz), 16.5 (d, J=6.0 Hz) ppm; HRMS (ESI) calcd for C₂₄H₃₆N₃O₇PNa⁺ [M+Na]⁺ 532.2183, found 532.2165.

### Diethyl [(1-[4-[bis(tert-butoxycarbonyl)amino]-3-fluorophenyl]-1H-pyrazol-3-yl)methyl]-phosphonate (67)

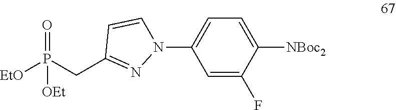

To a stirred solution of phosphonate S73 (0.640 g, 1.96 mmol, 1.0 equiv) in acetonitrile (19.6 mL) at 25° C. was added triethylamine (0.82 mL, 5.9 mmol, 3.0 equiv), followed by di-tert-butyl dicarbonate (1.35 mL, 5.88 mmol, 3.0 equiv), and 4-dimethylaminopyridine (72 mg, 0.59 mmol, 0.3 equiv), and the reaction mixture was heated to 70° C. After 12 h, the reaction mixture was allowed to cool to 25° C., and then quenched with saturated aqueous ammonium chloride solution (15 mL). The two phases were separated, and the aqueous layer was extracted with ethyl acetate (3×5 mL). The combined organic phases were dried with anhydrous sodium sulfate and concentrated under reduced pressure. The obtained residue was purified by column chromatography (silica gel, ethyl acetate) to afford pure phosphonate 67 (485 mg, 0.92 mmol, 47% yield) as a colorless oil. 67: R_{f}=0.56 (silica gel, ethyl acetate); FT-IR (neat) vₘₐₓ 3457, 3078, 2980, 2933, 2910, 1794, 1756, 1719, 1615, 1531, 1517, 1508, 1477, 1458, 1443, 1392, 1368, 1323, 1272, 1250, 1209, 1152, 1116, 1097, 1053, 1026, 966, 879, 868, 850, 829, 798, 778, 734, 681 cm⁻¹; ¹H NMR (600 MHz, CDCl₃) δ=7.64 (d, J=2.1 Hz, 1H), 7.33 (dd, J=9.2, 2.9 Hz, 1H), 7.19 (dd, J=8.8, 5.5 Hz, 1H), 7.03 (ddd, J=8.9, 7.5, 2.9 Hz, 1H), 6.50 (s, 1H), 4.13-4.08 (m, 4H), 3.26 (d, J=20.8 Hz, 2H), 1.31 (s, 18H), 1.31 (t, J=7.1 Hz, 6H) ppm; ¹³C NMR (151 MHz, CDCl₃) δ=161.9 (d, J=253.1 Hz), 150.8, 145.7 (d, J=6.8 Hz), 138.4 (d, J=10.8 Hz), 131.4 (d, J=9.3 Hz), 130.6 (d, J=1.6 Hz), 128.0 (d, J=3.5 Hz), 114.4 (d, J=22.5 Hz), 112.1 (d, J=26.2 Hz), 108.4 (d, J=2.7 Hz), 83.4, 62.3 (d, J=6.5 Hz), 27.9, 26.7 (d, J=141.9 Hz), 16.6 (d, J=6.0 Hz) ppm; HRMS (ESI) calcd for C₂₄H₃₅FN₃O₇PNa⁺ [M+Na]⁺ 550.2089, found 550.2080.

### Diethyl [(1-{4-[bis(tert-butoxycarbonyl)amino]-3-(trifluoromethyl)phenyl}-1H-pyrazol-3-yl)-methyl]phosphonate (68)

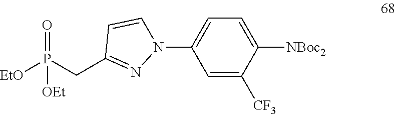

To a stirred solution of phosphonate S74 (0.30 g, 0.80 mmol, 1.0 equiv) in acetonitrile (8.0 mL) at 25° C. was added triethylamine (0.33 mL, 2.4 mmol, 3.0 equiv), followed by di-tert-butyl dicarbonate (0.55 mL, 2.40 mmol, 3.0 equiv), and 4-dimethylaminopyridine (29 mg, 0.24 mmol, 0.3 equiv), and the reaction mixture was heated to 70° C. After 12 h, the reaction mixture was allowed to cool to 25° C., and then quenched with saturated aqueous ammonium chloride solution (10 mL). The two phases were separated, and the aqueous layer was extracted with ethyl acetate (3×3 mL). The combined organic phases were dried with anhydrous sodium sulfate and concentrated under reduced pressure. The obtained residue was purified by column chromatography (silica gel, ethyl acetate) to afford pure phosphonate 68 (405 mg, 0.700 mmol, 88% yield) as a colorless oil. 68: R_{f}=0.56 (silica gel, ethyl acetate); FT-IR (neat) vₘₐₓ 3457, 3109, 2981, 2935, 1796, 1760, 1729, 1710, 1661, 1620, 1594, 1533, 1509, 1445, 1391, 1369, 1318, 1273, 1249, 1153, 1118, 1098, 1052, 1027, 961, 900, 875, 842, 802, 780, 732, 694, 669 cm⁻¹; ¹H NMR (600 MHz, CDCl₃) δ=8.02 (d, J=2.3 Hz, 1H), 7.91 (d, J=2.3 Hz, 1H), 7.84 (dd, J=8.6, 2.3 Hz, 1H), 7.31 (d, J=8.6 Hz, 1H), 6.55 (s, 1H), 4.15-4.11 (m, 4H), 3.32 (d, J=20.9 Hz, 2H), 1.37 (s, 18H), 1.31 (t, J=7.1 Hz, 6H) ppm; ¹³C NMR (151 MHz, CDCl₃) δ=150.6, 146.5 (d, J=7.2 Hz), 139.5, 134.9, 132.2, 128.7 (q, J=31.1 Hz), 127.9 (d, J=1.5 Hz), 123.0 (q, J=273.8 Hz), 121.8, 117.0 (q, J=5.2 Hz), 109.4 (d, J=3.1 Hz), 83.5, 62.4 (d, J=6.6 Hz), 27.9, 27.0 (d, J=141.6 Hz), 16.5 (6.0 Hz) ppm; HRMS (ESI) calcd for C₂₅H₃₅F₃N₃O₇PNa⁺ [M+Na]⁺ 600.2057, found 600.2044.

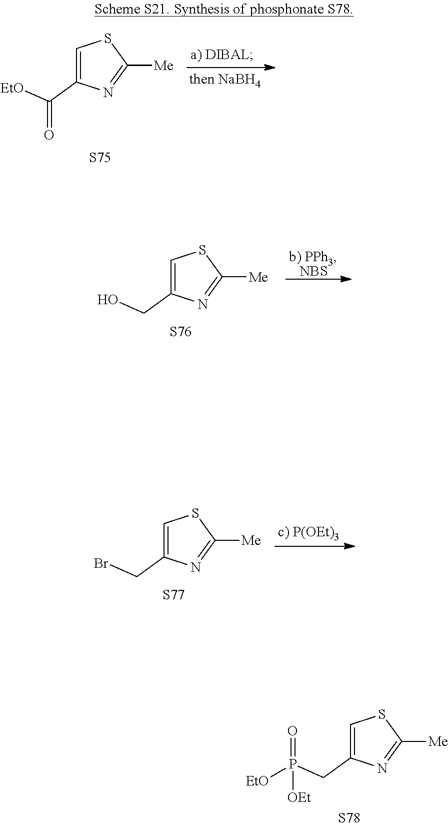

### (2-Methyl-1,3-thiazol-4-yl)methanol (S76)

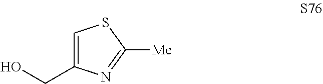

To a stirred solution of thiazolyl ester S75 (1.50 g, 8.76 mmol, 1.0 equiv) in dichloromethane (15 mL) at −78° C. was added diisobutylaluminum hydride (1.0 min dichloromethane, 13.1 mL, 13.1 mmol, 1.5 equiv) dropwise. After 5 min, the reaction mixture was quenched with methanol (15 mL), and allowed to warm to 0° C. Sodium borohydride (994 mg, 26.3 mmol, 3.0 equiv) was added, and after 10 min the reaction mixture was quenched with saturated aqueous potassium sodium tartrate solution (40 mL), and and allowed to warm to 25° C. The two phases were separated, and the aqueous layer was extracted with ethyl acetate (3×20 mL). The combined organic layers were dried with anhydrous sodium sulfate and concentrated under reduced pressure. The obtained residue was purified by flash column chromatography (silica gel, 50→100% ethyl acetate in hexanes) to afford pure hydroxymethyl thiazole 576⁸ (0.925 g, 7.16 mmol, 82% yield) as a colorless oil. S76: R_{f}=0.19 (silica gel, 20% hexanes in ethyl acetate); FT-IR (neat) vₘₐₓ 3277, 2924, 2862, 1531, 1475, 1436, 1376, 1324, 1267, 1188, 1130, 1066, 1029, 992, 945, 909, 857, 726 cm⁻¹; ¹H NMR (600 MHz, CDCl₃) δ=7.04 (s, 1H), 4.73 (s, 2H), 3.47 (br s, 1H), 2.72 (s, 3H) ppm; ¹³C NMR (151 MHz, CDCl₃) δ=167.4, 155.7, 114.6, 60.7, 19.0 ppm.

### 4-(Bromomethyl)-2-methyl-1,3-thiazole (S77)

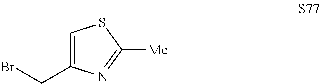

To a stirred solution of hydroxymethyl thiazole S76 (0.910 g, 7.04 mmol, 1.0 equiv) in dichloromethane (20 mL) at −78° C. was added triphenylphosphine (1.85 g, 7.04 mmol, 1.0 equiv), followed by N-bromosuccinimide (1.25 g, 7.04 mmol, 1.0 equiv). After 5 min, the reaction mixture was quenched with water (20 mL), and allowed to warm to 25° C. The two phases were separated, and the aqueous layer was extracted with ethyl acetate (3×20 mL). The combined organic layers were dried with anhydrous sodium sulfate and concentrated under reduced pressure. The obtained residue was purified by flash column chromatography (silica gel, 15→30% ethyl acetate in hexanes) to afford pure bromomethyl thiazole 577⁸ (0.820 g, 4.27 mmol, 61% yield) as a colorless oil. S77: R_{f}=0.38 (silica gel, 30% ethyl acetate in hexanes); FT-IR (neat) vₘₐₓ 3413, 3106, 2962, 2922, 2849, 1517, 1485, 1423, 1375, 1324, 1214, 1183, 1144, 1108, 1008, 955, 881, 853, 754, 702 cm⁻¹; ¹H NMR (600 MHz, CDCl₃) δ=7.13 (s, 1H), 4.54 (s, 2H), 2.72 (s, 3H) ppm; ¹³C NMR (151 MHz, CDCl₃) δ=167.2, 151.7, 117.4, 27.2, 19.4 ppm.

### Diethyl [(2-methyl-1,3-thiazol-4-yl)methyl]phosphonate (S78)

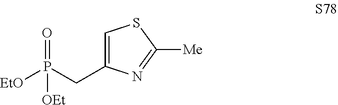 [SMILES: CCOP(=O)(Cc1csc(C)n1)OCC]

A stirred solution of bromomethyl thiazole S77 (0.808 g, 4.21 mmol, 1.0 equiv) in triethyl phosphite (1.75 g, 10.5 mmol, 2.5 equiv) was heated to 160° C. After 2 h, the triethyl phosphite was removed under a steady flow of nitrogen gas, and then the reaction mixture was allowed to cool to 25° C. The obtained residue was purified by flash column chromatography (silica gel, 1→5% methanol in ethyl acetate) to afford pure phosphonate 578⁸ (0.950 g, 3.81 mmol, 90% yield) as a colorless oil. S78: R_{f}=0.35 (silica gel, 5% methanol in ethyl acetate); FT-IR (neat) vₘₐₓ 3451, 2983, 2929, 1719, 1648, 1519, 1477, 1443, 1393, 1322, 1246, 1185, 1163, 1097, 1050, 1019, 950, 872, 842, 829, 807, 782, 727 cm⁻¹; ¹H NMR (600 MHz, CDCl₃) δ=7.05 (d, J=3.6 Hz, 1H), 4.11-4.06 (m, 4H), 3.35 (d, J=21.0 Hz, 2H), 2.68 (s, 3H), 1.28 (t, J=6.6 Hz, 6H) ppm; ¹³C NMR (151 MHz, CDCl₃) δ=165.6, 145.9 (d, J=8.0 Hz), 115.7 (d, J=7.4 Hz), 62.3 (d, J=6.6 Hz), 29.2 (d, J=140.1 Hz), 19.0, 16.4 (d, J=6.0 Hz) ppm.

### (1S,3S,7S,10R,11S,12S,16R)-8,8,10,12,16-Pentamethyl-3-[(1E)-1-(2-methyl-1,3-thiazol-4-yl)prop-1-en-2-yl]-7,11-bis[(triethylsilyl)oxy]-4-oxa-17-azabicyclo[14.1.0]heptadecane-5,9-dione (S79)

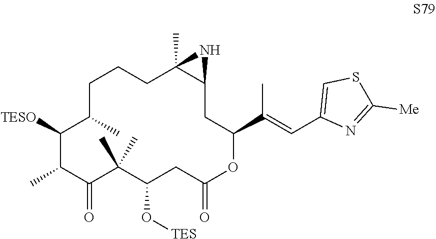

To a stirred solution of phosphonate S78 (102 mg, 0.409 mmol, 8.2 equiv) in tetrahydrofuran (1.0 mL) at −78° C. was added n-butyllithium (2.5 M in hexanes, 0.13 mL, 0.33 mmol, 6.5 equiv) dropwise. After 30 min, a solution of methyl ketone 55 (45 mg, 70 μmol, 1.0 equiv) in tetrahydrofuran (0.5 mL) was added, and the reaction mixture was allowed to slowly warm to 25° C., and stirred for an additional 20 min. Then the reaction mixture was quenched with saturated aqueous ammonium chloride solution (10 mL), and the two phases were separated. The aqueous layer was extracted with ethyl acetate (3×5 mL), and the combined organic layers were dried with anhydrous sodium sulfate and concentrated under reduced pressure. The obtained residue was purified by flash column chromatography (silica gel, 0→3% methanol in ethyl acetate) to afford pure protected epothilone S79 (12.6 mg, 17.1 μmol, 35% yield) as a colorless oil. S79: R_{f}=0.34 (silica gel, 2.5% ethyl acetate in hexanes); [α]_{D}²²=−9.3 (c=1.0, CH₂Cl₂); FT-IR (neat) vₘₐₓ 2953, 2876, 1742, 1695, 1552, 1459, 1412, 1381, 1241, 1181, 1157, 1109, 1056, 1019, 985, 915, 863, 836, 783, 745 cm⁻¹; NMR (600 MHz, C₆D₆) δ=6.72 (s, 1H), 6.54 (s, 1H), 5.41 (dd, J=8.4, 3.6 Hz, 1H), 4.24 (dd, J=9.0, 3.6 Hz, 1H), 4.16 (d, J=8.4 Hz, 1H), 3.07 (dq, J=7.2, 6.6 Hz, 1H), 2.72 (dd, J=16.2, 9.0 Hz, 1H), 2.61 (dd, J=16.2, 3.0 Hz, 1H), 2.29 (s, 3H), 2.27 (s, 3H), 2.09 (ddd, J=15.0, 3.6 3.6 Hz, 1H), 1.89-1.84 (m, 2H), 1.77-1.71 (m, 2H), 1.63-1.55 (m, 2H), 1.49-1.39 (m, 2H), 1.25-1.15 (m, 1H), 1.18 (d, J=6.6 Hz, 3H), 1.17 (s, 3H), 1.13 (d, J=6.6 Hz, 3H), 1.09-1.06 (m, 18H), 0.83 (s, 3H), 0.82-0.77 (m, 6H), 0.75-0.71 (m, 6H) ppm; NMR (151 MHz, C₆D₆) δ=214.4, 170.7, 164.4, 153.6, 138.3, 120.6, 116.8, 80.2, 79.3, 75.9, 53.5, 47.9, 41.8, 40.0, 39.3, 37.2, 35.1, 33.9, 31.8, 25.8, 25.1, 23.4, 23.0, 20.0, 18.9, 17.5, 14.9, 7.4, 7.3, 6.0, 5.8 ppm; HRMS (ESI) calcd for C₃₉H₇₁N₂O₅SSi₂⁺ [M+H]⁺ 735.4617, found 735.4600.

### (1S,3S,7S,10R,11S,12S,16R)-7,11-Dihydroxy-8,8,10,12,16-pentamethyl-3-[(1E)-1-(2-methyl-1,3-thiazol-4-yl)prop-1-en-2-yl]-4-oxa-17-azabicyclo[14.1.0]heptadecane-5,9-dione (10)

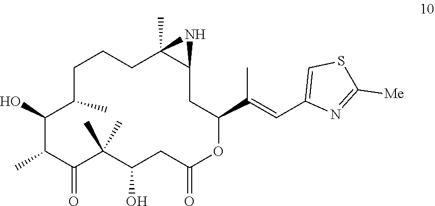

To a stirred solution of protected epothilone S79 (10.6 mg, 14.4 μmol, 1.0 equiv) in tetrahydrofuran (1.0 mL) at 0° C. was added hydrogen fluoride-pyridine complex (70% HF, 0.10 mL, 3.9 mmol, excess), and the reaction mixture was allowed to slowly warm to 25° C. After 4 h, the reaction mixture was quenched with saturated aqueous sodium bicarbonate solution (10 mL), and the two phases were separated. The aqueous layer was extracted with ethyl acetate (3×10 mL), and the combined organic layers were dried with anhydrous sodium sulfate and concentrated under reduced pressure. The obtained residue was purified by preparative thin layer chromatography (silica gel, 30% methanol in ethyl acetate) to afford pure epothilone 10 (5.2 mg, 10 μmol, 71% yield) as a white amorphous solid. Characterization for compound 10 is included above.

### Example 4 Biological Activity

A. Methods for Cytotoxicity Assay:

Cells were cultured in a T75 flask to ˜50-80% confluency and harvested with trypsin into a single cell suspension. Five hundred (500) cells per well were seeded in tissue culture plates in 50 μL/w ell culture media and incubated at 37° C. for 18-24 hours. Compounds were diluted as 400× final desired concentrations in DMSO. Serial dilutions in DMSO were then diluted in culture media for a final DMSO concentration of 0.25% and 50 μL/well of the final dilution was added to the cells (Vf=100 μL). Upon plating and treatment, cells were returned to the incubator for an additional 72 hours. CellTiter-Glo reagent was prepared per manufacturer's instructions and added at 100 μL/well to the cultures. CellTiter-Glo allows for relative enumeration of metabolically active cells by quantifying intracellular ATP concentrations. After 5 minutes of incubation with CellTiter-Glo at room temperature, 125 μL/w ell of the Cell Titer Glo/cell lysate solution was transferred into black assay plates, which were then read in a luminometer within 30 minutes. Luminescence readings obtained from cultures that did not receive any treatment (cell culture media only) were set as 100% control and all other luminescence values were normalized to these controls (e.g., Normalized RLU, relative luminescence unit).

B. Cell lines for Biological Assays

MES SA and MES SA/Dx cells are human uterine sarcoma. MES SA Dx cell line was generated from MES SA to achieve upregulation of MDR1. MES-SA/Dx cells exhibit marked cross-resistance to a number of chemotherapeutic agents (including daunorubicin, dactinomycin, vincristine, taxol, colchicine) and moderate cross-resistance to mitomycin C and melphalan. 293T cells are human embryonic kidney cell line.

C. Biological Assays

The synthesized aziridinyl epothilone analogues (8-40) were subjected to biological evaluation with regard to their cytotoxicity against several human cancer cell lines including several drug resistant cell lines (see Tables 3 and 4) and tubulin polymerization properties (Table 3). Epothilone D methylthiopyrazolyl analogue 73 (Scheme 6) and aziridinyl truncated epothilone intermediates 79 and 81 (Scheme 11) lacking the heterocyclic side chain, epothilones AD (1-4, Scheme 1), ixabepilone (5, Scheme 1), the tubulin stabilizing agent monomethyl auristatin E [MMAE, the payload of brentuximab vedotin (Adcetris®)] and N-acetyl calicheamicin γ₁¹ [NAC, the payload of gemtuzumab ozogamicin (Mylotarg®)] were also tested alongside a number of these synthesized compounds for comparison purposes (see Tables 3 and 4).

As can be seen in Table 3, there is considerable correlation (but not always) between tubulin polymerization and cytotoxic potencies for the synthesized compounds, with aziridinyl epothilone B analogues 9, 14, 17, 23, 34, 36, and 39 exhibiting significant potencies against an array of human cancer cell lines, including a highly drug resistant human ovarian cancer cell line [NCl/ADR-RES, GII₅₀=3.2-8.8 nM vs. paclitaxel: GI₅₀=4800 nM; ixabepilone (5): GI₅₀=1400 nM; see Table 3]. Table 4 shows the potencies of the synthesized aziridinyl epothilone B analogues against human uterine sarcoma cell line MES SA, human uterine sarcoma cell line with marked multidrug resistance MES SA DX, and human embryonic kidney cancer cell line HEK 293T. The most significant potencies in these studies were exhibited by analogues 10, 12, 13, 17, 24, 34, and 36-39, all in the subnanomolar range (IC₅₀ down to 0.02 nM vs. MMAE: IC₅₀≥0.068 nM; NAC: IC₅₀≥0.166 nM; paclitaxel: IC₅₀≥1.76 nM; see Table 4) against all three cell lines.

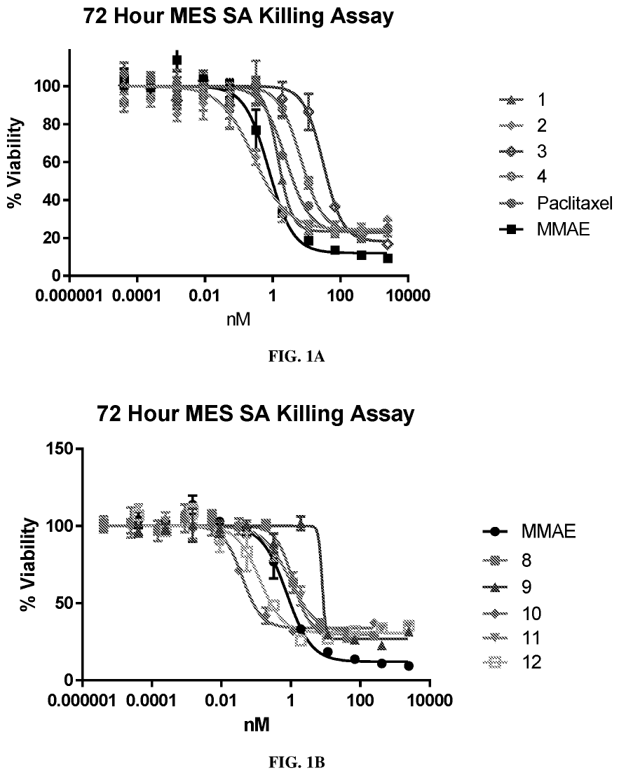

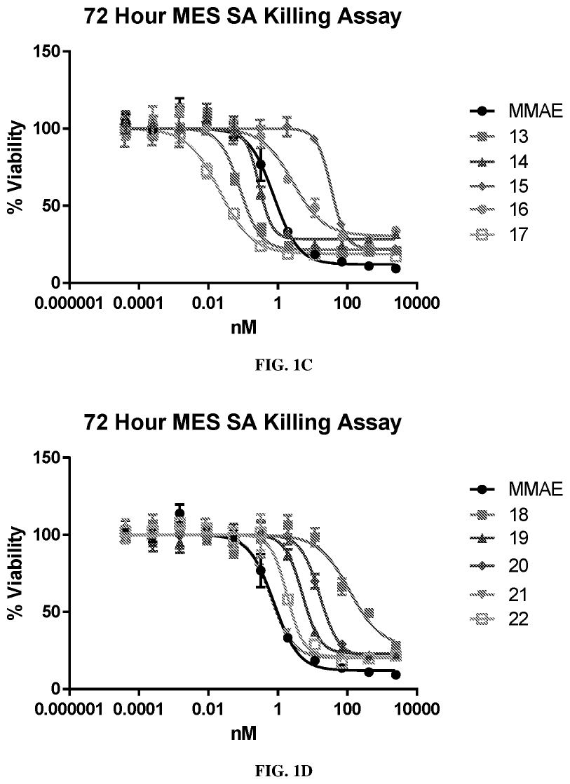

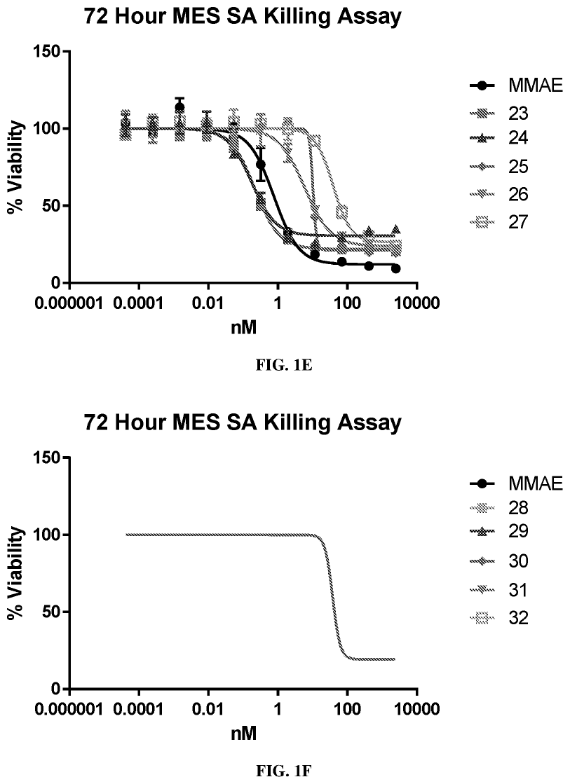

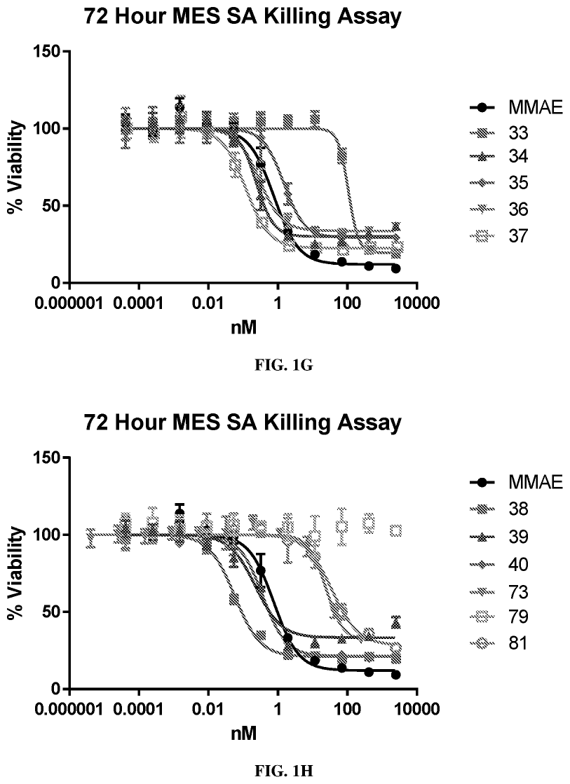

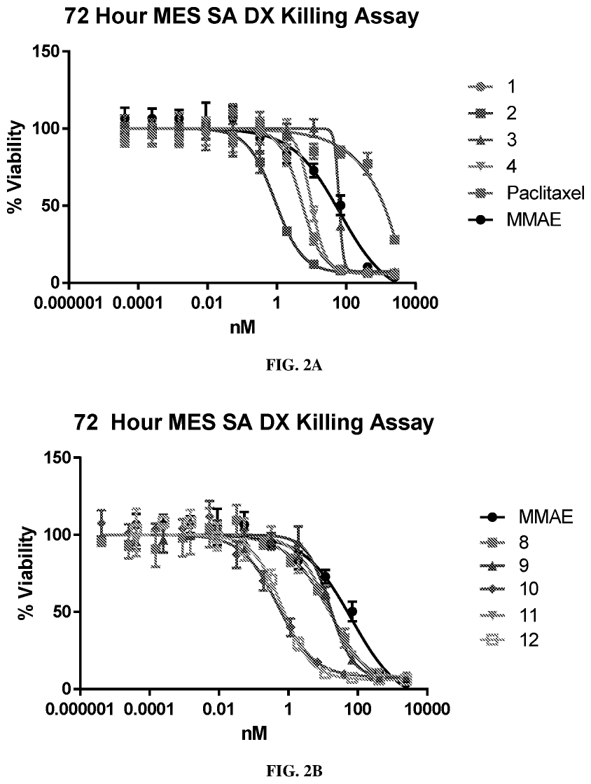

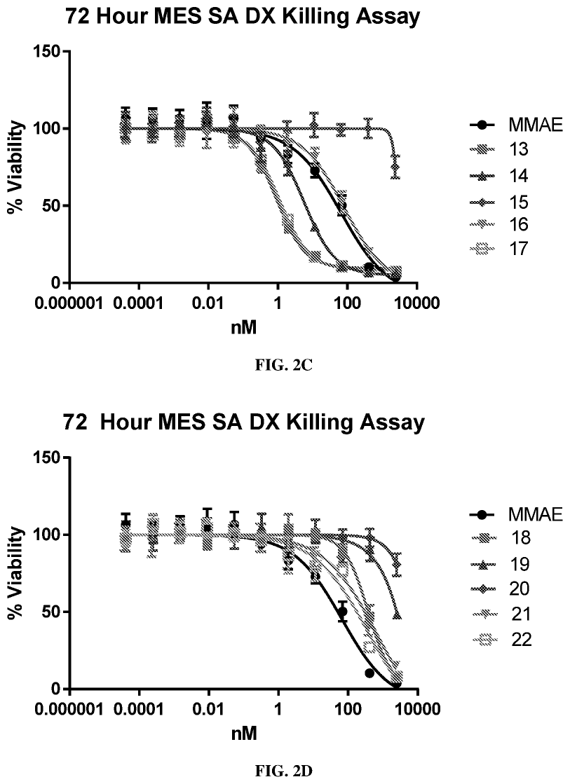

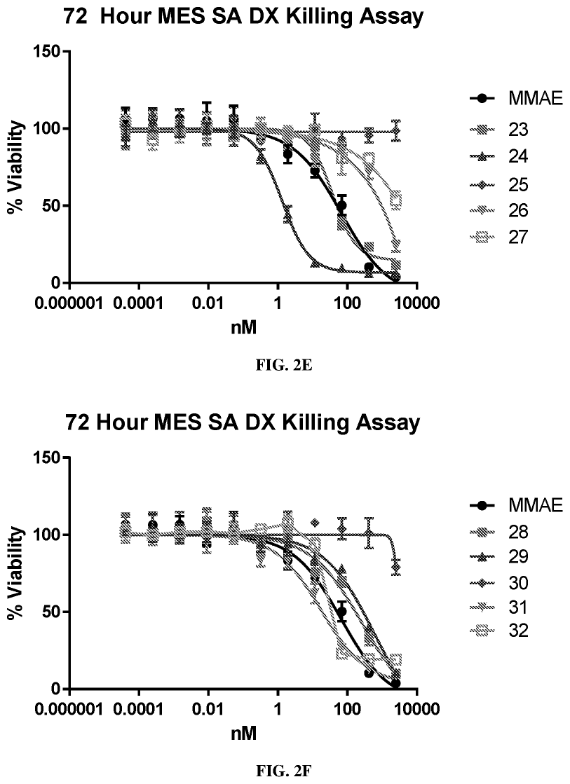

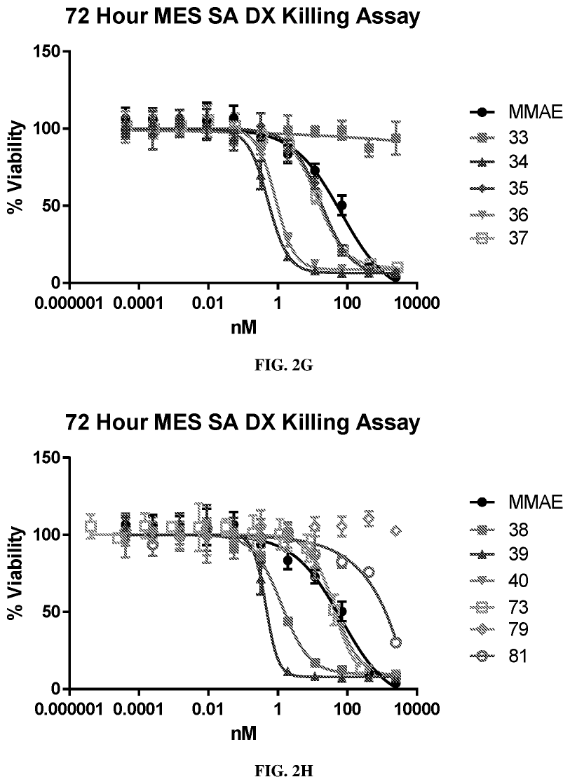

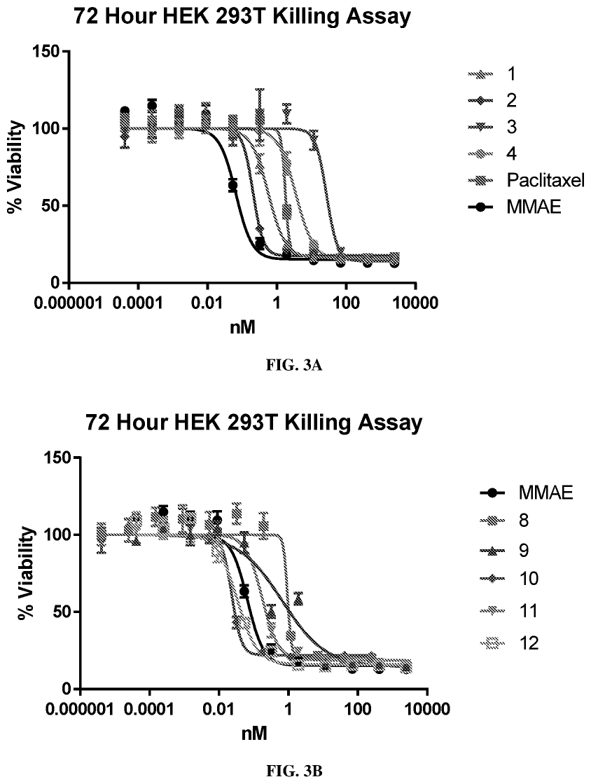

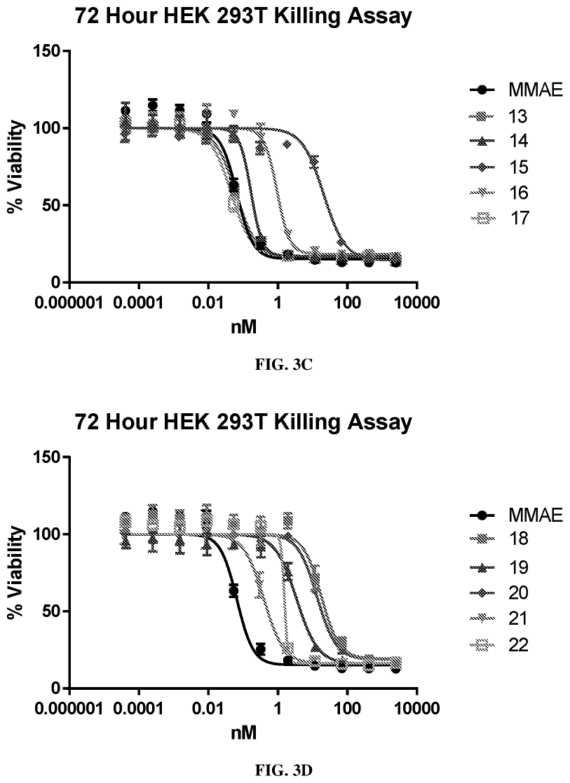

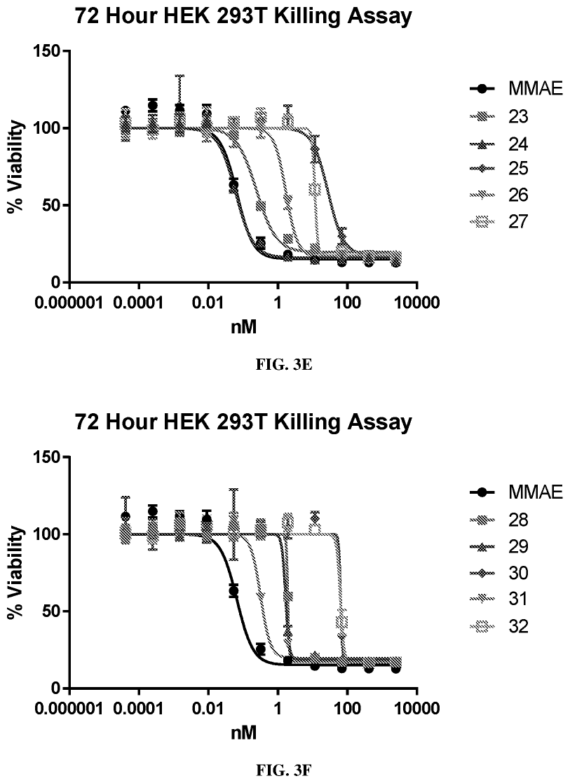

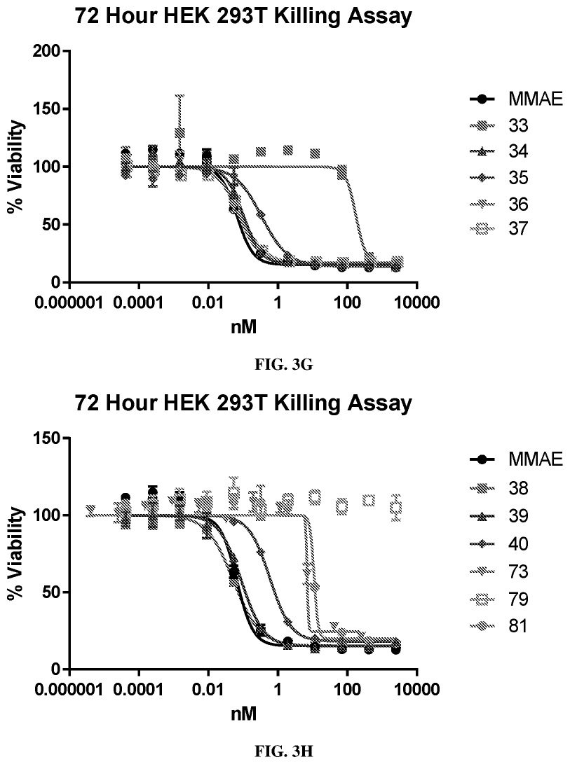

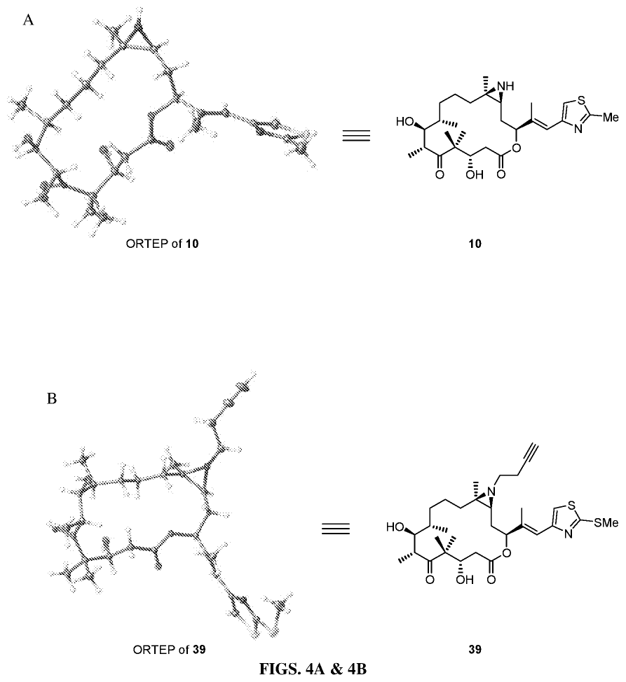

Many of the compounds of the present disclosure demonstrated low picomolar activities against the tested cell lines, including the MES SA DX cell line with marked multidrug resistance (IC₅₀ down to 0.51 nM vs. MMAE: IC₅₀=88.19 nM; NAC: IC₅₀=15.31 nM; paclitaxel: IC₅₀>400 nM; see Table 4). The high potencies of a number of these aziridinyl epothilone B analogues and their reactive chemical handles qualify them as promising payloads for antibody-drug conjugates. Killing plots for these compounds are shown in FIGS. 1A-3H.

The relatively high potencies of the 12,13-olefinic pyrazolyl epothilone D (lacking the epoxide moiety) analogue 73 (GI₅₀=5.0-52 nM, Table 3 and 0.17-39.18 nM, Table 4) is also noted. Also of note is the significant cytotoxicity of truncated methyl ketone aziridine epothilone 81 (as opposed to 79, see Tables 3 and 4) despite its lack of a heterocyclic side chain. These studies prompted the formulation of further structure-activity relationships (SARs) within the epothilone structural class, which confirmed and expanded previously developed SARs (Altmann et al., 2007). Thus, the C12-C13 aziridinyl epothilone B analogues proved to be generally more potent than their corresponding epoxide analogues, which in turn are known to be more potent than their olefinic precursors (with the exception of epothilone D analogue 73). These observations are in line with the hypothesis that the C12-C13 region of the molecule is not involved in critical binding interactions with β-tubulin, the biological target of the epothilones. This concept was previously confirmed by X-ray crystallographic analysis of an epothilone-tubulin complex (Nettles et al., 2004). A wide range of side chain variations on the nitrogen atom of the aziridine moiety is tolerated. The broad functional group tolerance of the aziridine substituents is also evident within those analogues exhibiting strong actions against multidrug resistant human cancer cell lines (e.g., 12, 14, 34, 36, and 39, see Tables 3 and 4).

In contrast to the C12-C13 region, it was found that the substituent tolerance for high potency was much more restricted in the side chain region of the molecule. Thus, some of the 20 heterocyclic side chains within a specific C12-C13 aziridinyl epothilone B family (i.e., N-hydroxyethyl aziridine) proved to be highly active, with those highly active compounds possessing the requisite basic nitrogen atom within the heterocycle in a nearly identical position. Despite the rather restricted range of tolerable side chain variations, however, these studies revealed a new and highly empowering heterocyclic structural motif, that of the methylthiooxazole included in the structure of analogue 17.

TABLE 3
Tubulin Assembly Induction (EC₅₀) and Cytotoxicity (GI₅₀) Assay Data

| | Tubulin | | Cancer cell lines GI₅₀ (nM)^{b} | | | |
|---|---|---|---|---|---|---|
| | enhancement EC₅₀ | | | NCI/ADR- | MDA-MB- | |
| Compound | (μM)^{a} | MCF-7^{c} | OVCAR-8^{d} | RES^{e} | 435^{f} | SNB-75^{g} |
| paclitaxel | 15 | 7.8 | 26 | 4800 | 5.0 | 15 |
| 1 | 22 | 17 | 20 | 78 | 16 | 26 |
| 2 | 4.5 | 9.2 | 15 | 19 | 7.0 | 8.0 |
| 3 | 18 | 70 | 110 | 110 | —^{h} | — |
| 4 | 18 | 14 | 13 | 17 | — | — |
| 5 | 4.5 | 7.0 | 35 | 1400 | — | — |
| 8 | >40 | 2.5 | 5.5 | 38 | — | — |
| 9 | 1.9 | 2.0 | 3.0 | 8.3 | — | — |
| 10 | 19 | 2.0 | 1.5 | 35 | — | — |
| 11 | 18 | 3.0 | 4.5 | 55 | — | — |
| 12 | 14 | 28 | 75 | 55 | 42 | 60 |
| 13 | >60 | 65 | 93 | 2800 | 20 | 130 |
| 14 | 5.0 | 4.0 | 16 | 8.8 | 4.5 | 11 |
| 15 | 13 | 330 | 290 | 3800 | 220 | 280 |
| 16 | 38 | 17 | 39 | 190 | 11 | 19 |
| 17 | 4.0 ± 1 | 7.5 | 25 | 6.5 | 3.5 | 13 |
| 18 | >60 | 1100 | 290 | 2300 | 120 | 290 |
| 19 | >60 | 40 | 38 | 4800 | 12 | 36 |
| 20 | 9.1 | 15 | 8.6 | 45 | 3.8 | 50 |
| 21 | >60 | 250 | 170 | 4000 | 30 | 98 |
| 22 | 17 | 7.5 | 18 | 240 | 4.0 | 18 |
| 23 | 4.6 | 78 | 10 | 7.5 | 12 | 10 |
| 24 | 7.9 | 11 | 23 | 630 | 3.5 | 12 |
| 25 | >60 | 32 | 130 | >5000 | 19 | 24 |
| 26 | 40 | 38 | 38 | 2700 | 5.5 | 50 |
| 27 | >60 | 140 | 580 | >5000 | 120 | 450 |
| 28 | 39 | 4.5 | 9.0 | 680 | 3.0 | 8.0 |
| 29 | 45 | 9.5 | 27 | 390 | 17 | 34 |
| 30 | >60 | 58 | 210 | 2800 | 48 | 100 |
| 31 | 19 | 13 | 70 | 50 | 11 | 65 |
| 32 | 43 | 25 | 58 | 170 | 11 | 68 |
| 33 | >60 | 140 | 1100 | >5000 | 160 | 450 |
| 34 | 5.4 | 13 | 15 | 3.2 | 7.3 | 22 |
| 35 | >60 | 39 | 19 | 30 | 5.5 | 32 |
| 36 | 4.0 | 5.5 | 18 | 7.0 | 3.5 | 23 |
| 37 | 6.1 | 14 | 63 | 70 | 15 | 23 |
| 38 | 5.1 | 18 | 15 | 18 | 9.5 | 16 |
| 39 | 3.5 | 30 | 18 | 7.0 | 3.5 | 31 |
| 40 | 19 | 3.5 | 100 | 140 | 3.5 | 14 |
| 73 | 9.3 | 5.0 | 22 | 52 | — | — |
| 79 | >60 | >5000 | >5000 | >5000 | >5000 | >5000 |
| 81 | >60 | 580 | 550 | >5000 | 200 | 360 |

^{a}EC₅₀ is the drug concentration yielding an unbound protein supernatant 50% that of controls.
^{b}GI₅₀ is the compound concentration required to decrease cell growth by 50%.
^{c}Human breast cancer cell line.
^{d}Human ovarian cancer cell line.
^{e}Highly drug resistant human ovarian cancer cell line.
^{f}Human melanoma cell line.
^{g}Human glioblastoma cell line.

TABLE 4
Cytotoxicity Data Against the Cancer Cell Lines MES
SA, MES SA DX, and HEK 293T For Epothilone Analogues

| | | Cancer cell lines IC₅₀ (nM)^{a} | |
|---|---|---|---|
| Compound | MES SA^{b} | MES SA DX^{c} | HEK 293T^{d} |
| MMAE | 0.096 | 88.19 | 0.068 |
| NAC | 0.364 | 15.31 | 0.166 |
| Paclitaxel | 2.47 | >400 | 1.76 |
| 1 | 1.46 | 5.12 | 0.57 |
| 2 | 0.28 | 1.16 | 0.26 |
| 3 | 31.47 | 62.05 | 27.32 |
| 4 | 6.84 | 9.70 | 3.55 |
| 8 | 1.14 | 16.95 | 0.95 |
| 9 | 8.01 | 15.73 | 0.67 |
| 10 | 0.04 | 0.51 | 0.02 |
| 11 | 0.94 | 13.71 | 0.17 |
| 12 | 0.13 | 0.66 | 0.03 |
| 13 | 0.078 | 0.850 | 0.058 |
| 14 | 0.28 | 5.66 | 0.17 |
| 15 | 36.35 | >1000 | 21.93 |
| 16 | 2.71 | 79.38 | 0.95 |
| 17 | 0.02 | 1.11 | 0.05 |
| 18 | 116.30 | 372.90 | 17.85 |
| 19 | 4.980 | >1000 | 3.387 |
| 20 | 16.14 | >2500 | 13.42 |
| 21 | 0.685 | 763.6 | 0.433 |
| 22 | 1.86 | 392.50 | 1.55 |
| 23 | 0.22 | 37.76 | 0.24 |
| 24 | 0.18 | 1.32 | 0.06 |
| 25 | 10.28 | >2500 | 27.71 |
| 26 | 5.90 | >400 | 1.72 |
| 27 | 38.86 | 2500 | 11.65 |
| 28 | 4.58 | 264.20 | 1.93 |
| 29 | 6.54 | >70 | 1.67 |
| 30 | 37.79 | >2500 | 63.97 |
| 31 | 1.41 | 17.15 | 0.32 |
| 32 | 25.83 | >400 | 63.21 |
| 33 | 106.80 | >2500 | 175.40 |
| 34 | 0.24 | 0.52 | 0.10 |
| 35 | 1.48 | 16.97 | 0.34 |
| 36 | 0.29 | 0.86 | 0.07 |
| 37 | 0.108 | 11.98 | 0.079 |
| 38 | 0.056 | 1.257 | 0.051 |
| 39 | 0.23 | 0.45 | 0.09 |
| 40 | 0.36 | 41.62 | 0.59 |
| 73 | 24.08 | 39.18 | 0.17 |
| 79 | >2500 | >2500 | >2500 |
| 81 | 36.13 | >400 | 11.08 |

^{a}IC₅₀ is the 50% inhibitory concentration of the compound against cell growth. For more details, see Cytotoxicity Methods section above.
^{b}Human uterine sarcoma cell line;
^{c}MES SA cell line with marked multidrug resistance;
^{d}Human embryonic kidney cancer cell line.

### *************

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference:
Altmann et al. Progress in the Chemistry of Organic Natural Products 90; 2009.
Altmann et al., ChemMedChem, 2:396, 2007.
Altmann et al., Chimia, 64:8, 2010.
Altmann et al., Methods and Principles in Medicinal Chemistry 60:83-125, 2014.
Altmann et al., Mol. Diversity, 15:383, 2011.
Amer et al., J. Org. Chem., 56:5210, 1991.
Anderson, N. G., Practical Process Research & Development—A Guide For Organic Chemists, 2^{nd} ed., Academic Press, New York, 2012.
Ando, J. Org. Chem., 64:6815, 1999.
Angelova et al., J. Cryst. Spectrosc., 22:253, 1992.
Austin-Ward and Villaseca, Rev. Med. Chil., 126(7):838-45, 1998.
Barclay et al. (eds.), The Leucocyte Antigen Facts Book, 1993, Academic Press.
Bisceglia & Orelli, Curr. Org. Chem., 19:744, 2015.
Blakemore, In Comprehensive Organic Synthesis, 2^{nd} ed; 729-817, 2014.
Borzilleri et al., J. Am. Chem. Soc. 122: 8890, 2000.
Bottin-Strzalko & Seyden-Penne, J. Bull. Soc. Chim. Fr, 161, 1984.
Brandt et al., J. Org. Chem., 63:1280, 1998.
Bukowski et al., Clin. Cancer Res., 4(10):2337-47, 1998.
Burkly et al.: TWEAKing tissue remodeling by a multifunctional cytokine: role of TWEAK/Fn14 pathway in health and disease. Cytokine 40:1-16 (2007).
Cachoux et al., Synlett, 1384, 2006.
Campbell et al., Cancer Res., 51(19):5329-5338 1991.
Carlomagno et al., Angew. Chem., Int. Ed., 42:2511, 2003.
Chari et al., Angew. Chem., Int. Ed., 53:3796, 2014.
Christodoulides et al., Microbiology, 144 (Pt 11):3027-37, 1998.
Davidson et al., J. Immunother., 21(5):389-98, 1998.
Denmark & Dorow, J. Am. Chem. Soc., 112:864, 1990.
Deschamps et al., Tetrahedron, 28:4209, 1972.
Gaugaz et al., ChemMedChem 9:2227, 2014.
Gokhale et al., Drug Dev. Ind. Pharm. 39:1315, 2013.
Gu & Tian, Topics in Current Chemistry 327; 197-238, 2012.
Hammick & Voaden, J. Chem. Soc., 3303, 1961
Hanibuchi et al., Int. J. Cancer, 78(4):480-485, 1998.
Harusawa et al., Synthesis, 1072, 2002.
Hellstrand et al., Acta Oncol., 37(4):347-353, 1998.
Hale et al., Pure Appl. Chem., 71:2019, 1999.
Homer et al, Chem. Ber., 92:2499, 1959.
Homer et al., Chem. Ber., 91:61, 1958.
Hui and Hashimoto, Infect. Immun., 66(11):5329-36, 1998.
Huttunen et al., Phamracol. Rev., 63:750, 2011.
Jat et al., Science, 343:61, 2014.
Johnson et al., Org. Lett., 2:1537, 2000.
Ju et al., Gene Ther., 7(19):1672-1679, 2000.
Kim et al., Org. Process Res. Dev. 15:797, 2011.
Kürti et al., In Strategic Applications of Named Reactions in Organic Synthesis. 212-213, 2005.
Kuzniewski et al., Org. Lett., 10:1183, 2008.
Lee et al., J. Am. Chem. Soc., 123:5249, 2001.
March's Advanced Organic Chemistry: Reactions, Mechanisms, and Structure, 2007.
Maryanoff & Reitz, Chem. Rev. 89:863, 1989.
Masuda & Okutani, Tetrahedron, 30:409, 1974.
Mitchell et al., Ann. NY Acad. Sci., 690:153-166, 1993.
Mitchell et al., J. Clin. Oncol., 8(5):856-869, 1990.
Mizuno et al., Angew Chem., Int. Ed., 37:515, 1998.
Modro et al., Can. J. Chem., 76:1344, 1998.
Morton et al., Arch. Surg., 127:392-399, 1992.
Narasaka et al., J. Chem. Soc., Chem. Commun., 102, 1993.
Nettles et al., Science, 305:866, 2004.
Nicolaou et al., Angew. Chem., Int. Ed., 37, 84, 1998.
Nicolaou et al., Angew. Chem., Int. Ed., 42:3515, 2003.
Nicolaou et al., Bioorg. Med. Chem., 7:665, 1999.
Nicolaou et al., Chem Med Chem, 10:1974, 2015.
Nicolaou et al., Chem. Biol., 5:365, 1998.
Nicolaou et al., Chem. Biol., 7:593, 2000.
Nicolaou et al., Chem. Eur. J., 3:1971, 1997.
Nicolaou et al., Chem Bio Chem, 2:69, 2001.
Nicolaou et al., Chem Med Chem, 1:41, 2006.
Nicolaou et al., In Classics in Total Synthesis II: More Targets, Strategies, Methods; Wiley-VCH: Weinheim, Germany, 161-210, 2003.
Nicolaou et al., J. Am. Chem. Soc., 119:7974, 1997.
Nicolaou et al., J. Am. Chem. Soc., 123:9313, 2001.
Nicolaou et al., Liebigs Ann./Recl., 1283, 1997.
Nicolaou et al., Tetrahedron 58:6413, 2002.
Onda et al., Cancer Res., 64:1419-1424, 2004.
PCT Publication No. 2008/121949
PCT Publication No. 2011/053435
PCT Publication No. 2014/087413
Peethambaram et al., Invest. New Drugs, 33:321, 2015.
Petrova et al., Phosphorus, Sulfur Silicon Relat. Elem., 47:457, 1990.
Petrova et al., Phosphorus, Sulfur Silicon Relat. Elem., 68:45, 1992.
Pfeiffer et al., Topics in Current Chemistry 286:5-33, 2009.
Pfeiffer et al., RSC Drug Discovery Series 25; 339-373, 2012.
Pietras et al., Oncogene, 17(17):2235-49, 1998.
Prota et al., Science, 339:587, 2013.
Qin et al., Proc. Natl. Acad. Sci. USA, 95(24):14411-14416, 1998.
Rautio et al., Nat. Rev. Drug Disc., 7:255, 2008.
Ravindranath and Morton, Intern. Rev. Immunol., 7: 303-329, 1991.
Reese et al., Angew. Chem., Int. Ed., 46:1864, 2007.
Regueiro-Ren et al., Org. Lett. 3:2693, 2001.
Remington's Pharmaceutical Sciences, 15^{th} Ed., 1035-1038 and 1570-1580, 1990.
Remington's Pharmaceutical Sciences, 15^{th} Ed., 3:624-652, 1990.
Rhoades, D. Synthesis of Natural and Designed Antitumor Agents: Epothilones and Thailanstatin A. Ph.D. Thesis, University of California, San Diego, August 2016.
Rosenberg et al., Ann. Surg. 210(4):474-548, 1989.
Rosenberg et al., N. Engl. J. Med., 319:1676, 1988.
Schiess et al., Org. Lett., 13:1436, 2011.
Schiess et al., RSC Drug Discovery Series 40, 78-108, 2015.
Schlosser, M. Pure Appl. Chem., 60:1627, 1988.
Sefkow et al., Bioorg. Med. Chem., Lett. 8:3025, 1998.
Sessa et al., Ann. Oncol., 18:1548, 2007.
Simeon et al., J. Med. Chem., 50:3256, 2007.
Srinivasarao et al., Nat. Rev. Drug Disc., 14:203, 2015.
Still-Gennari, C. Tetrahedron Lett., 24:4405, 1983.
Takaki et al., J. Org. Chem., 65:475, 2000.
Thompson (ed.), 1994, The Cytokine Handbook, Academic Press, San Diego.
Tsuge et al., Bull. Chem. Soc. Jpn., 61:2133, 1998.
U.S. Pat. No. 5,739,169
U.S. Pat. No. 5,801,005
U.S. Pat. No. 5,824,311
U.S. Pat. No. 5,830,880
U.S. Pat. No. 5,846,945
U.S. Pat. No. 6,232,287
U.S. Pat. No. 6,528,481
U.S. Pat. No. 7,452,964
U.S. Pat. No. 7,671,010
U.S. Pat. No. 7,781,565
U.S. Pat. No. 8,450,278
U.S. Pat. No. 8,507,445
U.S. Patent Publication No. 2004/0005647
U.S. Patent Publication No. 2006/0034925
U.S. Patent Publication No. 2006/0115537
U.S. Patent Publication No. 2006/0223114
U.S. Patent Publication No. 2006/0234299
U.S. Patent Publication No. 2007/0148095
U.S. Patent Publication No. 2012/0141550
U.S. Patent Publication No. 2013/0138032
U.S. Patent Publication No. 2014/0024610
US 20070275904 A1
US 20070276018 A1
US 2016057093, 2016
Vandat, L. Oncologist 13:214, 2008.
Vassilev & Dimitrov, Magn. Reson. Chem., 32:639, 1994.
Vassilev, Phosphorus, Sulfur Silicon Relat. Elem., 85:49, 1993.
Wadsworth et al., J. Am. Chem. Soc., 83:1733, 1961.
Wadsworth et al., J. Org. Chem., 30:680, 1965.
Weitman et al., Cancer Res., 52(12):3396-3401, 1992b.
Weitman et al., Cancer Res., 52(23):6708-6711, 1992a.
Winkles, Nat Rev Drug Discov 7:411-425 (2008).
Winthrop et al., Clin. Cancer Res., 9:3845s-3853s, 2003.
WO 02098868 A1
WO 03018002 A2
WO 2004014919 A1
WO 2007062288 A2
WO 2007140297 A2
WO 2007140298 A1
WO 2008147941 A1
WO 9825929 A1
WO 9954319 A1
WO 9967252 A2
WO 9967253 A2
Zarges et al., Chem. Ber., 124:861, 1991.
Zenzola et al., Chem., Int. Ed., 55:7203, 2016.
Zhou, Mol Cancer Ther. 10(7):1276-88, 2011.

1. A compound of the formula: 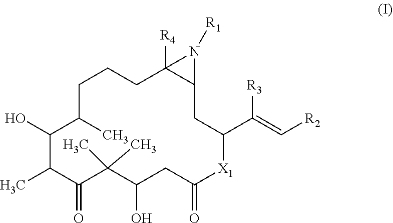
wherein:
X₁ is —O— or —NRₐ—; wherein
Rₐ is hydrogen or alkyl_{(C≤8)}, cycloalkyl_{(C≤8)}, or a substituted version of either of these groups;
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups;
R₂ is heteroaryl_{(C≤12)}, heteroarenediyl_{(C≤8)}R_{d}, or a substituted version of either of these groups; wherein:
R_{d} is alkyl_{(C≤12)}, aryl_{(C≤12)}, aralkyl_{(C≤12)}, heteroaryl_{(C≤12)}, heteroaralkyl_{(C≤12)}, or a substituted version of either of these groups;
R₃ is hydrogen or alkyl_{(C≤12)}, cycloalkyl_{(C≤12)}, or a substituted version of any of either group;
R₄ is alkyl_{(C≤12)}, cycloalkyl_{(C≤12)}, or a substituted version of any of either group;
or a pharmaceutically acceptable salt thereof.

2. The compound of claim 1 further defined as: 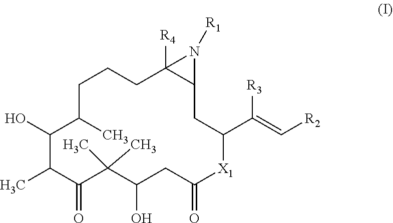
wherein:
X₁, R₃, and R₄ are as defined above;
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is 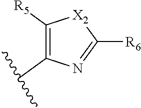
wherein:
X₂ is —O— or —NR_{b}—; wherein
R_{b} is hydrogen or alkyl_{(C≤8)}, cycloalkyl_{(C≤8)}, or a substituted version of either of these groups;
R₅ is hydrogen, amino, halo, hydroxy, alkyl_{(C1-3)}, substituted alkyl_{(C1-3)}, alkoxy_{(C1-3)}, or substituted alkoxy_{(C1-3)}; and
R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)}; or 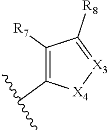
wherein:
X₃ and X₄ are each independently —O— or —NR_{c}—; wherein
R_{c} is absent, hydrogen or alkyl_{(C≤8)}, cycloalkyl_{(C≤8)}, aryl_{(C≤8)}, aralkyl_{(C≤8)}, heteroaryl_{(C≤8)}, heteroaralkyl_{(C≤8)}, or a substituted version of any of these groups; provided that when R_{c} is absent, then atom to which it is bound is a part of a double bond; and provided that when atom to which R_{c} is bound, then R_{c} is absent; and
R₇ and R₈ are each independently hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, substituted alkoxy_{(C1-3)}, alkylthio_{(C1-3)}, or substituted alkylthio_{(C1-3)}; or 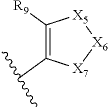
wherein:
X₅, X₆, and X₇ are each independently —O—, —S—, or —NR_{d}—; wherein
R_{d} is absent, hydrogen or alkyl_{(C≤8)}, cycloalkyl_{(C≤8)}, or a substituted version of either of these groups; provided that when R_{d} is absent, then atom to which it is bound is a part of a double bond; and provided that when atom to which R_{d} is bound, then R_{d} is absent; and
R₉ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; or
a compound wherein:
X₁, R₃, and R₄ are as defined above;
R₁ is hydrogen or alkyl_{(C≤8)}, cycloalkyl_{(C≤8)}, alkenyl_{(C≤8)}, alkynyl_{(C≤8)}, -alkanediyl_{(C≤6)}-cycloalkyl_{(C≤8)}, or a substituted version of any of these groups; and
R₂ is: 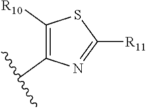
wherein:
R₁₀ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)};
R₁₁ is alkyl_{(C≤8)}, heteroaryl_{(C≤8)}, or substituted heteroaryl_{(C≤8)}; and
a compound wherein:
X₁, R₃, and R₄ are as defined above;
R₁ is cycloalkyl_{(C≤8)}, alkenyl_{(C≤8)}, alkynyl_{(C≤8)}, or a substituted version of any of these groups; and
R₂ is: 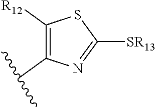
wherein:
R₁₂ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₃ is alkyl_{(C≤6)} or substituted alkyl_{(C≤6)};
provided that when R₂ is 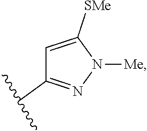 [SMILES: *c1cc(SC)n(C)n1]
then R₁ is not 2-hydroxyethyl;
or a pharmaceutically acceptable salt thereof.

3. The compound of either claim 1 or claim 2 further defined as: 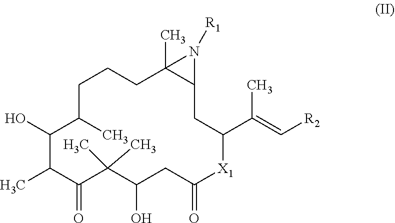
wherein:
R₁, R₂, and X₁ are as defined above;
or a pharmaceutically acceptable salt thereof.

4. The compound of claim 1 further defined as: 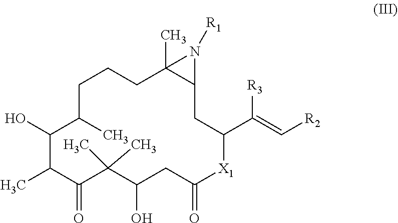
wherein:
R₁ and R₂ are as defined above;
or a pharmaceutically acceptable salt thereof.

5. The compound of claim 1 further defined as: 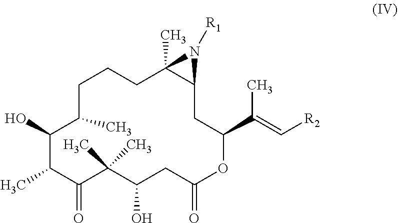
wherein:
R₁ and R₂ are as defined above;
or a pharmaceutically acceptable salt thereof.

6. The compound of claim 1 further defined as: 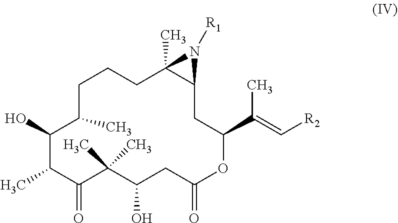
wherein:
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is 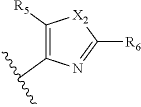
wherein:
X₂ is —O— or —NR_{b}—; wherein
R_{b} is hydrogen or alkyl_{(C≤8)}, cycloalkyl_{(C≤8)}, or a substituted version of either of these groups;
R₅ is hydrogen, amino, halo, hydroxy, alkyl_{(C1-3)}, substituted alkyl_{(C1-3)}, alkoxy_{(C1-3)}, or substituted alkoxy_{(C1-3)}; and
R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)}; or
or a pharmaceutically acceptable salt thereof.

7. The compound of claim 1 further defined as: 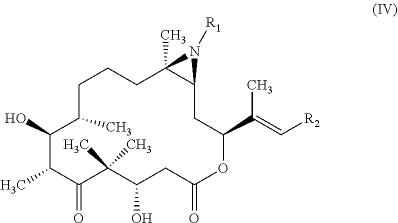
wherein:
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is 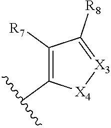
wherein:
X₃ and X₄ are each independently —O— or —NR_{c}—; wherein
R_{c} is absent, hydrogen or alkyl_{(C≤8)}, cycloalkyl_{(C≤8)}, aryl_{(C≤8)}, aralkyl_{(C≤8)}, heteroaryl_{(C≤8)}, heteroaralkyl_{(C≤8)}, or a substituted version of any of these groups; provided that when R_{c} is absent, then atom to which it is bound is a part of a double bond; and provided that when atom to which R_{c} is bound, then R_{c} is absent; and
R₇ and R₈ are each independently hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; or
provided that when R₂ is 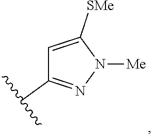 [SMILES: *c1cc(SC)n(C)n1]
then R₁ is not 2-hydroxyethyl;
or a pharmaceutically acceptable salt thereof.

8. The compound of claim 1 further defined as: 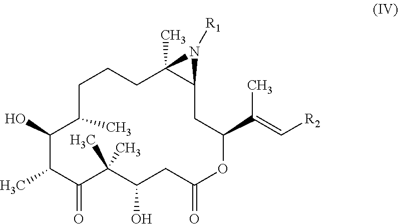
wherein:
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is 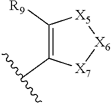
wherein:
X₅, X₆, and X₇ are each independently —O—, —S—, or —NR_{d}—; wherein
R_{d} is absent, hydrogen or alkyl_{(C≤8)}, cycloalkyl_{(C≤8)}, or a substituted version of either of these groups; provided that when R_{d} is absent, then atom to which it is bound is a part of a double bond; and provided that when atom to which R_{d} is bound, then R_{d} is absent; and
R₉ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)};
or a pharmaceutically acceptable salt thereof.

9. The compound of claim 1 further defined as: 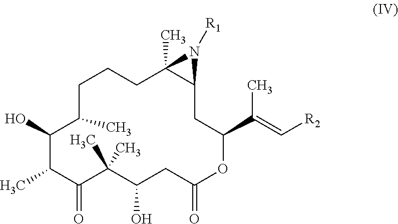
wherein:
R₁ is hydrogen or alkyl_{(C≤8)}, cycloalkyl_{(C≤8)}, alkenyl_{(C≤8)}, alkynyl_{(C≤8)}, alkanediyl_{(C≤6)}-cycloalkyl_{(C≤8)}, or a substituted version of any of these groups; and
R₂ is: 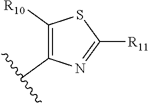
wherein:
R₁₀ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₁ is alkyl_{(C≤8)}, heteroaryl_{(C≤8)}, or substituted heteroaryl_{(C≤8)};
or a pharmaceutically acceptable salt thereof.

10. The compound of claim 1 further defined as: 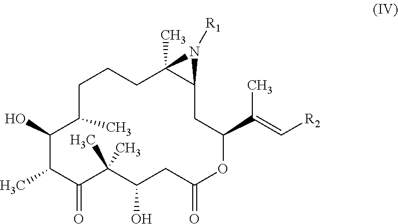
wherein:
R₁ is cycloalkyl_{(C≤8)}, alkenyl_{(C≤8)}, alkynyl_{(C≤8)}, or a substituted version of any of these groups; and
R₂ is: 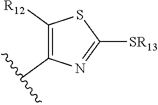
wherein:
R₁₂ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₃ is alkyl_{(C≤6)} or substituted alkyl_{(C≤6)};
or a pharmaceutically acceptable salt thereof.

11. The compound of claim 1, wherein R₃ is alkyl_{(C≤6)} or R₄ is alkyl_{(C≤6)}.

12. (canceled)

13. The compound of claim 1, wherein X₁ is —O—.

14. The compound of claim 1, wherein R₁ is hydrogen, alkyl_{(C≤6)}, substituted alkyl_{(C≤6)}, -alkanediyl_{(C≤6)}-cycloalkyl_{(C≤8)}, or alkyne_{(C≤6)}.

15. The compound of claim 1, wherein R₂ is: 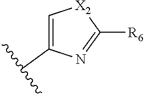
wherein:
X₂ is —O— or —NR_{b}—; wherein
R_{b} is hydrogen or alkyl_{(C≤8)}, cycloalkyl_{(C≤8)}, or a substituted version of either of these groups; and
R₆ is hydrogen, amino, halo, hydroxy, mercapto, alkyl_{(C≤6)}, substituted alkyl_{(C≤6)}, heteroaryl_{(C≤8)}, substituted heteroaryl_{(C≤8)}, heteroaralkyl_{(C≤8)}, substituted heteroaralkyl_{(C≤8)}, alkoxy_{(C≤6)}, substituted alkoxy_{(C≤6)}, alkylthio_{(C≤6)}, or substituted alkylthio_{(C≤6)},
R₂ is: 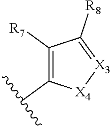
wherein:
X₃ and X₄ are each independently —O— or —NR_{c}—; wherein
R_{c} is absent, hydrogen or alkyl_{(C≤8)}, cycloalkyl_{(C≤8)}, aryl_{(C≤8)}, aralkyl_{(C≤8)}, heteroaryl_{(C≤8)}, heteroaralkyl_{(C≤8)}, or a substituted version of any of these groups; provided that when R_{c} is absent, then atom to which it is bound is a part of a double bond; and provided that when atom to which R_{c} is bound, then R_{c} is absent; and
R₇ and R₈ are each independently hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, substituted alkoxy_{(C1-3)}, alkylthio_{(C1-3)}, or substituted alkylthio_{(C1-3)}, R₂ is: 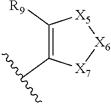
wherein:
X₅, X₆, and X₇ are each independently —O—, —S—, or —NR_{d}—; wherein
R_{d} is absent, hydrogen or alkyl_{(C≤8)}, cycloalkyl_{(C≤8)}, or a substituted version of either of these groups; provided that when R_{d} is absent, then atom to which it is bound is a part of a double bond; and provided that when atom to which R_{d} is bound, then R_{d} is absent; and
R₉ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}, R₂ is: 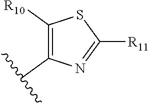
wherein:
R₁₀ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₁ is alkyl_{(C≤8)}, heteroaryl_{(C≤8)}, or substituted heteroaryl_{(C≤8)},
or R₂ is: 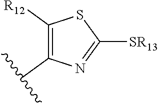
wherein:
R₁₂ is hydrogen, amino, halo, hydroxy, alkyl_{(C1-3)}, substituted alkyl_{(C1-3)}, aryl_{(C≤8)}, substituted aryl_{(C≤8)}, alkoxy_{(C1-3)}, or substituted alkoxy_{(C1-3)}; and
R₁₃ is alkyl_{(C≤6)} or substituted alkyl_{(C≤6)}.

16. The compound of claim 1, wherein the compound is further defined as: 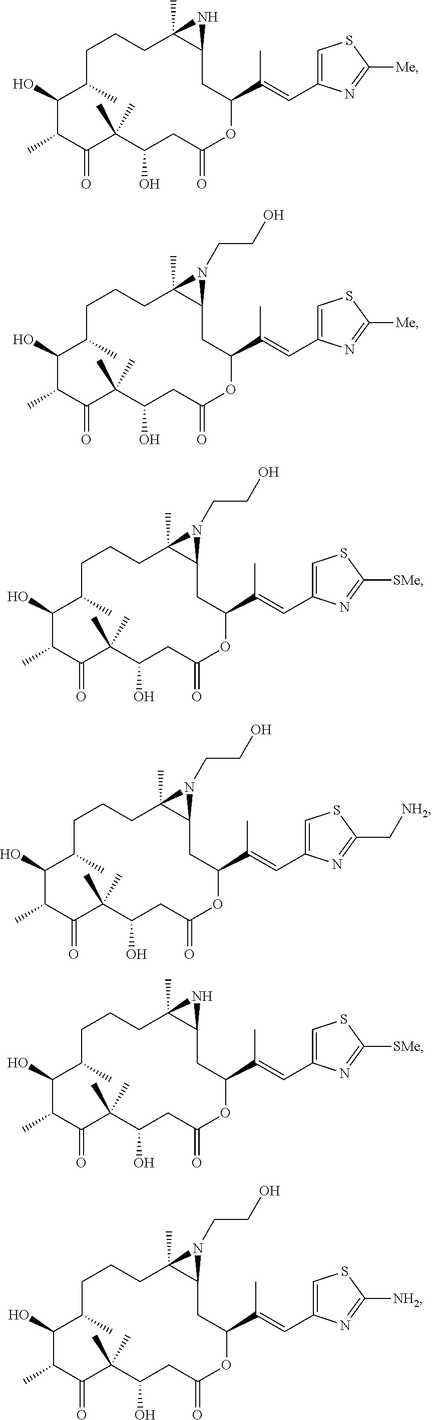 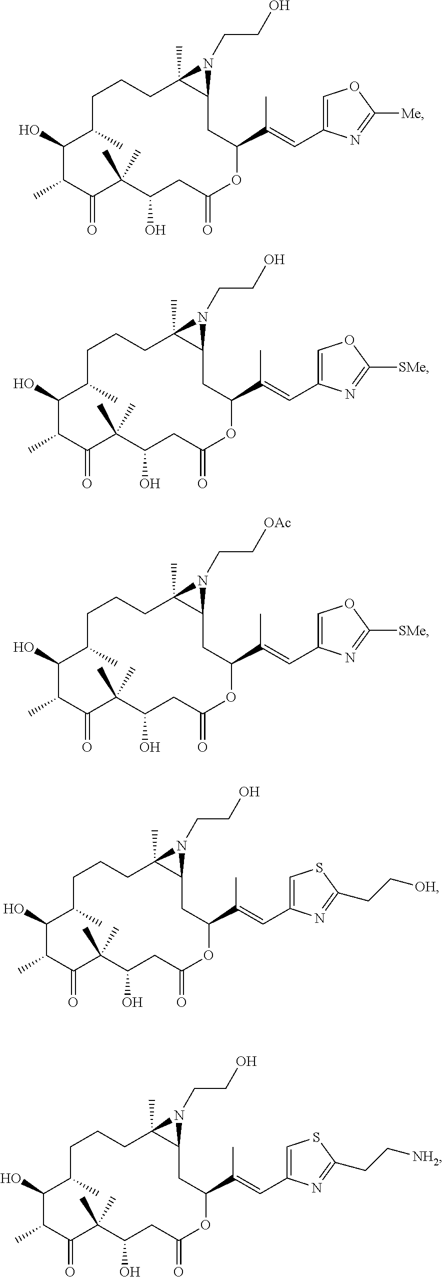 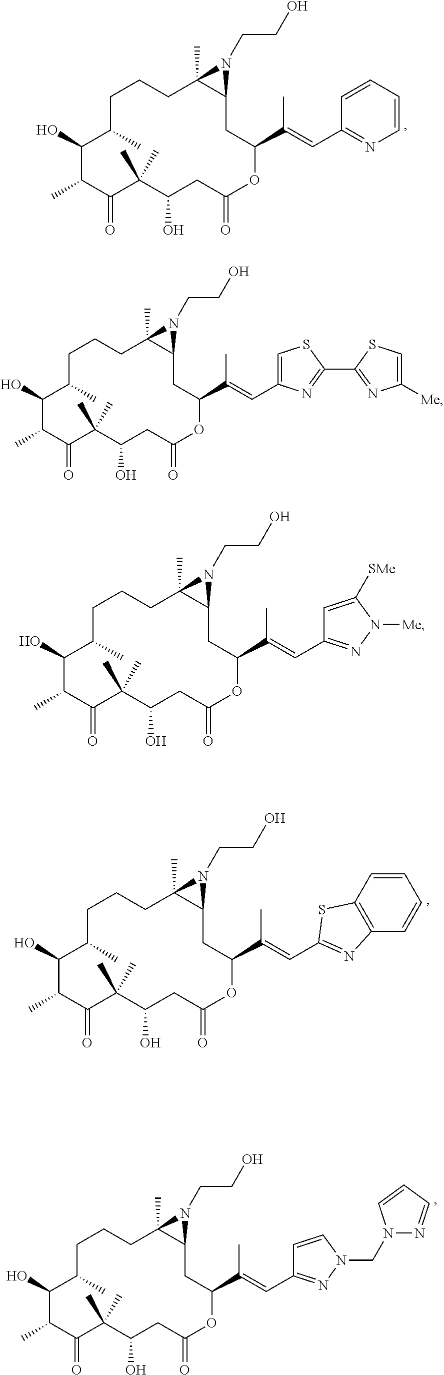 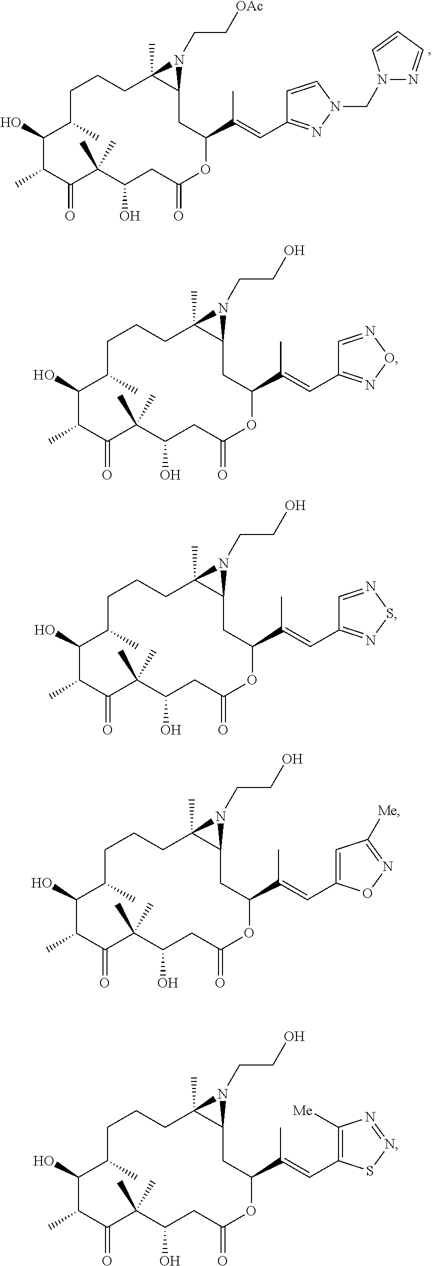 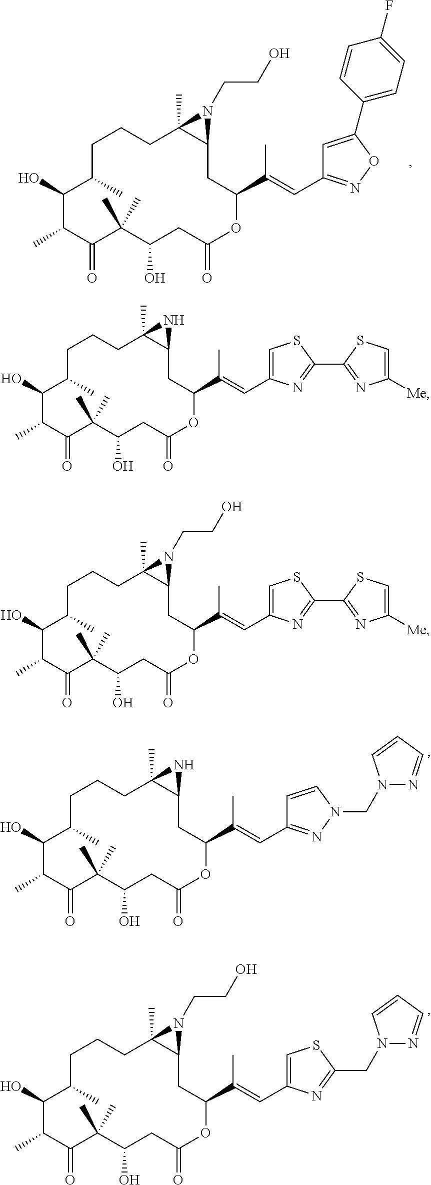 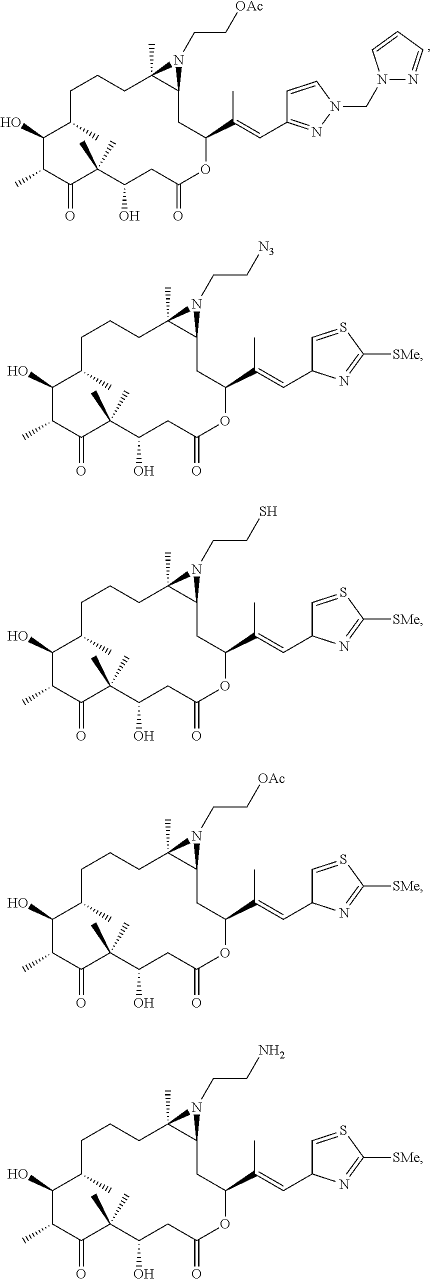 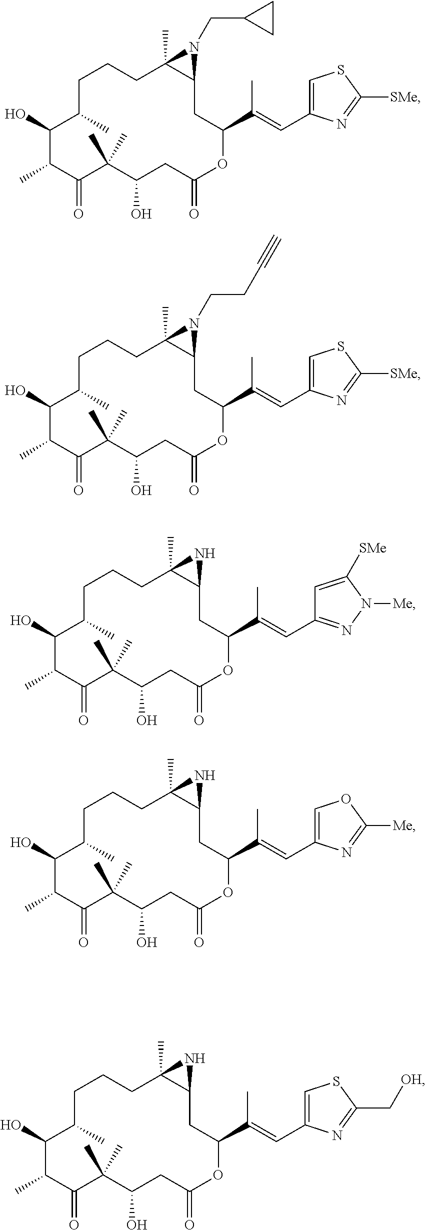 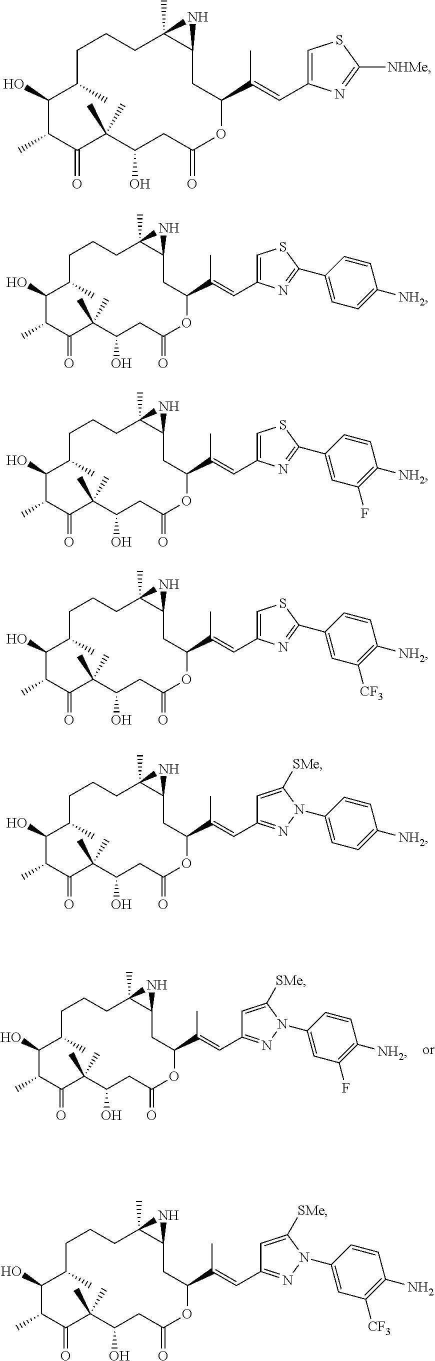
or a pharmaceutically acceptable salt thereof.

17. (canceled)

18. (canceled)

19. A pharmaceutical composition comprising:
(a) a compound of claim 1; and
(b) a pharmaceutically acceptable carrier.

20. A method of treating a disease or disorder in a patient comprising administering to the patient in need thereof a therapeutically effective amount of a compound of claim 1.

21. The method of claim 20, wherein the disease or disorder is cancer.

22. An antibody drug conjugate comprising:
(a) an antibody; and
(b) a compound of claim 1;
wherein the antibody and the compound are connected through a linker.

23. (canceled)

24. A method of preparing a compound of the formula: 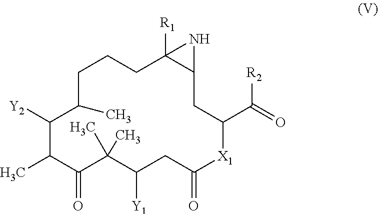
wherein:
X₁ is —O— or —NRₐ—; wherein
Rₐ is hydrogen, a monovalent amine protecting group, or alkyl_{(C≤8)}, cycloalkyl_{(C≤8)}, alkanediyl_{(C≤6)}-cycloalkyl_{(C≤8)}, aralkyl_{(C≤8)}, or a substituted version of either of these groups;
Y₁ and Y₂ are each independently amino, hydroxy, or alkoxy_{(C≤8)}, aralkoxy_{(C≤8)}, acyloxy_{(C≤8)}, alkylamino_{(C≤8)}, dialkylamino_{(C≤8)}, amido_{(C≤8)}, or a substituted version of any of these groups, or OR_{c}, wherein:
R_{c} is a hydroxy protecting group;
R₁ is alkyl_{(C≤12)}, cycloalkyl_{(C≤12)}, alkenyl_{(C≤12)}, alkynyl_{(C≤12)}, aryl_{(C≤12)}, or a substituted version of any of these groups; and
R₂ is hydrogen or alkyl_{(C≤12)}, cycloalkyl_{(C≤12)}, alkenyl_{(C≤12)}, alkynyl_{(C≤12)}, aryl_{(C≤12)}, or a substituted version of any of these groups; and
comprising reacting a compound of the formula: 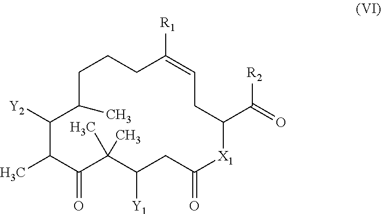
wherein:
X₁, Y₁, Y₂, R₁, and R₂ are as defined above;
with O-(2,4-dinitrophenyl)hydroxylamine in the presence of a Rh catalyst.

25. (canceled)
